(19) United States
(12) Patent Application Publication
(10) Pub. No.: US 2026/0242781 A1
(43) Pub. Date: Aug. 20, 2026

(54) CAS ENZYME AND SYSTEM AND USE THEREOF

(71) Applicant: Epigenic Therapeutics Pte. Ltd., Singapore (SG)

(72) Inventors: Zhengyan FENG, Shanghai (CN); Leilei WU, Shanghai (CN); Shaoshuai MAO, Shanghai (CN); Changqing YANG, Shanghai (CN); Qi ZHU, Shanghai (CN); Jingwen GUAN, Shanghai (CN); Ying ZANG, Shanghai (CN)

(21) Appl. No.: 19/489,576

(22) PCT Filed: Jun. 7, 2024
(86) PCT No.: PCT/CN2024/097935

§ 371 Date: Dec. 2, 2025

(30) Foreign Application Priority Data
Jun. 9, 2023 (CN) 202310684902.0

(51) Int. Cl. C12N 15/11; C12N 9/22; C12N 9/78; C12N 15/85

(52) U.S. Cl. CPC C12N 15/111; C12N 9/222; C12N 9/78; C12N 15/85; C07K 2319/09; C07K 2319/71; C07K 2319/80; C12N 2800/22; C12Y 305/04001

(57) ABSTRACT

The present application relates to the field of biomedicine, and specifically relates to a novel Cas enzyme, a system thereof and the use thereof.

### CROSS-REFERENCE TO RELATED APPLICATIONS

This application is a U.S. National Stage Application under 35 U.S.C. § 371 of International Application No. PCT/CN2024/097935, filed Jun. 7, 2024, which claims priority to, and the benefit of, Chinese Application No. 202310684902.0 filed Jun. 9, 2023, the content of each of which is incorporated by reference in its entirety.

### INCORPORATION BY REFERENCE OF SEQUENCE LISTING

The content of the electronic sequence listing (EPIG_004_01US_SeqList_ST26.xml; Size: 336,559 bytes; and Date of Creation: Dec. 1, 2025) is herein incorporated by reference in its entirety.

### TECHNICAL FIELD

The present application relates to the field of biomedicine, and specifically relates to a Cas enzyme, a system thereof and the use thereof.

### BACKGROUND

Recent advances in genome sequencing technologies and analytical methods have significantly accelerated the understanding of the genetic basis of biological activities in different fields, from prokaryotic biosynthetic pathways to human pathology. In order to fully understand and evaluate the huge amount of information generated by gene sequencing technology, it is necessary to improve the scale, efficiency and ease of use of genome and epigenome manipulation technology accordingly. These new genome and epigenome engineering techniques will accelerate the development of new applications in many fields, including biotechnology, agriculture, and human therapeutics.

Clustered regularly interspaced short palindromic repeats (CRISPR) and CRISPR-associated (Cas) genes, collectively referred to as CRISPR-Cas or CRISPR/Cas systems, are currently understood to provide immunity against phage infection in bacteria and archaea. The CRISPR-Cas system for adaptive immunity in prokaryotes is an extremely diverse set of protein effectors, non-coding elements, as well as loci structures, some examples of which have been engineered and adapted to generate important biotechnologies. Components of the system involved in host defense include one or more effector proteins capable of modifying DNA or RNA and RNA guide elements responsible for targeting the activity of these proteins to specific sequences on phage DNA or RNA. These guide elements can be reprogrammed to target alternative DNA or RNA targets.

CRISPR-Cas systems can be broadly divided into two categories: class 1 systems composed of multiple effector proteins, and class 2 systems composed of a single effector protein that complexes with an RNA guide to target DNA or RNA substrates. The single-subunit effector composition of class 2 systems provides a simpler set of components for engineering and application transformations, and has been an important source of programmable effectors to date. Characterization and engineering of class 2 CRISPR-Cas systems using CRISPR-Cas9 as an example paves the way for diverse and wide-ranging biotechnological applications in genome editing and other aspects. However, in addition to current CRISPR-Cas systems that enable new applications through their unique properties, there remains a need to develop powerful genome engineering tools, i.e. alternative programmable effectors and systems for modifying nucleic acids and polynucleotides (i.e. DNA, RNA or any hybrid, derivative or modification thereof).

### SUMMARY

In one aspect, the present application provides an isolated Cas enzyme, the isolated Cas enzyme comprises an amino acid sequence as set forth in any one of SEQ ID NOs: 1-58 or a sequence having at least about 80% identity to the amino acid sequence as set forth in any one of SEQ ID NOs: 1-58. For example, the Cas enzyme comprises a sequence having about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to the amino acid sequence as set forth in any one of SEQ ID NOs: 1-58. For example, the present application provides a Cas enzyme comprising the amino acid sequence shown in the following table:

| | Number in | |
|---|---|---|
| SEQ ID | the present | |
| NO: | application | Amino acid sequence (* may be any amino acid) |
| 1 | EpiCas001 | MNNNGETKCYGNNLDRFIGLREVNKTLRNELIPTDSTRENIEKN |
| | | GIISEDELRAEMGHELKAIMDDYYRNMIEETLSRKCNINWSTLF |
| | | AAMDSVARSANKKDADKILEKVQKEKREEIHKLFKEQGNFEKL |
| | | FKAELVSDVLPDFIGKYEGYSDEERISKLGTIKLFKKFMTSFKAY |
| | | FDNRKNVESKENISTSICHRVVNDNAFIFWDNLNAFKKIDKYAH |
| | | DAVETIEGEISGKLGEWKISQIYSVDFFGMVIPQSGITFYNEVSG |
| | | EINKYMNIYCQKEKQNSRQFKLKRLHKQILCIGETSFEIPEKYES |
| | | DEEVYQSVNDFIGMIKNNDIISRLEHLAENVNKYDADKIYINSK |
| | | SYENVSKCIGGTWNTIKDCLRLWYNENIRGSGKNKEKKVGKA |
| | | VEEDKYKSIAMLNQLLENYSDGDGRIYNVQLYVEHITKIIGDYK |
| | | PEELVYDANIRVIEQDEKAEEIKSVLDIYIAILHWIKAFIIEDAVE |
| | | KDTDFYYEIEEIFDKVQSIIPLYNMVRNYTTQKPYKQEKIKLKFN |
| | | TPTLAEGWHKSKEYSNNAIILQRDGKYYLGIFNAKNKPDKKIIE |
| | | GCSQKKNKADYAKMVYYLLPKPHMNLPHIFIKSKEAVKNYGL |
| | | SNYIVEGYDKDKHIKSSNGFDIKYCRDLIDYFKDCISKHPEWKN |
| | | FNFSFTDTALYNDIGEFYNEIKRQGYKIEWTYISEEEINRLDENG |
| | | QIYLFQIYNKDFAEGSKGTPNLHTLYLKNLFSEDNLKDPVFMLN |
| | | GNTEVFFRKSSIKKPVVHKKGSVLVNRNYIDLNGNRITIPEKEY |
| | | KEIYEYCNGIGNVNLSESAMKYYDMAKHYTATMDIVKDYRYT |
| | | VDKFFIHMPMTINFNAESNNNINELALNFVAKNDNIHIIGVDRG |
| | | ERNLIYISVIDSKGNIIKQKSYNVVNDYDYKSKLKEREYDRGDA |
| | | RKNWKRINNIKELKEGYLSQVVHEIAQLVIQYNAIIAMEDLNYG |
| | | FKRGRFKVERQVYQKFENMLINKLNYLVDKRKNADEAGGVLK |
| | | GYQLTYIPERLKNMGRQCGIIFYVPAAYTSKIDPTTGFVNLFNFN |
| | | AITSQEGRRKFINKFKHIRYNLNMNLFEFEFDYNDFITHNTQIAK |
| | | TEWSVYTNGPRIKKERVNGKWNDGYEADITEVMKQKLSDNGI |
| | | DYADGGDILTRIAEADDKLVSGIFEIFRLTVQLRNSKSESCGEDY |
| | | DRIISPVLNESNRFYDSDEYSGENEILPVDADANGAYCIALKGLY |
| | | EVKQIRENWVEGDKLSGDLLKISNADWFDFIQNKRYE* |
| 2 | EpiCas002 | MNIKFNKFTGVKPVSKTLRNALIPTEFTMVNLERNGIISADELRA |
| | | EKRQELKKIMDDYYRDLIERTLAENHSINWHILFELMKSKMHG |
| | | DEKDTAKKLENEQKSKREEIHRLFAEQEEFGKIFSAKLVSDILPG |
| | | FVASYAMYNDEDRTEKAEVVKLFNKFMSSFTEFFENRKNVFSK |
| | | ENISTSLCHRIVNDNALIFLNNMDSYKRIMENFKSESELVSKGLE |
| | | TMLGEWKISQIYSEDFFGMTMTQSGIEFYNDICGELNLVLNLYC |
| | | QKSKKNSARFKMKRLHKQILCTEKSSFEISYKYDNDKDVYTSV |
| | | NGFIKNIKNNNIIERLLKLGTETEQYDLNKIYISSQSYETVSIAIGG |
| | | TWETIRNCLTSWYDDNIKGSGKAKENKIKAVVKADKYQSIARV |
| | | DYLTETYNASGTDLQKAESYLSHIVQIIGDYEPEELYFDESIHLIE |
| | | NGDKISEIKKILDIYIDVVHWIKTFEISESVEKEPEFYYELDDIYD |
| | | KILDAVPLYNKVRNYVTQKPYNQEKFKLNFDSPTLANGWSKSK |
| | | ERDNNAIILSRDGKYYLGVFNVRNKPDKEILEGHSKPKNETDYA |
| | | KMVYNLLPGASKMLPKVFLSKKGIDNFKPSDYILEGYIKKKHIK |
| | | SSETFDIKYCRDLIDYFKNSISRHPEWKNFGFNFTETSKYNDISEF |
| | | YKEVEKQGYKIEWTYITEEDINKLDEAGQIYLFQIFNKDFAVGS |
| | | KGTPNLHTLYLKNLFSKENLDNVVLKLNGEAELFFRRSSIKTPVI |
| | | HKKGSILVNRCYVDADDGKRKPIPEKEYMEIYNYCNGKRDTEL |
| | | SESARKYYNISQRYEATMDIVKDYRYSVDKLFIHLPITINFKATE |
| | | NKNVNDMVLEYIAKSDDIHIIGIDRGERNLIYVSVIDMHGHIVM |
| | | QKSYNVVNEYDYKTKLKECEQGRDAARKNWKEIGKIKELKEG |
| | | YLSMVIHEITQLIIEYNAIVMMEDLNFGFKRGRFKVERQVYQKF |
| | | ENMLINKLNYLVDKHKAVDEPGGLLNGYQLTYIPDRMSELGR |
| | | QCGIIFYVPAAYTSKIDPTTGFFNAFRFNLLTSAESRRDFLNRFK |
| | | CIKYNGSMNMFEFDFDYNDFITHNTTIARTQWKVFTNGGRIKNI |
| | | ISKSGWNGTKKIDDITALMKEIFDSRKIDFSDGGNLLPEILAYEG |
| | | ADKDKFIVELLDIFKLTVQLRNSMPDGSDIEYDRIISPVINENNTF |
| | | YDSDMYTEPNSPLPIDADANGAYCIALKGLYEVNQIKENWKEG |
| | | ETFPKSVLKLNNANWFDFVQNKRYE* |
| 3 | EpiCas003 | MNLAFLNRGYNYGMINRIERGPFSELGDQQLLEQTRRLAANQR |
| | | CLEVHILDHLDEIDRRGLALRRGFSSLFDYAVRELRFSDAAAQR |
| | | RIQAMRLCRRHGCVRASLQSGELGMTSAAQLETTFAGAERAER |
| | | KSHTGHRNVGQGRGPDQDQDSVGSTRQLGLPAAPGEADEQGE |
| | | RGHRSAPEGAPPAAECSGDARERGHRSAPEGAAPAGEYTGEVA |
| | | ARTTPAVAVAVASRSGAQPPGGVHAGALLPRPELNGNAAPVQ |
| | | APSRAPVAAPGAPGAGAGAGPAAPASDEGPAAQLLPPTEFSAST |
| | | PEPEPVTAPAELRHTASRAPEPPRDDPFAELVPGTVSAAPLLHPQ |
| | | RQRELIEQAAGMSTRQVASLLAAAAPEVLPPRDTLRAVAPDRY |
| | | TLKVSIDQECEQGLRLLKDLMAHLDPRMSWGDLVARVVREAV |
| | | ARHDPRGGGRGQRRKGADSAGASPRRTPKETRAAGAAGGPAR |
| | | RVGESTGREPNRTGAAGAAGGPAQREDESSGRAQNRTRAAVA |
| | | PVAAARRRGGATSAPQSAPAVLVDGARAGGGPAGAPAAPPAP |
| | | PGDTAATERQNPIEVLTSAAPPSQSAPAAPSDGAHAGGAPAGAS |
| | | AATSAPQSAPAAQLDGARAGRVPSGAPAATSAPQFASGALLDG |
| | | SNAGAGVPGAPVATAAPQPALGGSPDGGPSGASAGASAGAAG |
| | | GASADGFSARAAGPGASVATSAPKSVLGGFPDGGPPDAAAGA |
| | | AAHTSREPVHSRRQSPRRRHIPAAVRRHIWHRDGGRCCYRDPL |
| | | TGRRCTSAHLLQIDHLVPVAEGGGPEPSNLALRCFAHHRMRHG |
| | | YAPAASPOPPI* |
| 4 | EpiCas004 | MDGLARVDRAVTACLAAPAWALPEEALVEALDEAHRLGQRL |
| | | AVLERVLVREVDGRGLARAQGATSTAGWLRDRLRVSIQSARR |
| | | LVADAGWLDAADDPGTTDDPGTTGDPSGTTDDPGTTGDPSGT |
| | | RTYASVEPGTGIGAAGAPSPADALPDAAGRTGLDGAVTTGPGP |
| | | GAGGAGGAGGADRAGAPGLSAAAGCGTAAAGPVLREALATG |
| | | RVNLEQARIIAGAVESVRKEAGPDTADKALYTLLADARRFEPR |
| | | ALRALADRILWHVAPDAAEEADRAALRRADDEAQRRRHLTLS |
| | | ATDDGRMRLRGLLDSETSATLLAALDPLTRPAGLGDDRTSGQR |
| | | RHDALGDICRLALRTGELPDNGGEPAQIVVTTEYAALTARLTA |
| | | VGLPAARQPGATSGTRSSIARPSPEPAPAERGPTEPTPAEQGSIES |
| | | TPAERRPTEHTPAERRRTERMPAERGAARRTQSGIGRLRRTTHE |
| | | RGGCGPRPPQRAAHNCGQPGRTGRAPAAGSGQRRPERPVPRIR |
| | | NGTAGTTGTATGRWQRPRGAPTETPTETAVSGGPPTPQSSPPAD |
| | | PSTLGTQRGSPTTDQRPASTDRAPATPVHEATAPAQDALAVQD |
| | | AAATVQRSPRLDHGCATADQQQDPGRDQDPGRDRAPGRDRHS |
| | | RGPDRDQWQARDRDHGRQGRGRLGPGRLDNDLRLTPATVRRL |
| | | ACDAAILPAVLGSQGQVLDLGRQRRLFTGPLRRALVLRDRGCA |
| | | FPGCDRPPRWCDGHHIQHWADGGATALHNAVLLCRHHHRVIH |
| | | QDDWTVRIAADGHPEFVPPAWIDPEQRPRRNRYHRRA* |
| 5 | EpiCas005 | MVVIECTVPDCEFKTTDASEAVAIALLANHNLVHRNEAQPRRL |
| | | PANTAPKGPKLDRPKIDVGVTMEEWNIFTRRWEVFRAGSGIDE |
| | | NSAPSQLFQCAGTELGDSILKANPNAVNESLTQLMEAMRSLAII |
| | | PIATCVLRTELLQMRQERDESFRSFTAKVRGKAETCAYNAKCS |
| | | CSRPVDYTDHVIRDVIINGLYDNEIRREVLGIDGILEKPINQVIAV |
| | | VESKEMARNALPPSPSLSAVSSFQRQKTNSPHPAPSPPQADQAK |
| | | EATCPGCQKTFKLFTEGARGWNKKPHQLCIDCHRVNRRARRPQ |
| | | RQPQQRSPQAPTVHANQAEPIAQISSLHGETKQDTRPTTHATTA |
| | | KRPVRLDHHIFSKGEWKRARFRDHPRVPITISLARKGRHRKETA |
| | | DTTPDIHAAVSAIADTGAQSDLWSLADFLACGFSREELQPIRPSL |
| | | TAANNSPISIEGAFFAKLSATSYSGQSTTSFSMVYVSSSVRDMYL |
| | | SYDSLLNLDLLPHCFPRHDDVTEASNERRPASQNKTRPNYTSPT |
| | | TNAARIVNEGCTKPASEHDVTCSCPQRETTPPRPSELPFPCTPAN |
| | | NTKMKAWLLNRYASSTFNTCPHRALPCMEGPPVEIHLDPTAKP |
| | | TACHTPANIPLHWQKRVYEDLLRDEALGVVERVPYGEPVTWC |
| | | HRMVVTRKHDGSPRRTVDLSPLNKYCQRETYATESPFQLARRI |
| | | PNDTWKTVTDAWNGYHSVPLRSSDRHLTTFITPFGRWRYTRAP |
| | | QGFLSSGDRYNRRFDAILAEFERKERCVDDTVHYDHDLEQHW |
| | | WRTIEFLTRVGQAGIVLNPDKFQFAGRSVDFAGFHVSDHTIEPL |
| | | PKYLDGIREFPTPTSTTDIRSWFGLVNQVSNYAQLRDLMAPFKP |
| | | FLSPRHKFLWSSELEEAFQASKEKIVKAIREGVEIFDIHKRTCLRP |
| | | DWSKRGIRYFLLQQHCSCSSGMPDCCPGGWGITLAGSRFLSPAE |
| | | QRYAAIEGEALAVAWGLEQTRYFTQGCDNLVIITDHKPLVKIFG |
| | | DRTLDEISNSRLFRLKQRTLPWRFEIHHLPGKSNYAADATSRHP |
| | | SQSNTEEKAPLCPANPPDAAEIALMTAIRDTAQELGAISWTLLA |
| | | QETAADARMGNLLHSLEQGTQLNKDDPTLANLGPIYESIYQEE |
| | | GVLMYQDRVVIPPSLRQRVLQSLHAAHQGTSSMEQRARSIVYW |
| | | PGMSNDIRVTREKCHDCNRNAPSQARTPPLPTAPPSTPFEAVFA |
| | | DFFNYKGYHYLVVGDRLSGWVEVLSSPATTNLAGSTGLIRHLR |
| | | TFFATFGVPEELSSDGGPEFTAKATEDFLRTWDVKQRLSSVSFP |
| | | QSNGRAEVAVKTAKRLLLSNTGPTGSLDQDRFLRAILQLRNTP |
| | | DPDCSLSPAQIIFGRPLRDTLAFVNRLEKFSNPHIRPLWRQAWA |
| | | AKEDALRTRISRTTESLREHARPLRPLAIGTRVFLQNQQGPNPN |
| | | KWDRSGVVVEAAGHDQYRVKVDGSGRVTLRNRRFLRAYTPA |
| | | LPAIPQAPSGAPAKTPSMAPDAPSSPSQTCHHTNRTATPATTPG |
| | | VPTPDLATAALALQVSPEEPDAAEEKQGPSTPEAMTLPSPPNAA |
| | | ARPRRTIRRPKQFEPETGTWTE* |
| 6 | EpiCas006 | MASIASLAPDGWLERKELSDGVSDDGLSDDESLLEERDTLETIR |
| | | DSLTATNPDEIDGLLGRPSLGSDGLPSPRTPSRRREFLGERKGSK |
| | | TRLSPTPPAAVPAPTCAPASTKNLWRGFALFAMAMACAALSLQ |
| | | RSPELVEYGPSRRLQTSFGERVDEAADGATSRRLQNSNQFCAA |
| | | RDGWEAFVGTARAGEAFGGEVCLLASTSTESSTLYGSHAAYSP |
| | | PMRVRAEFLAADDLTRQLGLVFGVPAWPDAANLPATGYACVL |
| | | EHKVTRWSSKSRLVLRRANADYDKNPDWINFKRSRWFDSEPN |
| | | AWYEATAAIDDTSAAIAGKVGADVSCEVRDDGGAVVASVRKE |
| | | DRWVMAHPGKVGFYSERDDLGERFVRALEVDGERAPEEPSFC |
| | | CYWSPTSDMCGDCPDPQTDGWCAASRARCEGCGGTFCDAPTP |
| | | PKEVPGRCCYDGAGCGSCSASDDDGFCAKSESNCGSCGGFFCS |
| | | LAPTPAPTRVPSPRPTTPSPTAAPTVAPGHPTAAPATYAPSSSEPS |
| | | SKPTTSTPSAQQGCACEEGPYEYDGKTFEGCTSYGWSVPWCGT |
| | | TNCGICDGLVSTGCWDECEETYAPSSSEPSPKPTTSTPSAAPQGF |
| | | PAIPLPTSATSPPTPTVFLTNADFRYGTYLITNPGVYVLREDIVFA |
| | | PETPKMMPPLDSETYPPDRGFWLGFFAAISVAADDVVVDLNGH |
| | | EIRSSREFLLRQRFFSIIELADKPFKANSGPPQFGALDAPLVPARR |
| | | VHVKSGTLGLSSHMGIHGNDNEDVTVTDVVIRDFETGGVQLNG |
| | | ASRVRLERVTVGPSLGHEHSAGPVPALATLSQATNLLRVAEGT |
| | | RHERQEEMVLLSAAVERFVQETLAGEDTSASVFASEAAGYAGL |
| | | PDGSATVWIPTTGLGGPHQTSELSSSVKSTSIRLIFGRIDCSRRVL |
| | | EKQPKSLCRNRRIREN* |
| 7 | EpiCas007 | MAGNHFKGSDSGRPAVPPRPNGAGKAGTPGGAGQGGSGKRVS |
| | | PGETAMFTALYEQSQKAKKTGRARGNVFAGGATFASQPSGAPS |
| | | VPANNAGAANAANGAGNVNAGKADNNTPSAGGAGLTGNLGT |
| | | IYTGASETGTFARLDDSGAEFAGSGSKEDYYDFGLNYGSDASSS |
| | | STSDGLVRHRHRKGERKKRHTGAIIAAVVAVLLVALGASGFML |
| | | LNSAKTVKSEAKEAVEIVGGLKDKVTSGDFSTLPDDAKKIDELC |
| | | DSMKAETSSPLWAAASFIPVYGSDINAARTMIDALSDVSSNALV |
| | | PMADNLSQATPGKLFQDGMINVSALQAVADSLSSSSKVFKSAN |
| | | EKVQGIGDTHISQVTELVDKVKDGFATLNGAVDAAEKVAPVLP |
| | | QMLGANGQTRNYLMYAMNNVEIRACGGFGGSQGLISVTDGQ |
| | | MSIGEFVPRIGLSEDEAVESVDEEDEALFGNHSNLYNSGNTYSP |
| | | DWPRNSQRVAALWKSQYGQDVDGVIGIDPVFLQYLLGLVGNV |
| | | SLPDGTVVDGTNAAKVLMHDVYWNYPVEESDGIFASVASAAF |
| | | DKILGGIGDVDVTKLVSAVERGAEEGRLIAWMRNDDEQNAIKE |
| | | TGIDASLPDPDDPSADPVAGVYFNNLSFSKLDWYLNADTQIGQ |
| | | GIKNGDGTCSYRITVTLTNIMTQEEAGKLPDYVAASAPDAARD |
| | | DERLNVSLFAPTGGNISDLTVEGTQFGLGAATWHGIPFYSGTVD |
| | | LHAGETTTITYTLTTSAEAGDKPLTLRQTPTCQAARDSASA* |
| 8 | EpiCas008 | MEAGRDFIAFGQDVQGTDCCRGAGGAGLAGPAGRSGAGAKN |
| | | VRPPCQTVGMEAGRDFIPFGQVVEGTDRDCCSGAGGAGLADA |
| | | GAAGVSGVGGVLAGVRQLCAGVVEDSALWDLGAASDFAGGV |
| | | EEISRAVEYLQLVAASAVDRARTQSAAAAAPGMSWTTGWRDE |
| | | DPASDENSGTTAPVPVEPPSAAATGSDDGYRNTVEFLRARLRIG |
| | | AAEARGRLALGAGVLPRQGLAGRSEAAVHAELGAAVAAGAV |
| | | GSRAAAVIEAALGRVRHVGDAAAAARMEHALTRTAAENDTDF |
| | | LARVARSWAEAMDQDGAEPSEELLRQRQGAFIRRQRNGLHHL |
| | | EIFATAEQFEQLVTVMNTATNPRTGTGSGTGAGHGAMNAPAA |
| | | DTPEDAGHGSEPATDGPAGPAAVQEPAQDWAPAATGGAGWET |
| | | GPAAGAGWIVGPAAVSASDTRAGVVSTSAAGATGDEFVSDPGP |
| | | GYGFGPVPDPGPGDHRAEAGDTGAGGLDRRTRAQKQLDGLVG |
| | | ACQISLAAGTLPASGGLRPQVMVTIDYRDLLARLGNGTGTTSDS |
| | | AAPAGTATDCPVHMAGTVDVAGTVDMAGTVDMAGTVGAAG |
| | | SGRRTGGLPGTGSLLFTGPVTDSTIRKIACDADIIPVLLGTADRV |
| | | LDIGRASRIFPPHIRKALIARDQGCAFPGCTIPASWCEAHHIDYW |
| | | SRGGSTGTENGTLLCSHHHHLIHKEHWTIRVRAGIPWFIPPPHID |
| | | PAQTPRRNNYFHPARLDPAA* |
| 9 | EpiCas009 | MPNRKVTLTAFSRMDHRVEALEEGLNEVRASIAGVQATMRES |
| | | YANIVALLEKHLGKSIATEGGTSEKKDKPESSEVKNNVTLEFRQ |
| | | SAKKVELPSFDGDDPAGWISRAEVYFRVQGTTPAVKVSLAQLC |
| | | MDGLTIHFFNSILRENEDLSWEELKEALLERYGGHGEGDVYEQ |
| | | LTDLKQEAIDDYITEFEYLVAEIPRLPEKQYRGYFLHGLKPEIKG |
| | | RVRSFAALSEMRRTKLLQVARVVEREVEGGSELKPNRGPRFGF |
| | | GSGSHKNGSQSDWVMVRRREGSRNEGVKSNTDGPKGDRKRIG |
| | | PRDRGYTQLTYNELMDYKQKGLCFKCKNPFGPMHRCPAKELR |
| | | VLIVDDVENEEDEERILAVEVGEEEDEKGEMSILNLHHITHETH |
| | | HTMKFQGTIRGVEVLILVDSGAIHNFISQKLVHQMDWPIEPTAE |
| | | MNVKLGNGFQISTQGVCRGLEMCIGDLKMNPEMHLFELGGIDV |
| | | VLGIDWLKTLGDTITNWKQQTMSFWKDKSWVTLKGHEGCRR |
| | | QTVALQSILNRPKPTSQGLLWEMDNVKHKGKGVNELSIQQQAE |
| | | MEVLFNKFEHVFQTPTGLPPKRNRDHAITLTDGHGPVNVRPYR |
| | | YPHHHKNEIEKQVKEMLTAGIIRHSTSSFSSPVILVKKKDGTWR |
| | | MCIDYRALNKATIPDKFPISVIEELLDELHGARYFSKLDLKSGYH |
| | | QVRVKDSDVGKTAFRTHEGHYEFLVMPFGLTNAPATFQGLMN |
| | | DIFRPLLRKNVLVFFDNILVCSKDWETHLHHLEEVLRLLAAHSL |
| | | VANQKKCYFGQRSIEYLGHMITEEGVAVDSRKVDSVLSWPQPK |
| | | NVKRVRGFLGLTGYYRKFIRDYGKIAKPLTELTKKDGFKWGVE |
| | | AQKAFDELKKRLTTAPVLTLPNFSKEFVIECDASGSGIGAILMQ |
| | | DKKPIAYFSKALGVRNLTKSAYEKELMVVVLAIQHWRPYLLGR |
| | | KFVVSTNQKSLKQLLQ* |
| 10 | EpiCas010 | MIKDFASLPPSHQHKLSLQCQLSTIDDHQTHVIPVLIDSGCTDSII |
| | | DEAFVRQHNISTKPLPTRVIVSNADGTKNRTGPLTEYVTLRLTV |
| | | AGRTELMDFLVTGQREARILLGHDWLRRTNPNINWQTNTITYP |
| | | SDDTEYHPEDGDRVIAVDLQSRIDKISVESNVSQTKSQQIAEESE |
| | | RAKSTLTFRDVVPPFYHHFEPVFTKASFDKLPPRRVWDHAIELL |
| | | PEAKGLDCKTYPLNRDEQIHLDEFLKENLETGRIRVSKSPYASPF |
| | | FFIKKKDGSLRPVQDYRKLNALTIKNRYTLPLISELINRLKGARF |
| | | FTKLDVRWGYNNVRIKEGDEHKAAFRTNRGLFEPLVMFFGLTN |
| | | SPATFQTMMDRLLRDLINEGSVIVYLDDIMIFTDSLEEHRKIVSK |
| | | VLQILADNQLYLKPEKCEFEKKEDEYLGMIISHDTIRMDPLKVQ |
| | | AVAEWPIPRTKKELQSFLGFSNFYRRFIRDYGKIAKPLTILTGKV |
| | | DWEWAEDQQRAFDVLKKTITTSPTLAIPNDEDPFKVECDASNY |
| | | ATGAVLSQQQEEKWRPVAFLSKSMNPAERNYAIWDKEMLAV |
| | | MHALEEWRHLLMGAKHQFTIFTDHQNLTYFRDPQKVNRRQAR |
| | | WMDTLARYDFKLEHVKGSANTKADLLSRRVDHPRGERDNEG |
| | | QVLLPQLAEANIYTMEDVLEEHYLDELLASQHKLDDYVVRKL |
| | | KEDPKVWKKRDGIVWIGNEKEDELTLGSFENKIYVPQDPKLRA |
| | | RIIEEEHDTSLSGHPGERKTHEGVNRSFWWPSLRRDIRKYVAGC |
| | | EICQRTKSHRQKPHAPLHPNPIPDRPWQIITTDLIGALPESNGYD |
| | | AIQVFVDRFSKMAHFVPTHVTLDLLGYAKQLRDSVIRLHGIPEK |
| | | IISDRGPQFMSNFTKDFYQTLGIEPGLSTGYHPQTDGQSERINQD |
| | | LEHYLRTFINHRQDDWADWLAIAEFTYNNHLHSATNQTPFFVN |
| | | YGSHPRKGTGQKPSGRSQAAEDFATSMTKLHEQTRQSLLLANQ |
| | | NLKKYYDRKRTEAREYHKGDKVWLEGTHISTDRPMKKLDDKR |
| | | FGPFTVKAKVGPSAYLLDVPRSWKAIHPVFNEILLSPYHTPEFPN |
| | | QPRTSKPPPIQVDGAVEYEAKEIIASRPTKLKGAKLDYFVHWKG |
| | | YPITERTWEPDHHLKNSPSLITTFHRKHPAAPRVIASAALRFRPY |
| | | ENFTTTTEKPRLFDWITNRFTDCGAKILGTRI* |
| 11 | EpiCas011 | MSITDEYFQLTTKYRAQYGERTIVLLQVGAFFEVYGLKNPRTK |
| | | VVTGSQIAEFAQICQLSVSEKNVCVEQDHVVMAGFRDYTLEKY |
| | | LQKLSENDYTAVVYTQTKEGKNVTRQLHAIYSAGTYISYDNDS |
| | | SQQITNHIMCIWIEKYQALDKSARLSCGVATANIFTGKSEIFEYQ |
| | | TAYYMNPTTFDELERCVSTYCPSEVLVLSTTLPQDVVQTIVQYV |
| | | GITHPSRHIFCASNMTDAVKNCTQQKYIHHMLTTFFGEESQQLC |
| | | AEFSNNEIATQAFCYLLHFVQEHNPDLVRKIEIPRFSSQSKTMLL |
| | | ANHTLRQLNIIDDLSASGKQAGQYSSVLSFLNKCASPMGRRLFQ |
| | | QQLIAPTFDQEWLQKEYNMTELMLQPDTEPMIPMLRKQLATIR |
| | | DIEKICRQIVLHKIYPDSIAHLYDSLQTIQQIHVCFAELPKVLEYL |
| | | HHSTEKIDQVLHTMQTYLLIDRCRGIQSVQSFPHSIIQPGVSSSLD |
| | | KIIQTYEENLQHFREIHAFFNKIMRAKEGGDAEYIKIHTTDKSGV |
| | | SLQITKKRATVLKSLLKGLADPKIQLSGGLYFCVEDIKESSASTS |
| | | ADEIEIPCLKNICKALLTSEESMNQEIAKSFQDFLTTFENLHLENL |
| | | EKCALFVAKLDVLHCKAYLASNYNYCKPTIVADHQKSFVDAR |
| | | DLRHVLIEHIQINETYVPNDFCLNLPDQPLGVLLYGTNAVGKTS |
| | | LIRALGIAVIMAQAGLYVPCSAFTYQPYQSIFSRILGNDNLFKGL |
| | | STFAVEMSELRMILRMADESSLILGDELCSGTETESALSIFTAGL |
| | | LHLHAKGASFVFATHFHEILKFEEMQQMPKLALKHMAVHYDP |
| | | QRDCLVYDRKLQDGPGNRMYGLEVCKSLYLPDDFLQSALAIR |
| | | NKYYPTSRGELTHHTSTYNTQKIRGICEMCKTNMGEEIHHLQPQ |
| | | KEANDKGFIGTIHKNHPANLMTLCEACHLKQHKSSITQKRKKT |
| | | TAGYAVL* |
| 12 | EpiCas012 | MEIFISDNTLNMSKKEDVEPVETNKIMKEYLDLTKQYTKIYGEK |
| | | TIVLLQVGAFFELYGLRDDKGTILESQIEDICIICQLNYADKKLM |
| | | YNSKQVIMAGFREYMLDKNIPKIIEAGYTAVVYVQEKDGKNIK |
| | | RVLHSVCSQSTYLSCETDSSPVMTNNIMCIWLDTFKPMDRNVS |
| | | TVSKTRDTIVYGVSTINIFTGKSSMFEYQTPFILNPTTFDELERYV |
| | | SVFSPSEILFVSSLEKEINRTILQYIGVKCPSIHIYDSRDMENKTIIA |
| | | CSKQVYVQHMLSTFFGEEAMNVCREFQMNTMATQAFCYLLN |
| | | YIQEHDKNIVRKISIPEFNNTSDRLLLANHTLKQLNIIDDSSIDGK |
| | | KTGNLSSVLSLLNKCCSPIGKRNFKMMLLNPSTNETWLQQEYD |
| | | MIEYMLQSENIYFIDMFRKQLSQIRDIEKICRQLLVLKVYPSSIYQ |
| | | LYKTIGVIHNLHICLAENKKITDYLCKDISKEQINSYHYINDLCL |
| | | QINEFLENHFVLDDCKNIQSLTSFENNIIKKGISQPLDDLIKHHYE |
| | | KIAIFEFVKKQLNEFMYSNGQNRDIEFIKTHETEKSGLFLQITKT |
| | | RANILKNYIKAKLEKDADYSFHNKELQFSFYLKDIKFGVASSAN |
| | | EEIHIPILQKICKELITIKDEIDVVIRETYLEILKKIEDKWLPSLEQL |
| | | ANYVANVDILQCKTYIAKNYKYCKPEIVKSEKSFVSAQELRHC |
| | | LIEHIQQNEIYVANDICLGNEKSGMLLYGTNAVGKTSFIRALGIS |
| | | IIMAQSGMYVPCSRFQFKPYTAIFSRILGNDNIFKGLSTFAVEMS |
| | | ELRIILKMADKRSLILGDELCSGTETESALSIFVAGLMDLHDKDA |
| | | SFIFATHFHEIINYDEIKMMNKLSLNHMSVIYDREKDALVYDRK |
| | | LKDGPGTKTYGLEVCKSLYLTDEFLDKAYAIRNKYFPETRGEL |
| | | NFKSSTYNVNKIRSICEVCKNEIAEETHHLEEQHTADTDGFIGHF |
| | | HKNHKANLVSICKNCHDKIHHDNSGKNTLNGKQRKKTTKGYD |
| | | FM* |
| 13 | EpiCas013 | MLLTEEYLEEQKKLELKYGKNVVLFMELGSFYEIYEANGIGKV |
| | | KEISNILNILQTKKNKNKPESQRNPLMCGVPSVSLDKHLNRLAQ |
| | | EEKWTIAIMSQTATDNGFERKITEIISPGTNVRNNIKDDTNSYLA |
| | | SLILDKNTKGIYSCGLSLIDVTTGNIKVYEGYGVHNDKEKILDE |
| | | VNLILENYNIKELILLDNLDSNKLYYKFPSINMKTSNKSIHYKIQ |
| | | LITETFNKDFSELGLEQSPYIIDSLVGLLEFLIDHNKSLVVKLNSIT |
| | | FINNKEFMYLGNNPLKQLNIIGDDFSVEKLINKGISAIGRRYIREQ |
| | | ILNPIINADEINNRYKYSLSLSKSNKLPTIEKSLKNIYDIERLSRKL |
| | | ELKTIKPFELGNLVTSLYASSDIIKNIKMLDTLTTFESYINLDLIK |
| | | DYNSIHWNFFKDEQLMTLKTELELIMDLSNYEFDSFIKYSDKDG |
| | | YYYEFTNKKYENLSFTSTIEHTRKLKNSIKLFTSELSSINTRRLVI |
| | | ESKMMEISNVLFKDVLDIFDITIINIEINKIQRIEFYLNNTKLLAKK |
| | | YCIPTILDDVENIFDVTNLRHPLVEHLEQEVFVPNSIKFNKENKG |
| | | YLLYGQNSSGKTVLSKSIGIAIILAQSGFFVPAESLAFTIKDSLFT |
| | | RIIGADNINKGLSTFAVEMLELKNILNRSTPDSMIIGDEISHGTET |
| | | TSGVSIVASAILHFNAPFIIATHLHQLDSINDIKEKPFIHFIHLALTF |
| | | DTSTNSLIYNRTIQKGKGSSIYGLEFAKSLDMPFDFLDKAYAIRK |
| | | EIADDISGLEQLTISKPSSYNSKLFLGTCYLCDNKGEEVHHIKHQ |
| | | HLADDDGFIDTLRKNHKSNLMVLCSSCHNKQHEKKI* |
| 14 | EpiCas014 | MSAKKGFSMIDEYFNIYNEKIKEYGKDIAVLIECGSFMEIYQIDN |
| | | NEEKIGNAYEISKILNICYANKNGDLSNNNRSYPNFVGFTTNSM |
| | | SKYLPILLENNYTVILVNQLETNGNKKGKLIQRGVTAVYSKCLQ |
| | | PLEMDTNADNQLLYISLDVKLIHSNVLKRNKTTMYELNTSICCV |
| | | NNFTNSIEIYENNYTFTKDCIENCLNDFDHLLYKFFSKDINIHLV |
| | | GENIINFESELLKFFNSRYDNMRFHNFDKQKHKEYIKTDYQNEY |
| | | FKKVYNHINFGLLTPIEYLNITTMPSLSIVNLMYTIDFIGRHDLK |
| | | YIRNLSIPKMMNDSDHLFLALDTLNQLNIDKGVFDIVNCTKTAI |
| | | GKRYLKSLLCKPLKNPNEIEYRYKLTDELESIINTNNEIDQLLSCI |
| | | IDFEKLHRKMSLEVLHPFEFEKLHTCYKNVNKLINVLSNYPLFH |
| | | QIIPNNVKLNVFNEYINSYMETFDFDKLKKYNLNTSKDEISNYF |
| | | LKGVIVELDEIQLKIDNITEEIERMRVKYNTICENNCKTKNPDYI |
| | | KIAYTETEGYSFSCTKIKYQTLINAIKESSNFKIKQTNNIVKFFTP |
| | | ELVDLSNKLLTFKDLLKNKINLHYISTLKLYYYNYHDLFTSMTT |
| | | FIQILDVVNSNLKCKNKYNYCRPIIDDNENDSSFFKAVGLRHPII |
| | | EVITQSNKYIPNDIVLDDNNIGILLYGLNSSGKSSLLRAIGIAVIL |
| | | AQCGLFVPCNSFIYKPFHTVISQVDLSDNMYIGKSSFITEMAGLK |
| | | KILDLSGKNTLVLADELCRGTETYSAISIVCSTMIKLIQSNTKFFF |
| | | TTHLHQIPQLKRINTLKSIDICHLSVTIKNSNIIFNRKIEKTSGDPL |
| | | YGLEVCKTIINDNDFINDAFEIRNELTRNNTQILSHKKSNYNKKK |
| | | ITSYCEICNSCNSLETHHITEQYLADKNGIMPDGNHKNNLYNLV |
| | | TLCHDCHLKVTLGKIKTRGYIQTINGRILDYNFTEDTTEKV* |
| 15 | EpiCas015 | MTTEHKKYEYLKFNKSLNEEYLLLQEKYEKIYNKDFTVVLMQ |
| | | VGSFHEIYATNDRGYDLHKLSNILNIIVSKKNKKNPEVSEKNPY |
| | | MIGFPILATPKFINILVDNGFHVIKIDQVTPPPNPKRAITQIYSPGS |
| | | YIEGISSYDSNYILSIYIEEIKQMNNSYILFAGLSIIDLSVGKCIIHE |
| | | TFSTKDDEKYSLDEVLKFIYNFNPKEIIVYYKNLITYSENELLTY |
| | | LELNDKKYLISEFSDNEYTKIIYQNNFLKEIYNLETCMTHIEELD |
| | | LERINNGRLSFILLLNYCKKMNSTILNNIYKPEFYLNTKYLHYG |
| | | NNALKQLNVQTYNSNEKSLEDVINYTSTSMGKRYLNFNICNPIS |
| | | DINILNDRYDKISELKEKNIDLSNKLKKILDIERLHRKISLQNLNP |
| | | HNFVDLHISYSTILSLYEDIKKTKLNKIYNEKVKMNLENYISYYS |
| | | RLFNLDEMEKYNLNDITNSFYNENINEKIDILQKSIKKEYKLIEN |
| | | LRDELEKFIDCKKKDYFNSFNEDKSPIQINYNEKDKYHFSTTSK |
| | | RGSILKNNIKNNSKYNYDSYKFKDQSNTMKITCTYVDNISDKLV |
| | | ILNDKIRPLIKNTYLNDLKEMYEKYNDLYLKINNIIAEIDFINSGS |
| | | ICSIRNKYCKPKIILKDKSFMNVKSIRHPIIEQICSNKYVPHNLNL |
| | | DDNQIGILLYGLNSAGKSSLMKSIGLNVILAQIGYFVSASEFVFS |
| | | PYKSLFTRISNIDNIYKGLSSFALELVELNAILKRSGSNTLVLSDE |
| | | VCKGTEYNSALIIVSAMIKMLVESNTSFISATHLHELTKLDAIKC |
| | | ISKIGIYHISVKYINNNIIFNRLLKKGNGTEEYGLDFAKYMIKNN |
| | | KFIDICNNMKKDINNITANKSKYNKKLIVDKCQICKSIKNLETHH |
| | | IEFQKNTDEYGFIIKKTLNHIHKDHISNLVILCSECHDDIHNNKII |
| | | MKGYEDTIKGSILKYKRSKCIEEKKYNDDEIKFIINKPNISQKKV |
| | | KDLFEKQFKKNITIKNISKIINNKY* |
| 16 | EpiCas016 | MYNIIYLKINIVMTLIEEYFEFQDKYTKIYGEDTIVLMEVGSFFEI |
| | | YQVGDLGKAKEIASVLNMILTKKNKNINEVTFKNPLMTGFPTV |
| | | SLEKNVSRINAQSKYTVVLVKQRGQSPNITREVDEILSPGIQYRI |
| | | NTFKNNFLANIYVEYYRKNMFGIGISVVDITTGENYLMDSVSLE |
| | | GFPDNPLSEIQQMVTKYGPSEVIVSMVDFESAESEATRIRVLLDE |
| | | MIGKYSWKSCDSANYKVSFLNEMFSEIYKVKSILTPIEYLGLEH |
| | | NQLAGNALIDSIGFIQKNNPCILDKMPRPEYINCAKYLTLNNDA |
| | | LSQLNVVDNNKKANRKGSLFELLNTTVTAAGGRLYGRRLSNPI |
| | | SSKEDLNAIYAEIDLFISDSKFAEVRREIAPILDINRIWRRLVMRK |
| | | GDPMDIWNLYTSIKILADMKNDFSWIPNLENLSNSAQSANKYIE |
| | | SIFDISRIEDSYLSNLEDNIFKKENPILDKLEAELAEIKYKSQIIAE |
| | | MFANHSFFTEKTKLVYTDKEGFYIETTPSRYKQYLGLNTAPKSD |
| | | ILAGFDVYKNKSKVRLTNDDLRKLSESKVIKIQKMSILIKEMFV |
| | | KEVDEMYSAIGDDVESISAFISHIDVMAANAYNSVNLSLFKPELI |
| | | DNEEGYSSVECHALRHPIVERLTHSEYIQNDVLFSNNKYGKVIF |
| | | GVNSTGKSTLLKATGLAIIMAQAGMHVPSKMKLSPYQSLYTRI |
| | | TSGDDLFSNKSTFTVEMLELKQILEKADHNSMVLADELSHGTE |
| | | TNSGVSILAATINTLSKRKATFLFTTHLHQITGVRSIMDNKSIDLS |
| | | HLSVNINPITGVIEYDRKLKDGAGESVYGILVAKGLGLADDFIN |
| | | DATSILNEVKSNRTQLDAITENNTNKYNSNKVNKNCEICGFQSS |
| | | ETHHIKEQADFKNQKYLEIQKNAKYNLVNVCNDCHKKIHSGKI |
| | | VNLMYKQTTEGVELKIT* |
| 17 | EpiCas017 | MPSLIKEYFKIQEESEKKFGDKTVIVMHVGGFYEVYSYEGLGK |
| | | ADVVANECNMMLTSKDKSKEISITNPQMCGFPLASLLRYMYFL |
| | | TSKGFTCVTVKQDEKVKEKRTIDRIFSPGTCVDDSESSIVASIYA |
| | | DDGCIAVSWIDLTSGKVMFEEIYNDNEAYRREELTRIIETCGCK |
| | | EIICMSKNGIMYDDAHEIDFCNIYEHISHQNDVISRAYPNDSTVS |
| | | PIEFIDMEMFHIARVSLILLIEWVEDHHQSLIQRIRIPERINRKSLA |
| | | LHNTSVHQLQLVSQDKKSLFDVINKTNTSMGKMLLKQTLLNPY |
| | | SDVSKLNGMYEDIERISKVDVEVVKKVLKNIGNVDRLARRIAN |
| | | KSINLEDIRHLILCFENGLDIIEHMRELSVHNMISENDVKRCYIA |
| | | WHSTIDMEKDVFREGVYDDVDILKKERDTIMYMMDANAKRFS |
| | | KTLGVNEGVKFELHKGTYGLITTPGRAKTLKNRVGNNVVFQA |
| | | DCKQKVVISTPDIQSLIHKLHGAIDAYDECMEEKMKEFVEILYS |
| | | YHEIICTFGTYIAQVDVLLSHYLVAKAFNYTKPVVKEGDKSYIT |
| | | GKNIRHAIVERLNGNTLYMGNDVDLSNEKGLLLYGVNGSGKS |
| | | CFSKSVGICVIMAQIGMFVPAETFEFCPYERIFTRISSEDNIYKGQ |
| | | SSFYVEMMEMNTILRFANEKSLIIGDEICKGTENISAISIVASMCN |
| | | MISKIGATFVFATHLHGLVDLEPIKQIIDTIQIKHIGVTIESGVVTY |
| | | DRHIKNGEGEHLYGLEIATHILGNKEFASLALETRNALLKKTPP |
| | | KKSKYNARVLKKVCHICGRKDEQLDTHHINYQCDAHENIKNT |
| | | KGNLVVLCKKCHDDEHHGFLSIKGWKDTSKGVILDYEFVSEK* |
| 18 | EpiCas018 | MPQLLVDEYIELTNEYRKKYGEKTIIWMQTGSFYEVYAWNEED |
| | | EQIKVSQNILQIRITTKKSGNNRSCLMAGFPDHSYKRFEKRLLKE |
| | | NYTIVYVDQIKKDPIERGVTKISSPGCVFDSDDDSKESILMSVLIE |
| | | IEDNDFYGYYTIYDSNIGEIRMLSIKNEENQTIDHIFDNIGDIISEN |
| | | RVNEVLLNIVSEQYIKLPETNSKILFHYNKIDKIDELLKPLLQNDI |
| | | LSKYFKKYLSIYENVIDSLELSKCLPQEIVNLIYMLDFLKEHEPIF |
| | | VKNLQRPIVDGPNQIKYMEFYNHALQKLQVISNEGNDLIKFIDK |
| | | TLTYGGSKKLKYLLKNPSNNIEELKKRYKSVDLFVNNRQLLNE |
| | | TKTYLKICDLHRFYRRFAIGKINVYNDIPRIVNINENINILINIFKN |
| | | VDNSIYWLPNDKIIQNFKDYCGDLIYTFCIDNCKSSGNVFNKDIV |
| | | PELDDLYLKSESLNQELESIRTYLCNLINEPIYLKNTDKDGFFFET |
| | | TKKRGTKIAKQLKENNKFDLEISNSTSVSKISSAGVKKISVQLCD |
| | | LQTKIIDLTQKKVSEKVLFYYDTYFESSIATIIESMAWLDVYYSY |
| | | SKSAIEWNYVKPNLVECENSYIDAKQLRHPIIEQLLSIEKQAFIPN |
| | | DIRIGFKESYLLYGVNSVGKSSLLKSVGLSVIMAQAGMFVPASD |
| | | YKLCPYDKLIVRIGNTDNLFEAHSSFICEIREANQCVSNTNNRSL |
| | | VLADEFCASTERESATQIVTSMLLWLNEKKSSFIFATHLFELIEA |
| | | PIVNVKICHLKVRIESDSLVFDRKLTSGPPTERNYGILVAKKIFK |
| | | NNKFLKMMDRCQTKTSTRTDINRSRYNRGVLLSECAICAYSPID |
| | | EMSLPLDTHHINMQCMANNDGFIGNYHKDIVSNLVVLCKKCHI |
| | | MAHNGKLNIKCWEQRDCGNVLNYEIN* |
| 19 | EpiCas019 | MLHPRLQEMLAARGIRRLYTHQAQAANLALGGAHVVVVTPTA |
| | | SGKTLCYQLPVLDRLLRRPOSRALYLFPTKALAQDQHHGLTQD |
| | | LLEAGTDAAAMIYDGDTPQAQRRAARRIAQVLITNPDMLHVGI |
| | | LPYHTQWRELLAELDYVVIDEMHAYRGVFGSHVAHVLRRLQR |
| | | LCAFYGSAPQFILTSATIANPAELATNLIGEPVEVVAESGAPSGE |
| | | RHVALVNPPVIEARLGIRRSPLVDARQLALSLAAQGEQVVCFTR |
| | | SRLAVEQLVIALRERAGRVGLAPEAIRGYRAGYLAAERREIEAG |
| | | LREGRVRCVVATSALELGIDIGGLTVAVLVGYPGSVASTWQRI |
| | | GRVGRGQAPSAAFLIASASPLDQYLMAHPEHLFGGGAEAAHIN |
| | | PDNLYVRSGHEQAALYELPMAEGEAAQDDDRRAILAALEDAG |
| | | EARLSRGRWFWSGQGFPAGEISLRSAEAARVSIVARRAEEGRDI |
| | | TIGEVERSSAPRWLHEEAIYLHEGQPYLVRTLDLEGGVAQVEPA |
| | | TTDYFTVASEREEIEIERVVASTDGERMTTAHGEIRLTRQVTGY |
| | | RRQRFETQEVLGWGVVDLPEQVILTSAFWLGVKPELVARLDAQ |
| | | GLWTADRGGARGPDWEQQRDRARLRDGYRCAHCGAPERPER |
| | | AHDVHHIVPFREFGWEPGRNDAYRYANRLANLVTLCPRCHQL |
| | | AERQVALGSTLSDMGHVLRYLAPLWLLCDPEDIGIHADSAGRR |
| | | AERPTLFIYDQLPGGAGLSDQLPRLIVPLLKSAAQVVSDCPCET |
| | | GCPACIGPVLAPAIGRKRRAVGLLEALAW* |
| 20 | EpiCas020 | MSWNEEITPLAALLQRWRQDPAFRRRVAAWARAPAREAQWA |
| | | PIPEDLHPRWREALRQAGFSMLYTHQVEAYRAARSGAHVALFT |
| | | GTASGKTLAFLLPILQALCEDPQTTALFIYPTKALAHDQLAVLR |
| | | AWLSALRLPAAALPYDGDTPGEARRFVRERGRLLLTNPDMLHL |
| | | GILPQHPRWAAFLSHLRFVVIDEAHVYRGVFGSHMVNVLRRLR |
| | | RLAAAYGASPQFLLASATVANGPELAEGLVEGSVRIIEEDGAPY |
| | | GERHFILYRPPLLDAATGLRQSALTAAAGLIWTLLEHGIPTIAFA |
| | | RSRMAVERLLRMVRSAWAERGGDPEEVHGYRGGYLPEERRAI |
| | | EKGLREGRIRAVIATSALELGMDIGTLEACVMVGYPGTIASVRQ |
| | | QAGRAGRRAPLSVAIMVLTATPLDEYLAGHPEFLFRNPPEAARF |
| | | DPDNLAILADHLRCAAFERPLDPGEPFARTLDTGWIVERLIADG |
| | | EPLYRNPTDGRVYFVADRYPAQGVSLRTSTGDRVKIRLGAGKG |
| | | GRIIGEVDRPSAPRLVHPGAVYLHEGAAYRIVSVDWEQGEAMA |
| | | EPFDGDEITEPIVEARWYLDAERARRTEPWGIGAWGEVTIVHQ |
| | | VVGYRRLRWPGGELLGWAEVETPEEILSTTAWWLTIELGLVER |
| | | LKEEGIQIGPLDYGPEWEAIRRQVRARDGYRCRVCGAPEPPGQR |
| | | HDVHHLRPLRTFRDPAEAYALENLITVCRRCHRRLEQMLGTRS |
| | | ALSGLAHLLHGLAPIFLMSDPADLGLLVDPESREGGGPTVLLYE |
| | | GVPGGVGFAAHLWAVQEALLEAALEVVRRCPCERGCPACVGP |
| | | PGEMPDLKGETRRLLECIFAADEAAASKVQ* |
| 21 | EpiCas021 | MADLERLLERLRRDPTFGPNVTAWRRREGRPARYGETPAWLD |
| | | PRLQEALRAGGIERLYTHQVAALQAVRQGEHTVVASGTASGKS |
| | | LCHHLPVLQALLEDPSDCALYLFPTKALAHDQEAVLRALVERIP |
| | | DLPDSAKPFPASYDGDTPGQARRAIRERVRIVLSNPDMLHAGIL |
| | | PSHPHWAPFLHHLRYVILDELHTYRGLFGSHIANLLRRLRRVCA |
| | | FYGSRPQFLCASATIANPGDLAGRLIGEPATVIEEDGAPRGPMH |
| | | LLLYNPPLLDPALGIRRSALQEARRLAVRLIGAGLQTVVFGRSR |
| | | RGVELLLTYLRQDLPPGLRPALRGYRGGYLPALRREIEAGLRSG |
| | | EVRGVVATNALELGVDIGTLAACIMAGYPGSIASTWQQAGRAG |
| | | RRDQPGLAVLVAGANPLDQYLVTHPDYFFGRSPEQARINPDNP |
| | | FILAAHLRCSAFELALEDGAPWGTPAPPDLLEDLVREGSLLHEG |
| | | GRYYWIGVGSPAREVSLRTASGDTVTIIEESSDGIGTIGQVDRPG |
| | | AAMLVHEQAVYIHEGTFYRVERLDWEAGLAYVRPAQVDYYT |
| | | QAHLRVDWRVLSEEAQAPQGRGSRHTGEVEVHAQATAYRRV |
| | | HLHTQESLGWGAISLPPQEFRTQAFWLTLPWPAQEHLRPDYGP |
| | | NWEEQRRRARERDRFTCRHCGAREEVAGREHDVHHLRPFRTF |
| | | GYIPGENDAYLEANRLENLVTLCRVCHARVDHGPRQETVAAL |
| | | QGLGHLLAHVAPLYLLCDRGDIRETTRLRALPEGRPVVLIYDDC |
| | | PGGAGLSPTLFSCADALLQTARERLQECPCAGGCPSCIGPPEEPA |
| | | RDKVIHILEFLLEEEAPPPGTA* |
| 22 | EpiCas022 | MNKLWQHPNPDWVPVSHFGELRYESIDVVMARWDWDKLIWG |
| | | KVRRFVFNLQKRIYKATKSGRYRKAKSLMYLLQNSYYATLLA |
| | | VRTVTTENFRGKCFCARAKTFTSEGKRTAGVDRVKANTPKRK |
| | | MALVKDMLDTIQRGWDKYRPMPAKRIYIPKANGKPKVSEAKL |
| | | RPLGIPTIKDRAMQAVTKIALEPFYEAKFESCSYGFRPAMGCHD |
| | | AIDRITAALLHKQKWVLDADIKGCFDNIDHKFLASQIDTHWRPI |
| | | VKRWLKAGYMEKSDYNQTNMGTPQGGTISPLLANIALDQMET |
| | | DLIEHLRGIKGWKKKIGVTTVSIVEKTEKPQKEKKTQKKPKKA |
| | | NNKGTKEPQKKQKNVYEGAKKSYKCRRNLLIDLVRYADDFVV |
| | | LHKDKEVIEESKRFIGKWLEVRGLTLSEEKTKIVHSTEGFDFLG |
| | | HHIRHYENRIKGTYKLKLLKGTPTEQRRAKATHVLRVEPTKDKI |
| | | KGHYRVIGDTIWKLKAATPSELIRILQPKISGWANYYKTVHSSE |
| | | AFGKLDHLLWKRLYQWAQRRHPNKGKRWVTNKYFATVNGR |
| | | RWTFAEIVGKGKDKKIVKHLKGYSKHKERTGSYARVGYDRSY |
| | | YDGDTAYWAERLSKGYGNITPSKAKMLKRQDGICPVCKCRFG |
| | | NEDLPEAHHIHPRHLGGEDKYTNLVLVHLHCHDKIHRENPGIK |
| | | GKLVSQTSPELQGFLNNGMAPAFQAEGMKPPANTSTVLRTGKG |
| | | AGIITPASDEMKSPVGTSTVLRTGKGAGIITPAPDDNKET* |
| 23 | EpiCas023 | MDTLQLSNPDGRVRSQHSLTGLPSDKKHRDHTDSSGRLVPSCY |
| | | RVINSSDMCDWDWDSIIWGKIRRLVFNLQKRIYKLTKAGKYRK |
| | | ARSLMYLLQHSHYAMLLGVRKVTTENKGKKTPGVDKVKVTTP |
| | | RQKMNLVVDVLDTIQMGWDKYEAKPAKRIYIPKANGKERPLGI |
| | | PTVLDRAMQAVTKSALEPFYEAKFESASYGFRPGMGCHDAIDK |
| | | IASVLLTKQKWILDADIKGFFDNIDHKFIVEQTDKHWRPIVKKW |
| | | LKAGYMEENKYNPTKNGTPQGGIISPLLANIALDQMETCLIEHL |
| | | RGIEGWKTRIGNTKISIITDKKTQKKYESRKHLQLSVIRYADDFV |
| | | VIHEEKEVIEESERFIKEWLGLRGLTLSEEKTKIGNETDGFDFLG |
| | | HHIRHYENRIKGTYKAKLINGTPTEQRKAVTPHVLRVEPTKAK |
| | | VNAHYQELSDIIGKMKATTPSELIGILQPKISGFANYYRTVHSSK |
| | | AFSRLDHLLWKRLYQWALRRHSNKGKRWVAAKYFCTVKGRK |
| | | WTFAEIKGKGKDREIVKSLKGYSKHKVRTGSHARVGYDRSYY |
| | | DGDTAYWAGRLSKGYGNISPSKAKLLKRQDGLCPVCKCALTN |
| | | EDLPEAHHIKPRQYGGLWEFKNMVLLHLHCHDKIHREYGEQK |
| | | RKLCKGTREYDELVKKGVFVSPEVEAKYGPDIAKMRKGPEVN |
| | | KGSSKILRLGSDPGIISRDKDTVPQETHSNGNNEDDELIQVKSW |
| | | KSGEIKMIPRSKMYRKTKRVRDKITNKWKLVKMWFCK* |
| 24 | EpiCas024 | MNILWQHPNPSEVNVFARAQKQFTSEWIPVSHLDRHRYESIESV |
| | | MAKWDWDKLVWGKVRRLVFNLQKRIYKATKAGRYSKARNL |
| | | MYLLQNSYYATLLAVRTVTTENKGKRTAGVDRVKVNTPKRK |
| | | MALVKDMLDTIQRGWDKYRPMPAKRIYIPKANGKLRPLGIPTI |
| | | KDRAMQAVTKIALEPYYEAKFESCSYGFRPAMGCHDAIDKIAG |
| | | TLLNKQKWVLDADIKGCFDNIDHKFLASQLLPLQHWRPIVKRW |
| | | LKAGYMAKSEATKKSEYYRTDMGTPQGGTISPLLANIALDQME |
| | | TDLIEHLRSLKGWKTKIGTTTIGIDINKKTKKSYKSRKNLKIDLV |
| | | RYADDFVVLHEEKEVIEESKRFIGKWLEIRGLTLSEEKTKIVHST |
| | | EGFDFLGHHIRHYENRIKGTYKLKLLNGTPTEQRRAKATHVLR |
| | | VEPTKEKIKGHYQDISDTIWKAKAATPVQLIALLQPKISGWANY |
| | | YKAVHSSEAFNKLDMLLWRRLYQWACRRHPHKGKRWVANK |
| | | YFATVSGRKWTFAEIVGKGKDRKIVKYLKGYSKHKERTGSHA |
| | | RVGYDRSYYDGDTVYWAERLSKGYGNITPSKAKMLKRQDGIC |
| | | PVCKCRFGNEDLPEAHHIHPRHLGGEDKYTNLVLVHLHCHDKI |
| | | HRENPGIKGKLVGQTSSELQGFLNNGMAPAFQAEGMKPIGTST |
| | | VLRIGKDAGIITPVSDGNKET* |
| 25 | EpiCas025 | MNILWQHPNPEWIPVSHLGRYRYESIESVMAKWDWDKLIWGK |
| | | VRRLVFNLQKRIYKATKSGRYSKARNLMYLLQNSYYATLLAV |
| | | RTVTTENKGKRTAGVDRVKANTPKRKMALVKDVLDTIQRGW |
| | | DKYRPMPAKRIYIPKANGKLRPLGIPTIKDRAMQAVTKIALEPF |
| | | YEAKFESCSYGFRPAMGCHDAIEKIAAALLKKQKWVLDADIKG |
| | | CFDNIDHKFLAVQIDTHWRPIVKRWLKAGYMEKSDYNQTNMG |
| | | TPQGGTISPLLANIALDQMETNLIEHLRGIKGWKKKIGTTTVSIV |
| | | VGGKKAKRPQKENEKSQKEPKKANNKGTKESQDKPKKVYEG |
| | | AEKSYKARKHLHIDLVRYADDFVVLHEDKEVIEESKRFIGKWL |
| | | EVRGLTLSEEKTKIVHSTDGFDFLGHHIRHYENRVKGTYKLKLL |
| | | NGTPTEQRRAKATHVLRVEPTKEKVKLHYREISDTIWKLKAAT |
| | | PVQLIAILQPKISGWANYYKTVHSSRAFGKLDMLLWRRLYQW |
| | | ACRRHPNKGKRWVANKYFATVNGRKWTFAQIVGKGKDRKIV |
| | | KYLKGYSKHKERTGSYAKVGYDRSYYDGDTAYWAERLSKGY |
| | | GNITPSKAKMLKRQDGICPVCKCRFGNEDLPEAHHIHPRHLGGE |
| | | DTYLNLVLVHLHCHDKIHRENKGIKGKKVSQTSPELQGVLNNG |
| | | MAPAFQANGMKTPTLTDTVFRVGEGAGIITPALDEKKPNADTS |
| | | TVLRTGKCAGIITPAPDDNKET* |
| 26 | EpiCas026 | MKQNGIDEITIKSSGEINPKSGTNTRILYEHQKNAMECLDIMNK |
| | | EKAYSTLVVLPTGGGKTYTASMWLLKNAIDRKKKILWIAHRQ |
| | | MLLEQAAESFQKFAYAEVIPHISSFHFRIVSGASSHDQTIHIRPTD |
| | | QLLIVSKDSIGRNLQRLDAWMAGENEIYMIVDEAHHSTAKTYR |
| | | KVISYIKEKVPNVKLIGLTATPFRTAEEEQGLLAKIYTDGVQNG |
| | | QAVHGDIGITYQIGLKELINRRILSKPVFESYYTDEFYGKELGLK |
| | | AWESIQHLDMIPDDLAKKMAESSARNKLIVETYQANQEKYGQT |
| | | ILFAVSVEHAIQLTAQFRKAGVAADYVVSSIKDMITGVTLSREE |
| | | NEKKINAYRVGDLQVLINVNILTEGVDLPKTQTVFLTRPTVSTIL |
| | | MTQMIGRALRGTAAGGTANAYIVSFIDAWDEHIAWVNPESLFV |
| | | GNNDFQENAYEQKQRDLRMIAISKIEEFARILDDSTDTSLLEKVP |
| | | FTQRIPVGMYAFTYLEENGGIEHAYQVMVYNSSQKAYENMMD |
| | | SLPDLFQSFHAEEEYLSEHQLMEMEKQCRDTFFCGEMVPPYEP |
| | | KDVIHILKYYAQYESVPKFYTFDEVDRSKLDVGMIAQHIWAED |
| | | MGERKKAEYIDKIWNDADDNMLRLFFGKKLYFLRQLDIELMKI |
| | | RYPDIYEETANVVYETRKLEELSLHDIGKVNPKLEMQLRDAAF |
| | | EKAKTSDGKYQCAVCGMKNRSKTCFHVDHIIPMNQGGKSVPE |
| | | NLQILCRQCNGIKGDQI* |
| 27 | EpiCas027 | MARDYIEVKSFGKVFPKKGANTRMPYEHQRDAMENLDIINKSN |
| | | KFSTMIVLPTGGGKTYTASMWLLRNAIDKKKKILWIAHRQILLD |
| | | QAAESFQKFAYSEVIPHIPFFQYRIVSGASCHDRVINIQALDNLLI |
| | | ISKDSIGRNLNGLDVWLKDEKEIFIIVDEAHHSTAKTYRKVIEYV |
| | | KSKVSNVKLIGLTATPFRTASDEQGLLAKIYEDGVENGKAIHGN |
| | | IGITYQIGLKELINHRILSKPIFESYYTDEMYGESLGLNSWKSIQH |
| | | LDNLPEDVAEQMADSAARNKLIVNTYKAKQKEYGQTIVFAIN |
| | | MVHAVHLSALFNKEGIKSEFIVSDVRDSVTGVRISREENERKLE |
| | | EYRSGNVKVLVNVNILTEGVDLPQTKTVFLARPTVSTVLMTQM |
| | | IGRALRGEAAGGTTLAYIVSFVDNWNEHIAWVNPDSLFSGNNE |
| | | FTDNPVEHVKHELRMIAISKIEEFASVLDDSVDTTMLEKVPFIQR |
| | | IPIGMYAFTYLEENGVDFSYQVMVYDSTRKAYEDLMASLPQLF |
| | | KIFDCKDEYPSEELLEEIAEHCRDTFFLGDMIPPYEEKDCLRILK |
| | | YYAQYEIPPQFYLFDDIDRNKLDVAVIAKYIWDEDMGQRRKTE |
| | | YINSIWENSDENLLRLFFGRKLYFWRQLDIELTKLSNPNIYKKEE |
| | | NVQLGIRKLEDLSLFEIGKVNPEMEKNLRDTAFEKAKDSEGNYI |
| | | CACCGQKRTDRIFFHVDHIIPMNKGGKTVPENLQILCRQCNGM |
| | | KGDKFA* |
| 28 | EpiCas028 | MVAINKRALSEEDIKMKYITPAIEKAGWDIKTQVRAEYTFTDGR |
| | | IIVRGNLVARGKKKRADYILSYKPNMPLAIIEAKDNKKPLGAG |
| | | MQQALEYGEILDIPFIYSSNGDGFLEHDKFTGKERVLSLDEFPSP |
| | | NELWERYKKGKGIQPHEEKIIKEDYFFDMSGKTPRYYQRIAINR |
| | | TIEAIAKGQKRILLVMATGTGKTYTAFQIIYRLWKSRTKKRILFL |
| | | ADRNILIDQTMANDFKHFGDKMIKVNRKNISKAHEIYLALYQS |
| | | MTGSEEWQDIYKQFSPDFFDLVIVDECHRGSARDNSAWREVLE |
| | | YFSSATQIGLTATPKETRDVSTSTYFGEPVYTYSLKQGIEDGFLA |
| | | PYKVVRVSLDKDLEGYRPAKGKVDKYGNLIEDRIYNVTDFDK |
| | | NLILEKRTEIVAKKITEFLKKTNRFDKTIVFCVDIEHAERMRQVL |
| | | INENSDLVKENPKYIMRITGDDEEGKAQLDNFIDPASKYPTIVTT |
| | | SKLLTTGVDAQTCKLIVLDSNINSMTEFKQIIGRGTRIKEEYGKY |
| | | FFTIMDFRGVTRLFADPEFDGDPVVVYEPKKGEDIVPPAEGDYG |
| | | DFGDSVDGTVGEPDSDENETGTRIKYVVDDVEVMVVNERVEY |
| | | YDKDGKLVTESLKDYTKKNIRNEYADLNEFINRWNSEDKKSAII |
| | | EELKERGVFLDAIKEEVGKDLDEFDLILHLAFDQKPLTRKERAY |
| | | NVRKRNYFSKYSDVAREVLEALLDKYMNEGITELESMEILKLD |
| | | PFRKIGTPKKIIKAFGGKEKYIQTIKELEREIYA* |
| 29 | EpiCas029 | MSNPGNSVLSTRYRDARTLVRRVCRLPGEDRAPFKEKVARLRE |
| | | HFEQFNVDVSELCQWFMGLRKAHAAAHAANAATSPAGFGPLG |
| | | DFLLVPALDGVDADESECDRWRLAVFDAVAGFRDLTAPIREAV |
| | | PPALHAAIDATARNGPHTNPKNVAMKRLFERLRTLDSAHRLVL |
| | | LKSAAEWVVARYRRGMENWVRQREEWEKEKTAWETARPQLT |
| | | PEVRERFTQVFRSLVWDRDKRPGVTNKRPRICPYERLKGNLDN |
| | | CIYAGOKGHGPLCWKYAGFKKTREEQFSRFNDRGFEKDAAEFL |
| | | ARRKDGVRRHEVLRQMFRGRSADEQRFTDNWSAYLRALSLNE |
| | | DTVVEHGRLPHCLRIGETFEKSKCEWNPHTELCKQYKRALDNP |
| | | DNGFDEETLRLEPLYRQWRRDYLAGPRKPSFRYPSSRDLPMPKI |
| | | FGAGFHEIDFDRSIVRLRLDDMSAGQWIEFGFTPWPRGYKPSRD |
| | | EIKAAGRVTSVHVHFIGTRARLGFRFSVRPKPSRFTCSQDELDEL |
| | | RSRCFPRQAQDQQFLDAARQRLLDSFPGGAEEAKRNMRLLAV |
| | | DLGETGACAAVYQGTTHQTDCPLAIIKINRLYAGRPDALEPDV |
| | | KGRPSKEKRKFDKGDPRGVRKEHVARHLARIAQKTAEITQHPR |
| | | RQPAVPQTVTPGAHDLRSLKRHTAWMIRDWTRLNAAQIVKVA |
| | | EDQRCNLIVFESLRGFLAPGYDKLCPKEEDKKRWLAMFAYGRV |
| | | RRKVVEKAVERGMRVVTVPYYKSSQVCSTCGAVQENTNLHRE |
| | | NKRDRKFICEKNTCAVELNSDANAARVLARIFWGEITLPSPDEK |
| | | TVRRS* |
| 30 | EpiCas030 | MTRIKYRQERTLVRGLQRLPNQDKAEFNKNVLGLRRHFEQFNL |
| | | NVAELCQWLISFKPENPGSVCETKLFWEFMLEPENFCPDTYDR |
| | | GPDWLRHEIFVLAAGWRTFEDIKAHNMPESLFESIKIASSKPRSK |
| | | TAEALFIRLKSLEASHVMVLLKSASEWLSTRYVRQSENWKQNE |
| | | QEWQKKKAAWEDKHPELTPAIRDKYNAIFVELGISEKRPHVCS |
| | | WKKLSENKDNCDWAGERKNIGDKWISHSDLCIKYHEFARKLR |
| | | SKQRQHFVDNANQYLELRRRYPQWTRDMAMNGLFKNVPLAR |
| | | NWFRNAWTNYLNALNILETTILENYSGHLPHCEKLSDECVFKK |
| | | HTDNCRRYKLLLGEKLSNQERELEETYREWRREFLAPPNKPFL |
| | | RYPSAQKLPTPKLFGRGYYDLDFTRHVVKLRLDDMPADNFVSF |
| | | GFKPWPRGYDKKPGEINITSVHVHFIGTRARVGFRFAVPHSDSR |
| | | FSVSQDKIDELRSGGFPGKSQDQEFLNEARQRLLDGMNENQKS |
| | | ALRIMAVDLGTHRAAAAFFTGCIFNKAKLLKLKKIDLLTEPKTD |
| | | TTKPEKLSADEKKIQREKGLTQHHVGKHFETLEARTKEIISKRQ |
| | | NMKMAPSDDTPDIVGDHDLRHLTSHIRRMIRDWVRLNARQITE |
| | | LAEEENVDLIVFESMRGFRAPGYDKLDLKKKRRLAFFAYGQIR |
| | | RKVAEKAVERGMRVITVPYFKSSQICAQCGRSQNDKNKLRDN |
| | | KWKQSFQCEFSDCNYKTHSDENAARVLGRVFWGEITLPTD* |
| 31 | EpiCas032 | MQQGCDSRFDTTIKEQEVSLFNTYTKYVDCVNKLPNAQLVDA |
| | | DFSLKLLSMVIANALEVLNGIKKTVDNDKKYLLLSGGFKLPEV |
| | | VDVLARLTSNVRQDGAIEIANFGLNSRAVRKRDILNSKKNELDE |
| | | IESELGALTNKIKKSAQKPKKEDINSKGRLTQERNSLVAEIKKLQ |
| | | AMNKYGLIFRRKNCDGDFEYFLALVDRDHMENLSDKSKFKSA |
| | | QSTGYQYMEYNCLKLKSLLQIIFESDSDNLSGVRKLERYGTVKP |
| | | LFLSKEHSRELEKSKTLIAWLDENIQKIVDTIAGTTNPKITYTEY |
| | | GNTDAAKNAADVWRDFERHFNRQSYSISWCHLNFEKLKTTKS |
| | | VELFQIANKDHSIHPDFTSDERDRKKLNRSSGRPNLFTTYLDNV |
| | | LTDEIGNHYQLLGEGVEYFKHKCGAEPSTPRRKSIDRYYVEFRV |
| | | AFVPNKSESWEETNSSVKNAISQSDSPKHFIGIDRGENAIANIVVI |
| | | DENNRAILKQDMSCLAIFSDITPHSAKKSYIFLTGDEPVYIATKS |
| | | ATDALTDKTEIKKLSDVMFTEYRDKLYKLLPPSEDKKVIPSKSS |
| | | PCLDEMNIEDLKKLISKLESPEYNPASRKMYLAKTNTPIIDYVYI |
| | | RRYNILLLQAKYMLGKKQVRVTDSNFEQEKIQRKEYLLHATNS |
| | | IKDNYSAYFVHHVCKLAEKYNAYIVFENLNKKNGDTREINFLS |
| | | GEQISGLTPKQQNQLKGVSFYVNMEQKLINKLNYLATKDAQSH |
| | | SLTLKGQFTQSIDYKGVITSMTNEQINLITSPKKQEQVEALKSGL |
| | | GKGKKNCYQIGCILYVDAANTSKECCNCGFSVNTALTRCERTD |
| | | EGLSFSGSSNTSYKWGDTRLTERGIDESFDLNNKSDGVKLSVRP |
| | | GKVVNAKLSTRDVYDPISCLACGFDGRGTGHDHLHENDNDAY |
| | | QKIKLLQTCDELAAFNIAKRGGKLIKGQEEV* |
| 32 | EpiCas033 | MNYSDFVGLYSISKTLRFELKPTPATKKLLDDSGVKETDKIRHE |
| | | HFKQAKKILDEVHREFINDALKNVSLSTSDLQDYIKLTLDKASK |
| | | KADVQKAQKKIFSSFEQAYKITNYEWQEKLDAKKFFLQSPGIQL |
| | | EIASQLAEKIAKNLEMNEDDVKNTLKQFDKFSTYFKGYAENRK |
| | | NCYSFDGKATEIANRTIAQNLITFYKNIEKFNKYYSGNSLLADD |
| | | VSKYFLLENYNQFITQQSIAQNYNVKIGELNYIINQERQKQKKD |
| | | AKANLPLFKVLYKQILGDATKEVPFEFIKDDAEYKAIIHNLKDK |
| | | MPKWIEEIEDHFVSQAKQLQNPEGVYLGKKAINTLLQKYISIDV |
| | | RPLINEELPELAKNKSSKTDDDESLRNQFISVKDFFEALDKVQE |
| | | QSETSILRSDESAALLPSQFIWNKLCNDIEKDVKVAKDSAPKIDT |
| | | SNKNSVKSALDSFNDLFRMYSYFELRYKNQPVAVMDEDAKFY |
| | | QIFNAEDTPFANEKDKRFSQVYDLIRNYLTKKQYSLDKWKLNF |
| | | DCSILLGGWDLNKEPEKHGVLLRHKQQYYLAIIDNSAKNAFDK |
| | | KENLDLYTAGADDWQKMEYKLLPGANKMLPKCLFSKSFRENF |
| | | GVPSDIDVLYKNDEYKKGDKFDINKMHKLIDFYKDAIKKYEG |
| | | WSMYEFVFSDTKTYQSIDQFYHEVDRFAYRLDFVNINNQVLSK |
| | | MNENGQIYLFEISNKDWLKEARGKANLHTQYFLNTFRGGDFKL |
| | | NGEAEIFFRPKSIEAKVDKERKSKHEIIENRRFSEDKILFHVPITEN |
| | | RNSKLSSNFAFNQHAIERIKTNGVNAVIGIDRGEKHLAYVAVVD |
| | | KNGKLLDKPVSLNQIHSICKGRDFPKDYFDELKKREDERRSNRQ |
| | | DWDAVRRIKDLKDGYVGHCVKQVVDLMVKHNAVVALENLNF |
| | | GFKQGRSKIERSTYNQFEQALLKKLQYVVDKNKDDDEIFGVQK |
| | | GVQLAPPDVAPGKISNHMGFVFYVDPSYTSAIDPKTGYRQHVR |
| | | LDDRVKTKNFSKFVDDAFDKIYFKDDKLIFEFNWRGLAKAQNG |
| | | LGVSKNKKTDIKQDKKWGITLDVPRTVFHKNVAGKSETEIVNP |
| | | QQLLICSLNEAGIKLNEDIKLQLVKNTNLSSKFVKSFVYCFNLA |
| | | NKLRNQIDGEDAIISPVYPNGFDSRNQKLDGKYKWNGDANGV |
| | | YNIARKGAILLQKLNKASTPKEFNCKVSSEEYDGFVGGEK* |
| 33 | EpiCas034 | MIFNCILTEHQETQNTKVLVEKFVNDFKDLMGDMQKQEEYLA |
| | | ENYGVDKSLYKIESIKNEGDEKSKMDTATFYIYPYFDFDKNLSI |
| | | EECIKRVDSFIGDKKLNRNVEELIGVSDNAGGFSFYFNNGLQLL |
| | | QNNRIDEIKNAILQASSLWNGKENELQERLEFLNKKAKQVLPV |
| | | RIVKNWHEYRSDIGGSIQSWVSNSLRQDIEIKKLLYGYKEKKVK |
| | | RGGEIEEHDIEGHIQEIEKVLGDAELGGYKQYFENKEDEGGIRQ |
| | | TEEVIAKVKQLKKHLDDEKEKIDTEQPKINPAILNDYRDVLADF |
| | | RNTFNHLYQKRYGLEEQDEGIEQKDEKGKKIQTVNKKYPKLFK |
| | | ELPKIPKFLGAVKIEEKGVYDKYICSYNGLKKGVEFLGGVTFTG |
| | | SKRIDDYYTYRNDSSKDKEEIEKARKRSIERVQRVLENILRLYRS |
| | | TDAKQLKGQKIKSLLAKYCKDISLLDGEYNYIYRNERYARDKR |
| | | GQKIELKRVDDELVEDDFIQGLISDLQSFEWRLDNIKQGKWYER |
| | | VAGWGALVEIEKIKIGLMAWFYEISVPHNFQEIEKNKLHFRKVE |
| | | AIVKRYNQDEVLDNKEKNTIIQQGIVSEMRGMIARMSKQEIIAR |
| | | YVVQPVNAEQKYPLVMDEGKYYIHPKEILDKDEIKEQPVNMIDI |
| | | SKEKDLTEEKNSRIKVRRDSLFAITSSKYQTQFLNNSLNRQLEK |
| | | WQNLNPQLSEYSFIVEDTHKIKWVQDGDSYTPNCDEVLLSRLF |
| | | VSVPFKIGVENKDKTVAIKQQNKFIGIDIGEYGVAYYGLDGSDF |
| | | SAKPFVAGFFFQPDMRKIREGIAKNKERQQAGTFTNSNTQIKRV |
| | | RDNATTKIRNQVHQLVVENNALPIYEKEVSAFESGSGKISAIYN |
| | | SIKRSDVYAESKADDMEHKLIWGNDKIMVGAEIGAYATSYMCS |
| | | VCYESIYAYIAKDDYEREYEVIDIVGEQKQIKDKWNATVKFDIE |
| | | GKEMYAYIEQKDKPVGKGDTFNGKGTINFVRKYERPPLDVVR |
| | | KIERLPSAIQQELNECTKKERVKSNGEKEKISVWENRRGSQGVF |
| | | RCAFCNKCSDADVQASMWIALKGYLQNVKNAVQKKENKEQY |
| | | KMYEGSIGKEVVGEIAKGFSQGGKEQVRFILDFAKDNSIMLR* |
| 34 | EpiCas035 | MATKTYRARLSSIDNRTQQAVVRTHYFFNHCLRQMIERYLDM |
| | | RKGKYGPFCKQLGDLLLTRSHTDAHGIMDQLTRGAPSTQTDQE |
| | | WAVLVKHHHREYGPLFLQHEGFAVVDGVVVHTKPQGRHEPTP |
| | | GRLAVTAKFWHQICDMACAFLKSNQELMAVWRKDKTEWLKT |
| | | KNAWEVDNPEFMTFWNGPYADFERACEAARVAAQEAAGQTP |
| | | TARKRDSRVRGKRIDRWHLWYEWLVAHPETVGWRQRAEAGD |
| | | FKPVPDAVQAAIRKKTKRQDKYVGKYLDWLRENNPELKALDS |
| | | LRRTYVRRYLRFKRPPTLTLPSANKHPYWFTLELNEFYKGADFE |
| | | KGCVKMRLIDCDADGLLEMKWFDVRMGCDHRLKPAWRKEKF |
| | | ASDGRYPPYLGGKIGRKLDRPAECAADRKAGVKGVKLVLDKG |
| | | RRELFFTLVEQDCPPRLKFKKTKNRRCTADNLFDAGDQRVPIRV |
| | | MSVDLGIRHIGAYAICEGTKQDDHWEMKYLKKGLLSDSSLPEL |
| | | GQIRLHDRHLKKLRSRQGDAPTGERNFVALQDHRTGMADDRF |
| | | KKAAHLLIETARRAGVDIIVFEKLDTLKPDAFDERWINRQLRDM |
| | | NRRQIVTHVAQQAGEFGLLCKDDISPWLTSQVCSRCYRPGWRF |
| | | SIKPKAPYKEQCSRAACQDFGYPIWDPGGHLFRCPHCGYKVNA |
| | | DINAAGNLAAKFFGHGFWFDNNLSRDKASGCFIWTDDQRRCF |
| | | DAREAFEQWAAEASRRQALKEAPF* |
| 35 | EpiCas036 | MATKSYRARILTDSRLAAALDRTHVVFVESLKQMINTYLRMQN |
| | | GKFGPDHKKLAQIMLSRSNTFAHGVMDQITRDQPTSTLDEEWT |
| | | DLARRIHKTTGPLFLQAERFATVKNRAIHTKSRGKVIPSPETLAV |
| | | PAKFWHQVCDSASAYIRSNRELMQQWRKDRAAWLKDKNEWQ |
| | | QKHPEFMQFYNGPYQNFLKLCDDDRITSQLAAEQQPTASKNNR |
| | | PRKTGKRFARWHLWYKWLSENPEIIEWRNKASASDFKTVTDD |
| | | VRKQIITKYPQQNKYITRLLDWLEDNNPELKTLENLRRTYVKKF |
| | | DSFKRPPTLTLPSPYRHPYWFTMELDQFYKKADFENGTIQLLLI |
| | | DEDDDGNWFFNWMPASLKPDPRLVPSWRAETFETEGRFPPYLG |
| | | GKIGKKLSRPAPTDAERKAGIAGAKLMIKNNRSELLFTVFEQDC |
| | | PPRVKWAKTKNRKCPADNAFSSDGKTRKPLRILSIDLGIRHIGA |
| | | FALTQGTRNDSAWQTESLKKGIINSPSIPPLRQVRRHDYDLKRK |
| | | RRRHGKPVKGQRSNANLQAHRTNMAQDRFKKGASAIVSLARE |
| | | HSADLILFENLHSLKFSAFDERWMNRQLRDMNRRHIVELVSEQ |
| | | APEFGITVKDDINPWMTSRICSNCNLPGFRFSMKKKNPYREKLP |
| | | REKCTDFGYPVWEPGGHLFRCPHCDHRVNADINAAANLANKF |
| | | FGLGYWNNGLKYDAETKTFTVHTDKKTPPLIFKPRPQFDLWAD |
| | | SVKTRKQLGPDPF* |
| 36 | EpiCas037 | MENKNCNFDKFTKQYQVSKTVRFALTPIGRTEEHIIEKQYIEAA |
| | | QRKDQAYKIVKPVIDKKLCVMIDDVLTHCEKQDWEKLNELTL |
| | | QYQNDKTSVEALGEQQGKIRKNISKEFENSIEYKNFFGKDSEKL |
| | | FKTFLPQCLEEINASEADKEAVKEFQKFKTYFNNFLTVRKDIFN |
| | | DDSKQNTIPYRIVNENFMIFAGNRKTFSNIVKLIPDASAEIAKEGI |
| | | ERQEWEFYDVQNIDSCFEPDSFQQCMSQLGIQKYNFIVGLVNSY |
| | | MNLYIQQHPEALEVKRSRLKLRMLHKQILSDRTNPSWLPEQFA |
| | | EGEEGEKQIYEAIQALEDDLNKNHFQEKFDNLLQLIDTENPRIYI |
| | | AASEMPRVSSALNYGWNGLNNVRMNVLQELNSEAKIEKILKQ |
| | | DVSLNELSDALNKYADIYKKEQVPTLQQYIEYGKELLQDCAVA |
| | | CNDYHASIRNNVERLNQNEKLIEVLKTYLDCYQRVMHFMNVF |
| | | VVGDELDKDVDFYAELDELIEDISQIIPLYNKVRNYVTRKAYSL |
| | | DKIRIMFEKSDFLGGWGQDFDTKKALIFEKDGLYYIGIIEKKYT |
| | | NTDVQYLYEEIEEGNKAVHFVYNFQKIDNKNFPRIFIYSKGTNY |
| | | APAVRKYNLPIQNIIDIYNERKFQTDYKKVNEQEYYESLEKMID |
| | | YFKDGIMKNETYRKFHFNWKPSNEYENIKEFYDDTDNSCFLLE |
| | | KEEINFNHLKEQADQGKIYLFQISSKDFNKGSTGTPNLQTMYW |
| | | RELFSDQNCKEGIIKLCGGASIYMRDVSIKQPIIHKKDSWLINKW |
| | | YMEEGQNKVIPDDTYVKFSQIVQGRMSEAELNVEEQQLWNSG |
| | | LIQKKKATHDITKDRRFTRKQYMLHAPLIINYKQQDIPTPRRFNE |
| | | EVRSFLKNNPNINIIGIaRGEKNLIYITVIDQKGNILSGMQKSFNQI |
| | | EEKGANSRIVDYYGKLQSVEDRHTEARKNWKQIGTIRELKEGY |
| | | LSQVVHEIAQLMIQYNAIIVMaNLNMGFKKGRMKVEKNIYQRF |
| | | EKMLIDKMNYLAFKKNIEGKAINPYEIGGVMNGYQLTNKFTNF |
| | | ADMGSQNGFIFYVPAAYTSVIDPVTGFVDVFQRAEFKTGDFLQ |
| | | KFEDISWDDKEQSFVFTFDYQKFKCNGTCYQNKWSLYADVNRI |
| | | ETVKKNNQVYRQEQCNPNQRLIELFDSKGIAYRDGHNLVNDLG |
| | | NYDSKLLSDILYNFRLILQLRNSMRDYSTGEVIDYIASPVMYHR |
| | | ERFDSQKKNPSLPLDADANGAYHIALKGLMCLQKINEYADPDG |
| | | KMDIKNMKITNEEWFKFMQTRRESNV* |
| 37 | EpiCas038 | MRRFFMTKMTDFTKLYQLSHTLKFELRPIGKTSENLQNSGLLEE |
| | | DFKRSQDYVEVKSVLDEVHKKFLQDVLSDCDLSWQELADALK |
| | | IFQVNKDRKVLEDVQKKYRKLLADRFNKNKFFKTLTEATPSKL |
| | | FKSLLAEPDMAERKVETFKRFACYFKGYQETRQNIYSAEAQQT |
| | | AAAYRAVNENFVKYLRVVELYENFRKNFPELAEEIGRRTAELT |
| | | NGELSQILRIENYNRFIAQSGIEYLNSLIAQINSAVNLYRQAHNV |
| | | KLSFVPLLFKQILSDREQAFTLERIEKDADLDEKLRAFCERHIGN |
| | | VEIRGETGDLFSRLKVLLTTLNDDCELYIDAASLSAIGNALTGN |
| | | WSCFRDAMTAYANETLKNKSLREKYCKQPVYSFSEIKSWRVV |
| | | RTDDDGNVQPVCLSDYWHGEKTLELFAAEKILRNRILEMVRVP |
| | | HDKNFRERKNDVALVKDYLDAVVEILHLLKPLNVGSENGGDL |
| | | DLLGILTEFYEALSPVIPLYNQIRNYVTQKPSDIGKIKLMENNPTL |
| | | ADGWDLNKEKDNTAVIFCRNGMYYLGIMNPKSKTDENSLATD |
| | | SSTDSYQKMVYKQLQNPNQMLPKVFFSAEKIKFFAPSEELLRRY |
| | | EAGEYKKGEKFDLAYCHELIDFFKRSIDRHEKWSKFEFKFSETS |
| | | AYSGINEFYREVAEQGYKMSFVNIPVKAVDQLVEDGKLFLFQI |
| | | WNKDFSEKSTGTPNKFTLYWKALFDRQNLSDVVFKLNGEAELF |
| | | LREPAITEPVVHRPGGKLVNKTVVAAVDGNGVAIRNSIPDAVY |
| | | TEIYKHVNGMEEGTLSPAAREHLNRFPRLDWEPGMGLEATVKR |
| | | TVVKDVKFQLVKDKRFTERKFSFHVPITVNFKSPRLKGAKLNE |
| | | MVWEFLKDNPDVNVIGIaRGERNLIYVNCIDLKGRILLQKSFNIV |
| | | GGFDYHQKLDLRERERGQARKSWNEIGTIKELKAGYLSGVVYE |
| | | IARLMVEYNAIVVMaDLNGGFKRSRTGIEKQIYQKFEKALIDKL |
| | | NYLVFKNESDWHVPGGLLNGYQLTGPLESFAKLGKQCGWLFY |
| | | VPAAYTSKIDPSTGFVSLFKLSNYTNTEKRKAFFENFESIVYQAE |
| | | QDAFVFAFDYKKFKDVENCWKTSWEVYSARRRLVYSRRSRQI |
| | | EEINPTRLIKRALDKRGVVLSDGFDLKHYITSVEAVNENAAFFK |
| | | DIFDAFGTTLQMRNSSGEEDYIESPVLNENGQFFDSRKAGKEWP |
| | | RDADSNGAYHIALKGLLRMKSGQTAKLKRLDWFEFVQRRGFIT |
| | | E* |
| 38 | EpiCas039 | MNDLSEFVGLFPVSKTIKWELKPVGMTAERMREAGFLDRDRSK |
| | | RDAYTVVKSMADRFYRGYIDERLSTLDLDWNHLGDAMSALKE |
| | | SGETKDKRELENIKKSFKDSISKHIFTKKDDEDIFSAKVLARLRG |
| | | TETDDEEVEALEKFDRFAGYFQDFFVNRRVFADTVRGSCAYRI |
| | | VEDNFPRHFENCRKYQIIRRDNPELILELKRSMEGYLETDPDVLF |
| | | SIDHFGKLLRQRDIDAYNRVVGSVFRSESDNSRGFNPILNEFHQ |
| | | RNPDSPKMLMQKLQKQILSEPDSGSFRMRNYENEGQMFEDLQ |
| | | NLWTGIETNRLLEIPASVLEEGEFDKSGVFISKAGVNTLSNWMF |
| | | SDWERIGGLLQIFKINEMGDSASKSRKKIDKWLDSDSFSLDVLN |
| | | RAVADETDIGIVPFLSRRLSDAAQQAREAYLGLEGCASMRREQ |
| | | YQGRVKTFVDSLLRYRDILKLFSYDPDLPLDRGFYDTFQFVYEE |
| | | MASAYRAYNRCRSFITRKEYSTEKFKMSFDCPHLADGWDLNK |
| | | ESSYNSFLFRRDGLYYLGIRNPAWRYEDFSVKADPECDDFYEK |
| | | AVYKQLQDPVKMLPKVFFSKKGLDKYRPPERILEGYENGLHRQ |
| | | GDSFDLGFCHELIDFYRDAISQNRDWDFFDFKFKETSEYSSISEF |
| | | YQDVESQGYQMRFIRIPAQTIDEMVDEGRLLLFQMYNKDYSRD |
| | | KTPGGKPNLHTMYWEAAMSEDNLRDVRIKINGYAELFWRDAS |
| | | IKDGTVHRKGSVLVNRIGKDRKPIPSEAYYDLCRYCNGIRDDLG |
| | | AGKGYEDQIVCKNSRYDIEKDRRYCHDRMFLHIPLTFNFKSDG |
| | | KTDVNRKVLEWIRRNDDVKFLGIaRGERNLIYLSLVDSDGRILW |
| | | QRSLNTIVQKDGRGNTRALDYHEKLAVREGERRNAQQSWGPM |
| | | DAIKNLKEGYVSAAVHEICRIAVEEKAVIVIaNLNYGFKNSRKK |
| | | VEKQTYQTFERMLINKLQNLAFKDAEPGSPGSVLAPYQLCGPID |
| | | SMEDIPNHCGMLIHVTAAFTSRIDPVTGFVSVFNLSDITNNAQR |
| | | ADFLSRMTSIRYNQDEDVFEFSFDYGDFRTRLTPKVRKWTVCT |
| | | YGDRYKYNRKDRTLVRISPTTMIKDALTKAGIRYEDGSDIRKEI |
| | | SGSDAVRQVFEAFEAILDMRVTTRDEDVIVSPVRDSSGRHFVSG |
| | | TSGPGLPQDADANGAYCIALKGLLLARKIRASDEDRLPLLNNN |
| | | DWVEFMQTRVM* |
| 39 | EpiCas040 | MIEQFVNRYPLSKTLRFGLIPQGNTEENFKIMQILEKDEALARN |
| | | YGKAKEIIDRYHKFFINSCLADFHLTDLEKYALLYYEPNKTEEE |
| | | VAAMDETAKTMRQAISGLFCKENKAIYDRLSKKDLIENFAMSV |
| | | IETDEERTVLESFRKCTVYFTGFNQNRLNMYTGEGKSTEIAHRLI |
| | | NQNLPKFLDNARVAKTILAALPKNDLDQIDVDFYDIFGINTQNIF |
| | | EIPFYNQTLTQAGIDRYNQIIGGYTCSDGRKIKGYNEYINHFIQE |
| | | TKQKLPKFKPLYKQILSDRETISFIPKAFQSDSEVLSALRNFYIKK |
| | | NVETDKSIRDVITELKKLFQEMDSFDEKGIYITNGVAITDLSQAV |
| | | CDNWFVIKKAMQAEYDAEHSSKNRNEDYEKKRGNDLKKVKS |
| | | YSLSALQRYMDAEMEDKKSLAEYIKTHTTQLCDLTASAYERVR |
| | | HLLETEYSPTKKLTADADAIGRIKDFLDPIMEVFHFAKIFLGSGE |
| | | EAEKDDVFYGIFSPLYDQLADIIEIYDKVRNYVTQKPYKTDKIRL |
| | | IFERGDFLSGWAQPTESQAKEAHLMEKDGRYYLFITVTAINKA |
| | | DWERALYGKDNFQCACHIEYYKQKPDNKNTPRLFIRSKGDHY |
| | | APAVEKYNLPIQEVIDLYDKGYYKAEFKKKDQKLYYTSLRKLI |
| | | DYFKLGFTKHENYKDFAFAWKESDAYENITEFYQDTMNACYR |
| | | LERVTVNWNGIMDLVEKGRGFLFQIYNKDFSDHTKGTPNLHT |
| | | MYFKMLFDERNLENGCYRLLGGAEMFYRKASLKLSETTIHPKG |
| | | VAIKNKNPRSDRAERVLDYDIIKDKRYTETQFSLHFPIMMNCLA |
| | | QGNPKLNLDIRKALLKSEHQYIIGIaRGERNLLYVCVINEKGEIV |
| | | EQMSLNEIIGDNGYTVDYHELLDRKEKERDAARKSWGTIENIK |
| | | ELKEGYMSQVIHKICELVLKYDAVIAMaDLNGGFMNSRIKIEKQ |
| | | VYQKFEKMLTEKLSYLVDKKADPMSDGGLLRAYQLTNKPDPN |
| | | KSVQHGMQDGIVFYIPAWCTSKIDPTTGFVDLLKPKYTNKEEA |
| | | KDFFKRFQKICFNEVENMFEFHFHYPDFQRGITAGNTDWTVYT |
| | | NGSRIRTFRNPEKNNQWDNTDIVLTDAFKALFAEYAIPCDADM |
| | | KEAILAQDSPKFFKELCGLLSFTLQMRNSITGRTDVDYIISPVKN |
| | | AAGRFYDSRKAASDLPCDADANGAYNIARKVLLFIRRLQAQEE |
| | | SNLMNKDLEKITNAQWLSFVQNGHE* |
| 40 | EpiCas041 | MTKMTDFTKLYQLSHTLKFELRPIGKTSENLQNSGLLEEDFKRS |
| | | QDYVEVKSVLDEVHKKFLQDVLSDCDLSWQELADALKIFQVN |
| | | KDRKVLEDVQKKYRKLLADRFNKNKFFKTLTEATPSKLFKSLL |
| | | AEPDMAERKVETFKRFACYFKGYQETRKNIYSAEAQQTAAAY |
| | | RAVNENFVKYLRVVELYENFRKNFPELAEEIGRRTAELTDGELS |
| | | QILRIENYNRFIAQSGIEYLNSLIAQINSAVNLYRQAHNVKLSFV |
| | | PLLFKQILSDREQVFTLERIEKDADLDEKLRAFCEQHIGNVEIRG |
| | | ETGDLFSRLKVLLTTLNDDCELCIDAASLSAIGNALSGNWSCFR |
| | | DAMTAYANETLKNKSLREKYCKQPVYSFSEIKSWRVVRTDDD |
| | | GNVQPVCLSDYWHGEKALELFATEKILRNKILEMVRVPHDKNF |
| | | RERKNDVALVKDYLDAVVEILHLLKPLNVGSENGGDLDLLGIL |
| | | TEFYEALSPVIPLYNQIRNYVTQKPSDIGKIKLMFSNPTLADGW |
| | | DLNKEKDNTAVIFYRNGMYYLGIMNPKSKTDFDNLATDSSSDC |
| | | YQKMVYKLLPGPNKMLPKVFFADKNIDFFAPSEELLRRYKAGE |
| | | HLKGEKFDLAYCHKLIDFFKRSITRHEEWSKFDFQFSETSAYAGI |
| | | DEFYREVAEQGYKMSFVNIPVKAVDQLVEDGKLFLFQIWNKDF |
| | | SEKSTGTPNKFTLYWKALFDRQNLSDVVFKLNGEAELFLREPAI |
| | | TEPVVHRSGEKLVNKTVVAAVDGNGMAIRNSIPDAVYTEIYKH |
| | | VNGMEEGTLSPAAREHLNRFPRLDWEPGMGLEATVKRTVVKD |
| | | VKFQLVKDKRFTERKFSFHVPITVNFQSPGLKGAKLNEMVWEF |
| | | LKDNPDVNVIGIaRGERNLIYVNCIDLKGRILLQKSFNIVGGFDY |
| | | HQKLDMRERERGQARKSWNEIGTIKELKAGYLSGIVYEIARLM |
| | | VEYNAIVVMaDLNGGFKRSRTGIEKQIYQKFEKALIDKLNYLVF |
| | | KSESDWHVPGGLLNGYQLTGPLESFAKLGKQCGWLFYVPAAY |
| | | TSKIDPSTGFVSLFKLSNYTNTEKRKAFFENFESIVYQTEQDAFV |
| | | FAFDYKKFKDVESCWKTSWEVYSARRRLVYGRRSRQIEEINPT |
| | | RLIKRALEQRGVVLSDGFDLKHYITSVEAVNENAAFFKDIFDAF |
| | | GTTLQMRNSSGEEDYIESPVLNENGQFFDSRKAGKEWPRDADS |
| | | NGAYHIALKGLLRMKSGQTAKLKRLDWFEFVQRRGFITE* |
| 41 | EpiCas042 | MRNRYSKILCLLLLFACCLPQEGNAFWPFKKKKKEDKRLRKLI |
| | | AQNLKAHPLVDKLKVKNAFGKNGYLETLPLGKEEKEAVKVFA |
| | | GFGGFFNNYNKNRENYFSTEEKSTAIANRIVNENFSKHFSNVEI |
| | | VTKIQKEVPELIQIVEAQFKGYDAIFTVNGYNMALSQAGIDTYN |
| | | EMVAIWNKEANLYAQKAGKLPDGHPLKKKRNYLLSALFKQIG |
| | | SEKEHLIQIDRFDGDEEVIEALTGVKKMLQEADVFEKLNMLVE |
| | | DMENWDYSKIYLSAQSLSNVSVFLNNLYEDERENSWNYLDNV |
| | | LREKWQIELQGKKKGTDLEEAIRKKKQSFYSIEELQEAVNAIEE |
| | | TDKCYNVSKWLLGAMKSERVIEEKKKDVEDFCTQWKNERNSL |
| | | KETDITALKEYLEQWIFLARYCKSFYANGIEKKEKDEAFYHILE |
| | | DVLYVLDEVIYFYNKVRNYVTKKPYSLEKMHLKFGHNELANG |
| | | WSVNKEGNYGTAILRRNGKYYLAITNSLNKKMSIPTQLESTGN |
| | | NYEKMVLNVFPNVFRMIPKCTTGRNDVKSCFERKEPNEYFFIDT |
| | | PEFVNPFKVTREEYELNKITYDGVKKWQSDYSKNTQDEKGYKE |
| | | AVTKWIQFCMRFLQSYKSTAIYDYSTLQQPEKYETVDSFYHDV |
| | | EKILYECHFEYVPANKIEQLEEEGRIFLFQIYNKDFSENRRPDSK |
| | | KNLHTLYWEALFSEENRKAKVIQLNGKAEIFRREKSIEHPIVHK |
| | | AGEVLVNKRTKDGEPIPDDIYKDLSNYFNGRNVTSEKEEYKEC |
| | | LDKVYTSTKKYDITKDKRFTETKYEFHVPITLNYQADGVKYLN |
| | | QKILHVLRDNPDVNIIGLaRGERNLISYVVLNREGKIVNNQQGSF |
| | | NIVGKMDYQKKLYQKEKNRDKERKTWKNIETIKDLKEGYISQV |
| | | VHELTDMAIRNNAIIVMaDLNFGFKRGRTKVERQVYQKFELAL |
| | | LKKLHYLVTDKTEGEAMLKPGGVLQGYQLAREVKTLKEIGKQ |
| | | CGCVFYVPPGYTSKIDPTTGFVDVFNMSGVTNREKKKAFFEKF |
| | | DNMFYDEKRDMFGFSFNYEKFTTYQSSYRNDWTVYSNGSKYV |
| | | WNSLKRTDELIDVTKELKLLFEKYAIDYRNEALFEQIMSQDTDK |
| | | NNADFWNKLFWYFRVLLRLRNSSDELDQIVSPVLNQNGEFFET |
| | | PKKITEKSYLSDYPMDADTNGAYHIALKGVYLIQEKIADESVDL |
| | | DNKLPKDFYKISNAEWFMFRQKEK* |
| 42 | EpiCas043 | MVSNKEKTPTYNSLTNLYSVSKTLRFELRPQYSTLDHINEDQIV |
| | | GKGEELKNHYKTFKKILDQVFSRIINDSLDKTYLDQKYISTYQD |
| | | LAFKHRDQLTDKDRAELKTLKETLKKQIDKSLDHKDKKAIFSD |
| | | PVKFLLDNESEFTDLIGDNRPSIEAFNRQKGYLSGYLQNRANIFD |
| | | HTTNETSVAFRIVEENLAIFLNNRITLQHFLEKVAGKDGLLKSLQ |
| | | EALFQLGFKLKLEDLLSLDYFNRTLSQSGIDQYNLLISGKTLEDG |
| | | AKLQGINEILNQYLKQHQEEKLHKIKLKQLYKQILSESKTESFTL |
| | | DFVEDNKGLAAMFLQFIDFANQLIEEKMPLLGMIQELKNSSVSA |
| | | ELLSRLHLNRKNIKRLSNFIYKDYGYIEQALEENFLSTVEGKITK |
| | | KVLGEHRKQDTFTIHEILVALQKQQYEKDGTLESADRLFLPSIV |
| | | DFVYQNLNCEYSTLLEKVRSEKQPLLELFKERQLLEDQDTESHS |
| | | SKYPDHPFNDHEIKIIKTTLDFYKNLQSNLALLQISDENLKLDSE |
| | | FYSEFDEFYQGLKNIIPVYNKSRNFLTKKPFSTEKTKLIFNSPQLL |
| | | DGWSKSKESDCLGTIFIKDGKYYIGIINSATNAKKTLFEPHNFAN |
| | | FDQSQYFEKMNLFFLSDLKRDFPKKYFSEKWHNQYPIPADLRE |
| | | KYDYYRIDEHKDERKNDLEYHHQLIAYYQDCLKKDTEWQIYQ |
| | | FKYKSPEEYSDINEFLSELTPNTYKMEFTKIPAEYIKKLVDDGKL |
| | | YFFQIYSKDFSEFAKGKPNLHTLYLKAVFDQKNAEEFNYNYKIS |
| | | GSAEIFYRPASIETRVTHPKNQPIKNKNKNNPKIESIFQYDLCKD |
| | | RRYMSDKFFLHLPVELNRTPLLPNDSLVNSMVNQVVSSRNQNY |
| | | FLGIaRGERHLIYLVLIDQSGKIIKQQTLNQITSSYQEKSNNQTVE |
| | | VVTDYHDLLNDKEKIRKKNLQEWQSVENIKELKAGYLSNVVN |
| | | EIGKLIAEYQPVIMLaNLNTGFKNSRIKIEKQVYQKFEKALIDKC |
| | | NYFMRKDIDPSTIGGLYHALQLTKEYSKQYNGKONGIIYYIPAS |
| | | YTSNIDPTTGFISAFVQTRYENVEKAKALIEKENDITYDADESLF |
| | | CFTVDYKKFSPEAKLWQQNIWQIYTNGDRIYTFRDKEEWQSKN |
| | | YILVEEFKDLFTKYQINYRNDLKTQILSQTDAGFFKQFLFLLRLT |
| | | LQMRNSRTADSNEADIDTKKRENDYIISPVKNQYGKFYDSRQD |
| | | YMDWPENADANGAYNIARKGLIMLKHLKEGLPEKRICDISTEE |
| | | WVQFVEELNK* |
| 43 | EpiCas044 | MTRVFIFCLLYLKKTKYAPGVHKEGKMKINAFINCYSMSKTLR |
| | | FKLVPEYETEKNLLEKGFLDRDKLRADDYDLMKKVIDKYHKH |
| | | FIDKALEGFKFDLLQDYAEAFYSQSADDDGKKLEEIKKKMCKE |
| | | LATCFSKQDEFKLLDKKELVEKLIPAAEFIEDEEKDIAKRFKGFT |
| | | TYFTGFNENRQNLYAAELKHGTIAFRLIEENLPAFLYNCKKGVK |
| | | IFEELDEGDVETLNNELGEILSIGNVKDILNVEYYNKTLTQNGID |
| | | VYNQIIGGYTQEDGTKIKGVNEYVNLYNQTHDKKLPSLAKLKK |
| | | QILSDSYSLSFLPAKFNDDSELLLSLKKFYSMVNEETGLSVEKAI |
| | | QEMRNVFLHIDDCDLHNVFIDAKFINKVSNDVFGNWSVLIDGIN |
| | | AEYEKLNPFNGKNLGNYEEKRKAFLNKIESYSVDALQAYSGKE |
| | | EKIADYVQKRAVELCDSVACAYENMSNKVINAREGKVKLYQD |
| | | DEKTEIIKTFLDAVQEFKKFAEMFCYDGTDGDTTFYGEFANYY |
| | | GQIAEIIPLYNKCRNYLTKKPYSEDKIKINFDNSQLLDGWDANK |
| | | EKDYLTVLLLKGNNYYLGILDKNHKNVLIKDVPEKGQGEPCFK |
| | | KMIYKLLPGPEKMLPKVVFSAKNIGFYNPDREILRIRENGTFKT |
| | | GDGFNIDDCHKLIDFYKECISKNEDWKTFGFRFKETSEYKNIGQ |
| | | FYNEVKEQGYKISFTDIPESYVKDLVNDGKLYLFRLANKDESPY |
| | | SKGKKNLHTMYFEGIFDPENIKEKVYALNGGGELFFRCASLNY |
| | | DKPTHPKNVPIKNKTYEFRTDNAKKETSTFEYDLIKDKRYTKD |
| | | QYTLHCPVTLNFKERGIERINDLVRQSLRESDDNYVIGIaRGERN |
| | | LIYISVIDGKGKIVEQFSMNNLSSGNDVSIDFHKMLETRERERDA |
| | | SRKNWNTIDNIKDLKQGYLSYVVKKICDLVVKYDAIVAMaDLN |
| | | VGFKHGREKFERQVYQKFEKALVDKMSYIVNKNASPHSAGGL |
| | | FRAYQLTNKKYNENEKQNGFIFYVRAWNTSKIDPTTGFVNMLP |
| | | LKYQSKEKSKEFFDKFEDIFYDENKDMFGFTFRYDDFGINIDHK |
| | | NEWTAYSNGERIITVRNSFGKWDKAKIALTPAFKKLFDDYNVD |
| | | CRGDVKRQIMNVDDKDFFVRLYKLLSYTMQLRNSDDADDYIL |
| | | SPVVNAEGMFFDSRNSDGSLPCDADANGAYHIAKKAMWAIGK |
| | | IKEADEESFKKTSLAIDNKTWLEFVQKA* |
| 44 | EpiCas045 | MVSNNENTPTYNSLTNLYSVSKTLRFELKPQYSTLDHIRDDQIV |
| | | DKGEDLKNHYKTFKKILDQVFSRIINDSLDKTYLDQKYISTYQD |
| | | LAFKHRDQLTEKDRAELKTLKETLKKQIDKSLDHKDKKAIFSDP |
| | | VKFLLDNESEFADLIGDNRPSIEAFNRQKGYLSGYLQNRANIFD |
| | | RTTNETSVAFRIVEENLAIFLNNRITLQHFLEKVTDKDGLLKSLK |
| | | EALSQLGFKLKLEDLLSLDYFNRTLSQSGIDQYNLLISGKTLENG |
| | | TKLQGINEILNQYLQQHQEEKLHKIKLKQLYKQILSESKTESFTL |
| | | DFVEDNKGLAAMLLQFIDFANQLIEEKMPLLGMIQELKNSSVSV |
| | | EFLSRLHLNRKNIKRLSNFIYKDYGYIEQSLEENFLSTIEGKITKK |
| | | VLEEHHKQDTFTFHEILVALQKQQYEKNGTLESADQLLLPGIVN |
| | | FIYQNLDCECSTLLEKARSEKQPLLDLFKEKQLLEDQNTESHAS |
| | | KYSDRPFNDHEIKTIKTTLDFYKNLQSNLAIFRISDENLKLDSEF |
| | | YSEFDEFYQGLKNIIPVYNKSRNFLTKKPFSTEKTKLIFNSPQLL |
| | | DGWSKSKESDCLGTIFIKDGKYYIGIINSVTNVKKTLFEPHNFAN |
| | | IDQSQYFEKMNLFFLSDLKRDFPKKYFSEKWHSQYPIPADLRKK |
| | | YDYYRIDEHKDERKNDLEYHHQLIAYYQDCLKKDTEWQIYQF |
| | | KYKSPEEYSDVNEFLSELTPSTYKMEFTKIPAEYIKKLVDDGKL |
| | | YFFQIYSKDFSEFAKGKPNLHTLYLKAVFDQKNAEEFNYNYKIS |
| | | GSAEIFYRPASIEVRVTHPKNQPIKNKNKNNPKTESVFQYDLYK |
| | | DRRYMSDKFFLHLPVELNRTPLLPNDSSVNSMVNQVVSSRNQN |
| | | YFLGIaRGERHLIYLVLIDQNGKIIKQQTLNQITSSYQEKSNNQTV |
| | | EVVTDYHDLLDDKEKLRKKNLQEWQSVENIKELKAGYLSNVV |
| | | NEIGKIIAEYQPVIMLaNLNTGFKNSRIKIEKQVYQKFEKALIDKF |
| | | NYFMRKDIDPSTIGGLYHALQLTKEYSKQYNGKQNGIIYYIPAS |
| | | YTSNIDPTTGFISAFVQTRYENVEKAKALIEKENDITYDADESLF |
| | | CFTADYKKFGPEAKLWQQNVWQIYTNGDRIYTFRDKEEWQSK |
| | | SYILVEEFKDLFTKYQINYHNDLKTQILSQTDAGFFKQFLFLLRL |
| | | TLQMRNSRTTDSNEADIDTKKRENDYIISPVKNQYGKFYDSRQ |
| | | DYMEWPENADANGAYNIARKGLIMLKHLKEGLPEKRICDISTE |
| | | EWVQFVEELNK* |
| 45 | EpiCas046 | MVSEKENTPTFNSLTNLYSVSKTLRFELRPQYSTLDHIKDDQIV |
| | | DKGEELKNHYKTFKKILDQVFSRIINDSLDKTYLDQKYISTYQD |
| | | LVFKHRDQLTDKDRAELKALKETLKKQIDKSLDHKDKKAIFSD |
| | | PVKFLIDNESDFADLIGDNRPSIEAFNRQKGYLSGYLQNRANIFD |
| | | HTTNETSVAFRIVEENLAIFLNNRLTLQHFLEKVADKDGLLKSL |
| | | QETLSQLGFKLKLEDLLSLDYFNRTLSQPGIDQYNLLISGKALED |
| | | GKKMQGINEILNQYLQQHQEEKLHKIKLKQLYKQILSESKTESF |
| | | TLDFVEDNKGLAAMLLQFIDFVNKLIEEKMPLLDMIQGLKDSS |
| | | VSSEFLSRLYFERKNIKRLSNFIYKDYGYIEQSLEENFLSTIEGKIT |
| | | KKALEEHRKQDTFTIHEILVALQKQQYEKDGTLESADHLLLPG |
| | | VVDFLYQNLDCKHSTLLEKVGSEKQPLLDLFNEKQLLEGQDAE |
| | | SHASKYSDRPFNDHEIKVVKTALDFYKNLQSNFAIFQITDENLK |
| | | LDSEFYSEFDEFYQGLKNIIPVYNKSRNFLTKKPFSTEKTKLIFN |
| | | NPQLLDGWSKSKESDCLSTIFIKDGKYYVGIINSATNAKKILFEP |
| | | YNFANLDQNQYFEKMNLFFLSDLKRDFPKKYFSEKWHNQHPV |
| | | PADLREKYDYYRIDEHKDERKNDLKYHHQLIAYYQDCLKKDT |
| | | EWQIYQFKYKAPEEYSDVNEFLSELTPNTYKMEFTKIPAEYIKK |
| | | LVDDGKLYFFQIYSKDFSEFAKGKPNLHTLYLKAVFDQRNAEE |
| | | FNYNYKISGSAEIFYRPASIETRVTHPKNQPIKNKNKNNPKAESV |
| | | FQYDLCKDRRYMSDKFFLHLPIELNRIPLLANDSSVNSMVNQV |
| | | VSSRNQNYFLGIaRGERHLIYLVLIDQNGRIIKQQTLNQITSSYQE |
| | | KANNQTVEVITDYHDLLNDKEKLRKKNLQEWQSVENIKELKA |
| | | GYLSNVVNEIGKIIVEYQPVIMLaNLNTGFKNSRIKIEKQVYQKF |
| | | EKALIDKFNYFMRKDLDSSAIGGLYHALQLTKEYSKQYNGKQN |
| | | GIIYYIPASYTSNIDPTTGFISAFIQTRYENVEKTKSLIEKFNDITY |
| | | DAEESLFCFSADYKKFSPEAKLWQQTIWQIYTNGDRIYTFKNKE |
| | | EWQSKSYILVEEFKDLFAKYHIDYCRDLKAQILSQTDASFFKQF |
| | | LFLLRLTLQMRNSRTTELNGTDADTKKRENDYIISPVKNQYGKF |
| | | YDSRKDYVDWPENADANGAYNIARKGLIMLKHLKEGLPEKRI |
| | | CDISTEEWVQFVEELNK* |
| 46 | EpiCas048 | MKSIYENFIGLESKNLTLRFALNPEAKTQENLKLYWDKLRDEER |
| | | DRAYPIVKKILDKEYQQLISEGLKLLENQNVLDWTELAEYIRTS |
| | | DLSKKKKEDKRLRKLIAQNLKAHPLVDKLKVKNAFGKNGYLE |
| | | TLPLGKEEKEAVKVFAGFGGFFNNYNKNRENYFSTEEKSTAIA |
| | | NRIVNENFSKHFSNVEIVTKIQKEVPELIQIVEAQFKGYDAIFTV |
| | | NGYNMALSQAGIDTYNEMVAIWNKEANLYAQKAGKLPDGHP |
| | | LKKKRNYLLSALFKQIGSEKEHLIQIDRFDGDEEVIEALTGVKK |
| | | MLQEADVFEKLNMLVEDMENWDYSKIYLSAQSLSNVSVFLNN |
| | | LYEDERENSWNYLDNVLREKWQIELQGKKKGTDLEEAIRKKK |
| | | QSFYSIEELQEAVNAIEETDKCYNVSKWLLGAMKSERVIEEKKK |
| | | DVEDFCTQWKNERNSLKETDITALKEYLEQWIFLARYCKSFYA |
| | | NGIEKKEKDEAFYHILEDVLYVLDEVIYFYNKVRNYVTKKPYS |
| | | LEKMHLKFGHNELANGWSVNKEGNYGTAILRRNGKYYLAITN |
| | | SLNKKMSIPTQLESTGNNYEKMVLNVFPNVFRMIPKCTTGRND |
| | | VKSCFERKEPNEYFFIDTPEFVNPFKVTREEYELNKITYDGVKK |
| | | WQSDYSKNTQDEKGYKEAVTKWIQFCMRFLQSYKSTAIYDYS |
| | | TLQQPEKYETVDSFYHDVEKILYECHFEYVPANKIEQLEEEGRIF |
| | | LFQIYNKDFSENRRPDSKKNLHTLYWEALFSEENRKAKVIQLN |
| | | GKAEIFRREKSIEHPIVHKAGEVLVNKRTKDGEPIPDDIYKDLSN |
| | | YFNGRNVTSEKEEYKECLDKVYTSTKKYDITKDKRFTETKYEF |
| | | HVPITLNYQADGVKYLNQKILHVLRDNPDVNIIGLaRGERNLISY |
| | | VVLNREGKIVNNQQGSFNIVGKMDYQKKLYQKEKNRDKERKT |
| | | WKNIETIKDLKEGYISQVVHELTDMAIRNNAIIVMaDLNFGFKR |
| | | GRTKVERQVYQKFELALLKKLHYLVTDKTEGEAMLKPGGVLQ |
| | | GYQLAREVKTLKEIGKQCGCVFYVPPGYTSKIDPTTGFVDVFN |
| | | MSGVTNREKKKAFFEKFDNMFYDEKRDMFGFSFNYEKFTTYQ |
| | | SSYRNDWTVYSNGSKYVWNSLKRTDELIDVTKELKLLFEKYAI |
| | | DYRNEALFEQIMSQDTDKNNADFWNKLFWYFRVLLRLRNSSD |
| | | ELDQIVSPVLNQNGEFFETPKKITEKSYLSDYPMDADTNGAYHI |
| | | ALKGVYLIQEKIADESVDLDNKLPKDFYKISNAEWFMFRQKEK |
| | | * |
| 47 | EpiCas049 | MEKFPRCSHLKVKQHCGPFVEWYEDLQSDRQKYVSLFFPWIGI |
| | | GYGERKDEGDDIDFISPEQTLERRKAELGKLEELVVRFVTVMLK |
| | | GGSSASSCYSLFYEDYKGKDTAKKSTSKVNFIKALPLLSKKEIE |
| | | DRVLGDNQLSLINLDEWMELGQSGLSADDIWDKVSQRLVTGE |
| | | RSLGVDVRHTLKIRCQRPRDRGYCVVLMEMVARQLRSCYECY |
| | | KNHLNELQELREKTTKQITESRDDLFSLVCDLATVLTEQDSGLS |
| | | ERTLFSGRKQALKPQGKPSKIQAIVDELRQPQYSPLLEADDKEL |
| | | RQAYKAWNSQRRLRDRETRRPYPCRPNWRNDYQVSLGLTGRG |
| | | GFWMWMDGSDVVVDLQDCGRLTCSPSHYFGDLAIEGQLHQY |
| | | DIRYRNKLKRRGKKLRKPPAYGEWIEATIKEIGIQRKGGDYYVY |
| | | LLPVISHDSTNFQAAKKFFAAAAPKQEIIDALPDEMKVLALDLN |
| | | ISNPVCSVKARIGRALDGPLSVLDYGQGEIVDGPKILVTDGPRC |
| | | VALIELHSDVFFVKDAIREYKRTHGVAFNGAGMDDEMLERLSS |
| | | IPSPSPSVRCQIQAWVAAIGKRLHLLHYGMRREGYKNLGEAIRL |
| | | LAAMDAYDSMLQSYQRIHLKKGQSLPRQAKNDHSRAGLRETIT |
| | | RRIAYAIAQEARDCNVVFVEDLKADFDADNDNNALLRLFAVKT |
| | | LIRYIQEALEKIGVGLVLVAKDGTSKTNPMNGDLGWRDRNNKE |
| | | HLYFELNGEAVRCHADLVASLNVLLRGLDHSVCPYSFVTKDTN |
| | | KKPGRADQKDKGYGKRLKRFFKDRFGSSNLRFVAKPDGEVTA |
| | | TTRRPKDALIGERVYYHGGKLVTSEVHYRMENEIKHLLTLKSP |
| | | AKELSLTEDGIRSYENFTHV |
| 48 | EpiCas050 | MVTPESQSDAKKAFADALFAGQEAAYGSFYAKYTPLPFPRKDS |
| | | ALGRLTAELGTIGDEKYAKALKLNGLDPDEWAGYRNRAIAGEP |
| | | PDALWEEIKVKVGVTNKATLASRGVGNLPTLRPDSKSYATALA |
| | | YMLSSSLSSWEECAKLHRARREEIAAKAAVPDSMTEEYALVCQ |
| | | LAQALEEAGSGLTAKMLNGAIREMAKTDSVYMVAITELADPK |
| | | YKPLLALDKEDRARLMDTWQAERKLSTSLAAPKRPLFDKDYQI |
| | | PFGMTGRGESFALRTDDSGRLWVKIGDHEEFSCAASDYFGNLK |
| | | VEALPSDKGYKITYRKMVKNRRRQRAGYKSSVGGPLIEGEVKQ |
| | | ILLQRRMNRRGKESFYVYLPHSIAVKDDRRHLSWFFKRADWK |
| | | DGKGLEELPDQLIVSGIDLNIDQPVTVTKAIVSKNYTEGEINCPY |
| | | GGGHLTGKPRSVCPDTPISQEAKLTHRKMRLLIEVIRESKACHK |
| | | AGTPLSSEGSERLKILYADISKPPCRNNRESIQVVYSSLKKTYRR |
| | | LKYELMQQGANDVSGCIRMLQLDDTHRALTACFDAFSGKPAL |
| | | AGRNFGVARKKTSKGTARLNFREHVSHLYANAVLRACHGSDV |
| | | VFIEDLSTSFDGNAHNSLKRLFAPGQLIDAITSLLESHGIAVVTV |
| | | DPSGTSRRHPITGDIWYRDSMKKRELVGLIDGELVSVNDDLAAS |
| | | INVLTKGLNHSVVPHKFKVDPSGRVNVKEDGGKRLKVFLREKG |
| | | LSGRLPVGSADAAKSKKAKALTVYPSSSGLLSDRQVFEVLEQIK |
| | | HLLAAGCKPRPVELMAGPGETYAAFSLKEVLEWSKSKSSEDLR |
| | | HTGLGDETG |
| 49 | EpiCas051 | MSNIKTTNILIPKQQDNYFEIENMLDRQNKIANDVFKLLLTYQG |
| | | HILDQHFFGEDSKEINFIQKRQELYLVAKQADPLVTEDVENLTC |
| | | DGFVNQIASKSNMSNDLFKAFEVAYEELSEMKSYVAGKSEAKK |
| | | LACKLKQWGVLPSFDNHIGEIKSGLSNAFGRFKSWYECDKMTQ |
| | | ENFQKHVDSKSAYLEEMGDVISDRFQEFANELGNVYNSKGLDN |
| | | FDYFGGKFLLFYEQILKPSFEKGEVCYSGSIEFSNGKKQQYSIYG |
| | | GIIDLLFKYKELWETKSWSKKVKRKIETVEQCTFSRFYNIFEDFK |
| | | YLRKNKQRAGFREFSSTSSASICLSNSNIIKYDITQVDGDRNNFY |
| | | LTINGKQIQCRYRTKKIKYNKTTVEKTVENKCYLDNLEIEKQIK |
| | | NDKPTGNYIFKFDVNKKRSNIATVNEPKIFKNRKGIYISLCMNID |
| | | LDDGKYDENKNIAFYLKTKYFNTYTKEADTLPLLKNKTVRIMG |
| | | VDTGIKPIYTYSILDYKNGKITDPNIHGKLEGKYEFTNYFNMKSL |
| | | KNVIRDLPKYFQTDDHKELFKSHLFNDLTFTRDELFDELKNFNT |
| | | LREYKSRKNKGCLIRREIKSYRHKIGRMKDTNFKSDVKDITNVF |
| | | GIVKYIDSYKQVCSSWNYIGETVETKTQPNFAFADSHLAKYRE |
| | | NLKDNMIKTLVFNIIDEAKKHDVDILAIEDESSEYSTSTKRDNVL |
| | | KALFAPSQIQSEIERCAKKEGIALMTVTRHYTSQVVFGKDIFGY |
| | | RPKENDKTKLKWIENGEIKFVDADINAAKNIAAKLALKDVSPY |
| | | TLDIEGDGKYFKSSKWDKSKVFKKVNDKFVNVSDQKPKNTKT |
| | | KMKIFLNGDNYITKEKKNELVEEIKNYKIKT |
| 50 | EpiCas052 | MKSIKSIKSIKTKVVKNNELKLIELSTWCSSICEQLERYIFILGGK |
| | | QIHDRDGVVVLDGAVERKIYCKKDKSLIAACEVVYKHFTDKSS |
| | | KSRTFGSWFLGGKSEGDNTNKGRKSTKEKTEKQIAKQIADKKE |
| | | LTDSLQLLWDKKLLPFPIDNKGYDFINTPRAKSYKWAITKTIHA |
| | | KIKSYNEQCVETKKEYDALNAEINTYKTILFSGYSEKDIDDLQK |
| | | FVDICEANNHRINYKFISFLKRKDLNFDEQTGKYRKEGKWIQHK |
| | | NGKEVKSKYSMKDEIVEALYKYKSLTKNDVSVLCNEHQKEDE |
| | | MGKVVHYNMKRYSDLLFRKKNKKEIPSYTKISLATSKIELGLN |
| | | NVKYNVEQVEDKLIWTICDQTGKDIQFVTVYTRKKEDNRING |
| | | KKGAGFYKGKHHQLEDLKIVPVGDIGTYDISFKVNGKRPFTGT |
| | | LKEPNIICRGGKVFVQMPININIDKTLNDARKKVLYAYRETYSG |
| | | SVNGKKQKMIKIENSKIAESLKSLGRDAIVLGVDLGLRGLATAV |
| | | VSHNGKNETVKSSQYIKGDIVEWEKYRVFNDNIREVKKYIFLTK |
| | | KSYTATTEEYTEFYKECSKPEQDYLDSLKTYKDKNVKLNELKY |
| | | TKNAWSVSKMFEDVSKMFETLKQDRLKYYDIFNMPYWAASLK |
| | | NYMSLMKSYNYVGVDIKVSKEYMSKYQSLYNNIKEDYAKKIG |
| | | SYIVQLAVAKNCDIIVLEELKSNLGSVDRKSKRDNEMSLMWNC |
| | | GRIKTHVENMAKDYGMFIDEVPEYGTSQVYHKTGNYGYRDED |
| | | NREIFWYEDNKDVAYIHADENAAINIAKRFLSQHTDNSSFSVIL |
| | | KGDAYYLNIASNSKRMRAAALKTFGDLNKPFKINANDKNGNL |
| | | YKKTRIFKSDSRWIGVNDKDLYIEHIKSLRNLRVRQ |
| 51 | EpiCas053 | MNHFTQNLAVTKIDSSVQTLYDDFQEDFRKWYSILCSWAKVPY |
| | | RVEDDNGNITHVHAKQRAAELSSLYSAQEQLLIERFMRVFLGA |
| | | SKEASSSYSLFYDKYAPNGAKKTAALKEFLGEIPTLDDSQVEKK |
| | | ANTNKNCKRITVKEWRDFAAQFNKLGIDAIWGKLKEKITSPDR |
| | | GVGLQVRGFVSERCKHPENAGYCRQLCQKLTEQVKSTVRICEL |
| | | YIQETADLQSEIGNKTKQPEVKKCFDLLCQLSDELRDVNSGLSG |
| | | RVLFQSIKDIQGGKDSDRKNKVCRTVMSDDKYLCLIDSDKKIV |
| | | QRAFQAWKLSITLKKRQEKLYPKQRSHQNDVPMPFGLTGLASF |
| | | DVSPDGSKFTFSEGSVETKPSLYFDGLRFQKNKHSFDMEYRVA |
| | | YASRRTKKGRRPKRVGRLRKATLKQVELFNKGGKFVIKLPHGI |
| | | ELPEHRAKLATFLMCADPKEKGYSKGGLVDEFRALAIDVNLSN |
| | | PLAACVADVSKHGDGPIQALDYGRAKLCVDPFFLVANTPVCGQ |
| | | LIKLCKECKAVVQAIKEFKECQAAGSDFSFETDLFLPAGHSPRH |
| | | RIQSKVQELSRVLRDWKFKLQNTNQVAEMIRLLELMDAYQSMI |
| | | SSYQRIHLKAGQTLPPQKKNSTKRANYRKDISRRLAFVVADYA |
| | | QSLNVDVVFGEDLKADFDRDKNTNSLSRLFAGGMFLQHLENA |
| | | LAIRGIAFVKCAKNGTSITDPVSGVFGWRDKFNKERLWIERDGE |
| | | LGWVNSDHAACLNVLLVGLMHSVVPYRVYVKAGRKTTDDVE |
| | | VFEEDHPIGPRKKRFFELCFGENKPKFEVVENRVILDNAGTYQG |
| | | FIYAHCLDAMQSEKQHWAEYDRIKELALAETNPKQFGLTPKKG |
| | | EGYRNFCQPDIHEQVEVVGNKRVAATKQILESLADEKIEAT |
| 52 | EpiCas054 | MSQEGEVKFKSNSDMKDRGVVTGYDYSLINAFCSKYTNLWEE |
| | | RDDYDSTYLDKGGRKRQACIMTDRYYISHMIKRLKYMTKKHV |
| | | NVPLFEQGKKAPCQLFGKNYMSYTLRQDEETGTFEFSFTDPTSE |
| | | SEGRAVKKFNASLAKNGYLNNLKIVESSNKDGVYKVSFNTAVE |
| | | NKDTLTIQTTYTGYIKEPRIRFNLEKNVTYLDLCISGIESDNDTFN |
| | | KILSEEQKENKEAIFKGFSSYFKASYAKGRDGSQKGSLDVDVFR |
| | | AMGVDLGLNPLAALSIYEVKKSDKGEDIGGQKVKIKHLTDHKS |
| | | SDINKANKDCRVKKAKRTLKFIEGVKGLISVANHHCNYLEDVE |
| | | ATGEGKEVNVGYMEMLCGDVIENHTPFKDLEDYMKYIENNLY |
| | | GVDRKEWKKKTSDWVVPSLIQQAKKKISEMRREYFDAEDKHY |
| | | HTTKVNHVCNLQLHKIQAVKEMISTLNSFSTLGLNDEEKQAHA |
| | | DNGRPCADLWEYINGLRNYLSKGIASIIINHALRNSVNIIFIEDLD |
| | | MKASSFDSKEENKLKSLWSASQIMDYLEKFAAKHTIAVQEVNK |
| | | FMTSQFSSENGTLGVRGASGNKSQFLYEDKGEVCDVDSDINAA |
| | | RNIAIRGITRHSNMPMFKVKVASDKEFILEMPKAAGMQKLSSM |
| | | MKYLDSDNPRYVKFVKANNGTLKASVLEKEEFNKLKKQAIAW |
| | | KKDNKDADSEVSILRSGKHFYIRKELEAIVKAKLCPEVEEETVK |
| | | AK |
| 53 | EpiCas055 | MATRTSHKSSKIRILPTGTTKKALVEYNLLFHDQVAKIMLDLLT |
| | | MRGVPYVLRDKDGEHEITATQIREAQWKLINSGRDSGDCIRLSQ |
| | | SIIDMMAQIYGGIMETGNPTKYRANLYLKKTNSGNEIGQGRSSA |
| | | SFMLFKDVSRGLSRPEFTRQIKDKNGKIKKPFAWATEERLMELY |
| | | DAVRNDEMQPIEVEAERKSLEPDVTMENLVKLRKVGILPLPLPG |
| | | GHLYQGRGLGKLLAMPFDAAVGRLKSHYQANLAAVEARAAL |
| | | DKVVADKKDNVPAPLYALFLEFAEECRQNGFGVSKRFAKKLH |
| | | GLNIPKTDFEPGDPFIVGLEIIKQYKYRPLWPQIGTLAGVYLAGV |
| | | KAEKTKGYAAITIADAYRSPALIRFGITNYARFEAEVNDQQGID |
| | | FSLQIPVDDQWTTVTVSGMPSNYFQNPELIRQTSNYALSFTKGN |
| | | RQQRRMGVILKEPLLTVRYSSDPEQEPRFYVHLAYNYDKTSYR |
| | | MIGLDKIRAFFSSNLSKGAKLPHGTPYGLRTLSIDLGIRPIISSHL |
| | | MELVSPHDIEAAQESRNGIEPIEVPDMGFARLVDSREMGGVWN |
| | | SGLVGRMIDASRRCKSARHMILVSKLMRDGKPLNTHQQFLIEK |
| | | YVKMYGVFLPPDDWARKDIIGEFIRQILADYRVIKDTMAKRNH |
| | | PDHKGKFPDIICSDVFRMLFLIKDVISMLKSWARYKRKTGDKEI |
| | | QGQNPTELNQYWRYYNNLKDDLLKKIVHSLVRYADEHGVHLL |
| | | LLEDITGNRDSTKHRKENSMFAIWSPTLIRTRLEEEFEACGMLV |
| | | QAINPSFTSQTHYMTGEFGFRDPRQKECLYFKREGRLYLIDSDG |
| | | NSSMGLQVRFWTRHRDLWTLKTVKARRDRAAKLLKLDPKRA |
| | | ECTFVAKPTFEYAKSYLKRNLASPVAAFVPDDNGNFILHKLGV |
| | | NAANQLTEGQERGETFFRHGTDWITHASHKKRMSAIEAMVEK |
| | | VKKKGDKMTEADLVRLKV |
| 54 | EpiCas056 | MSSDVVRPYNTKLLPDNRKHNMVVDTMNWCDNACSVFFDLF |
| | | VGLYGGVKIGDVTNFLTEKQDTTTDADLLCAINWFRAMPVSNP |
| | | DAVVGPEQLVVRFRDYSGHNPSPMASMYLSAPVNPRYTWVDC |
| | | RFLYQQMCQTLGANLDVDISTLVQTGLLPIQKDTQLNAYSFCS |
| | | NVFGEGVKEDPSAKVTSYTAAANALSGATNPIITYEDYRQILSS |
| | | AITNRPSRKGAKDILAADLDSTKTGVLPGNYVSQRIAMYQASA |
| | | ASKARGVVLPNRLRVKAYLQNLVGLNYICTTWAAPMLRAVGD |
| | | IKSKNSNNYRFVGERLERQQEIDNLLAVSNIRSAQAILQQFRAN |
| | | ESNQFYIKKRHIGKGLPSLYDMWDLMDEKEGIDNYIQSLKGDK |
| | | TERLPIRELLQIIYPHRKSVSAELFLSAAELTDVEDRQNRCMVHP |
| | | TVRGSLVINFGKSSTIFGAITPPDTLIKFRGEMVPAGQTGMIWFT |
| | | MHLVDGGKWVKHHIPCHSSRYFEEIYAWKKDLPTLPFPRRSLY |
| | | GYAIGNKISDTRKIDLRRKKASKQYLRTLENMTHNVEFDPTTQF |
| | | MVDQELNVTITARVKKIPPVRDVPVGSRIMGLNKNQTIRDCYSI |
| | | WERVEANTSGSFQHPVVQDVWLRLVADGHVTSTVNGPNGQID |
| | | QMSYSGPDVANCGNWMQERSNFITSVDDADQSIMKHFLWNNR |
| | | VKLYAWNCVYAGTLMKAISKQMTTANRNLFRNEILSFVGGRF |
| | | GCRLGSLAQVSLDLLRRMKSVISCYFSRNNAFTIEERSAFDKEL |
| | | MELFESLDNKMNNKRKEKVNRTLSSIFQVAKQHNVVRLVVTK |
| | | NLQTPDANVRSVMNQRSVDWCARKIEEKIVDLGNVIGIYVDLV |
| | | EALDLSHLDPFIYIAKGDVGKECQFDEVESSSLLPIHTSMFRDW |
| | | HGLVNKGRTTTPIYHSALRSFASHYGLRFDDLARIKAAELAKM |
| | | LPDQKVFIPHRGGRFFMSTYPVTSTSRPVSYDGRQRFLNSGDMV |
| | | APVNTMLRGFRRARS |
| 55 | EpiCas057 | MGITMSDTTFRPYKSKLNPNERKMPMVVDTMNWCDNACSVFF |
| | | DLFVGLYGGVKIGDVTNFLTEKQDTTTDADLLCAINWFRAMPV |
| | | SNPDAVVGPEQLVVRFRDYSGHNPSPMASMYLSAPVNPRYTW |
| | | VDCRFLYQQMCQTLGANLDVDISTLVQTGLLPIQKDTQLNAYS |
| | | FCSNVFGEGVKEDPSAKVTSYTAAANALSGATNPIITYEDYRQI |
| | | LSSAITNRPSRKGAKDILAADLDSTKTGVLPGNYVSQRIAMYQA |
| | | SAASKARGVVLPNRLRVKAYLQNLVGLNYICTTWAAPMLRAV |
| | | GDIKSKNSNNYRFVGERLERQQEIDNLLAVSNIRSAQAILQQFR |
| | | ANESNQFYIKKRHIGKGLPSLYDMWDLMDEKEGIDNYIQSLKG |
| | | DKTERLPIRELLQIIYPHRKSVSAELFLSAAELTDVEDRQNRCMV |
| | | HPTVRGSLVINFGKSSTIFGAITPPDTLIKFRGEMVPAGQTGMIW |
| | | FTMHLVDGGKWVKHHIPCHSSRYFEEIYAWKKDLPTLPFPRRS |
| | | LYGYAIGNKISDTRKIDLRRKKASKQYLRTLENMTHNVEFDPTT |
| | | QFMVDQELNVTITARVKKIPPVRDVPVGSRIMGLNKNQTIRDC |
| | | YSIWERVEANTSGSFQHPVVQDVWLRLVADGHVTSTVNGPNG |
| | | QIDQMSYSGPDVANCGNWMQERSNFITSVDDADQSIMKHFLW |
| | | NNRVKLYAWNCVYAGTLMKAISKOMTTANRNLFRNEILSFVG |
| | | GRFGCRLGSLAQVSLDLLRRMKSVISCYFSRNNAFTIEERSAFD |
| | | KELMELFESLDNKMNNKRKEKVNRTLSSIFQVAKQHNVVRLV |
| | | VTKNLQTPDANVRSVMNQRSVDWCARKIEEKIVDLGNVIGIYV |
| | | DLVEALDLSHLDPFIYIAKGDVGKECQFDEVESSSLLPIHTSMFR |
| | | DWHGLVNKGRTTTPIYHSALRSFASHYGLREDDLARIKAAELA |
| | | KMLPDQKVFIPHRGGRFFMSTYPVTSTSRPVSYDGRQRFLNSGD |
| | | MVAPVNTMLRGFRRARS |
| 56 | EpiCas058 | MGITMSDTTFRPYKSKLNPNERKMPMARNNKKLEGLVKSYKF |
| | | QAKNVDIMIDILEEYSEYYNRVSNWINDNLMTLTIGDIALKCNT |
| | | SQSVGYMQIALSDDYKQLPMYKLFEKKYPSTHCLNLLYAYITN |
| | | NNIDNYTGNIWGLSTTNYRKYGYINLVINNYRSKFANLNFNVK |
| | | RSKIDINSNVEEIEKQCLYERIRLGLIKIEDWKKHIDSLKRQEIINE |
| | | KALHRFNILSDYFNKNGESINERQSLLTIEQLQKENGCQMKNKS |
| | | MRIQVQNFKLNEKPNSLGYILQIHLNKKLFNIELLGNRQIRKGN |
| | | KDNYTTLVDIINNHGIDIIFKLENNKLYVILTSTDTTENKTITTPN |
| | | KICGIDINIKHALLNTSERDSMNIQGYVNLYKRVLENDNFVSLLS |
| | | KEEYETFSNMASYLTFCPIEFNYLYSRISNDEYTEKERLFSKILID |
| | | IQKEYENKKGCDLEKNYIHSVNKLRAKIIAYHKLKTIYTQKKQE |
| | | YDIKMGFVDDSTLSKDGMDKRRFESPFNNTDCAKQIKSKLDNV |
| | | LQDIEGCRKNIITYVYKIFETNGYVTIGLENLDNSNFEYHKPLCSI |
| | | KSLLNYHKLLNKTIEQVKENNSVADLIEKDYYIFTFDENKNIIDA |
| | | KFSDKGFYKINKSWFINQLIKVLHFASIKNYFITLSNNGKTQIVL |
| | | VPSEFTSQINSLTHKLPMIKNDKGENVRPTKFRVRKTQEKHYNG |
| | | LNADINATLNIKYIVENEEWRNVLCKSPNKKEYKDARGYNFPT |
| | | LIATQKHYNVSKQLEKLGGIEFIQ* |
| 57 | EpiCas059 | MEETRTPEKIACQQDRSAFFKLGKLPQKTTSAIYHLMRCSKRYI |
| | | DNVSETISNNIEKAAMTSDNHIFRSALKEEYKKIYREKFYPDVT |
| | | GQPFPDKGPDNTHIVERTVENLCGQYISALKGVMKVRKPKWQ |
| | | DDIFEDESDKVIAEMLMASGNEADIHPKLEKKIASAKKKDKMD |
| | | EWQKVLDRYDYIKSKTDIEWTYTTLLYDHIARECARSMFEDGN |
| | | KPKFCYTNGNKFAKQVVDELWGSERVEYEYEKYLHCRTLEAY |
| | | WNEENKYFFLENQKKSKKTYEARQIIDHFIENEDAVKAETEEY |
| | | MAKVAKEFGENGGCKFRTENILYLVNSVSINKKQDEERCPIFKV |
| | | TDIPQYAEAGIDRIGECKYVIKLKLFTVGERYGTTYMPEAFSGL |
| | | YDRRTNQRYDLIDRACKGSYVYLPIYISDKDSRKWSDRLKNTF |
| | | TMSAFSISERKTKKDGRVLCVNLPYTHIQHKPRNYFKVYPTYG |
| | | VDINVKHSLFSTNATNLNGYIDVNGELIDFYNSIGRPDKLKNINL |
| | | AVTLGDKGGTAGNHTNFLLVSDLGDWDDHGKKRNDRFVKRE |
| | | DGTYYSRPDLLADGLNFLKKKYSGDRKSYDYINTTIAFRNNQT |
| | | RLRCIDKEWNDRKRAYDEEHGKPEKGERSGFYPTEDAERLTME |
| | | RQALRREIAENTRQVIAYMLWLFQENGIKSIALEHLDCGSWKN |
| | | FRDQLKPKWFCNNCKYWAEKYGIPYEATKYYRSNRKYFDIED |
| | | GELIPENVKLNALGEAENDKRYFQTSIAKAVGMSSIKQMFSIVS |
| | | QNYDIQVNYTEQAYTSQTDSRNGTVDKDFRVSQECYIGSDGTK |
| | | MNCDFNAAVNIADRISDDTLREEFNKELPMKEFVKYGATQIVS |
| | | RNRRWNAENAERVTMAAFQKRKNADGGAHTAAVKPTSKTEE |
| | | SSLKLVAE |
| 58 | EpiCas060 | MIDRNETHLSKKSTRSVEFKLAVPNRIAPVIDEMLDLSITMSNAI |
| | | SKDISEHLTDWVQRAEVTKEQKRPYDYLYASEKEGIRYWYKHS |
| | | GLRDKMYFDFCSKYFGYPVLKNKNAHFLTRTIENVAAAYEGRF |
| | | TRFQEETKIRATDQSQLTEEEELLKKNIKEILNIMCSLKPGQTVD |
| | | QILVRRSKTGKNQEKYQKLLSFYVDVTSDEKSDKYINFDILFAD |
| | | MCYRNSATFILKTMEISVDDSGVVNFGMVSPLVELPTKIGSSEE |
| | | ATMRHILENRSVRLINEVNDMIFELTVAQYIKSSCRYRFVDELT |
| | | EKANSKDSVFHKNLLMYYTSHRDKVDEGYKQYLHSVNESFGK |
| | | KQIVWKNSTLKLNYEEKVEKDRQVPIKMISSIKQFADLGITSISG |
| | | CKYVLRVHMILDDEKKFKDQHLFCDLYDKRTGRRFDLLDHET |
| | | KGYAYIPIMVPENDRRTYDISDFDCQMTEISSRDGMYFAKLTRD |
| | | IDYVKPVNRFGEEQVCGVDINIRHAMIDVSERSIMNSEKYVDVE |
| | | GFIIDYFRNVKNLPSVADDLVTNRYLGEAEEAGKHTFFGLMSSI |
| | | NKSISRETGGLKNRFVKNVDGTYTILPDCFSEPLNVLMKKYEQG |
| | | SLQYRYVASVKALRVQLAQMHVLCMEHDDRRQVYDNLYGKP |
| | | YIDDHPYEDPDFVYAHRKLHQKIETNTQFIVSYAIQLIQEAGFSC |
| | | ISVEQIDKKNLGKMLNLKSAKVFYNGCIYASSENGQAIADTRYF |
| | | RTNCRYFDYPSDLNLFDIDHIKLNGVGEAYNINAKLMNSLGKY |
| | | VGFASFKHMFAHIGTAHDVQVVFVEPAFTSQVDSKTGELRADK |
| | | KEMRPYHHTYKHTDGTVSHADENGAINAKKIVTEEILRTRLLK |
| | | ENSVKDIMEYGGVQLERSGKSINGVRKVYEEVYAEKNGLV* |

In certain embodiments, the Cas enzyme has a catalytically active domain capable of binding to a target DNA strand and/or a catalytically active domain capable of cleaving the target DNA strand.

In certain embodiments, the catalytically active domain comprising an alteration of one or more amino acids such that the Cas enzyme has only the activity of binding to the target DNA strand, or has the activity of binding to the target DNA strand and the activity of cleaving a single strand of the target DNA.

In another aspect, the present application provides a fusion molecule, which comprises the Cas enzyme described in the present application and one or more heterologous functional domains.

In certain embodiments, the one or more heterologous functional domains are capable of regulating expression of one or more gene products.

In certain embodiments, the one or more heterologous functional domains are directly or indirectly fused to the Cas enzyme.

In certain embodiments, the one or more heterologous functional domains are selected from helicases, nucleases, helicase-nucleases, DNA methyltransferases, DNA hydroxymethylases, histone methylases, histone demethylases, histone acetyltransferases, histone deacetylases, phosphatases, kinases, transcriptional (co-) activators, transcriptional repressors, DNA binding proteins, DNA structural proteins, marker proteins, reporter proteins, fluorescent proteins, ligand binding proteins, signal peptides, subcellular localization sequences, antibody epitopes, and affinity purification tags.

In certain embodiments, the one or more heterologous functional domains have one or more of the following activities: methylase activity, demethylase activity, deaminase activity, transcriptional activation activity, transcriptional repression activity, transcriptional release factor activity, reverse transcriptase activity, histone modification activity, RNA cleavage activity, and nucleic acid binding activity.

In another aspect, the present application provides an engineered, programmable, non-naturally occurring CRISPR-Cas system, the system comprises the Cas enzyme of the present application or the fusion molecule of the present application, and one or more guide RNAs; the one or more guide RNAs target a locus of a nucleic acid molecule encoding one or more gene products in a cell, thereby directing the Cas enzyme or the fusion molecule to bind to and/or cleave the locus of the nucleic acid molecule encoding the one or more gene products; and the Cas enzyme or the fusion molecule does not naturally co-occur with the guide RNA.

In another aspect, the present application provides an engineered, non-naturally occurring vector system, the vector system comprises one or more vectors comprising: a) a first regulatory element operably linked to one or more guide RNAs capable of hybridizing to a target sequence in a locus of a nucleic acid molecule encoding one or more gene products, and b) a second regulatory element operably linked to the Cas enzyme of the present application or the fusion molecule of the present application, wherein the component a) and the component b) are located on the same vector or different vectors of the vector system, and the guide RNA targets the locus of the nucleic acid molecule encoding the one or more gene products in a cell, thereby directing the Cas enzyme or the fusion molecule to bind to and/or cleave the locus of the nucleic acid molecule encoding the one or more gene products; and the Cas enzyme or the fusion molecule does not naturally co-occur with the guide RNA.

In certain embodiments, the expression of the one or more gene products is altered.

In certain embodiments, the expression of the gene product is decreased or increased.

In certain embodiments, the gene product is a protein.

In certain embodiments, the cell is a eukaryotic cell.

In certain embodiments, the eukaryotic cell is a mammalian cell. For example, the mammalian cell includes, but are not limited to, cells of mice, monkeys, humans, farm animals, sports animals, and pets.

In certain embodiments, the mammalian cell is a human cell.

In certain embodiments, the Cas enzyme is codon-optimized for expression in eukaryotic cells.

In certain embodiments, the guide RNA comprises a guide sequence fused to a tracr sequence.

In certain embodiments, the guide RNA comprises a direct repeat sequence and a spacer sequence, and the spacer sequence binds to a nucleic acid molecule targeted by the guide RNA.

In certain embodiments, the direct repeat sequence is 10 to 70 nucleotides in length.

In certain embodiments, the direct repeat sequence is 31 to 36 nucleotides in length.

In certain embodiments, the direct repeat sequence comprises a nucleotide sequence as set forth in any one of SEQ ID NO: 63-88 and 90-99, or comprises a nucleotide sequence having at least 95% sequence identity to the nucleotide sequence as set forth in any one of SEQ ID NOs: 63-88 and 90-99.

In certain embodiments, the spacer sequence is 16 to 24 nucleotides in length.

In certain embodiments, the nucleic acid molecule targeted by the guide RNA comprises a nucleotide sequence capable of complementary pairing with the spacer sequence.

In certain embodiments, the vector or the Cas enzyme of the system further comprises one or more nuclear localization sequences (NLS).

In certain embodiments, the system is introduced into the cell by a delivery system, and the delivery system is selected from a virion, a liposome, a lipid nanoparticle, electroporation, microinjection, and conjugation.

In another aspect, the present application provides a method for altering the expression of one or more gene products, the method comprises introducing an engineered, non-naturally occurring CRISPR-Cas system into a cell comprising and expressing a nucleic acid molecule encoding the one or more gene products, and the system comprises the Cas enzyme of the present application or the fusion molecule of the present application, and one or more guide RNAs, and the one or more guide RNAs target a locus of the nucleic acid molecule encoding the one or more gene products, thereby directing the Cas enzyme or the fusion molecule to bind to and/or cleave the locus, thus altering the expression of the one or more gene products; and the Cas enzyme or the fusion molecule does not naturally co-occur with the guide RNA.

In another aspect, the present application provides a method for altering the expression of one or more gene products, wherein the method comprises introducing an engineered, non-naturally occurring vector system into a cell comprising and expressing a nucleic acid molecule encoding the one or more gene products, the system comprises one or more vectors, and the one or more vectors comprises: a) a first regulatory element operably linked to one or more guide RNAs capable of hybridizing to a target sequence in a locus of the nucleic acid molecule encoding the one or more gene products, and b) a second regulatory element operably linked to the Cas enzyme of the present application or the fusion molecule of the present application, wherein the component a) and the component b) are located on the same vector or different vectors of the vector system, and the guide RNA targets the locus of the nucleic acid molecule encoding the one or more gene products in the cell, thereby directing the Cas enzyme or the fusion molecule to bind to and/or cleave the locus, thus altering the expression of the one or more gene products; and the Cas enzyme or the fusion molecule does not naturally co-occur with the guide RNA.

In certain embodiments, the expression of the gene product is decreased or increased.

In certain embodiments, the gene product is a protein.

In certain embodiments, the cell is a eukaryotic cell.

In certain embodiments, the eukaryotic cell is a mammalian cell. For example, the mammalian cell includes, but are not limited to, cells of mice, monkeys, humans, farm animals, sports animals, and pets.

In certain embodiments, the mammalian cell is a human cell.

In certain embodiments, the Cas enzyme is codon-optimized for expression in eukaryotic cells.

In certain embodiments, the guide RNA comprises a guide sequence fused to a tracr sequence.

In certain embodiments, the guide RNA comprises a direct repeat sequence and a spacer sequence, and the spacer sequence binds to a nucleic acid molecule targeted by the guide RNA.

In certain embodiments, the direct repeat sequence is 10 to 70 nucleotides in length.

In certain embodiments, the direct repeat sequence is 31 to 36 nucleotides in length.

In certain embodiments, the direct repeat sequence comprises a nucleotide sequence as set forth in any one of SEQ ID NO: 63-88 and 90-99, or comprises a nucleotide sequence having at least 95% sequence identity to the nucleotide sequence as set forth in any one of SEQ ID NOs: 63-88 and 90-99.

In certain embodiments, the spacer sequence is 16 to 24 nucleotides in length.

In certain embodiments, the nucleic acid molecule targeted by the guide RNA comprises a nucleotide sequence capable of complementary pairing with the spacer sequence.

In certain embodiments, the vector or the Cas enzyme of the system further comprises one or more nuclear localization sequences (NLS).

In certain embodiments, the method comprises introducing the CRISPR-Cas system or the vector system into the cell by a delivery system, and the delivery system is selected from a virion, a liposome, a lipid nanoparticle, electroporation, microinjection, and conjugation.

In another aspect, the present application provides a nucleic acid encoding the Cas enzyme of the present application, the fusion molecule of the present application, or the CRISPR-Cas system of the present application.

In another aspect, the present application provides a cell comprising the Cas enzyme of the present application, the fusion molecule of the present application, the CRISPR-Cas system of the present application, the vector system of the present application and/or the nucleic acid of the present application.

In another aspect, the present application provides a kit comprising the Cas enzyme of the present application, the fusion molecule of the present application, the CRISPR-Cas system of the present application, the vector system of the present application, the nucleic acid of the present application and/or the cell of the present application.

In certain embodiments, the kit further comprises a container for placing the Cas enzyme, the fusion molecule, the CRISPR-Cas system, the vector system, the nucleic acid and/or the cell, and an instruction for use.

Those skilled in the art may easily discern other aspects and advantages of the present application from the following detailed description. Only exemplary embodiments of the present application are shown and described in the following detailed description. As those skilled in the art will realize, the contents of the present application enable those skilled in the art to make modifications to the specific embodiments disclosed without departing from the spirit and scope of the invention to which the present application relates. Accordingly, the drawings and descriptions in the specification of the present application are illustrative only and not limited.

### BRIEF DESCRIPTION OF DRAWINGS

The specific features of the invention to which the present application relates are as shown in the appended claims. The features and advantages of the invention to which the present application relates may be better understood by reference to the exemplary embodiments and the drawings described in detail below. The accompanying drawings are briefly described as follows:

FIGS. 1A-1B show a schematic process diagram and fluorescence results of the assay of cleavage activity of the Cas enzyme of the present application in eukaryotic cells, and the PAM motif required for gRNA binding of the Cas enzyme.

FIGS. 2A-2B show a schematic process diagram of the assay of cleavage activity of the Cas enzyme of the present application in eukaryotic cells, and the comparison results of green fluorescent cells of different Cas enzymes.

FIGS. 3A-3B show a schematic process diagram of the assay of the in vitro cleavage activity of the Cas enzyme of the present application and the PAM sequence of the Cas enzyme, as well as sequencing results.

FIG. 4 shows site information and amplification detection results of detecting the Cas enzyme activity of the present application at endogenous sites.

FIGS. 5A-5C show the GFP fluorescence results after cleavage activity assay of different variants of the Cas enzyme of the present application in eukaryotic cells.

FIGS. 6A-6B show the results of the proportion of green fluorescent cells after cleavage activity assay of the Cas enzyme of the present application combined with sgRNAs processed by different Direct Repeat (DR) region optimization schemes in eukaryotic cells.

FIG. 7 shows the results of the proportion of green fluorescent cells after cleavage activity assay of the Cas enzyme of the present application combined with different sgRNAs with different spacer lengths in eukaryotic cells.

FIGS. 8A-8B show the results of the proportion of green fluorescent cells after cleavage activity assay of different variants of the Cas enzyme of the present application in eukaryotic cells. FIG. 8C shows the results of cleavage activity assays (proportion of green fluorescent cells) of different variants of the Cas enzyme of the present application in different PAM reporter systems.

FIGS. 9A-9C show schematic structural diagrams of cytosine base editors comprising the Cas enzyme of the present application, and the results of testing the base editing efficiency thereof at two endogenous sites, EMX1 and VEGFA.

FIG. 10 shows the activation effect of the gene activation epigenetic tool comprising the Cas enzyme of the present application on detecting target site gene expression at the endogenous site of CXCR4.

### DETAILED DESCRIPTION

The embodiments of the invention of the present application will be illustrated below with specific examples. Those skilled in the art may easily understand other advantages and effects of the invention of the present application from the disclosure of the specification.

### DEFINITION OF TERMS

In the present application, the term “identity” is used interchangeably with “homology”, which generally refers to the relationship between two or more polypeptide molecules or two or more nucleic acid molecule sequences, which relationship is determined by comparing these sequences. In the art, “identity” also refers to the degree of relatedness of nucleic acid molecules or polypeptide sequences, as determined by the match between two or more nucleotides or two or more amino acids. In the present application, percent identity (%) of amino acid sequences is defined as the percentage of amino acid residues in a candidate sequence that are identical to the amino acid residues in a reference polypeptide sequence to the total number of residues, after aligning the sequences and introducing gaps, if necessary, to achieve the maximum percent sequence identity, and not considering any conservative substitutions as part of the sequence identity. Alignment for the purpose of determining percent amino acid sequence identity can be achieved in a variety of ways within the skill in the art, for example, using publicly available computer software such as BLAST, BLAST-2, ALIGN, or Megalign (DNASTAR) software. Those skilled in the art can determine appropriate parameters for aligning sequences, including any algorithm required to achieve maximum alignment over the full length of the sequences being compared. In certain embodiments, the calculation of the percent identity (%) of a polypeptide or nucleic acid sequence can also determine the total number of residues based on the type of sequence mutation. Types of mutations include insertions (extensions) at either or both ends of the sequence, deletions (truncations) at either or both ends of the sequence, substitutions/replacements of one or more amino acids/nucleotides, insertions within the sequence, and deletions within the sequence. Taking the amino acid sequence of a polypeptide as an example (the same applies to the nucleotide sequence), if the type of mutation is one or more of the following: substitution/replacement of one or more amino acids/nucleotides, insertion within the sequence, and deletion within the sequence, the total number of residues is calculated based on the larger one in the compared molecules. If the type of mutation also includes insertions (extensions) at either or both ends of the sequence or deletions (truncations) at either or both ends of the sequence or insertions within the sequence and deletions within the sequence, the number of amino acids inserted or deleted at either or both ends or within the sequence (e.g. less than 20 insertions or deletions at both ends) is not counted in the total number of residues. In calculating percent identity, the sequences being compared can be aligned in a manner that produces the greatest match between the sequences, and gaps in the alignment, if present, can be resolved by a specific algorithm.

In the present application, a “catalytically active domain” refers to an identifiable or determinable conserved structural entity within a Cas protein (enzyme) that exhibits a significant secondary structure content, and the conserved structure is the region where the Cas protein (enzyme) performs functions such as binding and/or cleaving polynucleotides. An exemplary catalytically active domain may be a Cas9 family protease having two catalytically active domains, one being HNH-like, which functions to cleave a single-stranded polynucleotide (target strand) paired with a guide RNA, and the other being RuvC-like, which functions to cleave the complementary strand of the target strand.

In the present application, the term “binding” (e.g., with respect to a target DNA binding (catalytically active) domain of a polypeptide or protease) generally refers to a non-covalent interaction between macromolecules (e.g., between proteins and nucleic acids). A macromolecule is referred to as “associated” or “interacting” or “bound” when in a state of non-covalent interaction (e.g., when molecule X is referred to as interacting with molecule Y, it is meant that molecule X binds molecule Y in a non-covalent manner). It will be appreciated that not all binding interaction components need to be sequence-specific (e.g., contact with phosphate residues in the DNA backbone), but some portions of the binding interaction may be sequence-specific.

In the application, the term “fusion molecule” generally refers to a molecule composed of at least two moieties (bipartite molecule) comprising an enzyme (protein or peptide) of the present application coupled to at least one additional moiety to form a single entity. The enzyme and the at least one additional moiety may be separated by a linker, or may be directly coupled. The at least one additional moiety may be fused to an enzyme of the present application at any amino acid other than the N-terminal amino acid, C-terminal amino acid, or terminal amino acids. The additional moiety may be fused to moieties already included in the fusion molecule. The optimal order and/or combination of assays for determining moieties in the fusion molecules of the present application are well known to those skilled in the art. Generally, when the fusion molecule comprises an enzyme of the present application and at least one additional peptide, the term does not include such fusion molecules in which the fusion produces a naturally occurring peptide.

In the present application, the term “heterologous” generally refers to a nucleotide or polypeptide sequence that is not found in a naturally occurring nucleic acid or protein, respectively. In certain embodiments of the present application, the term “heterologous functional domain” may refer to about or more than about 1, 2, 3, 4, 5, 6, 7, 8, 9, 10 or more domains other than the Cas enzyme of the present application, or a portion of the fusion molecule of the present application. Examples of heterologous functional domains that can be included in the fusion molecules of the present application or can be fused to the Cas enzymes of the present application include, but are not limited to, epitope tags, reporter gene sequences, and protein domains having one or more of the following activities: methylase activity, demethylase activity, transcriptional activation activity, transcriptional repression activity, transcriptional release factor activity, histone modification activity, RNA cleavage activity, and nucleic acid binding activity. Non-limiting examples of epitope tags include histidine (His) tags, V5 tags, FLAG tags, influenza virus hemagglutinin (HA) tags, Myc tags, VSV-G tags, and thioredoxin (Trx) tags. Examples of reporter genes include, but are not limited to, glutathione-S-transferase (GST), horseradish peroxidase (HRP), chloramphenicol acetyltransferase (CAT), beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). The Cas enzyme can be fused to a gene sequence encoding a protein or protein fragment that binds to a DNA molecule or binds to other cellular molecules including, but not limited to, maltose binding protein (MBP), S-tag, Lex A DNA binding domain (DBD) fusions, GAL4 DNA binding domain fusions, and herpes simplex virus (HSV) BP16 protein fusions. Additional domains that can form part of a fusion molecule comprising a Cas enzyme are described in US20110059502, which is incorporated herein by reference.

In the present application, the term “expression” generally refers to the process by which a DNA template is transcribed into a polynucleotide (e.g., into mRNA or other RNA transcript) and/or the process by which the transcribed mRNA is subsequently translated into a peptide, polypeptide or protein. The transcript and the encoded polypeptide may be collectively referred to as a “gene product”. If the polynucleotide is derived from genomic DNA, expression may include splicing of mRNA in eukaryotic cells.

In the present application, the terms “polynucleotide,” “nucleotide,” “nucleotide sequence,” “nucleic acid,” and “oligonucleotide” are used interchangeably. They generally refer to a polymeric form of nucleotides (either deoxyribonucleotides or ribonucleotides, or analogs thereof) of any length. A polynucleotide may have any three-dimensional structure and may perform any function, known or unknown. The following are non-limiting examples of polynucleotides: coding or non-coding regions of a gene or gene fragment, multiple loci (a locus) defined by linkage analysis, exons, introns, messenger RNA (mRNA), transfer RNA, ribosomal RNA, short hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. Polynucleotides may include one or more modified nucleotides, such as methylated nucleotides and nucleotide analogs. If present, modifications to the nucleotide structure may be conducted before or after polymer assembly. The sequence of nucleotides may be interrupted by non-nucleotide components. Polynucleotides may be further modified after polymerization, for example, by conjugation with a labeling moiety.

In the present application, the terms “non-naturally occurring” and “engineered” are used interchangeably. When they refer to nucleic acid molecules, polypeptides, or combinations thereof and systems thereof, it is generally meant that the nucleic acid molecules or polypeptides are at least substantially free from at least another component with which they are associated in nature or as found in nature.

In the present application, the term “vector” generally refers to a nucleic acid molecule that is capable of transporting another nucleic acid molecule to which it is linked. Vectors include, but are not limited to, single-stranded, double-stranded, or partially double-stranded nucleic acid molecules; nucleic acid molecules comprising one or more free ends, or no free ends (e.g., circular); nucleic acid molecules comprising DNA, RNA, or both; and a wide variety of other polynucleotides known in the art. One type of vector is a “plasmid”, which refers to a circular double-stranded DNA loop into which additional DNA fragments can be inserted, for example, by standard molecular cloning techniques. Another type of vector is a viral vector in which a virus-derived DNA or RNA sequence is present in a vector used to package viruses (e.g., retroviruses, replication defective retroviruses, adenoviruses, replication defective adenoviruses, and adeno-associated viruses). Viral vectors also include polynucleotides carried by a virus for transfection into a host cell. Certain vectors (e.g., bacterial vectors having a bacterial origin of replication and episomal mammalian vectors) are capable of autonomous replication in a host cell into which they are introduced. Other vectors (e.g., non-episomal mammalian vectors) are integrated into a host cell genome upon introduction into the host cell, and thereby replicate along with the host genome. Furthermore, certain vectors are capable of directing the expression of the genes to which they are operably linked. Such vectors are referred to herein as “expression vectors”. Common expression vectors used in recombinant DNA technology are typically in the form of plasmids. The recombinant expression vectors may comprise the nucleic acid of the present application in a form suitable for expression of the nucleic acid in a host cell, meaning that these recombinant expression vectors comprise one or more regulatory elements selected based on the host cell to be used for expression, and the regulatory elements is operably linked to the nucleic acid sequence to be expressed. In a recombinant expression vector, “operably linked” is intended to mean that a nucleotide sequence of interest is linked to the one or more regulatory elements in a manner that allows the expression of the nucleotide sequence (for example, in an in vitro transcription/translation system or in a host cell when the vector is introduced into the host cell).

In the present application, the term “regulatory element” is generally intended to include promoters, enhancers, internal ribosome entry sites (IRES), and other expression control elements (e.g., transcription termination signals such as polyadenylation signals and polyU sequences). Such regulatory sequences are described, for example, in Goeddel, GENE EXPRESSION TECHNOLOGY: METHODS IN ENZYMOLOGY 185, Academic Press, San Diego, CA (1990). In certain embodiments, regulatory elements may include those sequences that direct constitutive expression of a nucleotide sequence in many types of host cells as well as those sequences that direct expression of the nucleotide sequence only in certain host cells (e.g., tissue-specific regulatory sequences). A tissue-specific promoter may primarily direct expression in a desired tissue of interest, such as muscle, neurons, bone, skin, blood, specific organs (e.g. liver, pancreas), or specific cell types (e.g. lymphocytes). The regulatory elements may also direct expression in a timing-dependent manner, such as in a cell cycle-dependent or developmental stage-dependent manner, which may or may not be tissue- or cell type-specific. In certain embodiments, a vector may comprise one or more pol III promoters (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or a combination thereof. Examples of pol III promoters include, but are not limited to, the U6 and H1 promoters. Examples of pol II promoters include, but are not limited to, the reverse transcription Rous sarcoma virus (RSV) LTR promoter (optionally with an RSV enhancer), the cytomegalovirus (CMV) promoter (optionally with a CMV enhancer), the SV40 promoter, the dihydrofolate reductase promoter, the beta-actin promoter, the phosphoglycerol kinase (PGK) promoter, and the EFla promoter. The term “regulatory element” may also encompass enhancer elements such as WPRE, CMV enhancers, R-U5′ fragments in the LTR of HTLV-I, SV40 enhancers; and the intron sequence between exons 2 and 3 of rabbit β-globin (Proc. Natl. Acad. Sci. USA., Vol. 78 (3), pp. 1527-31, 1981).

Those skilled in the art will appreciate that the design of the expression vector may depend on factors such as the choice of the host cell to be transformed, the desired expression level, and the like. A vector can be introduced into a host cell to thereby produce a transcript, protein, or peptide, including a fusion molecule or enzyme encoded by a nucleic acid of the present application (e.g., a clustered regularly interspaced short palindromic repeat (CRISPR) transcript, protein, enzyme, mutant form thereof, fusion molecule or fusion protein thereof, etc.). Advantageous vectors include lentiviruses as well as adeno-associated viruses, and vectors of this type can also be selected to target specific types of cells.

In the present application, the term “codon optimization” generally refers to a method of modifying a nucleic acid sequence in order to enhance expression in a host cell of interest by replacing at least one codon of the native sequence, for example about or more than about 1, 2, 3, 4, 5, 10, 15, 20, 25, 50, or more codons, with a codon that is more frequently or most frequently used in a gene of the host cell, while maintaining the native amino acid sequence. Different species exhibit specific preferences for certain codons with specific amino acids. Codon preference (differences in codon usage between organisms) is often associated with the translation efficiency of messenger RNA (mRNA), which is believed to depend, among other things, on the nature of the codon being translated and the availability of a particular transport RNA (tRNA) molecule. The dominance of selected tRNAs within a cell generally reflects the codons most frequently used for peptide synthesis. Thus, genes can be tailored to optimal gene expression in a given organism based on codon optimization. Codon utilization tables are readily available, for example, in a Codon Usage Database (“Codon Usage Database”), and these tables can be adapted in different ways. For example, see Nakamura Y. (Nakamura Y.) et al., Codon usage tabulated from the international DNA sequence databases: status for the year 2000, Nucl. Acids Res. 28:292 (2000). In silico algorithms for codon optimization of specific sequences for expression in specific host cells are also available, such as Gene Forge (Aptagen, Jacobus, PA). In certain embodiments, one or more codons (e.g., 1, 2, 3, 4, 5, 10, 15, 20, 25, 50, or more, or all codons) in the sequence encoding the Cas enzyme correspond to the codon most frequently used for a particular amino acid.

In the present application, the term “guide RNA” is used interchangeably with “gRNA”, and generally refers to a group of nucleic acid molecules that facilitate the specific direction of an RNA-guided nuclease or other effector molecule (usually complexed with a gRNA molecule) onto a target sequence. In nature, crRNA and tracrRNA usually exist as two independent RNA molecules that make up gRNA. The term “tracrRNA” generally refers to a scaffold RNA that can bind to a Cas nuclease, and the term “crRNA”, also referred to as a CRISPR RNA, generally refers to a stretch of nucleotide sequence that is complementary to the target DNA being targeted. The crRNA and tracRNA may also be fused into a single strand, in which case the gRNA may also be referred to as a single-stranded guide RNA (sgRNA). The sgRNA has become the most common form of gRNA used by those skilled in the art in CRISPR technology, and thus the terms “sgRNA” and “gRNA” may have the same meaning herein. The sgRNA can be synthesized artificially or can be prepared in vitro or in vivo from a DNA template. The sgRNA can bind to a Cas nuclease or target a target DNA, and can direct the Cas nuclease to cleave a DNA site complementary to the gRNA.

As used in the present application, a crRNA typically comprises a spacer sequence that mediates target recognition and a direct repeat sequence (also referred to herein as a “Direct repeat” or “DR sequence”) that forms a complex with the CRISPR-Cas effector protein. In certain instances, a spacer sequence (also referred to as a guide sequence) is any polynucleotide sequence that has sufficient complementarity to a target sequence to hybridize to the target sequence and direct specific binding of the CRISPR-Cas system complex to the target sequence. In certain embodiments, the degree of complementarity between the spacer sequence and its corresponding target sequence when optimally aligned is at least 50%, at least 60%, at least 70%, at least 80%, at least 90%, at least 95%, at least 96%, at least 97%, at least 98%, or at least 99%. Determining the optimal alignment is within the capabilities of one of ordinary skill in the art. For example, there are publicly and commercially available alignment algorithms and programs such as, but not limited to, ClustalW, the Smith-Waterman algorithm in Matlab, Bowtie, Geneious, Biopython, and SeqMan.

In certain embodiments, the spacer sequence is at least 5, at least 10, at least 15, at least 16, at least 17, at least 18, at least 19, at least 20, at least 21, at least 22, at least 23, at least 24, at least 25, at least 26, at least 27, at least 28, at least 29, at least 30, at least 35, at least 40, at least 45, or at least 50 nucleotides in length. In certain embodiments, the spacer sequence is no more than 50, 45, 40, 35, 30, 25, 24, 23, 22, 21, 20, 15, 10, or fewer nucleotides in length. In certain embodiments, the spacer sequence is 10-30, 15-25, 15-22, 16-24, 19-25, or 19-22 nucleotides in length. In certain preferred embodiments, the spacer sequence is 20 nucleotides in length.

In certain embodiments, the direct repeat sequence is at least 10, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 40, at least 45, at least 50, at least 55, at least 56, at least 57, at least 58, at least 59, at least 60, at least 61, at least 62, at least 63, at least 64, at least 65, or at least 70 nucleotides in length. In certain embodiments, the direct repeat sequence is no more than 70, 65, 64, 63, 62, 61, 60, 59, 58, 57, 56, 55, 50, 45, 40, 35, 34, 33, 32, 31, 30, 29, 28, 27, 26, 25, 24, 23, 22, 21, 20, 15, 10, or fewer nucleotides in length. In certain embodiments, the direct repeat sequence is 55-70 nucleotides, e.g. 55-65 nucleotides, e.g. 60-65 nucleotides, e.g. 62-65 nucleotides, e.g. 63-64 nucleotides in length. In certain embodiments, the direct repeat sequence is 15-40 nucleotides, e.g. 15-25 nucleotides, e.g. 20-30 nucleotides, e.g. 22-36 nucleotides, e.g. 31 nucleotides in length.

In the present application, the terms “comprise” and “include” are used interchangeably, and generally refer to the inclusion of explicitly specified features, but not the exclusion of other elements. The term “at least” generally refers to cases where the stated number itself is included.

In the present application, the term “about” or “approximately” means within an acceptable error range for a particular value as determined by one of ordinary skill in the art, which depends in part on how the value is measured or determined, i.e., the limitations of the measurement system. For example, “about” or “approximately” may mean within 1 or more than 1 standard deviation according to practice in the art. Alternatively, “about” or “approximately” means a range of up to 10% or 20% (i.e., +10% or +20%).

In the present application, the term “selected from” generally means including the selected objects and all combinations thereof. For example, “selected from (:) A, B and C” is intended to include all combinations of A, B and C, for example, A, B, C, A+B, A+C, B+C or A+B+C. Without wishing to be bound by any theory, the following examples are only for illustrating the enzymes of the present application and their fusion molecules, and are not used to limit the scope of the invention of the present application.

### EXAMPLES

### Example 1

### Determination of the Cleavage Activity of the Cas Enzyme of the Present Application in Eukaryotic Cells

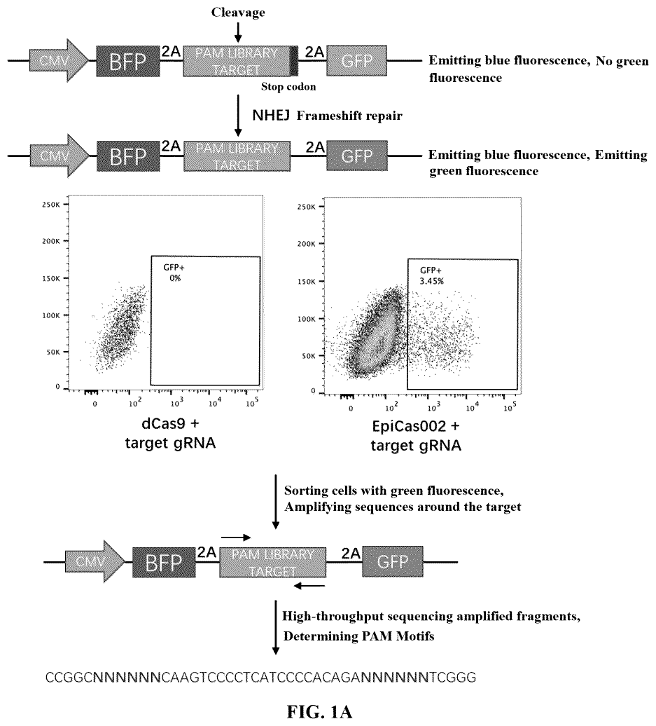

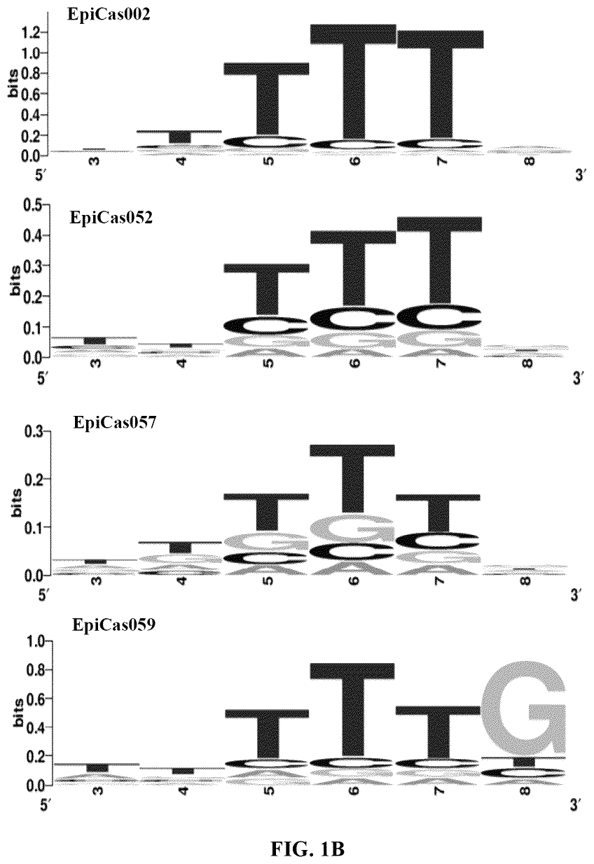

This example tested the activity of Cas enzymes provided in the present application using stably transfected 293T cells with a blue-green reporter system. As shown in FIG. 1, the reporter system carries a continuously expressed CMV promoter, a sequence encoding a blue fluorescent protein, a sequence encoding a green fluorescent protein, and a gRNA targeting sequence inserted in between; the gRNA targeting sequence is flanked by random N-base sequences that can be used to screen Cas enzymes with different protospacer adjacent motif (PAM) preferences.

The cleavage activity of the Cas enzyme of the present application would be determined by the fluorescence results displayed by the reporter system. The reporter system in which no cleavage occurs would stably express the blue fluorescent protein and emit blue fluorescence. However, the green fluorescent protein could not be expressed due to the presence of a stop codon before the sequence encoding the green fluorescent protein, and the reporter system would not emit green fluorescence. Only after DNA cleavage occurs near the gRNA targeting sequence, and the cell undergoes frameshift mutation in the process of nick repair, could the blue fluorescent protein be stably expressed and the green fluorescent protein be expressed at the same time. The fluorescence results of FIG. 1A indicated that after transfection with the Cas enzyme provided in the present application (SEQ ID NO: 2), the cells in the experimental group with cleavage activity would produce a population of cells clearly expressing green fluorescence.

The green fluorescent cell population was enriched by flow sorting, the genome was extracted and then the target region was amplified, and the PAM motif required for gRNA binding of the Cas enzyme provided in the present application could be determined by high-throughput sequencing (as shown in FIG. 1B).

In this example, an exemplary nucleotide sequence encoding the reporter system is shown below (blue and green fluorescent protein sequence in bold, linker sequence in italics, 2A cleavage peptide sequence underlined, and exemplary gRNA targeting sequence comprising random N bases (SEQ ID NO: 59) in bold and underlined):

(SEQ ID NO: 61)
ATGAGCGAGCTGATTAAGGAGAACATGCACATGAAGCTGTACATGGAGGGCA
CCGTGGACAACCATCACTTCAAGTGCACATCCGAGGGCGAAGGCAAGCCCTAC
GAGGGCACCCAGACCATGAGAATCAAGGTGGTCGAGGGCGGCCCTCTCCCCTT
CGCCTTCGACATCCTGGCTACTAGCTTCCTCTACGGCAGCAAGACCTTCATCAA
CCACACCCAGGGCATCCCCGACTTCTTCAAGCAGTCCTTCCCTGAGGGCTTCAC
ATGGGAGAGAGTCACCACATACGAAGACGGGGGCGTGCTGACCGCTACCCAGG
ACACCAGCCTCCAGGACGGCTGCCTCATCTACAACGTCAAGATCAGAGGGGTGA
ACTTCACATCCAACGGCCCTGTGATGCAGAAGAAAACACTCGGCTGGGAGGCC
TTCACCGAGACGCTGTACCCCGCTGACGGCGGCCTGGAAGGCAGAAACGACAT
GGCCCTGAAGCTCGTGGGCGGGAGCCATCTGATCGCAAACATCAAGACCACATA
TAGATCCAAGAAACCCGCTAAGAACCTCAAGATGCCTGGCGTCTACTATGTGGA
CTACAGACTGGAAAGAATCAAGGAGGCCAACAACGAGACCTACGTCGAGCAGC
ACGAGGTGGCAGTGGCCAGATACTGCGACCTCCCTAGCAAACTGGGGCACAAG
CTTAATGGATCCGGCGCAACAAACTTCtctctgctgaaacaagccggagatgTCGAAGAGAATCCT
GGACCGGCTGAGCAGGCTCGTTGGGAAGCAGCCTCCAAAGAAACCATCAAGAAAACCAC
CAAGCCCTGTCCCCGCTGCCATGTACCAGTGGAAAAAAATGGAGGCTGCATGCACATGAA
GTGTCCGCAGCCCCAGTGCAGGCTCGAGTGGTGCTGGAACTGTGGCTGCGAGcccCCGG
CNNNNNNCAAGTCCCCTCATCCCCACAGANNNNNNTCgggTtaGGGGACCACTGGTTC
GACGTGAGCGGTTCTGGCGAAGGGCGAGGTTCCTTGCTGACCTGTGGCGATGTAGAA
GAAAATCCCGGCCCCGGATCCGTGAGCAAGGGCGAGGAGCTGTTCACCGGGGTG
GTGCCCATCCTGGTCGAGCTGGACGGCGACGTAAACGGCCACAAGTTCAGCGT
GTCCGGCGAGGGCGAGGGCGATGCCACCTACGGCAAGCTGACCCTGAAGTTCA
TCTGCACCACCGGCAAGCTGCCCGTGCCCTGGCCCACCCTCGTGACCACCCTG
ACCTACGGCGTGCAGTGCTTCAGCCGCTACCCCGACCACATGAAGCAGCACGAC
TTCTTCAAGTCCGCCATGCCCGAAGGCTACGTCCAGGAGCGCACCATCTTCTTC
AAGGACGACGGCAACTACAAGACCCGCGCCGAGGTGAAGTTCGAGGGCGACAC
CCTGGTGAACCGCATCGAGCTGAAGGGCATCGACTTCAAGGAGGACGGCAACA
TCCTGGGGCACAAGCTGGAGTACAACTACAACAGCCACAACGTCTATATCATGG
CCGACAAGCAGAAGAACGGCATCAAGGTGAACTTCAAGATCCGCCACAACATCG
AGGACGGCAGCGTGCAGCTCGCCGACCACTACCAGCAGAACACCCCCATCGGC
GACGGCCCCGTGCTGCTGCCCGACAACCACTACCTGAGCACCCAGTCCGCCCT
GAGCAAAGACCCCAACGAGAAGCGCGATCACATGGTCCTGCTGGAGTTCGTGA
CCGCCGCCGGGATCACTCTCGGCATGGACGAGCTGTACAAGTAA.

### Example 2

### Determination of the Strength of the Cleavage Activity of the Cas Enzyme of the Present Application in Eukaryotic Cells

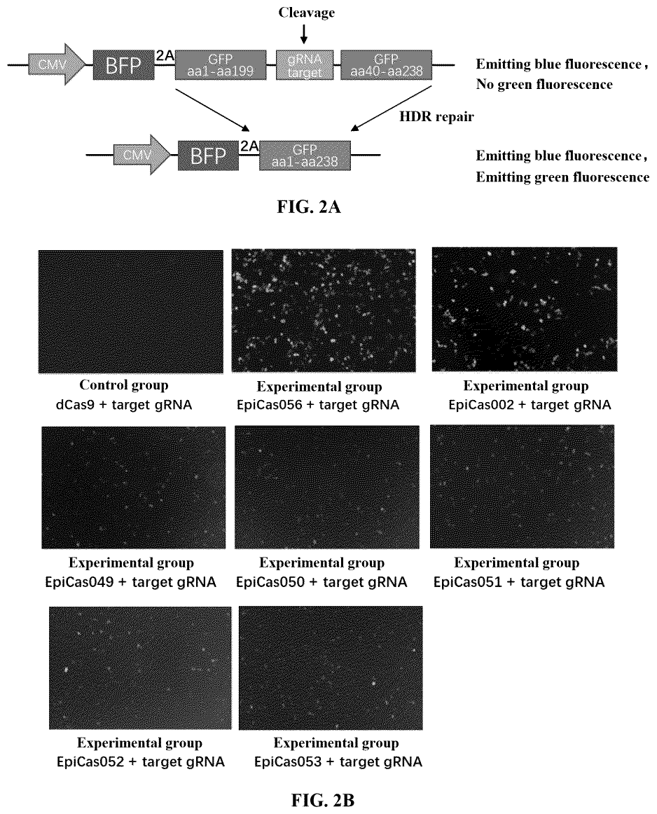

The Cas enzymes provided in the present application (SEQ ID NOs: 2, 47-51 and 54), the corresponding gRNA and the reporter plasmid with gRNA targeting site were co-transfected into HEK293T cells, and the reporter systems required for different Cas enzymes could be quickly constructed by modifying the targeting sequence on the reporter plasmid. As shown in FIG. 2A, the reporter system cells without cleavage only expressed blue fluorescent protein, and the green fluorescent protein could not be normally expressed because it was blocked. At this time, the reporter system only emitted blue fluorescence. After cleavage occurs at the gRNA targeting site, the cells would repair the DNA through homologous recombination, thereby obtaining a complete green fluorescent protein expression frame, which caused the reporter system to emit green fluorescence.

The Cas enzyme with stronger cleavage activity would produce a higher proportion of DNA cleavage, thereby obtaining more copies of the repaired green fluorescent protein, and the cells exhibited stronger green fluorescence. Therefore, this example quickly compared the cleavage activities of different Cas enzymes by comparing the proportion and fluorescence intensity of green fluorescent cells. The comparison results are shown in FIG. 2B.

In this example, the exemplary nucleotide sequence encoding the reporter system is shown below (blue and green fluorescent protein sequences in bold, linker sequence in italics, 2A cleavage peptide sequence underlined; exemplary gRNA targeting sequence (SEQ ID NO: 60) in bold and underlined, where the TTTG and TGG at both ends of the sequence would be set to different sequences according to the PAM preferences of different Cas enzymes):

(SEQ ID NO: 62)
ATGAGCGAGCTGATTAAGGAGAACATGCACATGAAGCTGTACATGGAGGGCA
CCGTGGACAACCATCACTTCAAGTGCACATCCGAGGGCGAAGGCAAGCCCTAC
GAGGGCACCCAGACCATGAGAATCAAGGTGGTCGAGGGCGGCCCTCTCCCCTT
CGCCTTCGACATCCTGGCTACTAGCTTCCTCTACGGCAGCAAGACCTTCATCAA
CCACACCCAGGGCATCCCCGACTTCTTCAAGCAGTCCTTCCCTGAGGGCTTCAC
ATGGGAGAGAGTCACCACATACGAAGACGGGGGCGTGCTGACCGCTACCCAGG
ACACCAGCCTCCAGGACGGCTGCCTCATCTACAACGTCAAGATCAGAGGGGTGA
ACTTCACATCCAACGGCCCTGTGATGCAGAAGAAAACACTCGGCTGGGAGGCC
TTCACCGAGACGCTGTACCCCGCTGACGGCGGCCTGGAAGGCAGAAACGACAT
GGCCCTGAAGCTCGTGGGCGGGAGCCATCTGATCGCAAACATCAAGACCACATA
TAGATCCAAGAAACCCGCTAAGAACCTCAAGATGCCTGGCGTCTACTATGTGGA
CTACAGACTGGAAAGAATCAAGGAGGCCAACAACGAGACCTACGTCGAGCAGC
ACGAGGTGGCAGTGGCCAGATACTGCGACCTCCCTAGCAAACTGGGGCACAAG
CTTAATGGATCCGGCGCAACAAACTTCtctctgctgaaacaagccggagatgTCGAAGAGAATCCT
GGACCGGCTgtgagcaagggcgaggagcTGTTCACCGGGGTGGTGCCCATCCTGGTCGAG
CTGGACGGCGACGTAAACGGCCACAAGTTCAGCGTGTCCGGCGAGGGCGAGGG
CGATGCCACCTACGGCAAGCTGACCCTGAAGTTCATCTGCACCACCGGCAAGCT
GCCCGTGCCCTGGCCCACCCTCGTGACCACCCTGACCTACGGCGTGCAGTGCT
TCAGCCGCTACCCCGACCACATGAAGCAGCACGACTTCTTCAAGTCCGCCATGC
CCGAAGGCTACGTCCAGGAGCGCACCATCTTCTTCAAGGACGACGGCAACTACA
AGACCCGCGCCGAGGTGAAGTTCGAGGGCGACACCCTGGTGAACCGCATCGAG
CTGAAGGGCATCGACTTCAAGGAGGACGGCAACATCCTGGGGCACAAGCTGGA
GTACAACTACAACAGCCACAACGTCTATATCATGGCCGACAAGCAGAAGAACGG
CATCAAGGTGAACTTCAAGATCCGCCACAACATCGAGGACGGCAGCGTGCAGCT
CGCCGACCACTACCAGCAGAACACCCCCATCGGCGACGGCCCCGTGctgctgcccgac
aaccacTGAtaaggatccTTTGAAGTCCCCTCATCCCCACAGTGGtaggatatcggcaagctgaccctga
aGTTCATCTGCACCACCGGCAAGCTGCCCGTGCCCTGGCCCACCCTCGTGACCA
CCCTGACCTACGGCGTGCAGTGCTTCAGCCGCTACCCCGACCACATGAAGCAGC
ACGACTTCTTCAAGTCCGCCATGCCCGAAGGCTACGTCCAGGAGCGCACCATCT
TCTTCAAGGACGACGGCAACTACAAGACCCGCGCCGAGGTGAAGTTCGAGGGC
GACACCCTGGTGAACCGCATCGAGCTGAAGGGCATCGACTTCAAGGAGGACGG
CAACATCCTGGGGCACAAGCTGGAGTACAACTACAACAGCCACAACGTCTATAT
CATGGCCGACAAGCAGAAGAACGGCATCAAGGTGAACTTCAAGATCCGCCACA
ACATCGAGGACGGCAGCGTGCAGCTCGCCGACCACTACCAGCAGAACACCCCC
ATCGGCGACGGCCCCGTGCTGCTGCCCGACAACCACTACCTGAGCACCCAGTC
CGCCCTGAGCAAAGACCCCAACGAGAAGCGCGATCACATGGTCCTGCTGGAGT
TCGTGACCGCCGCCGGGATCACTCTCGgcatggacgagctgtacAAGtaa.

### Example 3

### Determination of the In Vitro Cleavage Activity and PAM Sequence of the Cas Enzyme of the Present Application

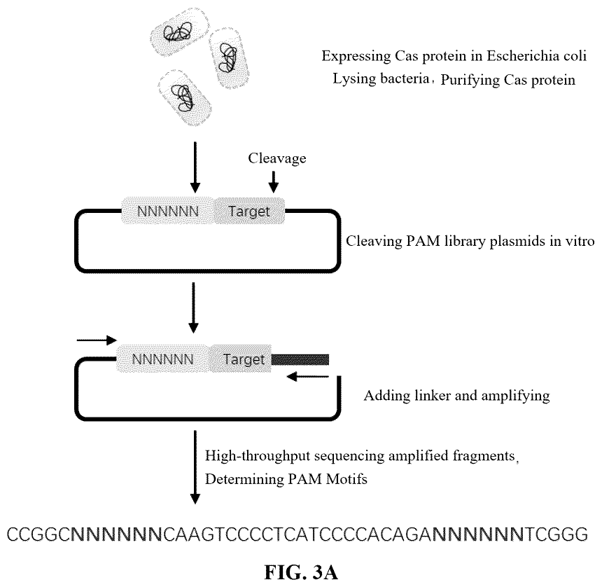

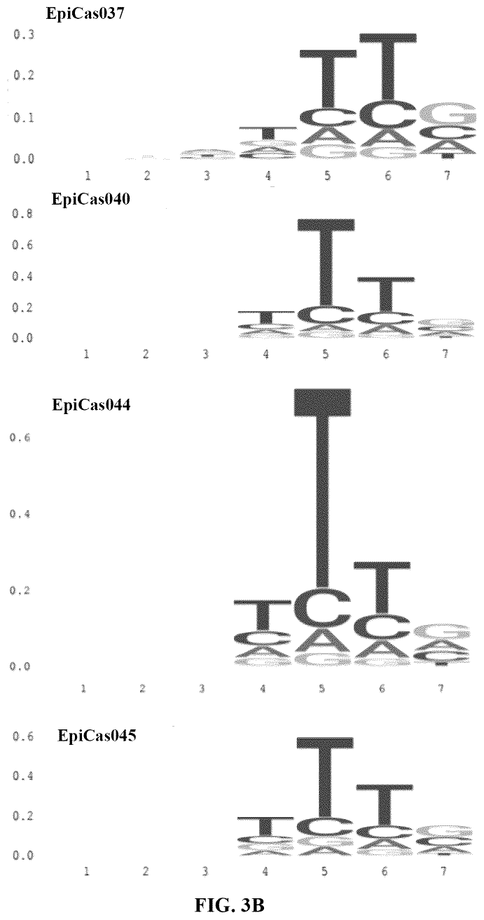

As shown in the experimental design flow chart of this embodiment in FIG. 3A, the Cas enzyme provided in the present application was expressed and purified by E. coli, and reacted with a plasmid library containing a PAM library together with an in vitro transcribed gRNA (the gRNA targeting sequence was SEQ ID NO: 59 flanked by random N-base sequences to determine PAM preference). The Cas enzyme having cleavage activity would cleave the plasmid library to form linearized DNA. By in vitro ligation, the linker fragment was ligated to the library fragment after linearization of DNA, and then the vector and the linker ligated library fragment were amplified by specific PCR primers and high-throughput sequencing was performed.

The analytical sequencing results shown in FIG. 3B indicated the preference of Cas enzymes with cleavage activity for PAM motifs. These results indicated that the four proteins EpiCas037 (SEQ ID NO: 36), EpiCas044 (SEQ ID NO: 43), EpiCas040 (SEQ ID NO: 39) and EpiCas045 (SEQ ID NO: 44) all had cleavage activity and preferred T-rich PAM motifs.

### Example 4

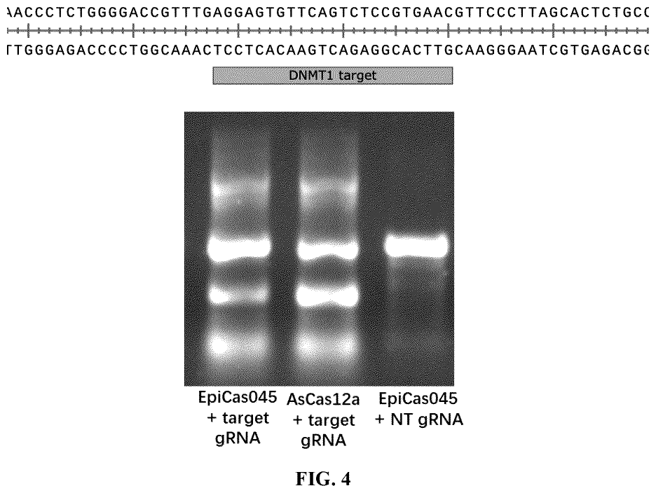

Detection of the activity of the Cas enzyme of the present application at endogenous sites According to the PAM motifs of different Cas enzymes determined in Example 3, eligible sites were found at endogenous sites of HEK293T, and gRNA (gRNA targeting sequence was SEQ ID NO: 89) was constructed. The Cas enzyme (SEQ ID NOs: 44) provided in the present application and the corresponding gRNA were co-transfected into HEK293T cells, and the transfection-positive cells were enriched by flow sorting 3 days after transfection, and the target region was amplified after lysing the cells. The amplified PCR fragment was reacted with T7 endonuclease. If the Cas enzyme cleaved the target site, various randomly repaired sequences would be formed, and these sequences would be cleaved by T7 enzyme to produce bands other than the target band. Therefore, the results shown in FIG. 4 indicated that the Cas enzyme had cleavage activity at the endogenous sites.

### Example 5

### Determination of the Strength of the Cleavage Activity of the Cas Enzyme Mutant of the Present Application in Eukaryotic Cells

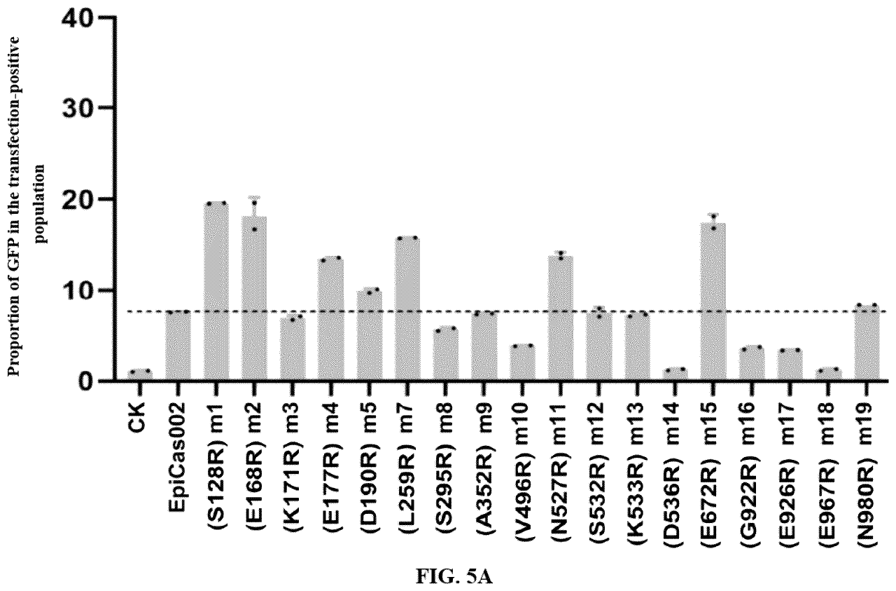

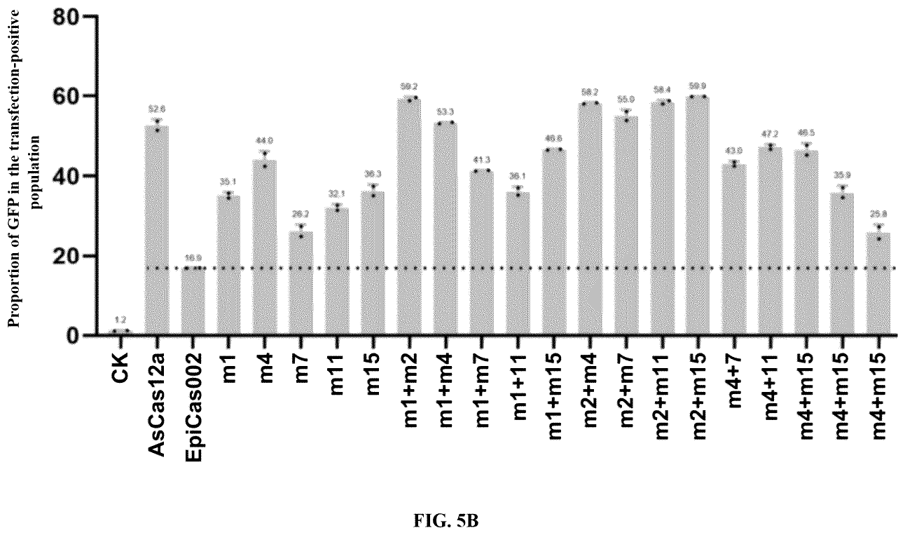

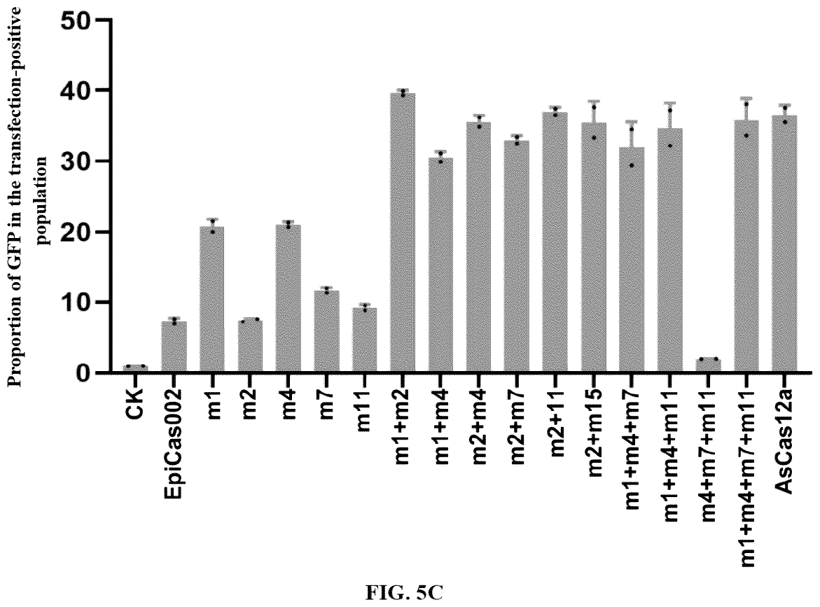

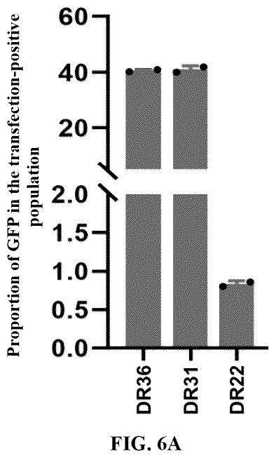

By site-directed mutagenesis, different sites of the Cas enzyme (SEQ ID NO: 2) provided in the present application were mutated to target amino acids (see the table below), and then the detection as described in Example 2 was performed. Among them, the DR (direct repeat) region sequence and spacer sequence of the gRNA were as set forth in SEQ ID NOs: 64 and 60, respectively. The results shown in FIG. 5A indicated that the activities of the m1, m2, m4, m5, m7, m11, and m15 mutants were significantly increased compared to the wild type. After superimposing these effective mutations, the activity detection by the same method was performed. The results shown in FIG. 5B indicated that the double mutants formed by superimposing two mutations selected from the following table could further enhance the activity, and the activity of some double mutants was comparable to that of AsCas12a; the results shown in FIG. 5C indicated that the triple mutants and quadruple mutants formed by further superimposing the effective single mutations and double mutations still maintain the original activities, and the activity is comparable to that of AsCas12a. The amino acid sequence of AsCas12a for control is as set forth in SEQ ID NO: 127.

| | |
|---|---|
| EpiCas002-m1 | S128R |
| EpiCas002-m2 | E168R |
| EpiCas002-m3 | K171R |
| EpiCas002-m4 | E177R |
| EpiCas002-m5 | D190R |
| EpiCas002-m6 | E248R |
| EpiCas002-m7 | L259R |
| EpiCas002-m8 | S295R |
| EpiCas002-m9 | A352R |
| EpiCas002-m10 | V496R |
| EpiCas002-m11 | N527R |
| EpiCas002-m12 | S532R |
| EpiCas002-m13 | K533R |
| EpiCas002-m14 | D536R |
| EpiCas002-m15 | E672R |
| EpiCas002-m16 | G922R |
| EpiCas002-m17 | E926R |
| EpiCas002-m18 | E967R |
| EpiCas002-m19 | N980R |

### Example 6

### Optimization of CRISPR/Cas Editing System

This example optimized an editing system comprising the Cas enzyme provided (SEQ ID NO: 57) and gRNA in the present application. The Cas enzyme activity was detected using the reporter system and detection method as described in Example 2.

### Grna Optimization:

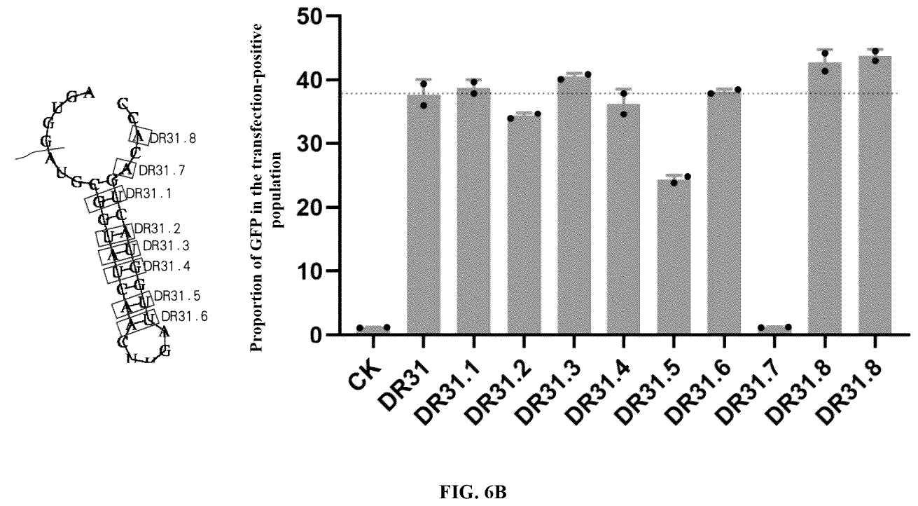

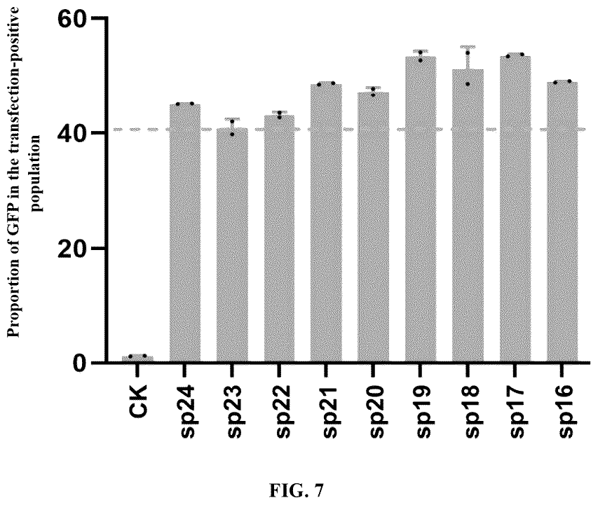

The DR (direct repeat) region of the gRNA was changed by molecular cloning to 36 nt (SEQ ID NO: 87), 31 nt (SEQ ID NO: 91), 22 nt (SEQ ID NO: 90). The results shown in FIG. 6A indicated that a DR of 31 nt length could maintain 100% Cas enzyme activity. When the base pairs of the DR region were altered by molecular cloning to improve its stability (the nucleotide sequences are as set forth in SEQ ID NOs: 92-99 for DRs of 31.1 to 31.8), the results shown in FIG. 6B indicated that most of the alterations had little effect on activity, whereas the alterations in 31.8 were the most effective in improving activity. In this example, the length of the spacer of the gRNA used to target DNA was changed by molecular cloning to improve its binding efficiency, thereby enhancing Cas enzyme activity. As shown in FIG. 7, when the spacer length varied from 24 nt to 16 nt (SEQ ID NOs: 100-103, 60 and 104-107), the Cas enzymes provided in the present application could maintain their activity.

### Cas Enzyme Mutant:

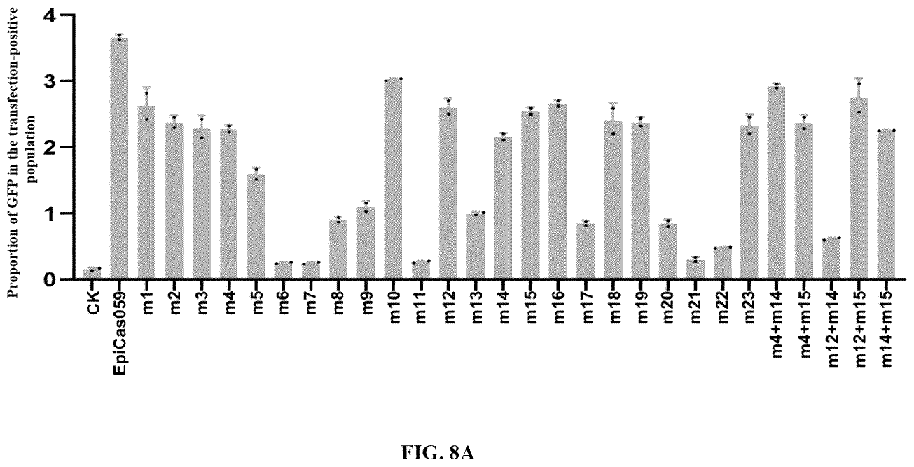

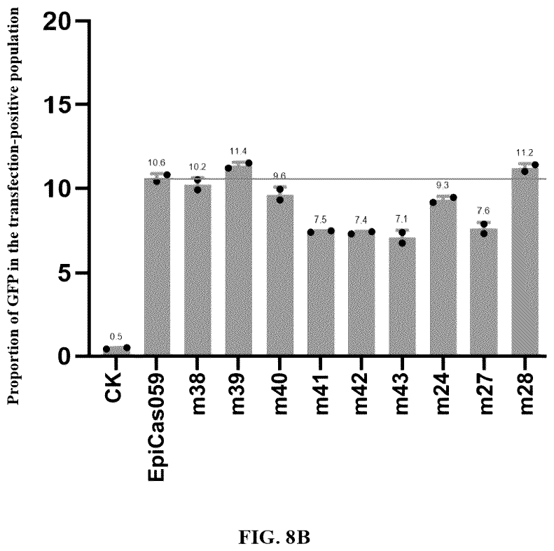

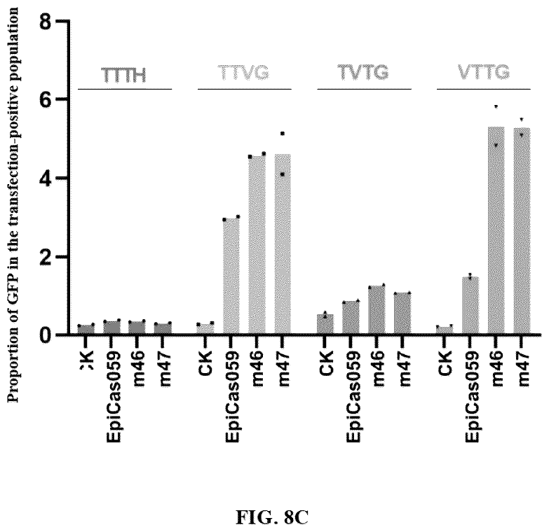

By site-directed mutagenesis, different sites of the Cas enzyme (SEQ ID NO: 57) provided in the present application were mutated to target amino acids (see the table below), and then the detection as described in Example 2 was performed. Among them, the DR (direct repeat) region sequence and spacer sequence of the gRNA were as set forth in SEQ ID NOs: 91 and 60, respectively. The results shown in FIGS. 8A-8B indicated that most single mutations had little effect on Cas enzyme activity, and if the single mutations with better activity optimization effect were superimposed, it could be found that the formed double mutants could still maintain high activity.

In this example, on the basis of the reporter system described in Example 2, TTTG was mutated into TTTH, TTVG, TVTG and VTTG respectively (H stands for A/C/T, V stands for A/C/G, and 3 plasmids were mixed in equal proportions and then transfected) to construct a new PAM reporter system. The results shown in FIG. 8C indicated that some mutants could broaden the range of PAM recognition, especially in the recognition of PAM targets of VTTG and TTVG. Compared with the wild type, the activity of the Cas enzyme provided in the present application was significantly improved.

| | |
|---|---|
| EpiCas059-m1 | K57R |
| EpiCas059-m2 | Y258R |
| EpiCas059-m3 | K264R |
| EpiCas059-m4 | K272R |
| EpiCas059-m5 | D621R |
| EpiCas059-m6 | Q649R |
| EpiCas059-m7 | E681R |
| EpiCas059-m8 | Y754R |
| EpiCas059-m9 | S767R |
| EpiCas059-m10 | Q770R |
| EpiCas059-m11 | D823R |
| EpiCas059-m12 | K850R |
| EpiCas059-m13 | V861R |
| EpiCas059-m14 | K186R |
| EpiCas059-m15 | T197R |
| EpiCas059-m16 | V242R |
| EpiCas059-m17 | K304R |
| EpiCas059-m18 | T319R |
| EpiCas059-m19 | E397R |
| EpiCas059-m20 | Y364R |
| EpiCas059-m21 | S421R |
| EpiCas059-m22 | V453R |
| EpiCas059-m23 | P627R |
| EpiCas059-m24 | K9R |
| EpiCas059-m25 | I10R |
| EpiCas059-m26 | C39R |
| EpiCas059-m27 | N53R |
| EpiCas059-m28 | H65R |
| EpiCas059-m29 | S69R |
| EpiCas059-m30 | E81R |
| EpiCas059-m31 | K95R |
| EpiCas059-m32 | D98R |
| EpiCas059-m33 | V103R |
| EpiCas059-m34 | G112R |
| EpiCas059-m35 | I115R |
| EpiCas059-m36 | G120R |
| EpiCas059-m37 | K123R |
| EpiCas059-m38 | A153N |
| EpiCas059-m39 | A153R |
| EpiCas059-m40 | T346S |
| EpiCas059-m41 | T346R |
| EpiCas059-m42 | D347N |
| EpiCas059-m43 | D347R |
| EpiCas059-m44 | D183Y/T458K/G528C |
| EpiCas059-m45 | E144K/S633N |
| EpiCas059-m46 | E144K/D183Y/T458K/G528C/S633N |
| EpiCas059-m47 | E144K/T458K |

### Example 7

Detection of the Effects of Inactivated Cas Enzyme (dCas) at Endogenous Sites
Base Editing Effect of dCas-CBE:

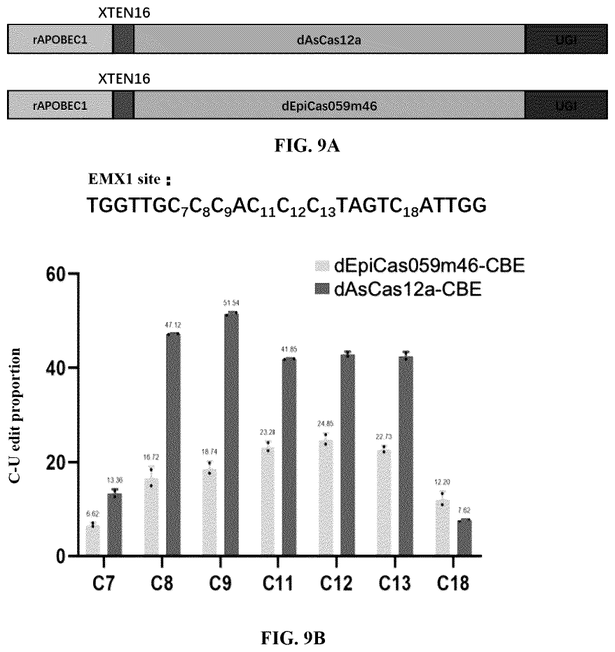

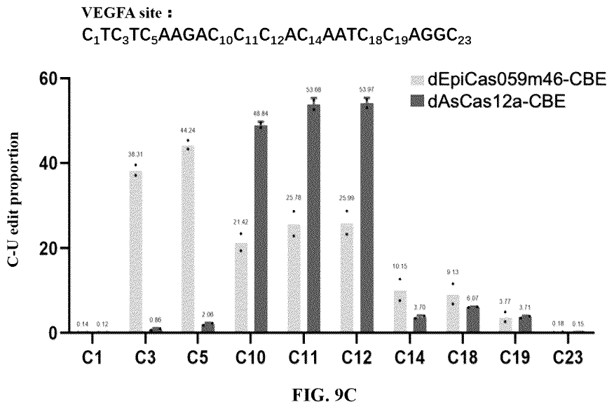

The Cas enzyme provided in the present application (SEQ ID NO: 57) was inactivated and then fused with rAPOBEC1 and UGI (FIG. 9A) to obtain a cytosine base editor based on the Cas enzyme provided in the present application. The cytosine base editor was co-transfected into HEK293T cells with the corresponding gRNA (the DR sequence is as set forth in SEQ ID NO: 91, and the spacer sequences targeting the EMX1 site and the VEGFA site are as set forth in SEQ ID NOs: 108 and 109, respectively). 48 hours after transfection, transfection-positive cells were sorted, the genome was extracted, the target site was amplified, and the base editing efficiency was determined by Sanger sequencing. The results are shown in FIGS. 9B-9C.

The amino acid sequence of the dEpiCas059m46-CBE editor is shown below (SEQ ID NO: 116, NLS in italics, rAPOBEC1 in italics and bold, dEpiCas059 in bold, UGI in italics and underlined, P2A cleavage peptide underlined, and mCherry marker in < >; the plasmid sequence is SEQ ID NO: 117):

MKRTADGSEFESPKKKRKVSSETGPVAVDPTLRRRIEPHEFEVFFDPRELRKETCLLY
EINWGGRHSIWRHTSQNTNKHVEVNFIEKFTTERYFCPNTRCSITWFLSWSPCGECSRAI
TEFLSRYPHVTLFIYIARLYHHADPRNRQGLRDLISSGVTIQIMTEQESGYCWRNFVNYS
PSNEAHWPRYPHLWVRLYVLELYCIILGLPPCLNILRRKQPQLTFFTIALQSCHYQRLPPH
ILWATGLKSGSETPGTSESATPESGSEETRTPEKIACQQDRSAFFKLGKLPQKTTSAIYH
LMRCSKRYIDNVSETISNNIEKAAMTSDNHIFRSALKEEYKKIYREKFYPDVTGQPFP
DKGPDNTHIVERTVENLCGQYISALKGVMKVRKPKWQDDIFEDESDKVIAKMLMA
SGNEADIHPKLEKKIASAKKKDKMDEWQKVLDRYYYIKSKTDIEWTYTTLLYDHIA
RECARSMFEDGNKPKFCYTNGNKFAKQVVDELWGSERVEYEYEKYLHCRTLEAY
WNEENKYFFLENQKKSKKTYEARQIIDHFIENEDAVKAETEEYMAKVAKEFGENGG
CKFRTENILYLVNSVSINKKQDEERCPIFKVTDIPQYAEAGIDRIGECKYVIKLKLFTV
GERYGTTYMPEAFSGLYDRRTNQRYDLIDRACKGSYVYLPIYISDKDSRKWSDRLK
NTFTMSAFSISERKTKKDGRVLCVNLPYKHIQHKPRNYFKVYPTYGVAINVKHSLFS
TNATNLNGYIDVNGELIDFYNSIGRPDKLKNINLAVTLGDKCGTAGNHTNFLLVSDL
GDWDDHGKKRNDRFVKREDGTYYSRPDLLADGLNFLKKKYSGDRKSYDYINTTIA
FRNNQTRLRCIDKEWNDRKRAYDEEHGKPEKGERNGFYPTEDAERLTMERQALRR
EIAENTRQVIAYMLWLFQENGIKSIALAHLDCGSWKNFRDQLKPKWFCNNCKYWA
EKYGIPYEATKYYRSNRKYFDIEDGELIPENVKLNALGEAENDKRYFQTSIAKAVGM
SSIKQMFSIVSQNYDIQVNYTEQAYTSQTDSRNGTVDKDFRVSQECYIGSDGTKMNC
AFNAAVNIADRISDDTLREEFNKELPMKEFVKYGATQIVSRNRRWNAENAERVTMA
AFQKRKNADGGAHTAAVKPTSKTEESSLKLVAEPKKKRKVSGGSTNLSDIIEKETGKQL
VIQESILMLPEEVEEVIGNKPESDILVHTAYDESTDENVMLLTSDAPEYKPWALVIQDSNGENKI
KMLSGGSPKKKRKVGSKRTGSGATNFSLLKQAGDVEENPGPVN<MVSKGEEDNMAIIKE
FMRFKVHMEGSVNGHEFEIEGEGEGRPYEGTQTAKLKVTKGGPLPFAWDILSPQFMYGS
KAYVKHPADIPDYLKLSFPEGFKWERVMNFEDGGVVTVTQDSSLQDGEFIYKVKLRGTN
FPSDGPVMQKKTMGWEASSERMYPEDGALKGEIKQRLKLKDGGHYDAEVKTTYKAKK
PVQLPGAYNVNIKLDITSHNEDYTIVEQYERAEGRHSTGGMDELYK>*

The amino acid sequence of the dAsCas12a-CBE editor is shown below (SEQ ID NO: 118, NLS in italics, rAPOBEC1 in italics and bold, dAsCas12a in bold, UGI in italics and underlined, P2A cleavage peptide underlined, and mCherry marker in < >; the plasmid sequence is SEQ ID NO: 119):

MKRTADGSEFESPKKKRKVSSETGPVAVDPTLRRRIEPHEFEVFFDPRELRKETCLLY
EINWGGRHSIWRHTSQNTNKHVEVNFIEKFTTERYFCPNTRCSITWFLSWSPCGECSRAI
TEFLSRYPHVTLFIYIARLYHHADPRNRQGLRDLISSGVTIQIMTEQESGYCWRNFVNYS
PSNEAHWPRYPHLWVRLYVLELYCIILGLPPCLNILRRKQPQLTFFTIALQSCHYQRLPPH
ILWATGLKSGSETPGTSESATPESGSSKLEKFTNCYSLSKTLRFKAIPVGKTQENIDNKR
LLVEDEKRAEDYKGVKKLLDRYYLSFINDVLHSIKLKNLNNYISLFRKKTRTEKENK
ELENLEINLRKEIAKAFKGNEGYKSLFKKDIIETILPEFLDDKDEIALVNSFNGFTTAF
TGFFDNRENMFSEEAKSTSIAFRCINENLTRYISNMDIFEKVDAIFDKHEVQEIKEKIL
NSDYDVEDFFEGEFFNFVLTQEGIDVYNAIIGGFVTESGEKIKGLNEYINLYNQKTKQ
KLPKFKPLYKQVLSDRESLSFYGEGYTSDEEVLEVFRNTLNKNSEIFSSIKKLEKLFK
NFDEYSSAGIFVKNGPAISTISKDIFGEWNVIRDKWNAEYDDIHLKKKAVVTEKYED
DRRKSFKKIGSFSLEQLQEYADADLSVVEKLKEIIIQKVDEIYKVYGSSEKLFDADFV
LEKSLKKNDAVVAIMKDLLDSVKSFENYIKAFFGEGKETNRDESFYGDFVLAYDILL
KVDHIYDAIRNYVTQKPYSKDKFKLYFQNPQFMGGWDKDKETDYRATILRYGSKY
YLAIMDKKYAKCLQKIDKDDVNGNYEKINYKLLPGPNKMLPKVFFSKKWMAYYNP
SEDIQKIYKNGTFKKGDMFNLNDCHKLIDFFKDSISRYPKWSNAYDFNFSETEKYKD
IAGFYREVEEQGYKVSFESASKKEVDKLVEEGKLYMFQIYNKDFSDKSHGTPNLHT
MYFKLLFDENNHGQIRLSGGAELFMRRASLKKEELVVHPANSPIANKNPDNPKKTT
TLSYDVYKDKRFSEDQYELHIPIAINKCPKNIFKINTEVRVLLKHDDNPYVIGIARGE
RNLLYIVVVDGKGNIVEQYSLNEIINNFNGIRIKTDYHSLLDKKEKERFEARQNWTSI
ENIKELKAGYISQVVHKICELVEKYDAVIALADLNSGFKNSRVKVEKQVYQKFEKM
LIDKLNYMVDKKSNPCATGGALKGYQITNKFESFKSMSTQNGFIFYIPAWLTSKIDPS
TGFVNLLKTKYTSIADSKKFISSFDRIMYVPEEDLFEFALDYKNFSRTDADYIKKWK
LYSYGNRIRIFRNPKKNNVFDWEEVCLTSAYKELFNKYGINYQQGDIRALLCEQSDK
AFYSSFMALMSLMLQMRNSITGRTDVAFLISPVKNSDGIFYDSRNYEAQENAILPKN
ADANGAYNIARKVLWAIGQFKKAEDEKLDKVKIAISNKEWLEYAQTSVKHPKKKRK
VSGGSTNLSDIIEKETGKQLVIQESILMLPEEVEEVIGNKPESDILVHTAYDESTDENVMLLTSD
APEYKPWALVIQDSNGENKIKMLSGGSPKKKRKVGSKRTGSGATNfSLLKQAGDVEENPGP
VN<MVSKGEEDNMAIIKEFMRFKVHMEGSVNGHEFEIEGEGEGRPYEGTQTAKLKVTK
GGPLPFAWDILSPQFMYGSKAYVKHPADIPDYLKLSFPEGFKWERVMNFEDGGVVTVTQ
DSSLQDGEFIYKVKLRGTNFPSDGPVMQKKTMGWEASSERMYPEDGALKGEIKQRLKL
KDGGHYDAEVKTTYKAKKPVQLPGAYNVNIKLDITSHNEDYTIVEQYERAEGRHSTGG
MDELYK>*

Gene Activation Effect of dCas-SPH:

The Cas enzyme provided in the present application (SEQ ID NO: 55) was subjected to inactivation treatment and then fused with 10×GCN4 to recruit a fusion peptide, i.e., scFV-P65-HSF1, thereby obtaining a gene activation tool based on the Cas enzyme provided in the present application. The principle of this tool was based on the fact that GCN4 could spontaneously recognize and bind to scFV, thereby enriching P65 and HSF1 effectors with transcriptional activation function near the target site of the Cas enzyme, and then activating gene expression at the target site.

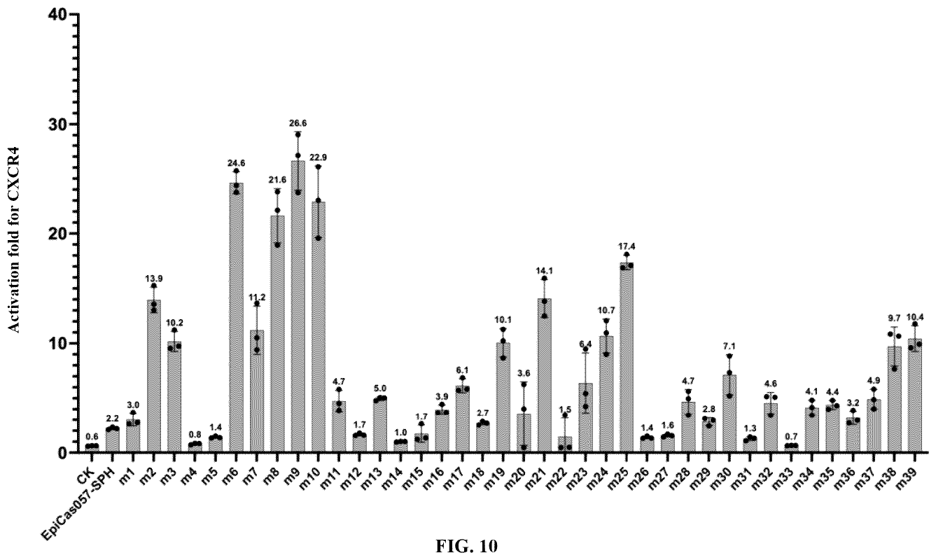

The transcription activation tool and gRNA targeting CXCR4 (spacer sequence is as set forth in SEQ ID NOs: 120-123, and the 4 gRNAs was mixed in equal proportions) were co-transfected into HEK293T cells. 48 hours after transfection, the cells were collected and stained with PE anti-human CXCR4 antibody (BioLegendg, 306506). The fluorescence intensity of the PE channel was detected by flow cytometry to reflect the expression intensity of CXCR4. The activation efficiency (MFI fold change) was calculated by dividing the mean fluorescence intensity of PE in the transfection-positive group by the mean fluorescence intensity of PE in the transfection-negative group, and used to represent the activation intensity of different activation tools (FIG. 10) and, therefore, the DNA binding efficacy of different tools.

The amino acid sequence of the scFV-P65-HSF1 fusion peptide is shown below (SEQ ID NO: 124, NLS in italics, P65 and HSF1 in italics and bold, scFV in bold, HA tag in italics and underlined, linker peptide underlined, and Flag marker in < >):

MGPDIVMTQSPSSLSASVGDRVTITCRSSTGAVTTSNYASWVQEKPGKLFKGLIG
GTNNRAPGVPSRFSGSLIGDKATLTISSLQPEDFATYFCALWYSNHWVFGQGTKVEL
KRGGGGSGGGGSGGGGSSGGGSEVKLLESGGGLVQPGGSLKLSCAVSGFSLTDYG
VNWVRQAPGRGLEWIGVIWGDGITDYNSALKDRFIISKDNGKNTVYLQMSKVRSD
DTALYYCVTGLFDYWGQGTLVTVSSYPYDVPDYAGGGGGSGGGGSGGGGSGGGGSPK
KKRKVAAAPSGQISNQALALAPSSAPVLAQTMVPSSAMVPLAQPPAPAPVLTPGPPQSLS
APVPKSTQAGEGTLSEALLHLQFDADEDLGALLGNSTDPGVFTDLASVDNSEFQQLLNQ
GVSMSHSTAEPMLMEYPEAITRLVTGSQRPPDPAPTPLGTSGLPNGLSGDEDFSSIADMD
FSALLSQISSSGQGGGGSGFSVDTSALLDLFSPSVTVPDMSLPDLDSSLASIQELLSPQEP
PRPPEAENSSPDSGKQLVHYTAQPLFLLDPGSVDTGSNDLPVLFELGEGSYFSEGDGFAE
DPTISLLTGSEPPKAKDPTVSASEGRGSLLT<CDYKDDDDK>

The amino acid sequence of the dEpiCas057-10×GCN4 fusion peptide is shown below (SEQ ID NO: 125, NLS in italics, dEpiCas057 in bold, and GCN4 in < >):

MKRTADGSEFESPKKKRKVHRRRRVREGITMSDTTFRPYKSKLNPNERKMPMVVD
TMNWCDNACSVFFDLFVGLYGGVKIGDVTNFLTEKQDTTTDADLLCAINWFRAMP
VSNPDAVVGPEQLVVRFRDYSGHNPSPMASMYLSAPVNPRYTWVDCRFLYQQMCQ
TLGANLDVDISTLVQTGLLPIQKDTQLNAYSFCSNVFGEGVKEDPSAKVTSYTAAAN
ALSGATNPIITYEDYRQILSSAITNRPSRKGAKDILAADLDSTKTGVLPGNYVSQRIA
MYQASAASKARGVVLPNRLRVKAYLQNLVGLNYICTTWAAPMLRAVGDIKSKNSN
NYRFVGERLERQQEIDNLLAVSNIRSAQAILQQFRANESNQFYIKKRHIGKGLPSLYD
MWDLMDEKEGIDNYIQSLKGDKTERLPIRELLQIIYPHRKSVSAELFLSAAELTDVE
DRQNRCMVHPTVRGSLVINFGKSSTIFGAITPPDTLIKFRGEMVPAGQTGMIWFTM
HLVDGGKWVKHHIPCHSSRYFEEIYAWKKDLPTLPFPRRSLYGYAIGNKISDTRKID
LRRKKASKQYLRTLENMTHNVEFDPTTQFMVDQELNVTITARVKKIPPVRDVPVGS
RIMGLNKNQTIRDCYSIWERVEANTSGSFQHPVVQDVWLRLVADGHVTSTVNGPNG
QIDQMSYSGPDVANCGNWMQERSNFITSVDDADQSIMKHFLWNNRVKLYAWNCVY
AGTLMKAISKQMTTANRNLFRNEILSFVGGRFGCRLGSLAQVSLDLLRRMKSVISC
YFSRNNAFTIEERSAFDKELMELFESLDNKMNNKRKEKVNRTLSSIFQVAKQHNVV
RLVVTKNLQTPDANVRSVMNQRSVDWCARKIEEKIVDLGNVIGIYVDLVEALDLSH
LDPFIYIAKGDVGKECQFDEVESSSLLPIHTSMFRDWHGLVNKGRTTTPIYHSALRSF
ASHYGLRFDDLARIKAAELAKMLPDQKVFIPHRGGRFFMSTYPVTSTSRPVSYDGR
QRFLNSGAMVAPVNTMLRGFRRARSHHHHHHPAAKRVKLDDYKDHDGDYKDHDID
YKDDDDKGSM<EELLSKNYHLENEVARLKK>GSGSG<EELLSKNYHLENEVARLKK>GS
GSG<EELLSKNYHLENEVARLKK>GSGSG<EELLSKNYHLENEVARLKK>GSGSG<EELL
SKNYHLENEVARLKK>GSGSG<EELLSKNYHLENEVARLKK>GSGSG<EELLSKNYHLE
NEVARLKK>GSGSG<EELLSKNYHLENEVARLKK>GSGSG<EELLSKNYHLENEVARLK
K>GSGSG<EELLSKNYHLENEVARLKK>GSGSGTAVNIGGGTGPMDLQRPQGGGGPKKK
RKVGSGVRLQRR*

The fusion peptides scFV-P65-HSF1 and dEpiCas057-10×GCN4 could be expressed together from the plasmid sequence as set forth in SEQ ID NO: 126.

The above experiments demonstrate that the Cas enzyme provided in the present application, which loses cleavage activity by mutation, is suitable for application scenarios of base editing and epigenetic editing, and is not limited to other application scenarios based on DNA targeting, such as gene activation, gene silencing, chromosome imaging, and the like.

1. An isolated Cas enzyme, wherein the Cas enzyme comprises an amino acid sequence as set forth in any one of SEQ ID NOs: 1-58 or a sequence having at least about 80% identity to the amino acid sequence as set forth in any one of SEQ ID NOs: 1-58.

2. The Cas enzyme according to claim 1, wherein the Cas enzyme has a catalytically active domain capable of binding to a target DNA strand and/or a catalytically active domain capable of cleaving the target DNA strand.

3. The Cas enzyme according to any one of claims 1-2, wherein the catalytically active domain comprises an alteration of one or more amino acids such that the Cas enzyme has only the activity of binding to the target DNA strand, or has the activity of binding to the target DNA strand and the activity of cleaving a single strand of the target DNA.

4. A fusion molecule, wherein the fusion molecule comprises the Cas enzyme according to any one of claims 1-3 and one or more heterologous functional domains.

5. The fusion molecule according to claim 4, wherein the one or more heterologous functional domains are capable of regulating the expression of one or more gene products.

6. The fusion molecule according to any one of claims 4-5, wherein the one or more heterologous functional domains are directly or indirectly fused to the Cas enzyme.

7. The fusion molecule according to any one of claims 4-6, wherein the one or more heterologous functional domains are selected from helicases, nucleases, helicase-nucleases, DNA methyltransferases, DNA hydroxymethylases, histone methylases, histone demethylases, histone acetyltransferases, histone deacetylases, phosphatases, kinases, transcriptional (co-) activators, transcriptional repressors, DNA binding proteins, DNA structural proteins, marker proteins, reporter proteins, fluorescent proteins, ligand binding proteins, signal peptides, subcellular localization sequences, antibody epitopes, and affinity purification tags.

8. The fusion molecule according to any one of claims 4-7, wherein the one or more heterologous functional domains have one or more of the following activities: methylase activity, demethylase activity, deaminase activity, transcriptional activation activity, transcriptional repression activity, transcriptional release factor activity, reverse transcriptase activity, histone modification activity, RNA cleavage activity, and nucleic acid binding activity.

9. An engineered, programmable, non-naturally occurring CRISPR-Cas system, wherein the system comprises the Cas enzyme according to any one of claims 1-3 or the fusion molecule according to any one of claims 4-8, and one or more guide RNAs; the one or more guide RNAs target a locus of a nucleic acid molecule encoding one or more gene products in a cell, thereby directing the Cas enzyme or the fusion molecule to bind to and/or cleave the locus of the nucleic acid molecule encoding the one or more gene products; and the Cas enzyme or the fusion molecule does not naturally co-occur with the guide RNA.

10. An engineered, non-naturally occurring vector system, wherein the vector system comprises one or more vectors, and the one or more vectors comprise:
a) a first regulatory element, the first regulatory element is operably linked to one or more guide RNAs, and the one or more guide RNAs are capable of hybridizing to a target sequence in a locus of a nucleic acid molecule encoding one or more gene products, and
b) a second regulatory element, the second regulatory element is operably linked to the Cas enzyme according to any one of claims 1-3 or the fusion molecule according to any one of claims 4-8,
wherein the component a) and the component b) are located on the same vector or different vectors of the vector system, and the guide RNA targets the locus of the nucleic acid molecule encoding the one or more gene products in a cell, thereby directing the Cas enzyme or the fusion molecule to bind to and/or cleave the locus of the nucleic acid molecule encoding the one or more gene products; and the Cas enzyme or the fusion molecule does not naturally co-occur with the guide RNA.

11. The system according to any one of claims 9-10, wherein the expression of the one or more gene products is altered.

12. The system according to any one of claims 9-11, wherein the expression of the gene product is decreased or increased.

13. The system according to any one of claims 9-12, wherein the gene product is a protein.

14. The system according to any one of claims 9-13, wherein the cell is a eukaryotic cell.

15. The system according to any one of claims 9-14, wherein the eukaryotic cell is a mammalian cell.

16. The system according to any one of claims 9-15, wherein the mammalian cell is a human cell.

17. The system according to any one of claims 9-16, wherein the Cas enzyme is codon-optimized for expression in a eukaryotic cell.

18. The system according to any one of claims 9-17, wherein the guide RNA comprises a guide sequence fused to a tracr sequence.

19. The system according to any one of claims 9-18, wherein the guide RNA comprises a direct repeat sequence and a spacer sequence, and the spacer sequence binds to a nucleic acid molecule targeted by the guide RNA.

20. The system according to claim 19, wherein the direct repeat sequence is 10 to 70 nucleotides in length.

21. The system according to claim 19 or 20, wherein the direct repeat sequence is 31 to 36 nucleotides in length.

22. The system according to any one of claims 19-21, wherein the direct repeat sequence comprises a nucleotide sequence as set forth in any one of SEQ ID NOs: 63-88 and 90-99, or comprises a nucleotide sequence having at least 95% sequence identity to the nucleotide sequence as set forth in any one of SEQ ID NOs: 63-88 and 90-99.

23. The system according to claim 19, wherein the spacer sequence is 16 to 24 nucleotides in length.

24. The system according to claim 19 or 23, wherein the nucleic acid molecule targeted by the guide RNA comprises a nucleotide sequence capable of complementary pairing with the spacer sequence.

25. The system according to any one of claims 9-24, wherein the vector or the Cas enzyme of the system further comprises one or more nuclear localization sequences (NLS).

26. The system according to any one of claims 9-25, wherein the system is introduced into the cell by a delivery system, and the delivery system is selected from a virion, a liposome, a lipid nanoparticle, electroporation, microinjection, and conjugation.

27. A method for altering the expression of one or more gene products, wherein the method comprises introducing an engineered, non-naturally occurring CRISPR-Cas system into a cell comprising and expressing a nucleic acid molecule encoding the one or more gene products, and the system comprises the Cas enzyme according to any one of claims 1-3 or the fusion molecule according to any one of claims 4-8, and one or more guide RNAs, and the one or more guide RNAs target a locus of the nucleic acid molecule encoding the one or more gene products, thereby directing the Cas enzyme or the fusion molecule to bind to and/or cleave the locus, thus altering the expression of the one or more gene products; and the Cas enzyme or the fusion molecule does not naturally co-occur with the guide RNA.

28. A method for altering the expression of one or more gene products, wherein the method comprises introducing an engineered, non-naturally occurring vector system into a cell comprising and expressing a nucleic acid molecule encoding the one or more gene products, the system comprises one or more vectors, and the one or more vectors comprises:
a) a first regulatory element, the first regulatory element is operably linked to one or more guide RNAs, and the one or more guide RNAs are capable of hybridizing to a target sequence in a locus of the nucleic acid molecule encoding the one or more gene products, and
b) a second regulatory element, the second regulatory element is operably linked to the Cas enzyme according to any one of claims 1-3 or the fusion molecule according to any one of claims 4-8,
wherein the component a) and the component b) are located on the same vector or different vectors of the vector system, and the guide RNA targets the locus of the nucleic acid molecule encoding the one or more gene products in the cell, thereby directing the Cas enzyme or the fusion molecule to bind to and/or cleave the locus, thus altering the expression of the one or more gene products; and the Cas enzyme or the fusion molecule does not naturally co-occur with the guide RNA.

29. The method according to claim 27 or 28, wherein the expression of the gene product is decreased or increased.

30. The method according to any one of claims 27-29, wherein the gene product is a protein.

31. The method according to any one of claims 27-30, wherein the cell is a eukaryotic cell.

32. The method according to any one of claims 27-31, wherein the eukaryotic cell is a mammalian cell.

33. The method according to any one of claims 27-32, wherein the mammalian cell is a human cell.

34. The method according to any one of claims 27-33, wherein the Cas enzyme is codon-optimized for expression in a eukaryotic cell.

35. The method according to any one of claims 27-34, wherein the guide RNA comprises a guide sequence fused to a tracr sequence.

36. The method according to any one of claims 27-35, wherein the guide RNA comprises a direct repeat sequence and a spacer sequence, and the spacer sequence binds to a nucleic acid molecule targeted by the guide RNA.

37. The method according to claim 36, wherein the direct repeat sequence is 10 to 70 nucleotides in length.

38. The method according to claim 36 or 37, wherein the direct repeat sequence is 31 to 36 nucleotides in length.

39. The method according to any one of claims 36-38, wherein the direct repeat sequence comprises a nucleotide sequence as set forth in any one of SEQ ID NO: 63-88 and 90-99, or comprises a nucleotide sequence having at least 95% sequence identity to the nucleotide sequence as set forth in any one of SEQ ID NOs: 63-88 and 90-99.

40. The method according to claim 36, wherein the spacer sequence is 16 to 24 nucleotides in length.

41. The method according to claim 36 or 40, wherein the nucleic acid molecule targeted by the guide RNA comprises a nucleotide sequence capable of complementary pairing with the spacer sequence.

42. The method according to any one of claims 27-41, wherein the vector or the Cas enzyme of the system further comprises one or more nuclear localization sequences (NLS).

43. The method according to any one of claims 27-42, wherein the method comprises introducing the CRISPR-Cas system or the vector system into the cell by a delivery system, and the delivery system is selected from a virion, a liposome, a lipid nanoparticle, electroporation, microinjection, and conjugation.

44. A nucleic acid, wherein the nucleic acid encodes the Cas enzyme according to any one of claims 1-3, the fusion molecule according to any one of claims 4-8, or the CRISPR-Cas system according to any one of claims 9 and 11-26.

45. A cell, wherein the cell comprises the Cas enzyme according to any one of claims 1-3, the fusion molecule according to any one of claims 4-8, the CRISPR-Cas system according to any one of claims 9 and 11-26, the vector system according to any one of claims 10-26 and/or the nucleic acid according to claim 44.

46. A kit, wherein the kit comprises the Cas enzyme according to any one of claims 1-3, the fusion molecule according to any one of claims 4-8, the CRISPR-Cas system according to any one of claims 9 and 11-26, the vector system according to any one of claims 10-26, the nucleic acid according to claim 44 and/or the cell according to claim 33.

47. The kit according to claim 46, wherein the kit further comprises a container for placing the Cas enzyme, the fusion molecule, the CRISPR-Cas system, the vector system, the nucleic acid and/or the cell, and an instruction for use.


SEQUENCE LISTING (127 sequences)

SEQ ID NO: 1 - protein, 1270 aa, synthetic construct
  REGION 1..1270: note = EpiCas001
  SITE 1270: note = misc_feature - Xaa =
MNNNGETKCYGNNLDRFIGLREVNKTLRNELIPTDSTRENIEKNGIISEDELRAEMGHELKAIMDDYYRNMIEETLSRKCNINWSTLFAAMDSVARSANKKDADKILEKVQKEKREEIHKLFKEQGNFEKLFKAELVSDVLPDFIGKYEGYSDEERISKLGTIKLFKKFMTSFKAYFDNRKNVFSKENISTSICHRVVNDNAFIFWDNLNAFKKIDKYAHDAVETIEGEISGKLGEWKISQIYSVDFFGMVIPQSGITFYNEVSGEINKYMNIYCQKEKQNSRQFKLKRLHKQILCIGETSFEIPEKYESDEEVYQSVNDFIGMIKNNDIISRLEHLAENVNKYDADKIYINSKSYENVSKCIGGTWNTIKDCLRLWYNENIRGSGKNKEKKVGKAVEEDKYKSIAMLNQLLENYSDGDGRIYNVQLYVEHITKIIGDYKPEELVYDANIRVIEQDEKAEEIKSVLDIYIAILHWIKAFIIEDAVEKDTDFYYEIEEIFDKVQSIIPLYNMVRNYTTQKPYKQEKIKLKFNTPTLAEGWHKSKEYSNNAIILQRDGKYYLGIFNAKNKPDKKIIEGCSQKKNKADYAKMVYYLLPKPHMNLPHIFIKSKEAVKNYGLSNYIVEGYDKDKHIKSSNGFDIKYCRDLIDYFKDCISKHPEWKNFNFSFTDTALYNDIGEFYNEIKRQGYKIEWTYISEEEINRLDENGQIYLFQIYNKDFAEGSKGTPNLHTLYLKNLFSEDNLKDPVFMLNGNTEVFFRKSSIKKPVVHKKGSVLVNRNYIDLNGNRITIPEKEYKEIYEYCNGIGNVNLSESAMKYYDMAKHYTATMDIVKDYRYTVDKFFIHMPMTINFNAESNNNINELALNFVAKNDNIHIIGVDRGERNLIYISVIDSKGNIIKQKSYNVVNDYDYKSKLKEREYDRGDARKNWKRINNIKELKEGYLSQVVHEIAQLVIQYNAIIAMEDLNYGFKRGRFKVERQVYQKFENMLINKLNYLVDKRKNADEAGGVLKGYQLTYIPERLKNMGRQCGIIFYVPAAYTSKIDPTTGFVNLFNFNAITSQEGRRKFINKFKHIRYNLNMNLFEFEFDYNDFITHNTQIAKTEWSVYTNGPRIKKERVNGKWNDGYEADITEVMKQKLSDNGIDYADGGDILTRIAEADDKLVSGIFEIFRLTVQLRNSKSESCGEDYDRIISPVLNESNRFYDSDEYSGENEILPVDADANGAYCIALKGLYEVKQIRENWVEGDKLSGDLLKISNADWFDFIQNKRYEX

SEQ ID NO: 2 - protein, 1265 aa, synthetic construct
  REGION 1..1265: note = EpiCas002
  SITE 1265: note = misc_feature - Xaa =
MNIKFNKFTGVKPVSKTLRNALIPTEFTMVNLERNGIISADELRAEKRQELKKIMDDYYRDLIERTLAENHSINWHILFELMKSKMHGDEKDTAKKLENEQKSKREEIHRLFAEQEEFGKIFSAKLVSDILPGFVASYAMYNDEDRTEKAEVVKLFNKFMSSFTEFFENRKNVFSKENISTSLCHRIVNDNALIFLNNMDSYKRIMENFKSESELVSKGLETMLGEWKISQIYSEDFFGMTMTQSGIEFYNDICGELNLVLNLYCQKSKKNSARFKMKRLHKQILCTEKSSFEISYKYDNDKDVYTSVNGFIKNIKNNNIIERLLKLGTETEQYDLNKIYISSQSYETVSIAIGGTWETIRNCLTSWYDDNIKGSGKAKENKIKAVVKADKYQSIARVDYLTETYNASGTDLQKAESYLSHIVQIIGDYEPEELYFDESIHLIENGDKISEIKKILDIYIDVVHWIKTFEISESVEKEPEFYYELDDIYDKILDAVPLYNKVRNYVTQKPYNQEKFKLNFDSPTLANGWSKSKERDNNAIILSRDGKYYLGVFNVRNKPDKEILEGHSKPKNETDYAKMVYNLLPGASKMLPKVFLSKKGIDNFKPSDYILEGYIKKKHIKSSETFDIKYCRDLIDYFKNSISRHPEWKNFGFNFTETSKYNDISEFYKEVEKQGYKIEWTYITEEDINKLDEAGQIYLFQIFNKDFAVGSKGTPNLHTLYLKNLFSKENLDNVVLKLNGEAELFFRRSSIKTPVIHKKGSILVNRCYVDADDGKRKPIPEKEYMEIYNYCNGKRDTELSESARKYYNISQRYEATMDIVKDYRYSVDKLFIHLPITINFKATENKNVNDMVLEYIAKSDDIHIIGIDRGERNLIYVSVIDMHGHIVMQKSYNVVNEYDYKTKLKECEQGRDAARKNWKEIGKIKELKEGYLSMVIHEITQLIIEYNAIVMMEDLNFGFKRGRFKVERQVYQKFENMLINKLNYLVDKHKAVDEPGGLLNGYQLTYIPDRMSELGRQCGIIFYVPAAYTSKIDPTTGFFNAFRFNLLTSAESRRDFLNRFKCIKYNGSMNMFEFDFDYNDFITHNTTIARTQWKVFTNGGRIKNIISKSGWNGTKKIDDITALMKEIFDSRKIDFSDGGNLLPEILAYEGADKDKFIVELLDIFKLTVQLRNSMPDGSDIEYDRIISPVINENNTFYDSDMYTEPNSPLPIDADANGAYCIALKGLYEVNQIKENWKEGETFPKSVLKLNNANWFDFVQNKRYEX

SEQ ID NO: 3 - protein, 826 aa, synthetic construct
  REGION 1..826: note = EpiCas003
  SITE 826: note = misc_feature - Xaa =
MNLAFLNRGYNYGMINRIERGPFSELGDQQLLEQTRRLAANQRCLEVHILDHLDEIDRRGLALRRGFSSLFDYAVRELRFSDAAAQRRIQAMRLCRRHGCVRASLQSGELGMTSAAQLETTFAGAERAERKSHTGHRNVGQGRGPDQDQDSVGSTRQLGLPAAPGEADEQGERGHRSAPEGAPPAAECSGDARERGHRSAPEGAAPAGEYTGEVAARTTPAVAVAVASRSGAQPPGGVHAGALLPRPELNGNAAPVQAPSRAPVAAPGAPGAGAGAGPAAPASDEGPAAQLLPPTEFSASTPEPEPVTAPAELRHTASRAPEPPRDDPFAELVPGTVSAAPLLHPQRQRELIEQAAGMSTRQVASLLAAAAPEVLPPRDTLRAVAPDRYTLKVSIDQECEQGLRLLKDLMAHLDPRMSWGDLVARVVREAVARHDPRGGGRGQRRKGADSAGASPRRTPKETRAAGAAGGPARRVGESTGREPNRTGAAGAAGGPAQREDESSGRAQNRTRAAVAPVAAARRRGGATSAPQSAPAVLVDGARAGGGPAGAPAAPPAPPGDTAATERQNPIEVLTSAAPPSQSAPAAPSDGAHAGGAPAGASAATSAPQSAPAAQLDGARAGRVPSGAPAATSAPQFASGALLDGSNAGAGVPGAPVATAAPQPALGGSPDGGPSGASAGASAGAAGGASADGFSARAAGPGASVATSAPKSVLGGFPDGGPPDAAAGAAAHTSREPVHSRRQSPRRRHIPAAVRRHIWHRDGGRCCYRDPLTGRRCTSAHLLQIDHLVPVAEGGGPEPSNLALRCFAHHRMRHGYAPAASPQPPIX

SEQ ID NO: 4 - protein, 807 aa, synthetic construct
  REGION 1..807: note = EpiCas004
  SITE 807: note = misc_feature - Xaa =
MDGLARVDRAVTACLAAPAWALPEEALVEALDEAHRLGQRLAVLERVLVREVDGRGLARAQGATSTAGWLRDRLRVSIQSARRLVADAGWLDAADDPGTTDDPGTTGDPSGTTDDPGTTGDPSGTRTYASVEPGTGIGAAGAPSPADALPDAAGRTGLDGAVTTGPGPGAGGAGGAGGADRAGAPGLSAAAGCGTAAAGPVLREALATGRVNLEQARIIAGAVESVRKEAGPDTADKALYTLLADARRFEPRALRALADRILWHVAPDAAEEADRAALRRADDEAQRRRHLTLSATDDGRMRLRGLLDSETSATLLAALDPLTRPAGLGDDRTSGQRRHDALGDICRLALRTGELPDNGGEPAQIVVTTEYAALTARLTAVGLPAARQPGATSGTRSSIARPSPEPAPAERGPTEPTPAEQGSIESTPAERRPTEHTPAERRRTERMPAERGAARRTQSGIGRLRRTTHERGGCGPRPPQRAAHNCGQPGRTGRAPAAGSGQRRPERPVPRIRNGTAGTTGTATGRWQRPRGAPTETPTETAVSGGPPTPQSSPPADPSTLGTQRGSPTTDQRPASTDRAPATPVHEATAPAQDALAVQDAAATVQRSPRLDHGCATADQQQDPGRDQDPGRDRAPGRDRHSRGPDRDQWQARDRDHGRQGRGRLGPGRLDNDLRLTPATVRRLACDAAILPAVLGSQGQVLDLGRQRRLFTGPLRRALVLRDRGCAFPGCDRPPRWCDGHHIQHWADGGATALHNAVLLCRHHHRVIHQDDWTVRIAADGHPEFVPPAWIDPEQRPRRNRYHRRAX

SEQ ID NO: 5 - protein, 1596 aa, synthetic construct
  REGION 1..1596: note = EpiCas005
  SITE 1596: note = misc_feature - Xaa =
MVVIECTVPDCEFKTTDASEAVAIALLANHNLVHRNEAQPRRLPANTAPKGPKLDRPKIDVGVTMEEWNIFTRRWEVFRAGSGIDENSAPSQLFQCAGTELGDSILKANPNAVNESLTQLMEAMRSLAIIPIATCVLRTELLQMRQERDESFRSFTAKVRGKAETCAYNAKCSCSRPVDYTDHVIRDVIINGLYDNEIRREVLGIDGILEKPINQVIAVVESKEMARNALPPSPSLSAVSSFQRQKTNSPHPAPSPPQADQAKEATCPGCQKTFKLFTEGARGWNKKPHQLCIDCHRVNRRARRPQRQPQQRSPQAPTVHANQAEPIAQISSLHGETKQDTRPTTHATTAKRPVRLDHHIFSKGEWKRARFRDHPRVPITISLARKGRHRKETADTTPDIHAAVSAIADTGAQSDLWSLADFLACGFSREELQPIRPSLTAANNSPISIEGAFFAKLSATSYSGQSTTSFSMVYVSSSVRDMYLSYDSLLNLDLLPHCFPRHDDVTEASNERRPASQNKTRPNYTSPTTNAARIVNEGCTKPASEHDVTCSCPQRETTPPRPSELPFPCTPANNTKMKAWLLNRYASSTFNTCPHRALPCMEGPPVEIHLDPTAKPTACHTPANIPLHWQKRVYEDLLRDEALGVVERVPYGEPVTWCHRMVVTRKHDGSPRRTVDLSPLNKYCQRETYATESPFQLARRIPNDTWKTVTDAWNGYHSVPLRSSDRHLTTFITPFGRWRYTRAPQGFLSSGDRYNRRFDAILAEFERKERCVDDTVHYDHDLEQHWWRTIEFLTRVGQAGIVLNPDKFQFAGRSVDFAGFHVSDHTIEPLPKYLDGIREFPTPTSTTDIRSWFGLVNQVSNYAQLRDLMAPFKPFLSPRHKFLWSSELEEAFQASKEKIVKAIREGVEIFDIHKRTCLRPDWSKRGIRYFLLQQHCSCSSGMPDCCPGGWGITLAGSRFLSPAEQRYAAIEGEALAVAWGLEQTRYFTQGCDNLVIITDHKPLVKIFGDRTLDEISNSRLFRLKQRTLPWRFEIHHLPGKSNYAADATSRHPSQSNTEEKAPLCPANPPDAAEIALMTAIRDTAQELGAISWTLLAQETAADARMGNLLHSLEQGTQLNKDDPTLANLGPIYESIYQEEGVLMYQDRVVIPPSLRQRVLQSLHAAHQGTSSMEQRARSIVYWPGMSNDIRVTREKCHDCNRNAPSQARTPPLPTAPPSTPFEAVFADFFNYKGYHYLVVGDRLSGWVEVLSSPATTNLAGSTGLIRHLRTFFATFGVPEELSSDGGPEFTAKATEDFLRTWDVKQRLSSVSFPQSNGRAEVAVKTAKRLLLSNTGPTGSLDQDRFLRAILQLRNTPDPDCSLSPAQIIFGRPLRDTLAFVNRLEKFSNPHIRPLWRQAWAAKEDALRTRISRTTESLREHARPLRPLAIGTRVFLQNQQGPNPNKWDRSGVVVEAAGHDQYRVKVDGSGRVTLRNRRFLRAYTPALPAIPQAPSGAPAKTPSMAPDAPSSPSQTCHHTNRTATPATTPGVPTPDLATAALALQVSPEEPDAAEEKQGPSTPEAMTLPSPPNAAARPRRTIRRPKQFEPETGTWTEX

SEQ ID NO: 6 - protein, 932 aa, synthetic construct
  REGION 1..932: note = EpiCas006
  SITE 932: note = misc_feature - Xaa =
MASIASLAPDGWLERKELSDGVSDDGLSDDESLLEERDTLETIRDSLTATNPDEIDGLLGRPSLGSDGLPSPRTPSRRREFLGERKGSKTRLSPTPPAAVPAPTCAPASTKNLWRGFALFAMAMACAALSLQRSPELVEYGPSRRLQTSFGERVDEAADGATSRRLQNSNQFCAARDGWEAFVGTARAGEAFGGEVCLLASTSTESSTLYGSHAAYSPPMRVRAEFLAADDLTRQLGLVFGVPAWPDAANLPATGYACVLEHKVTRWSSKSRLVLRRANADYDKNPDWINFKRSRWFDSEPNAWYEATAAIDDTSAAIAGKVGADVSCEVRDDGGAVVASVRKEDRWVMAHPGKVGFYSERDDLGERFVRALEVDGERAPEEPSFCCYWSPTSDMCGDCPDPQTDGWCAASRARCEGCGGTFCDAPTPPKEVPGRCCYDGAGCGSCSASDDDGFCAKSESNCGSCGGFFCSLAPTPAPTRVPSPRPTTPSPTAAPTVAPGHPTAAPATYAPSSSEPSSKPTTSTPSAQQGCACEEGPYEYDGKTFEGCTSYGWSVPWCGTTNCGICDGLVSTGCWDECEETYAPSSSEPSPKPTTSTPSAAPQGFPAIPLPTSATSPPTPTVFLTNADFRYGTYLITNPGVYVLREDIVFAPETPKMMPPLDSETYPPDRGFWLGFFAAISVAADDVVVDLNGHEIRSSREFLLRQRFFSIIELADKPFKANSGPPQFGALDAPLVPARRVHVKSGTLGLSSHMGIHGNDNEDVTVTDVVIRDFETGGVQLNGASRVRLERVTVGPSLGHEHSAGPVPALATLSQATNLLRVAEGTRHERQEEMVLLSAAVERFVQETLAGEDTSASVFASEAAGYAGLPDGSATVWIPTTGLGGPHQTSELSSSVKSTSIRLIFGRIDCSRRVLEKQPKSLCRNRRIRENX

SEQ ID NO: 7 - protein, 771 aa, synthetic construct
  REGION 1..771: note = EpiCas007
  SITE 771: note = misc_feature - Xaa =
MAGNHFKGSDSGRPAVPPRPNGAGKAGTPGGAGQGGSGKRVSPGETAMFTALYEQSQKAKKTGRARGNVFAGGATFASQPSGAPSVPANNAGAANAANGAGNVNAGKADNNTPSAGGAGLTGNLGTIYTGASETGTFARLDDSGAEFAGSGSKEDYYDFGLNYGSDASSSSTSDGLVRHRHRKGERKKRHTGAIIAAVVAVLLVALGASGFMLLNSAKTVKSEAKEAVEIVGGLKDKVTSGDFSTLPDDAKKIDELCDSMKAETSSPLWAAASFIPVYGSDINAARTMIDALSDVSSNALVPMADNLSQATPGKLFQDGMINVSALQAVADSLSSSSKVFKSANEKVQGIGDTHISQVTELVDKVKDGFATLNGAVDAAEKVAPVLPQMLGANGQTRNYLMYAMNNVEIRACGGFGGSQGLISVTDGQMSIGEFVPRIGLSEDEAVESVDEEDEALFGNHSNLYNSGNTYSPDWPRNSQRVAALWKSQYGQDVDGVIGIDPVFLQYLLGLVGNVSLPDGTVVDGTNAAKVLMHDVYWNYPVEESDGIFASVASAAFDKILGGIGDVDVTKLVSAVERGAEEGRLIAWMRNDDEQNAIKETGIDASLPDPDDPSADPVAGVYFNNLSFSKLDWYLNADTQIGQGIKNGDGTCSYRITVTLTNIMTQEEAGKLPDYVAASAPDAARDDERLNVSLFAPTGGNISDLTVEGTQFGLGAATWHGIPFYSGTVDLHAGETTTITYTLTTSAEAGDKPLTLRQTPTCQAARDSASAX

SEQ ID NO: 8 - protein, 744 aa, synthetic construct
  REGION 1..744: note = EpiCas008
  SITE 744: note = misc_feature - Xaa =
MEAGRDFIAFGQDVQGTDCCRGAGGAGLAGPAGRSGAGAKNVRPPCQTVGMEAGRDFIPFGQVVEGTDRDCCSGAGGAGLADAGAAGVSGVGGVLAGVRQLCAGVVEDSALWDLGAASDFAGGVEEISRAVEYLQLVAASAVDRARTQSAAAAAPGMSWTTGWRDEDPASDENSGTTAPVPVEPPSAAATGSDDGYRNTVEFLRARLRIGAAEARGRLALGAGVLPRQGLAGRSEAAVHAELGAAVAAGAVGSRAAAVIEAALGRVRHVGDAAAAARMEHALTRTAAENDTDFLARVARSWAEAMDQDGAEPSEELLRQRQGAFIRRQRNGLHHLEIFATAEQFEQLVTVMNTATNPRTGTGSGTGAGHGAMNAPAADTPEDAGHGSEPATDGPAGPAAVQEPAQDWAPAATGGAGWETGPAAGAGWIVGPAAVSASDTRAGVVSTSAAGATGDEFVSDPGPGYGFGPVPDPGPGDHRAEAGDTGAGGLDRRTRAQKQLDGLVGACQISLAAGTLPASGGLRPQVMVTIDYRDLLARLGNGTGTTSDSAAPAGTATDCPVHMAGTVDVAGTVDMAGTVDMAGTVGAAGSGRRTGGLPGTGSLLFTGPVTDSTIRKIACDADIIPVLLGTADRVLDIGRASRIFPPHIRKALIARDQGCAFPGCTIPASWCEAHHIDYWSRGGSTGTENGTLLCSHHHHLIHKEHWTIRVRAGIPWFIPPPHIDPAQTPRRNNYFHPARLDPAAX

SEQ ID NO: 9 - protein, 967 aa, synthetic construct
  REGION 1..967: note = EpiCas009
  SITE 967: note = misc_feature - Xaa =
MPNRKVTLTAFSRMDHRVEALEEGLNEVRASIAGVQATMRESYANIVALLEKHLGKSIATEGGTSEKKDKPESSEVKNNVTLEFRQSAKKVELPSFDGDDPAGWISRAEVYFRVQGTTPAVKVSLAQLCMDGLTIHFFNSILRENEDLSWEELKEALLERYGGHGEGDVYEQLTDLKQEAIDDYITEFEYLVAEIPRLPEKQYRGYFLHGLKPEIKGRVRSFAALSEMRRTKLLQVARVVEREVEGGSELKPNRGPRFGFGSGSHKNGSQSDWVMVRRREGSRNEGVKSNTDGPKGDRKRIGPRDRGYTQLTYNELMDYKQKGLCFKCKNPFGPMHRCPAKELRVLIVDDVENEEDEERILAVEVGEEEDEKGEMSILNLHHITHETHHTMKFQGTIRGVEVLILVDSGAIHNFISQKLVHQMDWPIEPTAEMNVKLGNGFQISTQGVCRGLEMCIGDLKMNPEMHLFELGGIDVVLGIDWLKTLGDTITNWKQQTMSFWKDKSWVTLKGHEGCRRQTVALQSILNRPKPTSQGLLWEMDNVKHKGKGVNELSIQQQAEMEVLFNKFEHVFQTPTGLPPKRNRDHAITLTDGHGPVNVRPYRYPHHHKNEIEKQVKEMLTAGIIRHSTSSFSSPVILVKKKDGTWRMCIDYRALNKATIPDKFPISVIEELLDELHGARYFSKLDLKSGYHQVRVKDSDVGKTAFRTHEGHYEFLVMPFGLTNAPATFQGLMNDIFRPLLRKNVLVFFDNILVCSKDWETHLHHLEEVLRLLAAHSLVANQKKCYFGQRSIEYLGHMITEEGVAVDSRKVDSVLSWPQPKNVKRVRGFLGLTGYYRKFIRDYGKIAKPLTELTKKDGFKWGVEAQKAFDELKKRLTTAPVLTLPNFSKEFVIECDASGSGIGAILMQDKKPIAYFSKALGVRNLTKSAYEKELMVVVLAIQHWRPYLLGRKFVVSTNQKSLKQLLQX

SEQ ID NO: 10 - protein, 1214 aa, synthetic construct
  REGION 1..1214: note = EpiCas010
  SITE 1214: note = misc_feature - Xaa =
MIKDFASLPPSHQHKLSLQCQLSTIDDHQTHVIPVLIDSGCTDSIIDEAFVRQHNISTKPLPTRVIVSNADGTKNRTGPLTEYVTLRLTVAGRTELMDFLVTGQREARILLGHDWLRRTNPNINWQTNTITYPSDDTEYHPEDGDRVIAVDLQSRIDKISVESNVSQTKSQQIAEESERAKSTLTFRDVVPPFYHHFEPVFTKASFDKLPPRRVWDHAIELLPEAKGLDCKTYPLNRDEQIHLDEFLKENLETGRIRVSKSPYASPFFFIKKKDGSLRPVQDYRKLNALTIKNRYTLPLISELINRLKGARFFTKLDVRWGYNNVRIKEGDEHKAAFRTNRGLFEPLVMFFGLTNSPATFQTMMDRLLRDLINEGSVIVYLDDIMIFTDSLEEHRKIVSKVLQILADNQLYLKPEKCEFEKKEDEYLGMIISHDTIRMDPLKVQAVAEWPIPRTKKELQSFLGFSNFYRRFIRDYGKIAKPLTILTGKVDWEWAEDQQRAFDVLKKTITTSPTLAIPNDEDPFKVECDASNYATGAVLSQQQEEKWRPVAFLSKSMNPAERNYAIWDKEMLAVMHALEEWRHLLMGAKHQFTIFTDHQNLTYFRDPQKVNRRQARWMDTLARYDFKLEHVKGSANTKADLLSRRVDHPRGERDNEGQVLLPQLAEANIYTMEDVLEEHYLDELLASQHKLDDYVVRKLKEDPKVWKKRDGIVWIGNEKEDELTLGSFENKIYVPQDPKLRARIIEEEHDTSLSGHPGERKTHEGVNRSFWWPSLRRDIRKYVAGCEICQRTKSHRQKPHAPLHPNPIPDRPWQIITTDLIGALPESNGYDAIQVFVDRFSKMAHFVPTHVTLDLLGYAKQLRDSVIRLHGIPEKIISDRGPQFMSNFTKDFYQTLGIEPGLSTGYHPQTDGQSERINQDLEHYLRTFINHRQDDWADWLAIAEFTYNNHLHSATNQTPFFVNYGSHPRKGTGQKPSGRSQAAEDFATSMTKLHEQTRQSLLLANQNLKKYYDRKRTEAREYHKGDKVWLEGTHISTDRPMKKLDDKRFGPFTVKAKVGPSAYLLDVPRSWKAIHPVFNEILLSPYHTPEFPNQPRTSKPPPIQVDGAVEYEAKEIIASRPTKLKGAKLDYFVHWKGYPITERTWEPDHHLKNSPSLITTFHRKHPAAPRVIASAALRFRPYENFTTTTEKPRLFDWITNRFTDCGAKILGTRIX

SEQ ID NO: 11 - protein, 977 aa, synthetic construct
  REGION 1..977: note = EpiCas011
  SITE 977: note = misc_feature - Xaa =
MSITDEYFQLTTKYRAQYGERTIVLLQVGAFFEVYGLKNPRTKVVTGSQIAEFAQICQLSVSEKNVCVEQDHVVMAGFRDYTLEKYLQKLSENDYTAVVYTQTKEGKNVTRQLHAIYSAGTYISYDNDSSQQITNHIMCIWIEKYQALDKSARLSCGVATANIFTGKSEIFEYQTAYYMNPTTFDELERCVSTYCPSEVLVLSTTLPQDVVQTIVQYVGITHPSRHIFCASNMTDAVKNCTQQKYIHHMLTTFFGEESQQLCAEFSNNEIATQAFCYLLHFVQEHNPDLVRKIEIPRFSSQSKTMLLANHTLRQLNIIDDLSASGKQAGQYSSVLSFLNKCASPMGRRLFQQQLIAPTFDQEWLQKEYNMTELMLQPDTEPMIPMLRKQLATIRDIEKICRQIVLHKIYPDSIAHLYDSLQTIQQIHVCFAELPKVLEYLHHSTEKIDQVLHTMQTYLLIDRCRGIQSVQSFPHSIIQPGVSSSLDKIIQTYEENLQHFREIHAFFNKIMRAKEGGDAEYIKIHTTDKSGVSLQITKKRATVLKSLLKGLADPKIQLSGGLYFCVEDIKFSSASTSADEIEIPCLKNICKALLTSEESMNQEIAKSFQDFLTTFENLHLENLEKCALFVAKLDVLHCKAYLASNYNYCKPTIVADHQKSFVDARDLRHVLIEHIQINETYVPNDFCLNLPDQPLGVLLYGTNAVGKTSLIRALGIAVIMAQAGLYVPCSAFTYQPYQSIFSRILGNDNLFKGLSTFAVEMSELRMILRMADESSLILGDELCSGTETESALSIFTAGLLHLHAKGASFVFATHFHEILKFEEMQQMPKLALKHMAVHYDPQRDCLVYDRKLQDGPGNRMYGLEVCKSLYLPDDFLQSALAIRNKYYPTSRGELTHHTSTYNTQKIRGICEMCKTNMGEEIHHLQPQKEANDKGFIGTIHKNHPANLMTLCEACHLKQHKSSITQKRKKTTAGYAVLX

SEQ ID NO: 12 - protein, 1028 aa, synthetic construct
  REGION 1..1028: note = EpiCas012
  SITE 1028: note = misc_feature - Xaa =
MEIFISDNTLNMSKKEDVEPVETNKIMKEYLDLTKQYTKIYGEKTIVLLQVGAFFELYGLRDDKGTILESQIEDICIICQLNYADKKLMYNSKQVIMAGFREYMLDKNIPKIIEAGYTAVVYVQEKDGKNIKRVLHSVCSQSTYLSCETDSSPVMTNNIMCIWLDTFKPMDRNVSTVSKTRDTIVYGVSTINIFTGKSSMFEYQTPFILNPTTFDELERYVSVFSPSEILFVSSLEKEINRTILQYIGVKCPSIHIYDSRDMENKTIIACSKQVYVQHMLSTFFGEEAMNVCREFQMNTMATQAFCYLLNYIQEHDKNIVRKISIPEFNNTSDRLLLANHTLKQLNIIDDSSIDGKKTGNLSSVLSLLNKCCSPIGKRNFKMMLLNPSTNETWLQQEYDMIEYMLQSENIYFIDMFRKQLSQIRDIEKICRQLLVLKVYPSSIYQLYKTIGVIHNLHICLAENKKITDYLCKDISKEQINSYHYINDLCLQINEFLENHFVLDDCKNIQSLTSFENNIIKKGISQPLDDLIKHHYEKIAIFEFVKKQLNEFMYSNGQNRDIEFIKTHETEKSGLFLQITKTRANILKNYIKAKLEKDADYSFHNKELQFSFYLKDIKFGVASSANEEIHIPILQKICKELITIKDEIDVVIRETYLEILKKIEDKWLPSLEQLANYVANVDILQCKTYIAKNYKYCKPEIVKSEKSFVSAQELRHCLIEHIQQNEIYVANDICLGNEKSGMLLYGTNAVGKTSFIRALGISIIMAQSGMYVPCSRFQFKPYTAIFSRILGNDNIFKGLSTFAVEMSELRIILKMADKRSLILGDELCSGTETESALSIFVAGLMDLHDKDASFIFATHFHEIINYDEIKMMNKLSLNHMSVIYDREKDALVYDRKLKDGPGTKTYGLEVCKSLYLTDEFLDKAYAIRNKYFPETRGELNFKSSTYNVNKIRSICEVCKNEIAEETHHLEEQHTADTDGFIGHFHKNHKANLVSICKNCHDKIHHDNSGKNTLNGKQRKKTTKGYDFMX

SEQ ID NO: 13 - protein, 856 aa, synthetic construct
  REGION 1..856: note = EpiCas013
  SITE 856: note = misc_feature - Xaa =
MLLTEEYLEEQKKLELKYGKNVVLFMELGSFYEIYEANGIGKVKEISNILNILQTKKNKNKPESQRNPLMCGVPSVSLDKHLNRLAQEEKWTIAIMSQTATDNGFERKITEIISPGTNVRNNIKDDTNSYLASLILDKNTKGIYSCGLSLIDVTTGNIKVYEGYGVHNDKEKILDEVNLILENYNIKELILLDNLDSNKLYYKFPSINMKTSNKSIHYKIQLITETFNKDFSELGLEQSPYIIDSLVGLLEFLIDHNKSLVVKLNSITFINNKEFMYLGNNPLKQLNIIGDDFSVEKLINKGISAIGRRYIREQILNPIINADEINNRYKYSLSLSKSNKLPTIEKSLKNIYDIERLSRKLELKTIKPFELGNLVTSLYASSDIIKNIKMLDTLTTFESYINLDLIKDYNSIHWNFFKDEQLMTLKTELELIMDLSNYEFDSFIKYSDKDGYYYEFTNKKYENLSFTSTIEHTRKLKNSIKLFTSELSSINTRRLVIESKMMEISNVLFKDVLDIFDITIINIEINKIQRIEFYLNNTKLLAKKYCIPTILDDVENIFDVTNLRHPLVEHLEQEVFVPNSIKFNKENKGYLLYGQNSSGKTVLSKSIGIAIILAQSGFFVPAESLAFTIKDSLFTRIIGADNINKGLSTFAVEMLELKNILNRSTPDSMIIGDEISHGTETTSGVSIVASAILHFNAPFIIATHLHQLDSINDIKEKPFIHFIHLALTFDTSTNSLIYNRTIQKGKGSSIYGLEFAKSLDMPFDFLDKAYAIRKEIADDISGLEQLTISKPSSYNSKLFLGTCYLCDNKGEEVHHIKHQHLADDDGFIDTLRKNHKSNLMVLCSSCHNKQHEKKIX

SEQ ID NO: 14 - protein, 984 aa, synthetic construct
  REGION 1..984: note = EpiCas014
  SITE 984: note = misc_feature - Xaa =
MSAKKGFSMIDEYFNIYNEKIKEYGKDIAVLIECGSFMEIYQIDNNEEKIGNAYEISKILNICYANKNGDLSNNNRSYPNFVGFTTNSMSKYLPILLENNYTVILVNQLETNGNKKGKLIQRGVTAVYSKCLQPLEMDTNADNQLLYISLDVKLIHSNVLKRNKTTMYELNTSICCVNNFTNSIEIYENNYTFTKDCIENCLNDFDHLLYKFFSKDINIHLVGENIINFESELLKFFNSRYDNMRFHNFDKQKHKEYIKTDYQNEYFKKVYNHINFGLLTPIEYLNITTMPSLSIVNLMYTIDFIGRHDLKYIRNLSIPKMMNDSDHLFLALDTLNQLNIDKGVFDIVNCTKTAIGKRYLKSLLCKPLKNPNEIEYRYKLTDELESIINTNNEIDQLLSCIIDFEKLHRKMSLEVLHPFEFEKLHTCYKNVNKLINVLSNYPLFHQIIPNNVKLNVFNEYINSYMETFDFDKLKKYNLNTSKDEISNYFLKGVIVELDEIQLKIDNITEEIERMRVKYNTICENNCKTKNPDYIKIAYTETEGYSFSCTKIKYQTLINAIKESSNFKIKQTNNIVKFFTPELVDLSNKLLTFKDLLKNKINLHYISTLKLYYYNYHDLFTSMTTFIQILDVVNSNLKCKNKYNYCRPIIDDNENDSSFFKAVGLRHPIIEVITQSNKYIPNDIVLDDNNIGILLYGLNSSGKSSLLRAIGIAVILAQCGLFVPCNSFIYKPFHTVISQVDLSDNMYIGKSSFITEMAGLKKILDLSGKNTLVLADELCRGTETYSAISIVCSTMIKLIQSNTKFFFTTHLHQIPQLKRINTLKSIDICHLSVTIKNSNIIFNRKIEKTSGDPLYGLEVCKTIINDNDFINDAFEIRNELTRNNTQILSHKKSNYNKKKITSYCEICNSCNSLETHHITEQYLADKNGIMPDGNHKNNLYNLVTLCHDCHLKVTLGKIKTRGYIQTINGRILDYNFTEDTTEKVX

SEQ ID NO: 15 - protein, 1018 aa, synthetic construct
  REGION 1..1018: note = EpiCas015
  SITE 1018: note = misc_feature - Xaa =
MTTEHKKYEYLKFNKSLNEEYLLLQEKYEKIYNKDFTVVLMQVGSFHEIYATNDRGYDLHKLSNILNIIVSKKNKKNPEVSEKNPYMIGFPILATPKFINILVDNGFHVIKIDQVTPPPNPKRAITQIYSPGSYIEGISSYDSNYILSIYIEEIKQMNNSYILFAGLSIIDLSVGKCIIHETFSTKDDEKYSLDEVLKFIYNFNPKEIIVYYKNLITYSENELLTYLELNDKKYLISEFSDNEYTKIIYQNNFLKEIYNLETCMTHIEELDLERINNGRLSFILLLNYCKKMNSTILNNIYKPEFYLNTKYLHYGNNALKQLNVQTYNSNEKSLFDVINYTSTSMGKRYLNFNICNPISDINILNDRYDKISELKEKNIDLSNKLKKILDIERLHRKISLQNLNPHNFVDLHISYSTILSLYEDIKKTKLNKIYNEKVKMNLENYISYYSRLFNLDEMEKYNLNDITNSFYNENINEKIDILQKSIKKEYKLIENLRDELEKFIDCKKKDYFNSFNEDKSPIQINYNEKDKYHFSTTSKRGSILKNNIKNNSKYNYDSYKFKDQSNTMKITCTYVDNISDKLVILNDKIRPLIKNTYLNDLKEMYEKYNDLYLKINNIIAEIDFINSGSICSIRNKYCKPKIILKDKSFMNVKSIRHPIIEQICSNKYVPHNLNLDDNQIGILLYGLNSAGKSSLMKSIGLNVILAQIGYFVSASEFVFSPYKSLFTRISNIDNIYKGLSSFALELVELNAILKRSGSNTLVLSDEVCKGTEYNSALIIVSAMIKMLVESNTSFISATHLHELTKLDAIKCISKIGIYHISVKYINNNIIFNRLLKKGNGTEEYGLDFAKYMIKNNKFIDICNNMKKDINNITANKSKYNKKLIVDKCQICKSIKNLETHHIEFQKNTDEYGFIIKKTLNHIHKDHISNLVILCSECHDDIHNNKIIMKGYEDTIKGSILKYKRSKCIEEKKYNDDEIKFIINKPNISQKKVKDLFEKQFKKNITIKNISKIINNKYX

SEQ ID NO: 16 - protein, 949 aa, synthetic construct
  REGION 1..949: note = EpiCas016
  SITE 949: note = misc_feature - Xaa =
MYNIIYLKINIVMTLIEEYFEFQDKYTKIYGEDTIVLMEVGSFFEIYQVGDLGKAKEIASVLNMILTKKNKNINEVTFKNPLMTGFPTVSLEKNVSRINAQSKYTVVLVKQRGQSPNITREVDEILSPGIQYRINTFKNNFLANIYVEYYRKNMFGIGISVVDITTGENYLMDSVSLEGFPDNPLSEIQQMVTKYGPSEVIVSMVDFESAESEATRIRVLLDEMIGKYSWKSCDSANYKVSFLNEMFSEIYKVKSILTPIEYLGLEHNQLAGNALIDSIGFIQKNNPCILDKMPRPEYINCAKYLTLNNDALSQLNVVDNNKKANRKGSLFELLNTTVTAAGGRLYGRRLSNPISSKEDLNAIYAEIDLFISDSKFAEVRREIAPILDINRIWRRLVMRKGDPMDIWNLYTSIKILADMKNDFSWIPNLENLSNSAQSANKYIESIFDISRIEDSYLSNLEDNIFKKENPILDKLEAELAEIKYKSQIIAEMFANHSFFTEKTKLVYTDKEGFYIETTPSRYKQYLGLNTAPKSDILAGFDVYKNKSKVRLTNDDLRKLSESKVIKIQKMSILIKEMFVKEVDEMYSAIGDDVESISAFISHIDVMAANAYNSVNLSLFKPELIDNEEGYSSVECHALRHPIVERLTHSEYIQNDVLFSNNKYGKVIFGVNSTGKSTLLKATGLAIIMAQAGMHVPSKMKLSPYQSLYTRITSGDDLFSNKSTFTVEMLELKQILEKADHNSMVLADELSHGTETNSGVSILAATINTLSKRKATFLFTTHLHQITGVRSIMDNKSIDLSHLSVNINPITGVIEYDRKLKDGAGESVYGILVAKGLGLADDFINDATSILNEVKSNRTQLDAITENNTNKYNSNKVNKNCEICGFQSSETHHIKEQADFKNQKYLEIQKNAKYNLVNVCNDCHKKIHSGKIVNLMYKQTTEGVELKITX

SEQ ID NO: 17 - protein, 880 aa, synthetic construct
  REGION 1..880: note = EpiCas017
  SITE 880: note = misc_feature - Xaa =
MPSLIKEYFKIQEESEKKFGDKTVIVMHVGGFYEVYSYEGLGKADVVANECNMMLTSKDKSKEISITNPQMCGFPLASLLRYMYFLTSKGFTCVTVKQDEKVKEKRTIDRIFSPGTCVDDSESSIVASIYADDGCIAVSWIDLTSGKVMFEEIYNDNEAYRREELTRIIETCGCKEIICMSKNGIMYDDAHEIDFCNIYEHISHQNDVISRAYPNDSTVSPIEFIDMEMFHIARVSLILLIEWVEDHHQSLIQRIRIPERINRKSLALHNTSVHQLQLVSQDKKSLFDVINKTNTSMGKMLLKQTLLNPYSDVSKLNGMYEDIERISKVDVEVVKKVLKNIGNVDRLARRIANKSINLEDIRHLILCFENGLDIIEHMRELSVHNMISENDVKRCYIAWHSTIDMEKDVFREGVYDDVDILKKERDTIMYMMDANAKRFSKTLGVNEGVKFELHKGTYGLITTPGRAKTLKNRVGNNVVFQADCKQKVVISTPDIQSLIHKLHGAIDAYDECMEEKMKEFVEILYSYHEIICTFGTYIAQVDVLLSHYLVAKAFNYTKPVVKEGDKSYITGKNIRHAIVERLNGNTLYMGNDVDLSNEKGLLLYGVNGSGKSCFSKSVGICVIMAQIGMFVPAETFEFCPYERIFTRISSEDNIYKGQSSFYVEMMEMNTILRFANEKSLIIGDEICKGTENISAISIVASMCNMISKIGATFVFATHLHGLVDLEPIKQIIDTIQIKHIGVTIESGVVTYDRHIKNGEGEHLYGLEIATHILGNKEFASLALETRNALLKKTPPKKSKYNARVLKKVCHICGRKDEQLDTHHINYQCDAHENIKNTKGNLVVLCKKCHDDEHHGFLSIKGWKDTSKGVILDYEFVSEKX

SEQ ID NO: 18 - protein, 927 aa, synthetic construct
  REGION 1..927: note = EpiCas018
  SITE 927: note = misc_feature - Xaa =
MPQLLVDEYIELTNEYRKKYGEKTIIWMQTGSFYEVYAWNEEDEQIKVSQNILQIRITTKKSGNNRSCLMAGFPDHSYKRFEKRLLKENYTIVYVDQIKKDPIERGVTKISSPGCVFDSDDDSKESILMSVLIEIEDNDFYGYYTIYDSNIGEIRMLSIKNEENQTIDHIFDNIGDIISENRVNEVLLNIVSEQYIKLPETNSKILFHYNKIDKIDELLKPLLQNDILSKYFKKYLSIYENVIDSLELSKCLPQEIVNLIYMLDFLKEHEPIFVKNLQRPIVDGPNQIKYMEFYNHALQKLQVISNEGNDLIKFIDKTLTYGGSKKLKYLLKNPSNNIEELKKRYKSVDLFVNNRQLLNETKTYLKICDLHRFYRRFAIGKINVYNDIPRIVNINENINILINIFKNVDNSIYWLPNDKIIQNFKDYCGDLIYTFCIDNCKSSGNVFNKDIVPELDDLYLKSESLNQELESIRTYLCNLINEPIYLKNTDKDGFFFETTKKRGTKIAKQLKENNKFDLEISNSTSVSKISSAGVKKISVQLCDLQTKIIDLTQKKVSEKVLFYYDTYFESSIATIIESMAWLDVYYSYSKSAIEWNYVKPNLVECENSYIDAKQLRHPIIEQLLSIEKQAFIPNDIRIGFKESYLLYGVNSVGKSSLLKSVGLSVIMAQAGMFVPASDYKLCPYDKLIVRIGNTDNLFEAHSSFICEIREANQCVSNTNNRSLVLADEFCASTERESATQIVTSMLLWLNEKKSSFIFATHLFELIEAPIVNVKICHLKVRIESDSLVFDRKLTSGPPTERNYGILVAKKIFKNNKFLKMMDRCQTKTSTRTDINRSRYNRGVLLSECAICAYSPIDEMSLPLDTHHINMQCMANNDGFIGNYHKDIVSNLVVLCKKCHIMAHNGKLNIKCWEQRDCGNVLNYEINX

SEQ ID NO: 19 - protein, 806 aa, synthetic construct
  REGION 1..806: note = EpiCas019
  SITE 806: note = misc_feature - Xaa =
MLHPRLQEMLAARGIRRLYTHQAQAANLALGGAHVVVVTPTASGKTLCYQLPVLDRLLRRPQSRALYLFPTKALAQDQHHGLTQDLLEAGTDAAAMIYDGDTPQAQRRAARRIAQVLITNPDMLHVGILPYHTQWRELLAELDYVVIDEMHAYRGVFGSHVAHVLRRLQRLCAFYGSAPQFILTSATIANPAELATNLIGEPVEVVAESGAPSGERHVALVNPPVIEARLGIRRSPLVDARQLALSLAAQGEQVVCFTRSRLAVEQLVIALRERAGRVGLAPEAIRGYRAGYLAAERREIEAGLREGRVRCVVATSALELGIDIGGLTVAVLVGYPGSVASTWQRIGRVGRGQAPSAAFLIASASPLDQYLMAHPEHLFGGGAEAAHINPDNLYVRSGHEQAALYELPMAEGEAAQDDDRRAILAALEDAGEARLSRGRWFWSGQGFPAGEISLRSAEAARVSIVARRAEEGRDITIGEVERSSAPRWLHEEAIYLHEGQPYLVRTLDLEGGVAQVEPATTDYFTVASEREEIEIERVVASTDGERMTTAHGEIRLTRQVTGYRRQRFETQEVLGWGVVDLPEQVILTSAFWLGVKPELVARLDAQGLWTADRGGARGPDWEQQRDRARLRDGYRCAHCGAPERPERAHDVHHIVPFREFGWEPGRNDAYRYANRLANLVTLCPRCHQLAERQVALGSTLSDMGHVLRYLAPLWLLCDPEDIGIHADSAGRRAERPTLFIYDQLPGGAGLSDQLPRLIVPLLKSAAQVVSDCPCETGCPACIGPVLAPAIGRKRRAVGLLEALAWX

SEQ ID NO: 20 - protein, 853 aa, synthetic construct
  REGION 1..853: note = EpiCas020
  SITE 853: note = misc_feature - Xaa =
MSWNEEITPLAALLQRWRQDPAFRRRVAAWARAPAREAQWAPIPEDLHPRWREALRQAGFSMLYTHQVEAYRAARSGAHVALFTGTASGKTLAFLLPILQALCEDPQTTALFIYPTKALAHDQLAVLRAWLSALRLPAAALPYDGDTPGEARRFVRERGRLLLTNPDMLHLGILPQHPRWAAFLSHLRFVVIDEAHVYRGVFGSHMVNVLRRLRRLAAAYGASPQFLLASATVANGPELAEGLVEGSVRIIEEDGAPYGERHFILYRPPLLDAATGLRQSALTAAAGLIWTLLEHGIPTIAFARSRMAVERLLRMVRSAWAERGGDPEEVHGYRGGYLPEERRAIEKGLREGRIRAVIATSALELGMDIGTLEACVMVGYPGTIASVRQQAGRAGRRAPLSVAIMVLTATPLDEYLAGHPEFLFRNPPEAARFDPDNLAILADHLRCAAFERPLDPGEPFARTLDTGWIVERLIADGEPLYRNPTDGRVYFVADRYPAQGVSLRTSTGDRVKIRLGAGKGGRIIGEVDRPSAPRLVHPGAVYLHEGAAYRIVSVDWEQGEAMAEPFDGDEITEPIVEARWYLDAERARRTEPWGIGAWGEVTIVHQVVGYRRLRWPGGELLGWAEVETPEEILSTTAWWLTIELGLVERLKEEGIQIGPLDYGPEWEAIRRQVRARDGYRCRVCGAPEPPGQRHDVHHLRPLRTFRDPAEAYALENLITVCRRCHRRLEQMLGTRSALSGLAHLLHGLAPIFLMSDPADLGLLVDPESREGGGPTVLLYEGVPGGVGFAAHLWAVQEALLEAALEVVRRCPCERGCPACVGPPGEMPDLKGETRRLLECIFAADEAAASKVQX

SEQ ID NO: 21 - protein, 845 aa, synthetic construct
  REGION 1..845: note = EpiCas021
  SITE 845: note = misc_feature - Xaa =
MADLERLLERLRRDPTFGPNVTAWRRREGRPARYGETPAWLDPRLQEALRAGGIERLYTHQVAALQAVRQGEHTVVASGTASGKSLCHHLPVLQALLEDPSDCALYLFPTKALAHDQEAVLRALVERIPDLPDSAKPFPASYDGDTPGQARRAIRERVRIVLSNPDMLHAGILPSHPHWAPFLHHLRYVILDELHTYRGLFGSHIANLLRRLRRVCAFYGSRPQFLCASATIANPGDLAGRLIGEPATVIEEDGAPRGPMHLLLYNPPLLDPALGIRRSALQEARRLAVRLIGAGLQTVVFGRSRRGVELLLTYLRQDLPPGLRPALRGYRGGYLPALRREIEAGLRSGEVRGVVATNALELGVDIGTLAACIMAGYPGSIASTWQQAGRAGRRDQPGLAVLVAGANPLDQYLVTHPDYFFGRSPEQARINPDNPFILAAHLRCSAFELALEDGAPWGTPAPPDLLEDLVREGSLLHEGGRYYWIGVGSPAREVSLRTASGDTVTIIEESSDGIGTIGQVDRPGAAMLVHEQAVYIHEGTFYRVERLDWEAGLAYVRPAQVDYYTQAHLRVDWRVLSEEAQAPQGRGSRHTGEVEVHAQATAYRRVHLHTQESLGWGAISLPPQEFRTQAFWLTLPWPAQEHLRPDYGPNWEEQRRRARERDRFTCRHCGAREEVAGREHDVHHLRPFRTFGYIPGENDAYLEANRLENLVTLCRVCHARVDHGPRQETVAALQGLGHLLAHVAPLYLLCDRGDIRETTRLRALPEGRPVVLIYDDCPGGAGLSPTLFSCADALLQTARERLQECPCAGGCPSCIGPPEEPARDKVIHILEFLLEEEAPPPGTAX

SEQ ID NO: 22 - protein, 765 aa, synthetic construct
  REGION 1..765: note = EpiCas022
  SITE 765: note = misc_feature - Xaa =
MNKLWQHPNPDWVPVSHFGELRYESIDVVMARWDWDKLIWGKVRRFVFNLQKRIYKATKSGRYRKAKSLMYLLQNSYYATLLAVRTVTTENFRGKCFCARAKTFTSEGKRTAGVDRVKANTPKRKMALVKDMLDTIQRGWDKYRPMPAKRIYIPKANGKPKVSEAKLRPLGIPTIKDRAMQAVTKIALEPFYEAKFESCSYGFRPAMGCHDAIDRITAALLHKQKWVLDADIKGCFDNIDHKFLASQIDTHWRPIVKRWLKAGYMEKSDYNQTNMGTPQGGTISPLLANIALDQMETDLIEHLRGIKGWKKKIGVTTVSIVEKTEKPQKEKKTQKKPKKANNKGTKEPQKKQKNVYEGAKKSYKCRRNLLIDLVRYADDFVVLHKDKEVIEESKRFIGKWLEVRGLTLSEEKTKIVHSTEGFDFLGHHIRHYENRIKGTYKLKLLKGTPTEQRRAKATHVLRVEPTKDKIKGHYRVIGDTIWKLKAATPSELIRILQPKISGWANYYKTVHSSEAFGKLDHLLWKRLYQWAQRRHPNKGKRWVTNKYFATVNGRRWTFAEIVGKGKDKKIVKHLKGYSKHKERTGSYARVGYDRSYYDGDTAYWAERLSKGYGNITPSKAKMLKRQDGICPVCKCRFGNEDLPEAHHIHPRHLGGEDKYTNLVLVHLHCHDKIHRENPGIKGKLVSQTSPELQGFLNNGMAPAFQAEGMKPPANTSTVLRTGKGAGIITPASDEMKSPVGTSTVLRTGKGAGIITPAPDDNKETX

SEQ ID NO: 23 - protein, 771 aa, synthetic construct
  REGION 1..771: note = EpiCas023
  SITE 771: note = misc_feature - Xaa =
MDTLQLSNPDGRVRSQHSLTGLPSDKKHRDHTDSSGRLVPSCYRVINSSDMCDWDWDSIIWGKIRRLVFNLQKRIYKLTKAGKYRKARSLMYLLQHSHYAMLLGVRKVTTENKGKKTPGVDKVKVTTPRQKMNLVVDVLDTIQMGWDKYEAKPAKRIYIPKANGKERPLGIPTVLDRAMQAVTKSALEPFYEAKFESASYGFRPGMGCHDAIDKIASVLLTKQKWILDADIKGFFDNIDHKFIVEQTDKHWRPIVKKWLKAGYMEENKYNPTKNGTPQGGIISPLLANIALDQMETCLIEHLRGIEGWKTRIGNTKISIITDKKTQKKYESRKHLQLSVIRYADDFVVIHEEKEVIEESERFIKEWLGLRGLTLSEEKTKIGNETDGFDFLGHHIRHYENRIKGTYKAKLLNGTPTEQRKAVTPHVLRVEPTKAKVNAHYQELSDIIGKMKATTPSELIGILQPKISGFANYYRTVHSSKAFSRLDHLLWKRLYQWALRRHSNKGKRWVAAKYFCTVKGRKWTFAEIKGKGKDREIVKSLKGYSKHKVRTGSHARVGYDRSYYDGDTAYWAGRLSKGYGNISPSKAKLLKRQDGLCPVCKCALTNEDLPEAHHIKPRQYGGLWEFKNMVLLHLHCHDKIHREYGEQKRKLCKGTREYDELVKKGVFVSPEVEAKYGPDIAKMRKGPEVNKGSSKILRLGSDPGIISRDKDTVPQETHSNGNNEDDELIQVKSWKSGEIKMIPRSKMYRKTKRVRDKITNKWKLVKMWFCKX

SEQ ID NO: 24 - protein, 706 aa, synthetic construct
  REGION 1..706: note = EpiCas024
  SITE 706: note = misc_feature - Xaa =
MNILWQHPNPSEVNVFARAQKQFTSEWIPVSHLDRHRYESIESVMAKWDWDKLVWGKVRRLVFNLQKRIYKATKAGRYSKARNLMYLLQNSYYATLLAVRTVTTENKGKRTAGVDRVKVNTPKRKMALVKDMLDTIQRGWDKYRPMPAKRIYIPKANGKLRPLGIPTIKDRAMQAVTKIALEPYYEAKFESCSYGFRPAMGCHDAIDKIAGTLLNKQKWVLDADIKGCFDNIDHKFLASQLLPLQHWRPIVKRWLKAGYMAKSEATKKSEYYRTDMGTPQGGTISPLLANIALDQMETDLIEHLRSLKGWKTKIGTTTIGIDINKKTKKSYKSRKNLKIDLVRYADDFVVLHEEKEVIEESKRFIGKWLEIRGLTLSEEKTKIVHSTEGFDFLGHHIRHYENRIKGTYKLKLLNGTPTEQRRAKATHVLRVEPTKEKIKGHYQDISDTIWKAKAATPVQLIALLQPKISGWANYYKAVHSSEAFNKLDMLLWRRLYQWACRRHPHKGKRWVANKYFATVSGRKWTFAEIVGKGKDRKIVKYLKGYSKHKERTGSHARVGYDRSYYDGDTVYWAERLSKGYGNITPSKAKMLKRQDGICPVCKCRFGNEDLPEAHHIHPRHLGGEDKYTNLVLVHLHCHDKIHRENPGIKGKLVGQTSSELQGFLNNGMAPAFQAEGMKPIGTSTVLRIGKDAGIITPVSDGNKETX

SEQ ID NO: 25 - protein, 747 aa, synthetic construct
  REGION 1..747: note = EpiCas025
  SITE 747: note = misc_feature - Xaa =
MNILWQHPNPEWIPVSHLGRYRYESIESVMAKWDWDKLIWGKVRRLVFNLQKRIYKATKSGRYSKARNLMYLLQNSYYATLLAVRTVTTENKGKRTAGVDRVKANTPKRKMALVKDVLDTIQRGWDKYRPMPAKRIYIPKANGKLRPLGIPTIKDRAMQAVTKIALEPFYEAKFESCSYGFRPAMGCHDAIEKIAAALLKKQKWVLDADIKGCFDNIDHKFLAVQIDTHWRPIVKRWLKAGYMEKSDYNQTNMGTPQGGTISPLLANIALDQMETNLIEHLRGIKGWKKKIGTTTVSIVVGGKKAKRPQKENEKSQKEPKKANNKGTKESQDKPKKVYEGAEKSYKARKHLHIDLVRYADDFVVLHEDKEVIEESKRFIGKWLEVRGLTLSEEKTKIVHSTDGFDFLGHHIRHYENRVKGTYKLKLLNGTPTEQRRAKATHVLRVEPTKEKVKLHYREISDTIWKLKAATPVQLIAILQPKISGWANYYKTVHSSRAFGKLDMLLWRRLYQWACRRHPNKGKRWVANKYFATVNGRKWTFAQIVGKGKDRKIVKYLKGYSKHKERTGSYAKVGYDRSYYDGDTAYWAERLSKGYGNITPSKAKMLKRQDGICPVCKCRFGNEDLPEAHHIHPRHLGGEDTYLNLVLVHLHCHDKIHRENKGIKGKKVSQTSPELQGVLNNGMAPAFQANGMKTPTLTDTVFRVGEGAGIITPALDEKKPNADTSTVLRTGKCAGIITPAPDDNKETX

SEQ ID NO: 26 - protein, 758 aa, synthetic construct
  REGION 1..758: note = EpiCas026
  SITE 758: note = misc_feature - Xaa =
MKQNGIDEITIKSSGEINPKSGTNTRILYEHQKNAMECLDIMNKEKAYSTLVVLPTGGGKTYTASMWLLKNAIDRKKKILWIAHRQMLLEQAAESFQKFAYAEVIPHISSFHFRIVSGASSHDQTIHIRPTDQLLIVSKDSIGRNLQRLDAWMAGENEIYMIVDEAHHSTAKTYRKVISYIKEKVPNVKLIGLTATPFRTAEEEQGLLAKIYTDGVQNGQAVHGDIGITYQIGLKELINRRILSKPVFESYYTDEFYGKELGLKAWESIQHLDMIPDDLAKKMAESSARNKLIVETYQANQEKYGQTILFAVSVEHAIQLTAQFRKAGVAADYVVSSIKDMITGVTLSREENEKKINAYRVGDLQVLINVNILTEGVDLPKTQTVFLTRPTVSTILMTQMIGRALRGTAAGGTANAYIVSFIDAWDEHIAWVNPESLFVGNNDFQENAYEQKQRDLRMIAISKIEEFARILDDSTDTSLLEKVPFTQRIPVGMYAFTYLEENGGIEHAYQVMVYNSSQKAYENMMDSLPDLFQSFHAEEEYLSEHQLMEMEKQCRDTFFCGEMVPPYEPKDVIHILKYYAQYESVPKFYTFDEVDRSKLDVGMIAQHIWAEDMGERKKAEYIDKIWNDADDNMLRLFFGKKLYFLRQLDIELMKIRYPDIYEETANVVYETRKLEELSLHDIGKVNPKLEMQLRDAAFEKAKTSDGKYQCAVCGMKNRSKTCFHVDHIIPMNQGGKSVPENLQILCRQCNGIKGDQIX

SEQ ID NO: 27 - protein, 755 aa, synthetic construct
  REGION 1..755: note = EpiCas027
  SITE 755: note = misc_feature - Xaa =
MARDYIEVKSFGKVFPKKGANTRMPYEHQRDAMENLDIINKSNKFSTMIVLPTGGGKTYTASMWLLRNAIDKKKKILWIAHRQILLDQAAESFQKFAYSEVIPHIPFFQYRIVSGASCHDRVINIQALDNLLIISKDSIGRNLNGLDVWLKDEKEIFIIVDEAHHSTAKTYRKVIEYVKSKVSNVKLIGLTATPFRTASDEQGLLAKIYEDGVENGKAIHGNIGITYQIGLKELINHRILSKPIFESYYTDEMYGESLGLNSWKSIQHLDNLPEDVAEQMADSAARNKLIVNTYKAKQKEYGQTIVFAINMVHAVHLSALFNKEGIKSEFIVSDVRDSVTGVRISREENERKLEEYRSGNVKVLVNVNILTEGVDLPQTKTVFLARPTVSTVLMTQMIGRALRGEAAGGTTLAYIVSFVDNWNEHIAWVNPDSLFSGNNEFTDNPVEHVKHELRMIAISKIEEFASVLDDSVDTTMLEKVPFIQRIPIGMYAFTYLEENGVDFSYQVMVYDSTRKAYEDLMASLPQLFKIFDCKDEYPSEELLEEIAEHCRDTFFLGDMIPPYEEKDCLRILKYYAQYEIPPQFYLFDDIDRNKLDVAVIAKYIWDEDMGQRRKTEYINSIWENSDENLLRLFFGRKLYFWRQLDIELTKLSNPNIYKKEENVQLGIRKLEDLSLFEIGKVNPEMEKNLRDTAFEKAKDSEGNYICACCGQKRTDRIFFHVDHIIPMNKGGKTVPENLQILCRQCNGMKGDKFAX

SEQ ID NO: 28 - protein, 781 aa, synthetic construct
  REGION 1..781: note = EpiCas028
  SITE 781: note = misc_feature - Xaa =
MVAINKRALSEEDIKMKYITPAIEKAGWDIKTQVRAEYTFTDGRIIVRGNLVARGKKKRADYILSYKPNMPLAIIEAKDNKKPLGAGMQQALEYGEILDIPFIYSSNGDGFLEHDKFTGKERVLSLDEFPSPNELWERYKKGKGIQPHEEKIIKEDYFFDMSGKTPRYYQRIAINRTIEAIAKGQKRILLVMATGTGKTYTAFQIIYRLWKSRTKKRILFLADRNILIDQTMANDFKHFGDKMIKVNRKNISKAHEIYLALYQSMTGSEEWQDIYKQFSPDFFDLVIVDECHRGSARDNSAWREVLEYFSSATQIGLTATPKETRDVSTSTYFGEPVYTYSLKQGIEDGFLAPYKVVRVSLDKDLEGYRPAKGKVDKYGNLIEDRIYNVTDFDKNLILEKRTEIVAKKITEFLKKTNRFDKTIVFCVDIEHAERMRQVLINENSDLVKENPKYIMRITGDDEEGKAQLDNFIDPASKYPTIVTTSKLLTTGVDAQTCKLIVLDSNINSMTEFKQIIGRGTRIKEEYGKYFFTIMDFRGVTRLFADPEFDGDPVVVYEPKKGEDIVPPAEGDYGDFGDSVDGTVGEPDSDENETGTRIKYVVDDVEVMVVNERVEYYDKDGKLVTESLKDYTKKNIRNEYADLNEFINRWNSEDKKSAIIEELKERGVFLDAIKEEVGKDLDEFDLILHLAFDQKPLTRKERAYNVRKRNYFSKYSDVAREVLEALLDKYMNEGITELESMEILKLDPFRKIGTPKKIIKAFGGKEKYIQTIKELEREIYAX

SEQ ID NO: 29 - protein, 821 aa, synthetic construct
  REGION 1..821: note = EpiCas029
  SITE 821: note = misc_feature - Xaa =
MSNPGNSVLSTRYRDARTLVRRVCRLPGEDRAPFKEKVARLREHFEQFNVDVSELCQWFMGLRKAHAAAHAANAATSPAGFGPLGDFLLVPALDGVDADESECDRWRLAVFDAVAGFRDLTAPIREAVPPALHAAIDATARNGPHTNPKNVAMKRLFERLRTLDSAHRLVLLKSAAEWVVARYRRGMENWVRQREEWEKEKTAWETARPQLTPEVRERFTQVFRSLVWDRDKRPGVTNKRPRICPYERLKGNLDNCIYAGQKGHGPLCWKYAGFKKTREEQFSRFNDRGFEKDAAEFLARRKDGVRRHEVLRQMFRGRSADEQRFTDNWSAYLRALSLNEDTVVEHGRLPHCLRIGETFEKSKCEWNPHTELCKQYKRALDNPDNGFDEETLRLEPLYRQWRRDYLAGPRKPSFRYPSSRDLPMPKIFGAGFHEIDFDRSIVRLRLDDMSAGQWIEFGFTPWPRGYKPSRDEIKAAGRVTSVHVHFIGTRARLGFRFSVRPKPSRFTCSQDELDELRSRCFPRQAQDQQFLDAARQRLLDSFPGGAEEAKRNMRLLAVDLGETGACAAVYQGTTHQTDCPLAIIKINRLYAGRPDALEPDVKGRPSKEKRKFDKGDPRGVRKEHVARHLARIAQKTAEITQHPRRQPAVPQTVTPGAHDLRSLKRHTAWMIRDWTRLNAAQIVKVAEDQRCNLIVFESLRGFLAPGYDKLCPKEEDKKRWLAMFAYGRVRRKVVEKAVERGMRVVTVPYYKSSQVCSTCGAVQENTNLHRENKRDRKFICEKNTCAVELNSDANAARVLARIFWGEITLPSPDEKTVRRSX

SEQ ID NO: 30 - protein, 773 aa, synthetic construct
  REGION 1..773: note = EpiCas030
  SITE 773: note = misc_feature - Xaa =
MTRIKYRQERTLVRGLQRLPNQDKAEFNKNVLGLRRHFEQFNLNVAELCQWLISFKPENPGSVCETKLFWEFMLEPENFCPDTYDRGPDWLRHEIFVLAAGWRTFEDIKAHNMPESLFESIKIASSKPRSKTAEALFIRLKSLEASHVMVLLKSASEWLSTRYVRQSENWKQNEQEWQKKKAAWEDKHPELTPAIRDKYNAIFVELGISEKRPHVCSWKKLSENKDNCDWAGERKNIGDKWISHSDLCIKYHEFARKLRSKQRQHFVDNANQYLELRRRYPQWTRDMAMNGLFKNVPLARNWFRNAWTNYLNALNILETTILENYSGHLPHCEKLSDECVFKKHTDNCRRYKLLLGEKLSNQERELEETYREWRREFLAPPNKPFLRYPSAQKLPTPKLFGRGYYDLDFTRHVVKLRLDDMPADNFVSFGFKPWPRGYDKKPGEINITSVHVHFIGTRARVGFRFAVPHSDSRFSVSQDKIDELRSGGFPGKSQDQEFLNEARQRLLDGMNENQKSALRIMAVDLGTHRAAAAFFTGCIFNKAKLLKLKKIDLLTEPKTDTTKPEKLSADEKKIQREKGLTQHHVGKHFETLEARTKEIISKRQNMKMAPSDDTPDIVGDHDLRHLTSHIRRMIRDWVRLNARQITELAEEENVDLIVFESMRGFRAPGYDKLDLKKKRRLAFFAYGQIRRKVAEKAVERGMRVITVPYFKSSQICAQCGRSQNDKNKLRDNKWKQSFQCEFSDCNYKTHSDENAARVLGRVFWGEITLPTDX

SEQ ID NO: 31 - protein, 950 aa, synthetic construct
  REGION 1..950: note = EpiCas032
  SITE 950: note = misc_feature - Xaa =
MQQGCDSRFDTTIKEQEVSLFNTYTKYVDCVNKLPNAQLVDADFSLKLLSMVIANALEVLNGIKKTVDNDKKYLLLSGGFKLPEVVDVLARLTSNVRQDGAIEIANFGLNSRAVRKRDILNSKKNELDEIESELGALTNKIKKSAQKPKKEDINSKGRLTQERNSLVAEIKKLQAMNKYGLIFRRKNCDGDFEYFLALVDRDHMENLSDKSKFKSAQSTGYQYMEYNCLKLKSLLQIIFESDSDNLSGVRKLERYGTVKPLFLSKEHSRELEKSKTLIAWLDENIQKIVDTIAGTTNPKITYTEYGNTDAAKNAADVWRDFERHFNRQSYSISWCHLNFEKLKTTKSVELFQIANKDHSIHPDFTSDERDRKKLNRSSGRPNLFTTYLDNVLTDEIGNHYQLLGEGVEYFKHKCGAEPSTPRRKSIDRYYVEFRVAFVPNKSESWEETNSSVKNAISQSDSPKHFIGIDRGENAIANIVVIDENNRAILKQDMSCLAIFSDITPHSAKKSYIFLTGDEPVYIATKSATDALTDKTEIKKLSDVMFTEYRDKLYKLLPPSEDKKVIPSKSSPCLDEMNIEDLKKLISKLESPEYNPASRKMYLAKTNTPIIDYVYIRRYNILLLQAKYMLGKKQVRVTDSNFEQEKIQRKEYLLHATNSIKDNYSAYFVHHVCKLAEKYNAYIVFENLNKKNGDTREINFLSGEQISGLTPKQQNQLKGVSFYVNMEQKLINKLNYLATKDAQSHSLTLKGQFTQSIDYKGVITSMTNEQINLITSPKKQEQVEALKSGLGKGKKNCYQIGCILYVDAANTSKECCNCGFSVNTALTRCERTDEGLSFSGSSNTSYKWGDTRLTERGIDESFDLNNKSDGVKLSVRPGKVVNAKLSTRDVYDPISCLACGFDGRGTGHDHLHENDNDAYQKIKLLQTCDELAAFNIAKRGGKLIKGQEEVX

SEQ ID NO: 32 - protein, 1213 aa, synthetic construct
  REGION 1..1213: note = EpiCas033
  SITE 1213: note = misc_feature - Xaa =
MNYSDFVGLYSISKTLRFELKPTPATKKLLDDSGVKETDKIRHEHFKQAKKILDEVHREFINDALKNVSLSTSDLQDYIKLTLDKASKKADVQKAQKKIFSSFEQAYKITNYEWQEKLDAKKFFLQSPGIQLEIASQLAEKIAKNLEMNEDDVKNTLKQFDKFSTYFKGYAENRKNCYSFDGKATEIANRTIAQNLITFYKNIEKFNKYYSGNSLLADDVSKYFLLENYNQFITQQSIAQNYNVKIGELNYIINQERQKQKKDAKANLPLFKVLYKQILGDATKEVPFEFIKDDAEYKAIIHNLKDKMPKWIEEIEDHFVSQAKQLQNPEGVYLGKKAINTLLQKYISIDVRPLINEELPELAKNKSSKTDDDESLRNQFISVKDFFEALDKVQEQSETSILRSDESAALLPSQFIWNKLCNDIEKDVKVAKDSAPKIDTSNKNSVKSALDSFNDLFRMYSYFELRYKNQPVAVMDEDAKFYQIFNAEDTPFANEKDKRFSQVYDLIRNYLTKKQYSLDKWKLNFDCSILLGGWDLNKEPEKHGVLLRHKQQYYLAIIDNSAKNAFDKKENLDLYTAGADDWQKMEYKLLPGANKMLPKCLFSKSFRENFGVPSDIDVLYKNDEYKKGDKFDINKMHKLIDFYKDAIKKYEGWSMYEFVFSDTKTYQSIDQFYHEVDRFAYRLDFVNINNQVLSKMNENGQIYLFEISNKDWLKEARGKANLHTQYFLNTFRGGDFKLNGEAEIFFRPKSIEAKVDKERKSKHEIIENRRFSEDKILFHVPITFNRNSKLSSNFAFNQHAIERIKTNGVNAVIGIDRGEKHLAYVAVVDKNGKLLDKPVSLNQIHSICKGRDFPKDYFDELKKREDERRSNRQDWDAVRRIKDLKDGYVGHCVKQVVDLMVKHNAVVALENLNFGFKQGRSKIERSTYNQFEQALLKKLQYVVDKNKDDDEIFGVQKGVQLAPPDVAPGKISNHMGFVFYVDPSYTSAIDPKTGYRQHVRLDDRVKTKNFSKFVDDAFDKIYFKDDKLIFEFNWRGLAKAQNGLGVSKNKKTDIKQDKKWGITLDVPRTVFHKNVAGKSETEIVNPQQLLICSLNEAGIKLNEDIKLQLVKNTNLSSKFVKSFVYCFNLANKLRNQIDGEDAIISPVYPNGFDSRNQKLDGKYKWNGDANGVYNIARKGAILLQKLNKASTPKEFNCKVSSEEYDGFVGGEKX

SEQ ID NO: 33 - protein, 1132 aa, synthetic construct
  REGION 1..1132: note = EpiCas034
  SITE 1132: note = misc_feature - Xaa =
MIFNCILTEHQETQNTKVLVEKFVNDFKDLMGDMQKQEEYLAENYGVDKSLYKIESIKNEGDEKSKMDTATFYIYPYFDFDKNLSIEECIKRVDSFIGDKKLNRNVEELIGVSDNAGGFSFYFNNGLQLLQNNRIDEIKNAILQASSLWNGKENELQERLEFLNKKAKQVLPVRIVKNWHEYRSDIGGSIQSWVSNSLRQDIEIKKLLYGYKEKKVKRGGEIEEHDIEGHIQEIEKVLGDAELGGYKQYFENKEDEGGIRQTEEVIAKVKQLKKHLDDEKEKIDTEQPKINPAILNDYRDVLADFRNTFNHLYQKRYGLEEQDEGIEQKDEKGKKIQTVNKKYPKLFKELPKIPKFLGAVKIEEKGVYDKYICSYNGLKKGVEFLGGVTFTGSKRIDDYYTYRNDSSKDKEEIEKARKRSIERVQRVLENILRLYRSTDAKQLKGQKIKSLLAKYCKDISLLDGEYNYIYRNERYARDKRGQKIELKRVDDELVEDDFIQGLISDLQSFEWRLDNIKQGKWYERVAGWGALVEIEKIKIGLMAWFYEISVPHNFQEIEKNKLHFRKVEAIVKRYNQDEVLDNKEKNTIIQQGIVSEMRGMIARMSKQEIIARYVVQPVNAEQKYPLVMDEGKYYIHPKEILDKDEIKEQPVNMIDISKEKDLTEEKNSRIKVRRDSLFAITSSKYQTQFLNNSLNRQLEKWQNLNPQLSEYSFIVEDTHKIKWVQDGDSYTPNCDEVLLSRLFVSVPFKIGVENKDKTVAIKQQNKFIGIDIGEYGVAYYGLDGSDFSAKPFVAGFFFQPDMRKIREGIAKNKERQQAGTFTNSNTQIKRVRDNATTKIRNQVHQLVVENNALPIYEKEVSAFESGSGKISAIYNSIKRSDVYAESKADDMEHKLIWGNDKIMVGAEIGAYATSYMCSVCYESIYAYIAKDDYEREYEVIDIVGEQKQIKDKWNATVKFDIEGKEMYAYIEQKDKPVGKGDTFNGKGTINFVRKYERPPLDVVRKIERLPSAIQQELNECTKKERVKSNGEKEKISVWENRRGSQGVFRCAFCNKCSDADVQASMWIALKGYLQNVKNAVQKKENKEQYKMYEGSIGKEVVGEIAKGFSQGGKEQVRFILDFAKDNSIMLRX

SEQ ID NO: 34 - protein, 745 aa, synthetic construct
  REGION 1..745: note = EpiCas035
  SITE 745: note = misc_feature - Xaa =
MATKTYRARLSSIDNRTQQAVVRTHYFFNHCLRQMIERYLDMRKGKYGPFCKQLGDLLLTRSHTDAHGIMDQLTRGAPSTQTDQEWAVLVKHHHREYGPLFLQHEGFAVVDGVVVHTKPQGRHEPTPGRLAVTAKFWHQICDMACAFLKSNQELMAVWRKDKTEWLKTKNAWEVDNPEFMTFWNGPYADFERACEAARVAAQEAAGQTPTARKRDSRVRGKRIDRWHLWYEWLVAHPETVGWRQRAEAGDFKPVPDAVQAAIRKKTKRQDKYVGKYLDWLRENNPELKALDSLRRTYVRRYLRFKRPPTLTLPSANKHPYWFTLELNEFYKGADFEKGCVKMRLIDCDADGLLEMKWFDVRMGCDHRLKPAWRKEKFASDGRYPPYLGGKIGRKLDRPAECAADRKAGVKGVKLVLDKGRRELFFTLVEQDCPPRLKFKKTKNRRCTADNLFDAGDQRVPIRVMSVDLGIRHIGAYAICEGTKQDDHWEMKYLKKGLLSDSSLPELGQIRLHDRHLKKLRSRQGDAPTGERNFVALQDHRTGMADDRFKKAAHLLIETARRAGVDIIVFEKLDTLKPDAFDERWINRQLRDMNRRQIVTHVAQQAGEFGLLCKDDISPWLTSQVCSRCYRPGWRFSIKPKAPYKEQCSRAACQDFGYPIWDPGGHLFRCPHCGYKVNADINAAGNLAAKFFGHGFWFDNNLSRDKASGCFIWTDDQRRCFDAREAFEQWAAEASRRQALKEAPFX

SEQ ID NO: 35 - protein, 4488 aa, synthetic construct
  REGION 1..4488: note = EpiCas036
  SITE 748: note = misc_feature - Xaa =
  SITE 1496: note = misc_feature - Xaa =
  SITE 2244: note = misc_feature - Xaa =
  SITE 2992: note = misc_feature - Xaa =
  SITE 3740: note = misc_feature - Xaa =
  SITE 4488: note = misc_feature - Xaa =
MATKSYRARILTDSRLAAALDRTHVVFVESLKQMINTYLRMQNGKFGPDHKKLAQIMLSRSNTFAHGVMDQITRDQPTSTLDEEWTDLARRIHKTTGPLFLQAERFATVKNRAIHTKSRGKVIPSPETLAVPAKFWHQVCDSASAYIRSNRELMQQWRKDRAAWLKDKNEWQQKHPEFMQFYNGPYQNFLKLCDDDRITSQLAAEQQPTASKNNRPRKTGKRFARWHLWYKWLSENPEIIEWRNKASASDFKTVTDDVRKQIITKYPQQNKYITRLLDWLEDNNPELKTLENLRRTYVKKFDSFKRPPTLTLPSPYRHPYWFTMELDQFYKKADFENGTIQLLLIDEDDDGNWFFNWMPASLKPDPRLVPSWRAETFETEGRFPPYLGGKIGKKLSRPAPTDAERKAGIAGAKLMIKNNRSELLFTVFEQDCPPRVKWAKTKNRKCPADNAFSSDGKTRKPLRILSIDLGIRHIGAFALTQGTRNDSAWQTESLKKGIINSPSIPPLRQVRRHDYDLKRKRRRHGKPVKGQRSNANLQAHRTNMAQDRFKKGASAIVSLAREHSADLILFENLHSLKFSAFDERWMNRQLRDMNRRHIVELVSEQAPEFGITVKDDINPWMTSRICSNCNLPGFRFSMKKKNPYREKLPREKCTDFGYPVWEPGGHLFRCPHCDHRVNADINAAANLANKFFGLGYWNNGLKYDAETKTFTVHTDKKTPPLIFKPRPQFDLWADSVKTRKQLGPDPFXMATKSYRARILTDSRLAAALDRTHVVFVESLKQMINTYLRMQNGKFGPDHKKLAQIMLSRSNTFAHGVMDQITRDQPTSTLDEEWTDLARRIHKTTGPLFLQAERFATVKNRAIHTKSRGKVIPSPETLAVPAKFWHQVCDSASAYIRSNRELMQQWRKDRAAWLKDKNEWQQKHPEFMQFYNGPYQNFLKLCDDDRITSQLAAEQQPTASKNNRPRKTGKRFARWHLWYKWLSENPEIIEWRNKASASDFKTVTDDVRKQIITKYPQQNKYITRLLDWLEDNNPELKTLENLRRTYVKKFDSFKRPPTLTLPSPYRHPYWFTMELDQFYKKADFENGTIQLLLIDEDDDGNWFFNWMPASLKPDPRLVPSWRAETFETEGRFPPYLGGKIGKKLSRPAPTDAERKAGIAGAKLMIKNNRSELLFTVFEQDCPPRVKWAKTKNRKCPADNAFSSDGKTRKPLRILSIDLGIRHIGAFALTQGTRNDSAWQTESLKKGIINSPSIPPLRQVRRHDYDLKRKRRRHGKPVKGQRSNANLQAHRTNMAQDRFKKGASAIVSLAREHSADLILFENLHSLKFSAFDERWMNRQLRDMNRRHIVELVSEQAPEFGITVKDDINPWMTSRICSNCNLPGFRFSMKKKNPYREKLPREKCTDFGYPVWEPGGHLFRCPHCDHRVNADINAAANLANKFFGLGYWNNGLKYDAETKTFTVHTDKKTPPLIFKPRPQFDLWADSVKTRKQLGPDPFXMATKSYRARILTDSRLAAALDRTHVVFVESLKQMINTYLRMQNGKFGPDHKKLAQIMLSRSNTFAHGVMDQITRDQPTSTLDEEWTDLARRIHKTTGPLFLQAERFATVKNRAIHTKSRGKVIPSPETLAVPAKFWHQVCDSASAYIRSNRELMQQWRKDRAAWLKDKNEWQQKHPEFMQFYNGPYQNFLKLCDDDRITSQLAAEQQPTASKNNRPRKTGKRFARWHLWYKWLSENPEIIEWRNKASASDFKTVTDDVRKQIITKYPQQNKYITRLLDWLEDNNPELKTLENLRRTYVKKFDSFKRPPTLTLPSPYRHPYWFTMELDQFYKKADFENGTIQLLLIDEDDDGNWFFNWMPASLKPDPRLVPSWRAETFETEGRFPPYLGGKIGKKLSRPAPTDAERKAGIAGAKLMIKNNRSELLFTVFEQDCPPRVKWAKTKNRKCPADNAFSSDGKTRKPLRILSIDLGIRHIGAFALTQGTRNDSAWQTESLKKGIINSPSIPPLRQVRRHDYDLKRKRRRHGKPVKGQRSNANLQAHRTNMAQDRFKKGASAIVSLAREHSADLILFENLHSLKFSAFDERWMNRQLRDMNRRHIVELVSEQAPEFGITVKDDINPWMTSRICSNCNLPGFRFSMKKKNPYREKLPREKCTDFGYPVWEPGGHLFRCPHCDHRVNADINAAANLANKFFGLGYWNNGLKYDAETKTFTVHTDKKTPPLIFKPRPQFDLWADSVKTRKQLGPDPFXMATKSYRARILTDSRLAAALDRTHVVFVESLKQMINTYLRMQNGKFGPDHKKLAQIMLSRSNTFAHGVMDQITRDQPTSTLDEEWTDLARRIHKTTGPLFLQAERFATVKNRAIHTKSRGKVIPSPETLAVPAKFWHQVCDSASAYIRSNRELMQQWRKDRAAWLKDKNEWQQKHPEFMQFYNGPYQNFLKLCDDDRITSQLAAEQQPTASKNNRPRKTGKRFARWHLWYKWLSENPEIIEWRNKASASDFKTVTDDVRKQIITKYPQQNKYITRLLDWLEDNNPELKTLENLRRTYVKKFDSFKRPPTLTLPSPYRHPYWFTMELDQFYKKADFENGTIQLLLIDEDDDGNWFFNWMPASLKPDPRLVPSWRAETFETEGRFPPYLGGKIGKKLSRPAPTDAERKAGIAGAKLMIKNNRSELLFTVFEQDCPPRVKWAKTKNRKCPADNAFSSDGKTRKPLRILSIDLGIRHIGAFALTQGTRNDSAWQTESLKKGIINSPSIPPLRQVRRHDYDLKRKRRRHGKPVKGQRSNANLQAHRTNMAQDRFKKGASAIVSLAREHSADLILFENLHSLKFSAFDERWMNRQLRDMNRRHIVELVSEQAPEFGITVKDDINPWMTSRICSNCNLPGFRFSMKKKNPYREKLPREKCTDFGYPVWEPGGHLFRCPHCDHRVNADINAAANLANKFFGLGYWNNGLKYDAETKTFTVHTDKKTPPLIFKPRPQFDLWADSVKTRKQLGPDPFXMATKSYRARILTDSRLAAALDRTHVVFVESLKQMINTYLRMQNGKFGPDHKKLAQIMLSRSNTFAHGVMDQITRDQPTSTLDEEWTDLARRIHKTTGPLFLQAERFATVKNRAIHTKSRGKVIPSPETLAVPAKFWHQVCDSASAYIRSNRELMQQWRKDRAAWLKDKNEWQQKHPEFMQFYNGPYQNFLKLCDDDRITSQLAAEQQPTASKNNRPRKTGKRFARWHLWYKWLSENPEIIEWRNKASASDFKTVTDDVRKQIITKYPQQNKYITRLLDWLEDNNPELKTLENLRRTYVKKFDSFKRPPTLTLPSPYRHPYWFTMELDQFYKKADFENGTIQLLLIDEDDDGNWFFNWMPASLKPDPRLVPSWRAETFETEGRFPPYLGGKIGKKLSRPAPTDAERKAGIAGAKLMIKNNRSELLFTVFEQDCPPRVKWAKTKNRKCPADNAFSSDGKTRKPLRILSIDLGIRHIGAFALTQGTRNDSAWQTESLKKGIINSPSIPPLRQVRRHDYDLKRKRRRHGKPVKGQRSNANLQAHRTNMAQDRFKKGASAIVSLAREHSADLILFENLHSLKFSAFDERWMNRQLRDMNRRHIVELVSEQAPEFGITVKDDINPWMTSRICSNCNLPGFRFSMKKKNPYREKLPREKCTDFGYPVWEPGGHLFRCPHCDHRVNADINAAANLANKFFGLGYWNNGLKYDAETKTFTVHTDKKTPPLIFKPRPQFDLWADSVKTRKQLGPDPFXMATKSYRARILTDSRLAAALDRTHVVFVESLKQMINTYLRMQNGKFGPDHKKLAQIMLSRSNTFAHGVMDQITRDQPTSTLDEEWTDLARRIHKTTGPLFLQAERFATVKNRAIHTKSRGKVIPSPETLAVPAKFWHQVCDSASAYIRSNRELMQQWRKDRAAWLKDKNEWQQKHPEFMQFYNGPYQNFLKLCDDDRITSQLAAEQQPTASKNNRPRKTGKRFARWHLWYKWLSENPEIIEWRNKASASDFKTVTDDVRKQIITKYPQQNKYITRLLDWLEDNNPELKTLENLRRTYVKKFDSFKRPPTLTLPSPYRHPYWFTMELDQFYKKADFENGTIQLLLIDEDDDGNWFFNWMPASLKPDPRLVPSWRAETFETEGRFPPYLGGKIGKKLSRPAPTDAERKAGIAGAKLMIKNNRSELLFTVFEQDCPPRVKWAKTKNRKCPADNAFSSDGKTRKPLRILSIDLGIRHIGAFALTQGTRNDSAWQTESLKKGIINSPSIPPLRQVRRHDYDLKRKRRRHGKPVKGQRSNANLQAHRTNMAQDRFKKGASAIVSLAREHSADLILFENLHSLKFSAFDERWMNRQLRDMNRRHIVELVSEQAPEFGITVKDDINPWMTSRICSNCNLPGFRFSMKKKNPYREKLPREKCTDFGYPVWEPGGHLFRCPHCDHRVNADINAAANLANKFFGLGYWNNGLKYDAETKTFTVHTDKKTPPLIFKPRPQFDLWADSVKTRKQLGPDPFX

SEQ ID NO: 36 - protein, 1292 aa, synthetic construct
  REGION 1..1292: note = EpiCas037
  SITE 1292: note = misc_feature - Xaa =
MENKNCNFDKFTKQYQVSKTVRFALTPIGRTEEHIIEKQYIEAAQRKDQAYKIVKPVIDKKLCVMIDDVLTHCEKQDWEKLNELTLQYQNDKTSVEALGEQQGKIRKNISKEFENSIEYKNFFGKDSEKLFKTFLPQCLEEINASEADKEAVKEFQKFKTYFNNFLTVRKDIFNDDSKQNTIPYRIVNENFMIFAGNRKTFSNIVKLIPDASAEIAKEGIERQEWEFYDVQNIDSCFEPDSFQQCMSQLGIQKYNFIVGLVNSYMNLYIQQHPEALEVKRSRLKLRMLHKQILSDRTNPSWLPEQFAEGEEGEKQIYEAIQALEDDLNKNHFQEKFDNLLQLIDTENPRIYIAASEMPRVSSALNYGWNGLNNVRMNVLQELNSEAKIEKILKQDVSLNELSDALNKYADIYKKEQVPTLQQYIEYGKELLQDCAVACNDYHASIRNNVERLNQNEKLIEVLKTYLDCYQRVMHFMNVFVVGDELDKDVDFYAELDELIEDISQIIPLYNKVRNYVTRKAYSLDKIRIMFEKSDFLGGWGQDFDTKKALIFEKDGLYYIGIIEKKYTNTDVQYLYEEIEEGNKAVHFVYNFQKIDNKNFPRIFIYSKGTNYAPAVRKYNLPIQNIIDIYNERKFQTDYKKVNEQEYYESLEKMIDYFKDGIMKNETYRKFHFNWKPSNEYENIKEFYDDTDNSCFLLEKEEINFNHLKEQADQGKIYLFQISSKDFNKGSTGTPNLQTMYWRELFSDQNCKEGIIKLCGGASIYMRDVSIKQPIIHKKDSWLINKWYMEEGQNKVIPDDTYVKFSQIVQGRMSEAELNVEEQQLWNSGLIQKKKATHDITKDRRFTRKQYMLHAPLIINYKQQDIPTPRRFNEEVRSFLKNNPNINIIGIARGEKNLIYITVIDQKGNILSGMQKSFNQIEEKGANSRIVDYYGKLQSVEDRHTEARKNWKQIGTIRELKEGYLSQVVHEIAQLMIQYNAIIVMANLNMGFKKGRMKVEKNIYQRFEKMLIDKMNYLAFKKNIEGKAINPYEIGGVMNGYQLTNKFTNFADMGSQNGFIFYVPAAYTSVIDPVTGFVDVFQRAEFKTGDFLQKFEDISWDDKEQSFVFTFDYQKFKCNGTCYQNKWSLYADVNRIETVKKNNQVYRQEQCNPNQRLIELFDSKGIAYRDGHNLVNDLGNYDSKLLSDILYNFRLILQLRNSMRDYSTGEVIDYIASPVMYHRERFDSQKKNPSLPLDADANGAYHIALKGLMCLQKINEYADPDGKMDIKNMKITNEEWFKFMQTRRESNVX

SEQ ID NO: 37 - protein, 1261 aa, synthetic construct
  REGION 1..1261: note = EpiCas038
  SITE 1261: note = misc_feature - Xaa =
MRRFFMTKMTDFTKLYQLSHTLKFELRPIGKTSENLQNSGLLEEDFKRSQDYVEVKSVLDEVHKKFLQDVLSDCDLSWQELADALKIFQVNKDRKVLEDVQKKYRKLLADRFNKNKFFKTLTEATPSKLFKSLLAEPDMAERKVETFKRFACYFKGYQETRQNIYSAEAQQTAAAYRAVNENFVKYLRVVELYENFRKNFPELAEEIGRRTAELTNGELSQILRIENYNRFIAQSGIEYLNSLIAQINSAVNLYRQAHNVKLSFVPLLFKQILSDREQAFTLERIEKDADLDEKLRAFCERHIGNVEIRGETGDLFSRLKVLLTTLNDDCELYIDAASLSAIGNALTGNWSCFRDAMTAYANETLKNKSLREKYCKQPVYSFSEIKSWRVVRTDDDGNVQPVCLSDYWHGEKTLELFAAEKILRNRILEMVRVPHDKNFRERKNDVALVKDYLDAVVEILHLLKPLNVGSENGGDLDLLGILTEFYEALSPVIPLYNQIRNYVTQKPSDIGKIKLMFNNPTLADGWDLNKEKDNTAVIFCRNGMYYLGIMNPKSKTDFNSLATDSSTDSYQKMVYKQLQNPNQMLPKVFFSAEKIKFFAPSEELLRRYEAGEYKKGEKFDLAYCHELIDFFKRSIDRHEKWSKFEFKFSETSAYSGINEFYREVAEQGYKMSFVNIPVKAVDQLVEDGKLFLFQIWNKDFSEKSTGTPNKFTLYWKALFDRQNLSDVVFKLNGEAELFLREPAITEPVVHRPGGKLVNKTVVAAVDGNGVAIRNSIPDAVYTEIYKHVNGMEEGTLSPAAREHLNRFPRLDWEPGMGLEATVKRTVVKDVKFQLVKDKRFTERKFSFHVPITVNFKSPRLKGAKLNEMVWEFLKDNPDVNVIGIARGERNLIYVNCIDLKGRILLQKSFNIVGGFDYHQKLDLRERERGQARKSWNEIGTIKELKAGYLSGVVYEIARLMVEYNAIVVMADLNGGFKRSRTGIEKQIYQKFEKALIDKLNYLVFKNESDWHVPGGLLNGYQLTGPLESFAKLGKQCGWLFYVPAAYTSKIDPSTGFVSLFKLSNYTNTEKRKAFFENFESIVYQAEQDAFVFAFDYKKFKDVENCWKTSWEVYSARRRLVYSRRSRQIEEINPTRLIKRALDKRGVVLSDGFDLKHYITSVEAVNENAAFFKDIFDAFGTTLQMRNSSGEEDYIESPVLNENGQFFDSRKAGKEWPRDADSNGAYHIALKGLLRMKSGQTAKLKRLDWFEFVQRRGFITEX

SEQ ID NO: 38 - protein, 1228 aa, synthetic construct
  REGION 1..1228: note = EpiCas039
  SITE 1228: note = misc_feature - Xaa =
MNDLSEFVGLFPVSKTIKWELKPVGMTAERMREAGFLDRDRSKRDAYTVVKSMADRFYRGYIDERLSTLDLDWNHLGDAMSALKESGETKDKRELENIKKSFKDSISKHIFTKKDDEDIFSAKVLARLRGTETDDEEVEALEKFDRFAGYFQDFFVNRRVFADTVRGSCAYRIVEDNFPRHFENCRKYQIIRRDNPELILELKRSMEGYLETDPDVLFSIDHFGKLLRQRDIDAYNRVVGSVFRSESDNSRGFNPILNEFHQRNPDSPKMLMQKLQKQILSEPDSGSFRMRNYENEGQMFEDLQNLWTGIETNRLLEIPASVLEEGEFDKSGVFISKAGVNTLSNWMFSDWERIGGLLQIFKINEMGDSASKSRKKIDKWLDSDSFSLDVLNRAVADETDIGIVPFLSRRLSDAAQQAREAYLGLEGCASMRREQYQGRVKTFVDSLLRYRDILKLFSYDPDLPLDRGFYDTFQFVYEEMASAYRAYNRCRSFITRKEYSTEKFKMSFDCPHLADGWDLNKESSYNSFLFRRDGLYYLGIRNPAWRYEDFSVKADPECDDFYEKAVYKQLQDPVKMLPKVFFSKKGLDKYRPPERILEGYENGLHRQGDSFDLGFCHELIDFYRDAISQNRDWDFFDFKFKETSEYSSISEFYQDVESQGYQMRFIRIPAQTIDEMVDEGRLLLFQMYNKDYSRDKTPGGKPNLHTMYWEAAMSEDNLRDVRIKINGYAELFWRDASIKDGTVHRKGSVLVNRIGKDRKPIPSEAYYDLCRYCNGIRDDLGAGKGYEDQIVCKNSRYDIEKDRRYCHDRMFLHIPLTFNFKSDGKTDVNRKVLEWIRRNDDVKFLGIARGERNLIYLSLVDSDGRILWQRSLNTIVQKDGRGNTRALDYHEKLAVREGERRNAQQSWGPMDAIKNLKEGYVSAAVHEICRIAVEEKAVIVIANLNYGFKNSRKKVEKQTYQTFERMLINKLQNLAFKDAEPGSPGSVLAPYQLCGPIDSMEDIPNHCGMLIHVTAAFTSRIDPVTGFVSVFNLSDITNNAQRADFLSRMTSIRYNQDEDVFEFSFDYGDFRTRLTPKVRKWTVCTYGDRYKYNRKDRTLVRISPTTMIKDALTKAGIRYEDGSDIRKEISGSDAVRQVFEAFEAILDMRVTTRDEDVIVSPVRDSSGRHFVSGTSGPGLPQDADANGAYCIALKGLLLARKIRASDEDRLPLLNNNDWVEFMQTRVMX

SEQ ID NO: 39 - protein, 1247 aa, synthetic construct
  REGION 1..1247: note = EpiCas040
  SITE 1247: note = misc_feature - Xaa =
MIEQFVNRYPLSKTLRFGLIPQGNTEENFKIMQILEKDEALARNYGKAKEIIDRYHKFFINSCLADFHLTDLEKYALLYYEPNKTEEEVAAMDETAKTMRQAISGLFCKENKAIYDRLSKKDLIENFAMSVIETDEERTVLESFRKCTVYFTGFNQNRLNMYTGEGKSTEIAHRLINQNLPKFLDNARVAKTILAALPKNDLDQIDVDFYDIFGINTQNIFEIPFYNQTLTQAGIDRYNQIIGGYTCSDGRKIKGYNEYINHFIQETKQKLPKFKPLYKQILSDRETISFIPKAFQSDSEVLSALRNFYIKKNVETDKSIRDVITELKKLFQEMDSFDEKGIYITNGVAITDLSQAVCDNWFVIKKAMQAEYDAEHSSKNRNEDYEKKRGNDLKKVKSYSLSALQRYMDAEMEDKKSLAEYIKTHTTQLCDLTASAYERVRHLLETEYSPTKKLTADADAIGRIKDFLDPIMEVFHFAKIFLGSGEEAEKDDVFYGIFSPLYDQLADIIEIYDKVRNYVTQKPYKTDKIRLIFERGDFLSGWAQPTESQAKEAHLMEKDGRYYLFITVTAINKADWERALYGKDNFQCACHIEYYKQKPDNKNTPRLFIRSKGDHYAPAVEKYNLPIQEVIDLYDKGYYKAEFKKKDQKLYYTSLRKLIDYFKLGFTKHENYKDFAFAWKESDAYENITEFYQDTMNACYRLERVTVNWNGIMDLVEKGRGFLFQIYNKDFSDHTKGTPNLHTMYFKMLFDERNLENGCYRLLGGAEMFYRKASLKLSETTIHPKGVAIKNKNPRSDRAERVLDYDIIKDKRYTETQFSLHFPIMMNCLAQGNPKLNLDIRKALLKSEHQYIIGIARGERNLLYVCVINEKGEIVEQMSLNEIIGDNGYTVDYHELLDRKEKERDAARKSWGTIENIKELKEGYMSQVIHKICELVLKYDAVIAMADLNGGFMNSRIKIEKQVYQKFEKMLTEKLSYLVDKKADPMSDGGLLRAYQLTNKPDPNKSVQHGMQDGIVFYIPAWCTSKIDPTTGFVDLLKPKYTNKEEAKDFFKRFQKICFNEVENMFEFHFHYPDFQRGITAGNTDWTVYTNGSRIRTFRNPEKNNQWDNTDIVLTDAFKALFAEYAIPCDADMKEAILAQDSPKFFKELCGLLSFTLQMRNSITGRTDVDYIISPVKNAAGRFYDSRKAASDLPCDADANGAYNIARKVLLFIRRLQAQEESNLMNKDLEKITNAQWLSFVQNGHEX

SEQ ID NO: 40 - protein, 1256 aa, synthetic construct
  REGION 1..1256: note = EpiCas041
  SITE 1256: note = misc_feature - Xaa =
MTKMTDFTKLYQLSHTLKFELRPIGKTSENLQNSGLLEEDFKRSQDYVEVKSVLDEVHKKFLQDVLSDCDLSWQELADALKIFQVNKDRKVLEDVQKKYRKLLADRFNKNKFFKTLTEATPSKLFKSLLAEPDMAERKVETFKRFACYFKGYQETRKNIYSAEAQQTAAAYRAVNENFVKYLRVVELYENFRKNFPELAEEIGRRTAELTDGELSQILRIENYNRFIAQSGIEYLNSLIAQINSAVNLYRQAHNVKLSFVPLLFKQILSDREQVFTLERIEKDADLDEKLRAFCEQHIGNVEIRGETGDLFSRLKVLLTTLNDDCELCIDAASLSAIGNALSGNWSCFRDAMTAYANETLKNKSLREKYCKQPVYSFSEIKSWRVVRTDDDGNVQPVCLSDYWHGEKALELFATEKILRNKILEMVRVPHDKNFRERKNDVALVKDYLDAVVEILHLLKPLNVGSENGGDLDLLGILTEFYEALSPVIPLYNQIRNYVTQKPSDIGKIKLMFSNPTLADGWDLNKEKDNTAVIFYRNGMYYLGIMNPKSKTDFDNLATDSSSDCYQKMVYKLLPGPNKMLPKVFFADKNIDFFAPSEELLRRYKAGEHLKGEKFDLAYCHKLIDFFKRSITRHEEWSKFDFQFSETSAYAGIDEFYREVAEQGYKMSFVNIPVKAVDQLVEDGKLFLFQIWNKDFSEKSTGTPNKFTLYWKALFDRQNLSDVVFKLNGEAELFLREPAITEPVVHRSGEKLVNKTVVAAVDGNGMAIRNSIPDAVYTEIYKHVNGMEEGTLSPAAREHLNRFPRLDWEPGMGLEATVKRTVVKDVKFQLVKDKRFTERKFSFHVPITVNFQSPGLKGAKLNEMVWEFLKDNPDVNVIGIARGERNLIYVNCIDLKGRILLQKSFNIVGGFDYHQKLDMRERERGQARKSWNEIGTIKELKAGYLSGIVYEIARLMVEYNAIVVMADLNGGFKRSRTGIEKQIYQKFEKALIDKLNYLVFKSESDWHVPGGLLNGYQLTGPLESFAKLGKQCGWLFYVPAAYTSKIDPSTGFVSLFKLSNYTNTEKRKAFFENFESIVYQTEQDAFVFAFDYKKFKDVESCWKTSWEVYSARRRLVYGRRSRQIEEINPTRLIKRALEQRGVVLSDGFDLKHYITSVEAVNENAAFFKDIFDAFGTTLQMRNSSGEEDYIESPVLNENGQFFDSRKAGKEWPRDADSNGAYHIALKGLLRMKSGQTAKLKRLDWFEFVQRRGFITEX

SEQ ID NO: 41 - protein, 1234 aa, synthetic construct
  REGION 1..1234: note = EpiCas042
  SITE 1234: note = misc_feature - Xaa =
MRNRYSKILCLLLLFACCLPQEGNAFWPFKKKKKEDKRLRKLIAQNLKAHPLVDKLKVKNAFGKNGYLETLPLGKEEKEAVKVFAGFGGFFNNYNKNRENYFSTEEKSTAIANRIVNENFSKHFSNVEIVTKIQKEVPELIQIVEAQFKGYDAIFTVNGYNMALSQAGIDTYNEMVAIWNKEANLYAQKAGKLPDGHPLKKKRNYLLSALFKQIGSEKEHLIQIDRFDGDEEVIEALTGVKKMLQEADVFEKLNMLVEDMENWDYSKIYLSAQSLSNVSVFLNNLYEDERENSWNYLDNVLREKWQIELQGKKKGTDLEEAIRKKKQSFYSIEELQEAVNAIEETDKCYNVSKWLLGAMKSERVIEEKKKDVEDFCTQWKNERNSLKETDITALKEYLEQWIFLARYCKSFYANGIEKKEKDEAFYHILEDVLYVLDEVIYFYNKVRNYVTKKPYSLEKMHLKFGHNELANGWSVNKEGNYGTAILRRNGKYYLAITNSLNKKMSIPTQLESTGNNYEKMVLNVFPNVFRMIPKCTTGRNDVKSCFERKEPNEYFFIDTPEFVNPFKVTREEYELNKITYDGVKKWQSDYSKNTQDEKGYKEAVTKWIQFCMRFLQSYKSTAIYDYSTLQQPEKYETVDSFYHDVEKILYECHFEYVPANKIEQLEEEGRIFLFQIYNKDFSENRRPDSKKNLHTLYWEALFSEENRKAKVIQLNGKAEIFRREKSIEHPIVHKAGEVLVNKRTKDGEPIPDDIYKDLSNYFNGRNVTSEKEEYKECLDKVYTSTKKYDITKDKRFTETKYEFHVPITLNYQADGVKYLNQKILHVLRDNPDVNIIGLARGERNLISYVVLNREGKIVNNQQGSFNIVGKMDYQKKLYQKEKNRDKERKTWKNIETIKDLKEGYISQVVHELTDMAIRNNAIIVMADLNFGFKRGRTKVERQVYQKFELALLKKLHYLVTDKTEGEAMLKPGGVLQGYQLAREVKTLKEIGKQCGCVFYVPPGYTSKIDPTTGFVDVFNMSGVTNREKKKAFFEKFDNMFYDEKRDMFGFSFNYEKFTTYQSSYRNDWTVYSNGSKYVWNSLKRTDELIDVTKELKLLFEKYAIDYRNEALFEQIMSQDTDKNNADFWNKLFWYFRVLLRLRNSSDELDQIVSPVLNQNGEFFETPKKITEKSYLSDYPMDADTNGAYHIALKGVYLIQEKIADESVDLDNKLPKDFYKISNAEWFMFRQKEKX

SEQ ID NO: 42 - protein, 1293 aa, synthetic construct
  REGION 1..1293: note = EpiCas043
  SITE 1293: note = misc_feature - Xaa =
MVSNKEKTPTYNSLTNLYSVSKTLRFELRPQYSTLDHINEDQIVGKGEELKNHYKTFKKILDQVFSRIINDSLDKTYLDQKYISTYQDLAFKHRDQLTDKDRAELKTLKETLKKQIDKSLDHKDKKAIFSDPVKFLLDNESEFTDLIGDNRPSIEAFNRQKGYLSGYLQNRANIFDHTTNETSVAFRIVEENLAIFLNNRITLQHFLEKVAGKDGLLKSLQEALFQLGFKLKLEDLLSLDYFNRTLSQSGIDQYNLLISGKTLEDGAKLQGINEILNQYLKQHQEEKLHKIKLKQLYKQILSESKTESFTLDFVEDNKGLAAMFLQFIDFANQLIEEKMPLLGMIQELKNSSVSAELLSRLHLNRKNIKRLSNFIYKDYGYIEQALEENFLSTVEGKITKKVLGEHRKQDTFTIHEILVALQKQQYEKDGTLESADRLFLPSIVDFVYQNLNCEYSTLLEKVRSEKQPLLELFKERQLLEDQDTESHSSKYPDHPFNDHEIKIIKTTLDFYKNLQSNLALLQISDENLKLDSEFYSEFDEFYQGLKNIIPVYNKSRNFLTKKPFSTEKTKLIFNSPQLLDGWSKSKESDCLGTIFIKDGKYYIGIINSATNAKKTLFEPHNFANFDQSQYFEKMNLFFLSDLKRDFPKKYFSEKWHNQYPIPADLREKYDYYRIDEHKDERKNDLEYHHQLIAYYQDCLKKDTEWQIYQFKYKSPEEYSDINEFLSELTPNTYKMEFTKIPAEYIKKLVDDGKLYFFQIYSKDFSEFAKGKPNLHTLYLKAVFDQKNAEEFNYNYKISGSAEIFYRPASIETRVTHPKNQPIKNKNKNNPKIESIFQYDLCKDRRYMSDKFFLHLPVELNRTPLLPNDSLVNSMVNQVVSSRNQNYFLGIARGERHLIYLVLIDQSGKIIKQQTLNQITSSYQEKSNNQTVEVVTDYHDLLNDKEKIRKKNLQEWQSVENIKELKAGYLSNVVNEIGKLIAEYQPVIMLANLNTGFKNSRIKIEKQVYQKFEKALIDKCNYFMRKDIDPSTIGGLYHALQLTKEYSKQYNGKQNGIIYYIPASYTSNIDPTTGFISAFVQTRYENVEKAKALIEKFNDITYDADESLFCFTVDYKKFSPEAKLWQQNIWQIYTNGDRIYTFRDKEEWQSKNYILVEEFKDLFTKYQINYRNDLKTQILSQTDAGFFKQFLFLLRLTLQMRNSRTADSNEADIDTKKRENDYIISPVKNQYGKFYDSRQDYMDWPENADANGAYNIARKGLIMLKHLKEGLPEKRICDISTEEWVQFVEELNKX

SEQ ID NO: 43 - protein, 1245 aa, synthetic construct
  REGION 1..1245: note = EpiCas044
  SITE 1245: note = misc_feature - Xaa =
MTRVFIFCLLYLKKTKYAPGVHKEGKMKINAFINCYSMSKTLRFKLVPEYETEKNLLEKGFLDRDKLRADDYDLMKKVIDKYHKHFIDKALEGFKFDLLQDYAEAFYSQSADDDGKKLEEIKKKMCKELATCFSKQDEFKLLDKKELVEKLIPAAEFIEDEEKDIAKRFKGFTTYFTGFNENRQNLYAAELKHGTIAFRLIEENLPAFLYNCKKGVKIFEELDEGDVETLNNELGEILSIGNVKDILNVEYYNKTLTQNGIDVYNQIIGGYTQEDGTKIKGVNEYVNLYNQTHDKKLPSLAKLKKQILSDSYSLSFLPAKFNDDSELLLSLKKFYSMVNEETGLSVEKAIQEMRNVFLHIDDCDLHNVFIDAKFINKVSNDVFGNWSVLIDGINAEYEKLNPFNGKNLGNYEEKRKAFLNKIESYSVDALQAYSGKEEKIADYVQKRAVELCDSVACAYENMSNKVINAREGKVKLYQDDEKTEIIKTFLDAVQEFKKFAEMFCYDGTDGDTTFYGEFANYYGQIAEIIPLYNKCRNYLTKKPYSEDKIKINFDNSQLLDGWDANKEKDYLTVLLLKGNNYYLGILDKNHKNVLIKDVPEKGQGEPCFKKMIYKLLPGPEKMLPKVVFSAKNIGFYNPDREILRIRENGTFKTGDGFNIDDCHKLIDFYKECISKNEDWKTFGFRFKETSEYKNIGQFYNEVKEQGYKISFTDIPESYVKDLVNDGKLYLFRLANKDFSPYSKGKKNLHTMYFEGIFDPENIKEKVYALNGGGELFFRCASLNYDKPTHPKNVPIKNKTYEFRTDNAKKETSTFEYDLIKDKRYTKDQYTLHCPVTLNFKERGIERINDLVRQSLRESDDNYVIGIARGERNLIYISVIDGKGKIVEQFSMNNLSSGNDVSIDFHKMLETRERERDASRKNWNTIDNIKDLKQGYLSYVVKKICDLVVKYDAIVAMADLNVGFKHGREKFERQVYQKFEKALVDKMSYIVNKNASPHSAGGLFRAYQLTNKKYNENEKQNGFIFYVRAWNTSKIDPTTGFVNMLPLKYQSKEKSKEFFDKFEDIFYDENKDMFGFTFRYDDFGINIDHKNEWTAYSNGERIITVRNSFGKWDKAKIALTPAFKKLFDDYNVDCRGDVKRQIMNVDDKDFFVRLYKLLSYTMQLRNSDDADDYILSPVVNAEGMFFDSRNSDGSLPCDADANGAYHIAKKAMWAIGKIKEADEESFKKTSLAIDNKTWLEFVQKAX

SEQ ID NO: 44 - protein, 1293 aa, synthetic construct
  REGION 1..1293: note = EpiCas045
  SITE 1293: note = misc_feature - Xaa =
MVSNNENTPTYNSLTNLYSVSKTLRFELKPQYSTLDHIRDDQIVDKGEDLKNHYKTFKKILDQVFSRIINDSLDKTYLDQKYISTYQDLAFKHRDQLTEKDRAELKTLKETLKKQIDKSLDHKDKKAIFSDPVKFLLDNESEFADLIGDNRPSIEAFNRQKGYLSGYLQNRANIFDRTTNETSVAFRIVEENLAIFLNNRITLQHFLEKVTDKDGLLKSLKEALSQLGFKLKLEDLLSLDYFNRTLSQSGIDQYNLLISGKTLENGTKLQGINEILNQYLQQHQEEKLHKIKLKQLYKQILSESKTESFTLDFVEDNKGLAAMLLQFIDFANQLIEEKMPLLGMIQELKNSSVSVEFLSRLHLNRKNIKRLSNFIYKDYGYIEQSLEENFLSTIEGKITKKVLEEHHKQDTFTFHEILVALQKQQYEKNGTLESADQLLLPGIVNFIYQNLDCECSTLLEKARSEKQPLLDLFKEKQLLEDQNTESHASKYSDRPFNDHEIKTIKTTLDFYKNLQSNLAIFRISDENLKLDSEFYSEFDEFYQGLKNIIPVYNKSRNFLTKKPFSTEKTKLIFNSPQLLDGWSKSKESDCLGTIFIKDGKYYIGIINSVTNVKKTLFEPHNFANIDQSQYFEKMNLFFLSDLKRDFPKKYFSEKWHSQYPIPADLRKKYDYYRIDEHKDERKNDLEYHHQLIAYYQDCLKKDTEWQIYQFKYKSPEEYSDVNEFLSELTPSTYKMEFTKIPAEYIKKLVDDGKLYFFQIYSKDFSEFAKGKPNLHTLYLKAVFDQKNAEEFNYNYKISGSAEIFYRPASIEVRVTHPKNQPIKNKNKNNPKTESVFQYDLYKDRRYMSDKFFLHLPVELNRTPLLPNDSSVNSMVNQVVSSRNQNYFLGIARGERHLIYLVLIDQNGKIIKQQTLNQITSSYQEKSNNQTVEVVTDYHDLLDDKEKLRKKNLQEWQSVENIKELKAGYLSNVVNEIGKIIAEYQPVIMLANLNTGFKNSRIKIEKQVYQKFEKALIDKFNYFMRKDIDPSTIGGLYHALQLTKEYSKQYNGKQNGIIYYIPASYTSNIDPTTGFISAFVQTRYENVEKAKALIEKFNDITYDADESLFCFTADYKKFGPEAKLWQQNVWQIYTNGDRIYTFRDKEEWQSKSYILVEEFKDLFTKYQINYHNDLKTQILSQTDAGFFKQFLFLLRLTLQMRNSRTTDSNEADIDTKKRENDYIISPVKNQYGKFYDSRQDYMEWPENADANGAYNIARKGLIMLKHLKEGLPEKRICDISTEEWVQFVEELNKX

SEQ ID NO: 45 - protein, 1293 aa, synthetic construct
  REGION 1..1293: note = EpiCas046
  SITE 1293: note = misc_feature - Xaa =
MVSEKENTPTFNSLTNLYSVSKTLRFELRPQYSTLDHIKDDQIVDKGEELKNHYKTFKKILDQVFSRIINDSLDKTYLDQKYISTYQDLVFKHRDQLTDKDRAELKALKETLKKQIDKSLDHKDKKAIFSDPVKFLIDNESDFADLIGDNRPSIEAFNRQKGYLSGYLQNRANIFDHTTNETSVAFRIVEENLAIFLNNRLTLQHFLEKVADKDGLLKSLQETLSQLGFKLKLEDLLSLDYFNRTLSQPGIDQYNLLISGKALEDGKKMQGINEILNQYLQQHQEEKLHKIKLKQLYKQILSESKTESFTLDFVEDNKGLAAMLLQFIDFVNKLIEEKMPLLDMIQGLKDSSVSSEFLSRLYFERKNIKRLSNFIYKDYGYIEQSLEENFLSTIEGKITKKALEEHRKQDTFTIHEILVALQKQQYEKDGTLESADHLLLPGVVDFLYQNLDCKHSTLLEKVGSEKQPLLDLFNEKQLLEGQDAESHASKYSDRPFNDHEIKVVKTALDFYKNLQSNFAIFQITDENLKLDSEFYSEFDEFYQGLKNIIPVYNKSRNFLTKKPFSTEKTKLIFNNPQLLDGWSKSKESDCLSTIFIKDGKYYVGIINSATNAKKILFEPYNFANLDQNQYFEKMNLFFLSDLKRDFPKKYFSEKWHNQHPVPADLREKYDYYRIDEHKDERKNDLKYHHQLIAYYQDCLKKDTEWQIYQFKYKAPEEYSDVNEFLSELTPNTYKMEFTKIPAEYIKKLVDDGKLYFFQIYSKDFSEFAKGKPNLHTLYLKAVFDQRNAEEFNYNYKISGSAEIFYRPASIETRVTHPKNQPIKNKNKNNPKAESVFQYDLCKDRRYMSDKFFLHLPIELNRIPLLANDSSVNSMVNQVVSSRNQNYFLGIARGERHLIYLVLIDQNGRIIKQQTLNQITSSYQEKANNQTVEVITDYHDLLNDKEKLRKKNLQEWQSVENIKELKAGYLSNVVNEIGKIIVEYQPVIMLANLNTGFKNSRIKIEKQVYQKFEKALIDKFNYFMRKDLDSSAIGGLYHALQLTKEYSKQYNGKQNGIIYYIPASYTSNIDPTTGFISAFIQTRYENVEKTKSLIEKFNDITYDAEESLFCFSADYKKFSPEAKLWQQTIWQIYTNGDRIYTFKNKEEWQSKSYILVEEFKDLFAKYHIDYCRDLKAQILSQTDASFFKQFLFLLRLTLQMRNSRTTELNGTDADTKKRENDYIISPVKNQYGKFYDSRKDYVDWPENADANGAYNIARKGLIMLKHLKEGLPEKRICDISTEEWVQFVEELNKX

SEQ ID NO: 46 - protein, 1295 aa, synthetic construct
  REGION 1..1295: note = EpiCas048
  SITE 1295: note = misc_feature - Xaa =
MKSIYENFIGLESKNLTLRFALNPEAKTQENLKLYWDKLRDEERDRAYPIVKKILDKEYQQLISEGLKLLENQNVLDWTELAEYIRTSDLSKKKKEDKRLRKLIAQNLKAHPLVDKLKVKNAFGKNGYLETLPLGKEEKEAVKVFAGFGGFFNNYNKNRENYFSTEEKSTAIANRIVNENFSKHFSNVEIVTKIQKEVPELIQIVEAQFKGYDAIFTVNGYNMALSQAGIDTYNEMVAIWNKEANLYAQKAGKLPDGHPLKKKRNYLLSALFKQIGSEKEHLIQIDRFDGDEEVIEALTGVKKMLQEADVFEKLNMLVEDMENWDYSKIYLSAQSLSNVSVFLNNLYEDERENSWNYLDNVLREKWQIELQGKKKGTDLEEAIRKKKQSFYSIEELQEAVNAIEETDKCYNVSKWLLGAMKSERVIEEKKKDVEDFCTQWKNERNSLKETDITALKEYLEQWIFLARYCKSFYANGIEKKEKDEAFYHILEDVLYVLDEVIYFYNKVRNYVTKKPYSLEKMHLKFGHNELANGWSVNKEGNYGTAILRRNGKYYLAITNSLNKKMSIPTQLESTGNNYEKMVLNVFPNVFRMIPKCTTGRNDVKSCFERKEPNEYFFIDTPEFVNPFKVTREEYELNKITYDGVKKWQSDYSKNTQDEKGYKEAVTKWIQFCMRFLQSYKSTAIYDYSTLQQPEKYETVDSFYHDVEKILYECHFEYVPANKIEQLEEEGRIFLFQIYNKDFSENRRPDSKKNLHTLYWEALFSEENRKAKVIQLNGKAEIFRREKSIEHPIVHKAGEVLVNKRTKDGEPIPDDIYKDLSNYFNGRNVTSEKEEYKECLDKVYTSTKKYDITKDKRFTETKYEFHVPITLNYQADGVKYLNQKILHVLRDNPDVNIIGLARGERNLISYVVLNREGKIVNNQQGSFNIVGKMDYQKKLYQKEKNRDKERKTWKNIETIKDLKEGYISQVVHELTDMAIRNNAIIVMADLNFGFKRGRTKVERQVYQKFELALLKKLHYLVTDKTEGEAMLKPGGVLQGYQLAREVKTLKEIGKQCGCVFYVPPGYTSKIDPTTGFVDVFNMSGVTNREKKKAFFEKFDNMFYDEKRDMFGFSFNYEKFTTYQSSYRNDWTVYSNGSKYVWNSLKRTDELIDVTKELKLLFEKYAIDYRNEALFEQIMSQDTDKNNADFWNKLFWYFRVLLRLRNSSDELDQIVSPVLNQNGEFFETPKKITEKSYLSDYPMDADTNGAYHIALKGVYLIQEKIADESVDLDNKLPKDFYKISNAEWFMFRQKEKX

SEQ ID NO: 47 - protein, 882 aa, synthetic construct
  REGION 1..882: note = EpiCas049
MEKFPRCSHLKVKQHCGPFVEWYEDLQSDRQKYVSLFFPWIGIGYGERKDEGDDIDFISPEQTLERRKAELGKLEELVVRFVTVMLKGGSSASSCYSLFYEDYKGKDTAKKSTSKVNFIKALPLLSKKEIEDRVLGDNQLSLINLDEWMELGQSGLSADDIWDKVSQRLVTGERSLGVDVRHTLKIRCQRPRDRGYCVVLMEMVARQLRSCYECYKNHLNELQELREKTTKQITESRDDLFSLVCDLATVLTEQDSGLSERTLFSGRKQALKPQGKPSKIQAIVDELRQPQYSPLLEADDKELRQAYKAWNSQRRLRDRETRRPYPCRPNWRNDYQVSLGLTGRGGFWMWMDGSDVVVDLQDCGRLTCSPSHYFGDLAIEGQLHQYDIRYRNKLKRRGKKLRKPPAYGEWIEATIKEIGIQRKGGDYYVYLLPVISHDSTNFQAAKKFFAAAAPKQEIIDALPDEMKVLALDLNISNPVCSVKARIGRALDGPLSVLDYGQGEIVDGPKILVTDGPRCVALIELHSDVFFVKDAIREYKRTHGVAFNGAGMDDEMLERLSSIPSPSPSVRCQIQAWVAAIGKRLHLLHYGMRREGYKNLGEAIRLLAAMDAYDSMLQSYQRIHLKKGQSLPRQAKNDHSRAGLRETITRRIAYAIAQEARDCNVVFVEDLKADFDADNDNNALLRLFAVKTLIRYIQEALEKIGVGLVLVAKDGTSKTNPMNGDLGWRDRNNKEHLYFELNGEAVRCHADLVASLNVLLRGLDHSVCPYSFVTKDTNKKPGRADQKDKGYGKRLKRFFKDRFGSSNLRFVAKPDGEVTATTRRPKDALIGERVYYHGGKLVTSEVHYRMENEIKHLLTLKSPAKELSLTEDGIRSYENFTHV

SEQ ID NO: 48 - protein, 831 aa, synthetic construct
  REGION 1..831: note = EpiCas050
MVTPESQSDAKKAFADALFAGQEAAYGSFYAKYTPLPFPRKDSALGRLTAELGTIGDEKYAKALKLNGLDPDEWAGYRNRAIAGEPPDALWEEIKVKVGVTNKATLASRGVGNLPTLRPDSKSYATALAYMLSSSLSSWEECAKLHRARREEIAAKAAVPDSMTEEYALVCQLAQALEEAGSGLTAKMLNGAIREMAKTDSVYMVAITELADPKYKPLLALDKEDRARLMDTWQAERKLSTSLAAPKRPLFDKDYQIPFGMTGRGESFALRTDDSGRLWVKIGDHEEFSCAASDYFGNLKVEALPSDKGYKITYRKMVKNRRRQRAGYKSSVGGPLIEGEVKQILLQRRMNRRGKESFYVYLPHSIAVKDDRRHLSWFFKRADWKDGKGLEELPDQLIVSGIDLNIDQPVTVTKAIVSKNYTEGEINCPYGGGHLTGKPRSVCPDTPISQEAKLTHRKMRLLIEVIRESKACHKAGTPLSSEGSERLKILYADISKPPCRNNRESIQVVYSSLKKTYRRLKYELMQQGANDVSGCIRMLQLDDTHRALTACFDAFSGKPALAGRNFGVARKKTSKGTARLNFREHVSHLYANAVLRACHGSDVVFIEDLSTSFDGNAHNSLKRLFAPGQLIDAITSLLESHGIAVVTVDPSGTSRRHPITGDIWYRDSMKKRELVGLIDGELVSVNDDLAASINVLTKGLNHSVVPHKFKVDPSGRVNVKEDGGKRLKVFLREKGLSGRLPVGSADAAKSKKAKALTVYPSSSGLLSDRQVFEVLEQIKHLLAAGCKPRPVELMAGPGETYAAFSLKEVLEWSKSKSSEDLRHTGLGDETG

SEQ ID NO: 49 - protein, 865 aa, synthetic construct
  REGION 1..865: note = EpiCas051
MSNIKTTNILIPKQQDNYFEIENMLDRQNKIANDVFKLLLTYQGHILDQHFFGEDSKEINFIQKRQELYLVAKQADPLVTEDVFNLTCDGFVNQIASKSNMSNDLFKAFEVAYEELSEMKSYVAGKSEAKKLACKLKQWGVLPSFDNHIGEIKSGLSNAFGRFKSWYECDKMTQENFQKHVDSKSAYLEEMGDVISDRFQEFANELGNVYNSKGLDNFDYFGGKFLLFYEQILKPSFEKGEVCYSGSIEFSNGKKQQYSIYGGIIDLLFKYKELWETKSWSKKVKRKIETVEQCTFSRFYNIFEDFKYLRKNKQRAGFREFSSTSSASICLSNSNIIKYDITQVDGDRNNFYLTINGKQIQCRYRTKKIKYNKTTVEKTVENKCYLDNLEIEKQIKNDKPTGNYIFKFDVNKKRSNIATVNEPKIFKNRKGIYISLCMNIDLDDGKYDENKNIAFYLKTKYFNTYTKEADTLPLLKNKTVRIMGVDTGIKPIYTYSILDYKNGKITDPNIHGKLEGKYEFTNYFNMKSLKNVIRDLPKYFQTDDHKELFKSHLFNDLTFTRDELFDELKNFNTLREYKSRKNKGCLIRREIKSYRHKIGRMKDTNFKSDVKDITNVFGIVKYIDSYKQVCSSWNYIGETVETKTQPNFAFADSHLAKYRENLKDNMIKTLVFNIIDEAKKHDVDILAIEDFSSEYSTSTKRDNVLKALFAPSQIQSEIERCAKKEGIALMTVTRHYTSQVVFGKDIFGYRPKENDKTKLKWIENGEIKFVDADINAAKNIAAKLALKDVSPYTLDIEGDGKYFKSSKWDKSKVFKKVNDKFVNVSDQKPKNTKTKMKIFLNGDNYITKEKKNELVEEIKNYKIKT

SEQ ID NO: 50 - protein, 900 aa, synthetic construct
  REGION 1..900: note = EpiCas052
MKSIKSIKSIKTKVVKNNELKLIELSTWCSSICEQLERYIFILGGKQIHDRDGVVVLDGAVERKIYCKKDKSLIAACEVVYKHFTDKSSKSRTFGSWFLGGKSEGDNTNKGRKSTKEKTEKQIAKQIADKKELTDSLQLLWDKKLLPFPIDNKGYDFINTPRAKSYKWAITKTIHAKIKSYNEQCVETKKEYDALNAEINTYKTILFSGYSEKDIDDLQKFVDICEANNHRINYKFISFLKRKDLNFDEQTGKYRKEGKWIQHKNGKEVKSKYSMKDEIVEALYKYKSLTKNDVSVLCNEHQKEDEMGKVVHYNMKRYSDLLFRKKNKKEIPSYTKISLATSKIELGLNNVKYNVEQVEDKLIWTICDQTGKDIQFVTVYTRKKEDNRTNGKKGAGFYKGKHHQLEDLKIVPVGDIGTYDISFKVNGKRPFTGTLKEPNIICRGGKVFVQMPININIDKTLNDARKKVLYAYRETYSGSVNGKKQKMIKIENSKIAESLKSLGRDAIVLGVDLGLRGLATAVVSHNGKNETVKSSQYIKGDIVEWEKYRVFNDNIREVKKYIFLTKKSYTATTEEYTEFYKECSKPEQDYLDSLKTYKDKNVKLNELKYTKNAWSVSKMFEDVSKMFETLKQDRLKYYDIFNMPYWAASLKNYMSLMKSYNYVGVDIKVSKEYMSKYQSLYNNIKEDYAKKIGSYIVQLAVAKNCDIIVLEELKSNLGSVDRKSKRDNEMSLMWNCGRIKTHVENMAKDYGMFIDEVPEYGTSQVYHKTGNYGYRDEDNREIFWYEDNKDVAYIHADENAAINIAKRFLSQHTDNSSFSVILKGDAYYLNIASNSKRMRAAALKTFGDLNKPFKINANDKNGNLYKKTRIFKSDSRWIGVNDKDLYIEHIKSLRNLRVRQ

SEQ ID NO: 51 - protein, 903 aa, synthetic construct
  REGION 1..903: note = EpiCas053
MNHFTQNLAVTKIDSSVQTLYDDFQEDFRKWYSILCSWAKVPYRVEDDNGNITHVHAKQRAAELSSLYSAQEQLLIERFMRVFLGASKEASSSYSLFYDKYAPNGAKKTAALKEFLGEIPTLDDSQVEKKANTNKNCKRITVKEWRDFAAQFNKLGIDAIWGKLKEKITSPDRGVGLQVRGFVSERCKHPENAGYCRQLCQKLTEQVKSTVRICELYIQETADLQSEIGNKTKQPEVKKCFDLLCQLSDELRDVNSGLSGRVLFQSIKDIQGGKDSDRKNKVCRTVMSDDKYLCLIDSDKKIVQRAFQAWKLSITLKKRQEKLYPKQRSHQNDVPMPFGLTGLASFDVSPDGSKFTFSEGSVETKPSLYFDGLRFQKNKHSFDMEYRVAYASRRTKKGRRPKRVGRLRKATLKQVELFNKGGKFVIKLPHGIELPEHRAKLATFLMCADPKEKGYSKGGLVDEFRALAIDVNLSNPLAACVADVSKHGDGPIQALDYGRAKLCVDPFFLVANTPVCGQLIKLCKECKAVVQAIKEFKECQAAGSDFSFETDLFLPAGHSPRHRIQSKVQELSRVLRDWKFKLQNTNQVAEMIRLLELMDAYQSMISSYQRIHLKAGQTLPPQKKNSTKRANYRKDISRRLAFVVADYAQSLNVDVVFGEDLKADFDRDKNTNSLSRLFAGGMFLQHLENALAIRGIAFVKCAKNGTSITDPVSGVFGWRDKFNKERLWIERDGELGWVNSDHAACLNVLLVGLMHSVVPYRVYVKAGRKTTDDVEVFEEDHPIGPRKKRFFELCFGENKPKFEVVENRVILDNAGTYQGFIYAHCLDAMQSEKQHWAEYDRIKELALAETNPKQFGLTPKKGEGYRNFCQPDIHEQVEVVGNKRVAATKQILESLADEKIEAT

SEQ ID NO: 52 - protein, 737 aa, synthetic construct
  REGION 1..737: note = EpiCas054
MSQEGEVKFKSNSDMKDRGVVTGYDYSLINAFCSKYTNLWEERDDYDSTYLDKGGRKRQACIMTDRYYISHMIKRLKYMTKKHVNVPLFEQGKKAPCQLFGKNYMSYTLRQDEETGTFEFSFTDPTSESEGRAVKKFNASLAKNGYLNNLKIVESSNKDGVYKVSFNTAVENKDTLTIQTTYTGYIKEPRIRFNLEKNVTYLDLCISGIESDNDTFNKILSEEQKENKEAIFKGFSSYFKASYAKGRDGSQKGSLDVDVFRAMGVDLGLNPLAALSIYEVKKSDKGEDIGGQKVKIKHLTDHKSSDINKANKDCRVKKAKRTLKFIEGVKGLISVANHHCNYLEDVEATGEGKEVNVGYMEMLCGDVIENHTPFKDLEDYMKYIENNLYGVDRKEWKKKTSDWVVPSLIQQAKKKISEMRREYFDAEDKHYHTTKVNHVCNLQLHKIQAVKEMISTLNSFSTLGLNDEEKQAHADNGRPCADLWEYINGLRNYLSKGIASIIINHALRNSVNIIFIEDLDMKASSFDSKEENKLKSLWSASQIMDYLEKFAAKHTIAVQEVNKFMTSQFSSENGTLGVRGASGNKSQFLYEDKGEVCDVDSDINAARNIAIRGITRHSNMPMFKVKVASDKEFILEMPKAAGMQKLSSMMKYLDSDNPRYVKFVKANNGTLKASVLEKEEFNKLKKQAIAWKKDNKDADSEVSILRSGKHFYIRKELEAIVKAKLCPEVEEETVKAK

SEQ ID NO: 53 - protein, 972 aa, synthetic construct
  REGION 1..972: note = EpiCas055
MATRTSHKSSKIRILPTGTTKKALVEYNLLFHDQVAKIMLDLLTMRGVPYVLRDKDGEHEITATQIREAQWKLINSGRDSGDCIRLSQSIIDMMAQIYGGIMETGNPTKYRANLYLKKTNSGNEIGQGRSSASFMLFKDVSRGLSRPEFTRQIKDKNGKIKKPFAWATEERLMELYDAVRNDEMQPIEVEAERKSLEPDVTMENLVKLRKVGILPLPLPGGHLYQGRGLGKLLAMPFDAAVGRLKSHYQANLAAVEARAALDKVVADKKDNVPAPLYALFLEFAEECRQNGFGVSKRFAKKLHGLNIPKTDFEPGDPFIVGLEIIKQYKYRPLWPQIGTLAGVYLAGVKAEKTKGYAAITIADAYRSPALIRFGITNYARFEAEVNDQQGIDFSLQIPVDDQWTTVTVSGMPSNYFQNPELIRQTSNYALSFTKGNRQQRRMGVILKEPLLTVRYSSDPEQEPRFYVHLAYNYDKTSYRMIGLDKIRAFFSSNLSKGAKLPHGTPYGLRTLSIDLGIRPIISSHLMELVSPHDIEAAQESRNGIEPIEVPDMGFARLVDSREMGGVWNSGLVGRMIDASRRCKSARHMILVSKLMRDGKPLNTHQQFLIEKYVKMYGVFLPPDDWARKDIIGEFIRQILADYRVIKDTMAKRNHPDHKGKFPDIICSDVFRMLFLIKDVISMLKSWARYKRKTGDKEIQGQNPTELNQYWRYYNNLKDDLLKKIVHSLVRYADEHGVHLLLLEDITGNRDSTKHRKENSMFAIWSPTLIRTRLEEEFEACGMLVQAINPSFTSQTHYMTGEFGFRDPRQKECLYFKREGRLYLIDSDGNSSMGLQVRFWTRHRDLWTLKTVKARRDRAAKLLKLDPKRAECTFVAKPTFEYAKSYLKRNLASPVAAFVPDDNGNFILHKLGVNAANQLTEGQERGETFFRHGTDWITHASHKKRMSAIEAMVEKVKKKGDKMTEADLVRLKV

SEQ ID NO: 54 - protein, 1007 aa, synthetic construct
  REGION 1..1007: note = EpiCas056
MSSDVVRPYNTKLLPDNRKHNMVVDTMNWCDNACSVFFDLFVGLYGGVKIGDVTNFLTEKQDTTTDADLLCAINWFRAMPVSNPDAVVGPEQLVVRFRDYSGHNPSPMASMYLSAPVNPRYTWVDCRFLYQQMCQTLGANLDVDISTLVQTGLLPIQKDTQLNAYSFCSNVFGEGVKEDPSAKVTSYTAAANALSGATNPIITYEDYRQILSSAITNRPSRKGAKDILAADLDSTKTGVLPGNYVSQRIAMYQASAASKARGVVLPNRLRVKAYLQNLVGLNYICTTWAAPMLRAVGDIKSKNSNNYRFVGERLERQQEIDNLLAVSNIRSAQAILQQFRANESNQFYIKKRHIGKGLPSLYDMWDLMDEKEGIDNYIQSLKGDKTERLPIRELLQIIYPHRKSVSAELFLSAAELTDVEDRQNRCMVHPTVRGSLVINFGKSSTIFGAITPPDTLIKFRGEMVPAGQTGMIWFTMHLVDGGKWVKHHIPCHSSRYFEEIYAWKKDLPTLPFPRRSLYGYAIGNKISDTRKIDLRRKKASKQYLRTLENMTHNVEFDPTTQFMVDQELNVTITARVKKIPPVRDVPVGSRIMGLNKNQTIRDCYSIWERVEANTSGSFQHPVVQDVWLRLVADGHVTSTVNGPNGQIDQMSYSGPDVANCGNWMQERSNFITSVDDADQSIMKHFLWNNRVKLYAWNCVYAGTLMKAISKQMTTANRNLFRNEILSFVGGRFGCRLGSLAQVSLDLLRRMKSVISCYFSRNNAFTIEERSAFDKELMELFESLDNKMNNKRKEKVNRTLSSIFQVAKQHNVVRLVVTKNLQTPDANVRSVMNQRSVDWCARKIEEKIVDLGNVIGIYVDLVEALDLSHLDPFIYIAKGDVGKECQFDEVESSSLLPIHTSMFRDWHGLVNKGRTTTPIYHSALRSFASHYGLRFDDLARIKAAELAKMLPDQKVFIPHRGGRFFMSTYPVTSTSRPVSYDGRQRFLNSGDMVAPVNTMLRGFRRARS

SEQ ID NO: 55 - protein, 1011 aa, synthetic construct
  REGION 1..1011: note = EpiCas057
MGITMSDTTFRPYKSKLNPNERKMPMVVDTMNWCDNACSVFFDLFVGLYGGVKIGDVTNFLTEKQDTTTDADLLCAINWFRAMPVSNPDAVVGPEQLVVRFRDYSGHNPSPMASMYLSAPVNPRYTWVDCRFLYQQMCQTLGANLDVDISTLVQTGLLPIQKDTQLNAYSFCSNVFGEGVKEDPSAKVTSYTAAANALSGATNPIITYEDYRQILSSAITNRPSRKGAKDILAADLDSTKTGVLPGNYVSQRIAMYQASAASKARGVVLPNRLRVKAYLQNLVGLNYICTTWAAPMLRAVGDIKSKNSNNYRFVGERLERQQEIDNLLAVSNIRSAQAILQQFRANESNQFYIKKRHIGKGLPSLYDMWDLMDEKEGIDNYIQSLKGDKTERLPIRELLQIIYPHRKSVSAELFLSAAELTDVEDRQNRCMVHPTVRGSLVINFGKSSTIFGAITPPDTLIKFRGEMVPAGQTGMIWFTMHLVDGGKWVKHHIPCHSSRYFEEIYAWKKDLPTLPFPRRSLYGYAIGNKISDTRKIDLRRKKASKQYLRTLENMTHNVEFDPTTQFMVDQELNVTITARVKKIPPVRDVPVGSRIMGLNKNQTIRDCYSIWERVEANTSGSFQHPVVQDVWLRLVADGHVTSTVNGPNGQIDQMSYSGPDVANCGNWMQERSNFITSVDDADQSIMKHFLWNNRVKLYAWNCVYAGTLMKAISKQMTTANRNLFRNEILSFVGGRFGCRLGSLAQVSLDLLRRMKSVISCYFSRNNAFTIEERSAFDKELMELFESLDNKMNNKRKEKVNRTLSSIFQVAKQHNVVRLVVTKNLQTPDANVRSVMNQRSVDWCARKIEEKIVDLGNVIGIYVDLVEALDLSHLDPFIYIAKGDVGKECQFDEVESSSLLPIHTSMFRDWHGLVNKGRTTTPIYHSALRSFASHYGLRFDDLARIKAAELAKMLPDQKVFIPHRGGRFFMSTYPVTSTSRPVSYDGRQRFLNSGDMVAPVNTMLRGFRRARS

SEQ ID NO: 56 - protein, 754 aa, synthetic construct
  REGION 1..754: note = EpiCas058
  SITE 754: note = misc_feature - Xaa =
MARNNKKLEGLVKSYKFQAKNVDIMIDILEEYSEYYNRVSNWINDNLMTLTIGDIALKCNTSQSVGYMQIALSDDYKQLPMYKLFEKKYPSTHCLNLLYAYITNNNIDNYTGNIWGLSTTNYRKYGYINLVINNYRSKFANLNFNVKRSKIDINSNVEEIEKQCLYERIRLGLIKIEDWKKHIDSLKRQEIINEKALHRFNILSDYFNKNGESINERQSLLTIEQLQKFNGCQMKNKSMRIQVQNFKLNEKPNSLGYILQIHLNKKLFNIELLGNRQIRKGNKDNYTTLVDIINNHGIDIIFKLENNKLYVILTSTDTTFNKTITTPNKICGIDINIKHALLNTSERDSMNIQGYVNLYKRVLENDNFVSLLSKEEYETFSNMASYLTFCPIEFNYLYSRISNDEYTEKERLFSKILIDIQKEYENKKGCDLEKNYIHSVNKLRAKIIAYHKLKTIYTQKKQEYDIKMGFVDDSTLSKDGMDKRRFESPFNNTDCAKQIKSKLDNVLQDIEGCRKNIITYVYKIFETNGYVTIGLENLDNSNFEYHKPLCSIKSLLNYHKLLNKTIEQVKENNSVADLIEKDYYIFTFDENKNIIDAKFSDKGFYKINKSWFINQLIKVLHFASIKNYFITLSNNGKTQIVLVPSEFTSQINSLTHKLPMIKNDKGENVRPTKFRVRKTQEKHYNGLNADINATLNIKYIVENEEWRNVLCKSPNKKEYKDARGYNFPTLIATQKHYNVSKQLEKLGGIEFIQX

SEQ ID NO: 57 - protein, 911 aa, synthetic construct
  REGION 1..911: note = EpiCas059
MEETRTPEKIACQQDRSAFFKLGKLPQKTTSAIYHLMRCSKRYIDNVSETISNNIEKAAMTSDNHIFRSALKEEYKKIYREKFYPDVTGQPFPDKGPDNTHIVERTVENLCGQYISALKGVMKVRKPKWQDDIFEDESDKVIAEMLMASGNEADIHPKLEKKIASAKKKDKMDEWQKVLDRYDYIKSKTDIEWTYTTLLYDHIARECARSMFEDGNKPKFCYTNGNKFAKQVVDELWGSERVEYEYEKYLHCRTLEAYWNEENKYFFLENQKKSKKTYEARQIIDHFIENEDAVKAETEEYMAKVAKEFGENGGCKFRTENILYLVNSVSINKKQDEERCPIFKVTDIPQYAEAGIDRIGECKYVIKLKLFTVGERYGTTYMPEAFSGLYDRRTNQRYDLIDRACKGSYVYLPIYISDKDSRKWSDRLKNTFTMSAFSISERKTKKDGRVLCVNLPYTHIQHKPRNYFKVYPTYGVDINVKHSLFSTNATNLNGYIDVNGELIDFYNSIGRPDKLKNINLAVTLGDKGGTAGNHTNFLLVSDLGDWDDHGKKRNDRFVKREDGTYYSRPDLLADGLNFLKKKYSGDRKSYDYINTTIAFRNNQTRLRCIDKEWNDRKRAYDEEHGKPEKGERSGFYPTEDAERLTMERQALRREIAENTRQVIAYMLWLFQENGIKSIALEHLDCGSWKNFRDQLKPKWFCNNCKYWAEKYGIPYEATKYYRSNRKYFDIEDGELIPENVKLNALGEAENDKRYFQTSIAKAVGMSSIKQMFSIVSQNYDIQVNYTEQAYTSQTDSRNGTVDKDFRVSQECYIGSDGTKMNCDFNAAVNIADRISDDTLREEFNKELPMKEFVKYGATQIVSRNRRWNAENAERVTMAAFQKRKNADGGAHTAAVKPTSKTEESSLKLVAE

SEQ ID NO: 58 - protein, 913 aa, synthetic construct
  REGION 1..913: note = EpiCas060
  SITE 913: note = misc_feature - Xaa =
MIDRNETHLSKKSTRSVEFKLAVPNRIAPVIDEMLDLSITMSNAISKDISEHLTDWVQRAEVTKEQKRPYDYLYASEKEGIRYWYKHSGLRDKMYFDFCSKYFGYPVLKNKNAHFLTRTIENVAAAYEGRFTRFQEETKIRATDQSQLTEEEELLKKNIKEILNIMCSLKPGQTVDQILVRRSKTGKNQEKYQKLLSFYVDVTSDEKSDKYINFDILFADMCYRNSATFILKTMEISVDDSGVVNFGMVSPLVELPTKIGSSEEATMRHILENRSVRLINEVNDMIFELTVAQYIKSSCRYRFVDELTEKANSKDSVFHKNLLMYYTSHRDKVDEGYKQYLHSVNESFGKKQIVWKNSTLKLNYEEKVEKDRQVPIKMISSIKQFADLGITSISGCKYVLRVHMILDDEKKFKDQHLFCDLYDKRTGRRFDLLDHETKGYAYIPIMVPENDRRTYDISDFDCQMTEISSRDGMYFAKLTRDIDYVKPVNRFGEEQVCGVDINIRHAMIDVSERSIMNSEKYVDVEGFIIDYFRNVKNLPSVADDLVTNRYLGEAEEAGKHTFFGLMSSINKSISRETGGLKNRFVKNVDGTYTILPDCFSEPLNVLMKKYEQGSLQYRYVASVKALRVQLAQMHVLCMEHDDRRQVYDNLYGKPYIDDHPYEDPDFVYAHRKLHQKIETNTQFIVSYAIQLIQEAGFSCISVEQIDKKNLGKMLNLKSAKVFYNGCIYASSENGQAIADTRYFRTNCRYFDYPSDLNLFDIDHIKLNGVGEAYNINAKLMNSLGKYVGFASFKHMFAHIGTAHDVQVVFVEPAFTSQVDSKTGELRADKKEMRPYHHTYKHTDGTVSHADENGAINAKKIVTEEILRTRLLKENSVKDIMEYGGVQLERSGKSINGVRKVYEEVYAEKNGLVX

SEQ ID NO: 59 - DNA, 22 bp, synthetic construct
  misc_feature 1..22: note = sgRNA target-1
  source 1..22: mol_type = other DNA
caagtcccctcatccccacaga

SEQ ID NO: 60 - DNA, 20 bp, synthetic construct
  misc_feature 1..20: note = sgRNA target-2/Example5 spacer/Example6 spacer-20nt
  source 1..20: mol_type = other DNA
aagtcccctcatccccacag

SEQ ID NO: 61 - DNA, 1790 bp, synthetic construct
  misc_feature 1..1790: note = Example1 reporter
  misc_feature 939: note = n =
  misc_feature 940: note = n =
  misc_feature 941: note = n =
  misc_feature 942: note = n =
  misc_feature 943: note = n =
  misc_feature 944: note = n =
  misc_feature 967: note = n =
  misc_feature 968: note = n =
  misc_feature 969: note = n =
  misc_feature 970: note = n =
  misc_feature 971: note = n =
  misc_feature 972: note = n =
  source 1..1790: mol_type = other DNA
atgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaatggatccggcgcaacaaacttctctctgctgaaacaagccggagatgtcgaagagaatcctggaccggctgagcaggctcgttgggaagcagcctccaaagaaaccatcaagaaaaccaccaagccctgtccccgctgccatgtaccagtggaaaaaaatggaggctgcatgcacatgaagtgtccgcagccccagtgcaggctcgagtggtgctggaactgtggctgcgagcccccggcnnnnnncaagtcccctcatccccacagannnnnntcgggttaggggaccactggttcgacgtgagcggttctggcgaagggcgaggttccttgctgacctgtggcgatgtagaagaaaatcccggccccggatccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 62 - DNA, 2013 bp, synthetic construct
  misc_feature 1..2013: note = Example2 reporter
  source 1..2013: mol_type = other DNA
atgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaatggatccggcgcaacaaacttctctctgctgaaacaagccggagatgtcgaagagaatcctggaccggctgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactgataaggatcctttgaagtcccctcatccccacagtggtaggatatcggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 63 - DNA, 81 bp, synthetic construct
  misc_feature 1..81: note = EpiCas001-DR
  source 1..81: mol_type = other DNA
gttgaataaccttaaggttattcgactaaaatggataaaatgaacaaaaaaattgatttaaagtataattattcaccctat

SEQ ID NO: 64 - DNA, 19 bp, synthetic construct
  misc_feature 1..19: note = EpiCas002-DR
  source 1..19: mol_type = other DNA
aatttctactattgtagat

SEQ ID NO: 65 - DNA, 20 bp, synthetic construct
  misc_feature 1..20: note = EpiCas005-DR
  source 1..20: mol_type = other DNA
taatttctactattgtagat

SEQ ID NO: 66 - DNA, 20 bp, synthetic construct
  misc_feature 1..20: note = EpiCas032/033-DR
  source 1..20: mol_type = other DNA
aatttctactaatgtagata

SEQ ID NO: 67 - DNA, 58 bp, synthetic construct
  misc_feature 1..58: note = EpiCas034-DR(1)
  source 1..58: mol_type = other DNA
aaacaaagtattagaacaatgaaatttccattgttgccttatgatgcggacagtaaga

SEQ ID NO: 68 - DNA, 14 bp, synthetic construct
  misc_feature 1..14: note = EpiCas034-DR(2)
  source 1..14: mol_type = other DNA
acggagagaaaggc

SEQ ID NO: 69 - DNA, 81 bp, synthetic construct
  misc_feature 1..81: note = EpiCas035-DR
  source 1..81: mol_type = other DNA
ttacagaaattatatcctatgtattcacacgcttattgcttaaaaacacaatgtcccgccggggtaaccgcgaatggacac

SEQ ID NO: 70 - DNA, 36 bp, synthetic construct
  misc_feature 1..36: note = EpiCas037-DR
  source 1..36: mol_type = other DNA
atctacaatagtagaaattcttaggttcttataaag

SEQ ID NO: 71 - DNA, 42 bp, synthetic construct
  misc_feature 1..42: note = EpiCas038-DR
  source 1..42: mol_type = other DNA
tagatctacaacagtagaaattagggaagattgcaaaacctt

SEQ ID NO: 72 - DNA, 36 bp, synthetic construct
  misc_feature 1..36: note = EpiCas039-DR
  source 1..36: mol_type = other DNA
gtccaaaactcatttcgaatttctactgatgtagat

SEQ ID NO: 73 - DNA, 36 bp, synthetic construct
  misc_feature 1..36: note = EpiCas040-DR
  source 1..36: mol_type = other DNA
atctacaacagtagaaattataagggagtattaatc

SEQ ID NO: 74 - DNA, 36 bp, synthetic construct
  misc_feature 1..36: note = EpiCas041-DR
  source 1..36: mol_type = other DNA
gtttaggcagcttctctaatttctactgttgtagat

SEQ ID NO: 75 - DNA, 36 bp, synthetic construct
  misc_feature 1..36: note = EpiCas042-DR
  source 1..36: mol_type = other DNA
ctttgaaagaatataatttctactgaaagtgtagat

SEQ ID NO: 76 - DNA, 36 bp, synthetic construct
  misc_feature 1..36: note = EpiCas043/045-DR
  source 1..36: mol_type = other DNA
gtttaataattagaaataatttctactgttgtagat

SEQ ID NO: 77 - DNA, 36 bp, synthetic construct
  misc_feature 1..36: note = EpiCas044-DR
  source 1..36: mol_type = other DNA
gtttataaaccttatttaatttctactgttgtagat

SEQ ID NO: 78 - DNA, 36 bp, synthetic construct
  misc_feature 1..36: note = EpiCas046-DR
  source 1..36: mol_type = other DNA
gtttaacgattagaaataatttctactgttgtagat

SEQ ID NO: 79 - DNA, 36 bp, synthetic construct
  misc_feature 1..36: note = EpiCas047-DR
  source 1..36: mol_type = other DNA
atctacaacagtagaaattatttctaattattaaac

SEQ ID NO: 80 - DNA, 36 bp, synthetic construct
  misc_feature 1..36: note = EpiCas048-DR
  source 1..36: mol_type = other DNA
atctacactttcagtagaaattatattcttccaaag

SEQ ID NO: 81 - DNA, 37 bp, synthetic construct
  misc_feature 1..37: note = EpiCas049/055-DR
  source 1..37: mol_type = other DNA
agtgctggttctcgtcgctaggggagagtcaaagcac

SEQ ID NO: 82 - DNA, 36 bp, synthetic construct
  misc_feature 1..36: note = EpiCas050/053-DR
  source 1..36: mol_type = other DNA
gtgccttggcgatccgccgtagcaggtcgccagcac

SEQ ID NO: 83 - DNA, 36 bp, synthetic construct
  misc_feature 1..36: note = EpiCas051/054-DR
  source 1..36: mol_type = other DNA
gtgtcagaagacttataaaatgagtcttattgtgac

SEQ ID NO: 84 - DNA, 36 bp, synthetic construct
  misc_feature 1..36: note = EpiCas052-DR
  source 1..36: mol_type = other DNA
gtgtcaacgcatcctctatagttgaggaagcaacac

SEQ ID NO: 85 - DNA, 23 bp, synthetic construct
  misc_feature 1..23: note = EpiCas056/057-DR(1)
  source 1..23: mol_type = other DNA
agaaatccgtccttggttgacgg

SEQ ID NO: 86 - DNA, 23 bp, synthetic construct
  misc_feature 1..23: note = EpiCas056/057-DR(2)
  source 1..23: mol_type = other DNA
agaaatccgtcctacgttgacgg

SEQ ID NO: 87 - DNA, 36 bp, synthetic construct
  misc_feature 1..36: note = EpiCas058/059/-DR/Example6 DR-36nt
  source 1..36: mol_type = other DNA
agtggatgcggtatcaacttgattggtactgacacc

SEQ ID NO: 88 - DNA, 36 bp, synthetic construct
  misc_feature 1..36: note = EpiCas060-DR
  source 1..36: mol_type = other DNA
ggtatcactcatcccattatggataggtgaaccatt

SEQ ID NO: 89 - DNA, 23 bp, synthetic construct
  misc_feature 1..23: note = sgRNA target-3
  source 1..23: mol_type = other DNA
cctcactcctgctcggtgaattt

SEQ ID NO: 90 - DNA, 22 bp, synthetic construct
  misc_feature 1..22: note = Example6 DR-22nt
  source 1..22: mol_type = other DNA
caacttgattggtactgacacc

SEQ ID NO: 91 - DNA, 31 bp, synthetic construct
  misc_feature 1..31: note = Example6 DR-31nt
  source 1..31: mol_type = other DNA
atgcggtatcaacttgattggtactgacacc

SEQ ID NO: 92 - DNA, 31 bp, synthetic construct
  misc_feature 1..31: note = Example6 DR-31.1nt
  source 1..31: mol_type = other DNA
atgcggtatcaacttgattggtaccgacacc

SEQ ID NO: 93 - DNA, 31 bp, synthetic construct
  misc_feature 1..31: note = Example6 DR-31.2nt
  source 1..31: mol_type = other DNA
atgcgggatcaacttgattggtcctgacacc

SEQ ID NO: 94 - DNA, 31 bp, synthetic construct
  misc_feature 1..31: note = Example6 DR-31.3nt
  source 1..31: mol_type = other DNA
atgcggtgtcaacttgattggcactgacacc

SEQ ID NO: 95 - DNA, 31 bp, synthetic construct
  misc_feature 1..31: note = Example6 DR-31.4nt
  source 1..31: mol_type = other DNA
atgcggtaccaacttgattggtactgacacc

SEQ ID NO: 96 - DNA, 31 bp, synthetic construct
  misc_feature 1..31: note = Example6 DR-31.5nt
  source 1..31: mol_type = other DNA
atgcggtatcgacttgatcggtactgacacc

SEQ ID NO: 97 - DNA, 31 bp, synthetic construct
  misc_feature 1..31: note = Example6 DR-31.6nt
  source 1..31: mol_type = other DNA
atgcggtatcagcttgactggtactgacacc

SEQ ID NO: 98 - DNA, 30 bp, synthetic construct
  misc_feature 1..30: note = Example6 DR-31.7nt
  source 1..30: mol_type = other DNA
atgcggtatcagcttgactggtactgcacc

SEQ ID NO: 99 - DNA, 31 bp, synthetic construct
  misc_feature 1..31: note = Example6 DR-31.8nt
  source 1..31: mol_type = other DNA
acgcggtatcaacttgattggtactgacgcc

SEQ ID NO: 100 - DNA, 24 bp, synthetic construct
  misc_feature 1..24: note = Example6 Spacer-24nt
  source 1..24: mol_type = other DNA
aagtcccctcatccccacagtggt

SEQ ID NO: 101 - DNA, 23 bp, synthetic construct
  misc_feature 1..23: note = Example6 Spacer-23nt
  source 1..23: mol_type = other DNA
aagtcccctcatccccacagtgg

SEQ ID NO: 102 - DNA, 22 bp, synthetic construct
  misc_feature 1..22: note = Example6 Spacer-22nt
  source 1..22: mol_type = other DNA
aagtcccctcatccccacagtg

SEQ ID NO: 103 - DNA, 21 bp, synthetic construct
  misc_feature 1..21: note = Example6 Spacer-21nt
  source 1..21: mol_type = other DNA
aagtcccctcatccccacagt

SEQ ID NO: 104 - DNA, 19 bp, synthetic construct
  misc_feature 1..19: note = Example6 Spacer-19nt
  source 1..19: mol_type = other DNA
aagtcccctcatccccaca

SEQ ID NO: 105 - DNA, 18 bp, synthetic construct
  misc_feature 1..18: note = Example6 Spacer-18nt
  source 1..18: mol_type = other DNA
aagtcccctcatccccac

SEQ ID NO: 106 - DNA, 17 bp, synthetic construct
  misc_feature 1..17: note = Example6 Spacer-17nt
  source 1..17: mol_type = other DNA
aagtcccctcatcccca

SEQ ID NO: 107 - DNA, 16 bp, synthetic construct
  misc_feature 1..16: note = Example6 Spacer-16nt
  source 1..16: mol_type = other DNA
aagtcccctcatcccc

SEQ ID NO: 108 - DNA, 23 bp, synthetic construct
  misc_feature 1..23: note = Example7 EMX1 Spacer-23nt
  source 1..23: mol_type = other DNA
tggttgcccaccctagtcattgg

SEQ ID NO: 109 - DNA, 23 bp, synthetic construct
  misc_feature 1..23: note = Example7 VEGFA Spacer-23nt
  source 1..23: mol_type = other DNA
ctctcaagacccacaatccaggc

SEQ ID NO: 110 - DNA, 8207 bp, synthetic construct
  misc_feature 1..8207: note = EpiCas002 plamid DNA
  source 1..8207: mol_type = other DNA
tgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcataatacgactcactatagggcggccgccaaaaaacccctcaagacccgtttagaggccccaaggggttatgctagatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaggagagccaccatgaaacggactgctgacggcagcgaatttgagagccccaagaagaagaggaaagtcaacatcaagttcaacaagttcaccggcgtgaagcccgtgtctaagaccctgagaaacgctctgatccccaccgagttcaccatggtcaacctggaacggaacggcatcatcagcgccgatgagctgagagccgagaagcggcaagagctgaagaaaatcatggacgactactaccgggacctgatcgagagaaccctggccgagaaccacagcatcaactggcacatcctgttcgagctgatgaagtccaagatgcacggcgacgagaaggacaccgccaagaagctggaaaacgagcagaagtctaagcgggaagagatccaccggctgttcgccgagcaagaggaattcggcaagatcttcagcgccaagctggtgtccgatatcctgcctggctttgtggccagctacgccatgtacaacgacgaggacagaaccgagaaggccgaggtggtcaagctgtttaacaagtttatgagcagcttcaccgagtttttcgagaaccggaagaacgtgttcagcaaagagaacatcagcaccagcctgtgccaccggatcgtgaacgacaacgccctgatcttcctgaacaacatggacagctacaagcggatcatggaaaacttcaagagcgagagcgaactggtgtccaaaggcctggaaaccatgctcggagagtggaagatctcccagatctacagcgaggacttcttcggcatgaccatgacacagagcggcatcgagttctacaacgacatctgcggcgagctgaacctggtcctgaacctgtactgccagaaaagcaagaagaacagcgcccggttcaagatgaagcggctgcacaagcagatcctgtgcaccgagaagtccagcttcgagatcagctataagtacgacaacgacaaggatgtgtacaccagcgtgaacggcttcattaagaatatcaagaacaacaacatcatcgagcggctgctgaagctgggcaccgagacagagcagtacgacctgaacaagatctacatcagctcccagagctacgagacagtgtctatcgccatcggcggcacctgggagacaatcagaaactgcctgacctcttggtacgacgacaacatcaaaggcagcggcaaggccaaagaaaacaagatcaaggccgtggtcaaggccgacaagtaccagagcattgccagagtggactacctgaccgagacatacaatgccagcggcaccgatctgcagaaagccgagagctacctgagccacatcgtgcagatcatcggcgactacgagcccgaggaactgtacttcgacgagagcatccacctgattgagaacggcgacaagatcagcgagatcaagaagatcctggacatctacatcgacgtggtgcactggatcaagacctttgagatctccgagagcgtggaaaaagagcctgagttttactacgagctggacgacatctacgacaagattctggacgccgtgcctctgtacaacaaagtgcggaactacgtgacccagaagccgtacaatcaagagaagttcaagctcaacttcgacagccccacactggccaacggctggtccaagtccaaagagagagacaacaacgccatcatcctgagccgcgacggcaagtactatctgggcgtgttcaacgtgcggaacaagcccgacaaagagatcctcgagggccacagcaagcccaagaacgagacagactacgccaagatggtctacaacctgctgccaggcgccagcaagatgctgcctaaggtgttcctgagcaagaagggcatcgacaacttcaagcccagcgactacatcctggaaggctacatcaagaaaaagcacatcaagagcagcgaaaccttcgacatcaagtactgcagggatctgatcgattacttcaagaacagcatcagcagacaccccgagtggaaaaacttcggcttcaacttcaccgaaaccagcaagtacaacgatatcagcgagttttacaaagaggtcgagaagcagggctacaagatcgagtggacctacatcaccgaagaggacatcaacaagctggatgaggccggccagatctatctgttccagatcttcaacaaggatttcgccgtgggcagcaagggcacccctaatctgcacaccctgtacctgaagaacctgttctctaaagaaaacctggacaacgtggtgctcaagctgaacggcgaggccgagctgttctttcggagaagctccatcaagacccctgtgatccacaaaaagggcagcatcctggtcaaccggtgctacgtggacgccgacgatggcaagagaaagcccattcctgagaaagagtacatggaaatctacaactactgcaacggcaagcgggacaccgagctgtctgaaagcgcccggaagtactacaacatcagccagagatacgaggccaccatggacatcgtgaaggactaccggtacagcgtggacaaactgttcatccatctgccgatcaccatcaatttcaaggccaccgagaacaagaacgtcaacgacatggtgctcgagtatatcgccaagagcgacgacatccacatcatcggcatcgaccggggcgagagaaacctgatctacgtgtccgtgatcgacatgcacggacacatcgtgatgcagaaaagctacaacgtcgtcaacgagtacgactacaagaccaagctgaaagagtgcgagcagggcagagatgccgccagaaagaactggaaagagatcgggaagatcaaagaactcaaagagggctacctcagcatggtcatccacgagatcacccagctgatcatcgagtacaacgcaatcgtgatgatggaagatttgaatttcgggttcaagcgcggcaggttcaaggtggaaagacaggtgtaccagaaattcgagaacatgctgatcaacaaactgaactacctggtggacaagcacaaagccgtggatgagcctggcggcctgctgaatggataccagctgacctacattcccgaccggatgtccgaactgggcagacagtgtggcatcatcttctacgtgccagccgcctacacctccaagatcgatcctaccaccggctttttcaacgccttccggtttaacctgctgacctccgccgagagcagacgggacttcctgaatcggttcaagtgcatcaagtataacggcagcatgaatatgttcgagttcgacttcgattacaacgatttcatcacccacaacaccacaatcgcccggacacagtggaaggtgttcaccaacggcggcaggattaagaacatcatctccaagagcggctggaacggcacaaagaagatcgacgatatcacagccctgatgaaggaaatcttcgactcccgcaagatcgacttcagcgacggcggaaatctgctgcctgagatcctggcctatgagggcgccgacaaggacaagttcatcgtggaactgctggatatcttcaagctgaccgtgcagctgcggaacagcatgcctgatggctccgacatcgagtatgaccggatcatcagccccgtgatcaacgagaacaacaccttctacgacagcgacatgtacaccgagcctaacagccctctgcctatcgacgccgatgccaatggcgcctactgtatcgccctgaagggcctgtacgaagtgaatcaaatcaaagagaattggaaagaaggcgagacattccccaagtccgtgctgaaactgaacaatgccaattggttcgatttcgtgcagaacaagagatatgagcatcatcatcaccatcaccctgctgctaagagagtgaaactggatgactacaaagaccatgacggtgattataaagatcatgatatcgattacaaggatgacgatgacaagggatccaaaagaactggaagcggagctactaacttcagcctgctgaagcaggctggagacgtggaggagaaccctggacctgttaacatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaagctcgagcctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatg

SEQ ID NO: 111 - DNA, 7842 bp, synthetic construct
  misc_feature 1..7842: note = EpiCas002 gRNA plamid DNA
  source 1..7842: mol_type = other DNA
gaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacaggggagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaatttctactattgtagataagtcccctcatccccacagtttttttaggcagctgtagatctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagcgctgccaccatggcgcgtaaggtcgatctcacctcctgcgatcgcgagccgatccacatccccggcagcattcagccgtgcggctgcctgctagcctgcgacgcgcaggcggtgcggatcacgcgcattacggaaaatgccggcgcgttctttggacgcgaaactccgcgggtcggtgagctactcgccgattacttcggcgagaccgaagcccatgcgctgcgcaacgcactggcgcagtcctccgatccaaagcgaccggcgctgatcttcggttggcgcgacggcctgaccggccgcaccttcgacatctcactgcatcgccatgacggtacatcgatcatcgagttcgagcctgcggcggccgaacaggccgacaatccgctgcggctgacgcggcagatcatcgcgcgcaccaaagaactgaagtcgctcgaagagatggccgcacgggtgccgcgctatctgcaggcgatgctcggctatcaccgcgtgatgttgtaccgcttcgcggacgacggctccgggatggtgatcggcgaggcgaagcgcagcgacctggagagctttctcggtcagcactttccggcgtcgctggtcccgcagcaggcgcggctactgtacttgaagaacgcgatccgcgtggtctcggattcgcgcggcatcagcagccggatcgtgcccgagcacgacgcctccggcgccgcgctcgatctgtcgttcgcgcacctgcgcagcatctcgccctgccatctcgaatttctgcggaacatgggcgtcagcgcctcgatgtcgctgtcgatcatcattgacggcacgctatggggattgatcatctgtcatcattacgagccgcgtgccgtgccgatggcgcagcgcgtcgcggccgaaatgttcgccgacttcttatcgctgcacttcaccgccgcccaccaccaacgcggatccggcgagggcagaggtagcctgctgacctgcggcgacgtggaggagaaccccggccccaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgaccgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaa

SEQ ID NO: 112 - DNA, 7160 bp, synthetic construct
  misc_feature 1..7160: note = EpiCas057 plamid DNA
  source 1..7160: mol_type = other DNA
tgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcataatacgactcactatagggcggccgccaaaaaacccctcaagacccgtttagaggccccaaggggttatgctagatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaggagagccaccatgaaacggactgctgacggcagcgaatttgagagccccaagaagaagaggaaagtcggatccgaagagacacggacccctgagaagatcgcctgccagcaggatagaagcgccttcttcaagctgggcaagctgccccagaaaaccaccagcgccatctaccacctgatgcggtgtagcaagcggtacatcgacaacgtgtccgagacaatcagcaacaacatcgagaaggccgccatgaccagcgacaaccacatcttcagaagcgccctgaaagaagagtacaagaagatctaccgcgagaagttctaccccgacgtgaccggccagccttttccagataagggccccgacaacacccacatcgtggaacggaccgtggaaaacctgtgcggccagtacatctctgccctgaagggcgtgatgaaagtgcggaagcccaagtggcaggacgacatcttcgaggacgagagcgacaaagtgatcgccgagatgctgatggccagcggcaacgaggccgacattcaccccaagctggaaaagaagattgccagcgccaagaaaaaggacaagatggacgagtggcagaaggtgctggacagatacgactacatcaagagcaagaccgacatcgagtggacctacaccacactgctgtacgaccacattgcccgcgagtgcgccagatccatgttcgaggatggcaacaagcctaagttctgctacaccaacggcaacaagttcgccaaacaggtggtggacgagctgtggggctctgagagagtggaatacgagtacgagaagtacctgcactgcagaaccctggaagcctactggaacgaggaaaacaagtacttcttcctggaaaatcaaaagaagtccaagaaaacctacgaggcccggcagatcatcgaccacttcatcgagaacgaggacgccgtgaaggccgaaaccgaagagtatatggccaaggtggccaaagagttcggcgagaacggcggctgcaagttcagaaccgagaacatcctgtacctggtcaacagcgtgtccatcaacaagaagcaggacgaggaacggtgccccatcttcaaagtgaccgacattccccagtatgccgaggccggcatcgatagaatcggcgagtgcaaatacgtgatcaagctgaagctgttcaccgtgggcgagagatacggcaccacctatatgcctgaggccttcagcggcctgtacgacagaaggaccaaccagagatacgacctgatcgacagagcctgcaagggcagctacgtgtacctgcctatctacatcagcgacaaggacagccggaagtggagcgacagactgaagaacaccttcaccatgtccgccttcagcatcagcgagcgcaagaccaagaaagacggcagagtgctgtgcgtgaacctgccttacacacacatccagcacaagccccggaactacttcaaggtgtaccccacctacggcgtggacatcaacgtgaagcacagcctgttcagcaccaacgccaccaacctgaacggctacatcgacgtgaacggcgagctgatcgacttctacaacagcatcggcagacccgataagctcaagaacatcaacctggccgtgacactgggcgacaaaggcggcacagctggcaaccacaccaactttctgctggtgtccgacctcggcgactgggatgatcacggcaagaagcggaacgacagattcgtgaagagagaggacggcacctactacagcagacctgacctgctggccgacggcctgaacttcctgaagaagaagtatagcggcgaccggaagtcctacgattatatcaacaccacaatcgccttccggaacaaccagaccagactgagatgcatcgacaaagagtggaacgatcggaagcgggcctacgatgaggaacacggcaagcctgagaagggcgaaagaagcggcttctaccctaccgaggatgccgagcggctgacaatggaaagacaggccctgcggagagagattgccgagaacaccagacaagtgattgcctacatgctgtggctgttccaagagaacggcatcaagtctatcgccctggaacacctggattgcggcagctggaagaacttccgggaccagctgaagccaaagtggttctgcaacaactgcaagtactgggccgagaagtatggcatcccttacgaggccaccaagtactacagaagcaaccggaagtactttgacatcgaggacggggagctgattcccgagaatgtgaagctgaacgccctgggcgaagccgagaacgacaagagatacttccagacctctatcgccaaggccgtgggcatgagcagcatcaagcagatgttcagcatcgtgtcccagaactacgacatccaagtgaactacaccgagcaggcctacaccagccagaccgacagcagaaatggcaccgtggacaaggacttcagagtgtcccaagagtgctacatcggcagcgacggcaccaagatgaactgcgactttaacgccgccgtgaatatcgccgaccggatctccgatgacaccctgcgggaagagttcaacaaagaactgcccatgaaggaatttgtgaagtacggcgccacacagatcgtgtctcggaacagaagatggaacgccgaaaacgccgagagggtcacaatggccgccttccagaagagaaagaacgctgacggcggagcccataccgccgctgtgaagcctacatccaagaccgaggaaagcagcctgaagctggtggccgaaggatcccatcatcatcaccatcaccctgctgctaagagagtgaaactggatgactacaaagaccatgacggtgattataaagatcatgatatcgattacaaggatgacgatgacaagggatccaaaagaactggaagcggagctactaacttcagcctgctgaagcaggctggtgacgtggaggagaaccctggacctgttaacatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaagctcgagcctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatg

SEQ ID NO: 113 - DNA, 7854 bp, synthetic construct
  misc_feature 1..7854: note = EpiCas057 gRNA plamid DNA
  source 1..7854: mol_type = other DNA
gaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacaggggagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgatgcggtatcaacttgattggtactgacaccaagtcccctcatccccacagtttttttaggcagctgtagatctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagcgctgccaccatggcgcgtaaggtcgatctcacctcctgcgatcgcgagccgatccacatccccggcagcattcagccgtgcggctgcctgctagcctgcgacgcgcaggcggtgcggatcacgcgcattacggaaaatgccggcgcgttctttggacgcgaaactccgcgggtcggtgagctactcgccgattacttcggcgagaccgaagcccatgcgctgcgcaacgcactggcgcagtcctccgatccaaagcgaccggcgctgatcttcggttggcgcgacggcctgaccggccgcaccttcgacatctcactgcatcgccatgacggtacatcgatcatcgagttcgagcctgcggcggccgaacaggccgacaatccgctgcggctgacgcggcagatcatcgcgcgcaccaaagaactgaagtcgctcgaagagatggccgcacgggtgccgcgctatctgcaggcgatgctcggctatcaccgcgtgatgttgtaccgcttcgcggacgacggctccgggatggtgatcggcgaggcgaagcgcagcgacctggagagctttctcggtcagcactttccggcgtcgctggtcccgcagcaggcgcggctactgtacttgaagaacgcgatccgcgtggtctcggattcgcgcggcatcagcagccggatcgtgcccgagcacgacgcctccggcgccgcgctcgatctgtcgttcgcgcacctgcgcagcatctcgccctgccatctcgaatttctgcggaacatgggcgtcagcgcctcgatgtcgctgtcgatcatcattgacggcacgctatggggattgatcatctgtcatcattacgagccgcgtgccgtgccgatggcgcagcgcgtcgcggccgaaatgttcgccgacttcttatcgctgcacttcaccgccgcccaccaccaacgcggatccggcgagggcagaggtagcctgctgacctgcggcgacgtggaggagaaccccggccccaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgaccgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaa

SEQ ID NO: 114 - DNA, 7770 bp, synthetic construct
  misc_feature 1..7770: note = EpiCas059 plamid DNA
  source 1..7770: mol_type = other DNA
tgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcataatacgactcactatagggcggccgccaaaaaacccctcaagacccgtttagaggccccaaggggttatgctagatctgcttagggttaggcgttttgcgctgctgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgagaaatccgtccttggttgacgggaagtcccctcatccccacagtttttttaggcagctgtagatctcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaggagagccaccatgaaacggactgctgacggcagcgaatttgagagccccaagaagaagaggaaagtcggcatcaccatgagcgacaccaccttcagaccctacaagagcaagctgaaccccaacgagcggaagatgcctatggtggtggacaccatgaattggtgcgacaacgcctgcagcgtgttcttcgatctgttcgtgggcctgtacggcggcgtgaagatcggcgacgtgaccaatttcctgaccgagaagcaggacaccaccaccgatgccgatctgctgtgcgccatcaactggttcagagccatgcctgtgtctaaccccgatgccgttgtgggacctgagcagctggttgtgcggttcagagactacagcggccacaatccatctccaatggccagcatgtacctgagcgcccctgtgaaccctagatacacctgggtcgactgccggttcctgtaccagcagatgtgtcagaccctgggcgccaacctggatgtggacatctctaccctggtgcagaccggcctgctgcctatccagaaggatacacagctgaacgcctacagcttctgcagcaacgtgttcggcgagggcgtgaaagaagatcccagcgccaaagtgaccagctacacagccgctgccaatgctctgagcggcgccaccaatcctatcatcacctacgaggactaccggcagatcctgtccagcgccatcaccaacagacccagcagaaagggcgccaaggatattctggccgccgatctggatagcaccaagacaggtgttctgcccggcaactacgtgtcccagagaatcgccatgtaccaggcctctgccgcctctaaagctagaggcgtggtgctgcccaaccggctgagagtgaaagcctacctgcagaacctcgtgggactgaactacatctgcaccacctgggccgctcctatgctgagagccgtgggcgacatcaagagcaagaacagcaacaactaccgcttcgtgggcgagagactggaacggcagcaagagatcgataacctgctggccgtgtccaacatcagaagcgcccaggccatcctgcagcagttcagggccaatgagagcaaccagttctacatcaagaagcggcacatcggcaagggcctgcctagcctgtacgacatgtgggatctgatggacgagaaagagggcatcgacaactacatccagagcctgaagggcgacaagaccgagaggctgccaatcagagagctgctgcagatcatctaccctcacagaaagagcgtgtccgccgagctgtttctgtctgccgccgaactgaccgacgtggaagatagacagaaccggtgcatggtgcaccctacagtgcggggctctctggtcatcaacttcggcaagagcagcaccatcttcggcgctatcacccctcctgacacactgatcaagttccggggcgaaatggtgcctgctggacagacaggcatgatctggttcacaatgcacctggtggacggcggcaaatgggtcaagcaccacattccttgccacagcagccggtacttcgaggaaatctacgcctggaagaaggacctgcctacactgccctttccacggcggagcctgtatggctatgccatcggcaacaagatcagcgacacccggaagatcgacctgcggagaaagaaggccagcaagcagtacctgcggaccctggaaaacatgacccacaacgtggaatttgaccccaccacacagttcatggtggaccaagagctgaacgtgaccatcaccgccagagtgaagaaaatccctcctgtgcgggatgtgcccgtgggcagcagaattatgggcctgaacaagaaccagaccatccgggactgctacagcatctgggagagagtggaagccaacaccagcggcagctttcagcatcccgtggtccaggatgtgtggctgagactggtggccgatggccacgtgacctctacagtgaatggccccaacggccagatcgaccagatgagctactctggccccgatgtggccaactgcggcaactggatgcaagagcggagcaacttcatcaccagcgtggacgacgccgaccagtctatcatgaagcactttctgtggaacaaccgcgtgaagctgtatgcctggaactgcgtgtacgccggcacactgatgaaggccatctctaagcagatgaccaccgccaaccggaacctgttcagaaacgagatcctgagcttcgttggcggcagattcggctgcagactgggatctctggcccaggtttcactggacctgctgcggaggatgaagtccgtgatcagctgctacttcagccggaacaacgccttcaccatcgaggaaagaagcgccttcgacaaagaactgatggaactgttcgagagcctggacaacaagatgaacaacaagcggaaagagaaagtcaaccggacactcagcagcatcttccaggtggccaagcagcacaatgttgtgcggctggtggtcaccaagaacctgcagacccctgatgccaatgtgcggagcgtgatgaatcagagatccgtggattggtgcgctcggaagattgaagagaagatcgtggacctgggcaacgtgatcggaatctatgtggatctggtggaagccctggatctgagccatctggaccccttcatctatatcgccaagggcgacgtgggcaaagagtgccagtttgacgaggtggaaagcagctccctgctgccaattcacaccagcatgtttcgcgactggcacggcctggttaacaagggcagaaccaccacacctatctaccacagcgccctgaggagcttcgccagccactacggcctgagattcgatgacctggccagaatcaaggccgctgagctggccaaaatgctgccagaccagaaggtgttcatcccacacagaggcggacggttcttcatgagcacataccccgtgaccagcaccagcagacccgtgtcttatgatggccggcagcggttcctgaacagcggcgatatggtggccccagtgaacaccatgctgcgcggattcagaagggccagatctggatcccatcatcatcaccatcaccctgctgctaagagagtgaaactggatgactacaaagaccatgacggtgattataaagatcatgatatcgattacaaggatgacgatgacaagggatccaaaagaactggaagcggagctactaacttcagcctgctgaagcaggctggagacgtggaggagaaccctggacctgttaacatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaagctcgagcctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatg

SEQ ID NO: 115 - DNA, 7846 bp, synthetic construct
  misc_feature 1..7846: note = EpiCas059 gRNA plamid DNA
  source 1..7846: mol_type = other DNA
gaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacaggggagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgagaaatccgtccttggttgacggaagtcccctcatccccacagtttttttaggcagctgtagatctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagcgctgccaccatggcgcgtaaggtcgatctcacctcctgcgatcgcgagccgatccacatccccggcagcattcagccgtgcggctgcctgctagcctgcgacgcgcaggcggtgcggatcacgcgcattacggaaaatgccggcgcgttctttggacgcgaaactccgcgggtcggtgagctactcgccgattacttcggcgagaccgaagcccatgcgctgcgcaacgcactggcgcagtcctccgatccaaagcgaccggcgctgatcttcggttggcgcgacggcctgaccggccgcaccttcgacatctcactgcatcgccatgacggtacatcgatcatcgagttcgagcctgcggcggccgaacaggccgacaatccgctgcggctgacgcggcagatcatcgcgcgcaccaaagaactgaagtcgctcgaagagatggccgcacgggtgccgcgctatctgcaggcgatgctcggctatcaccgcgtgatgttgtaccgcttcgcggacgacggctccgggatggtgatcggcgaggcgaagcgcagcgacctggagagctttctcggtcagcactttccggcgtcgctggtcccgcagcaggcgcggctactgtacttgaagaacgcgatccgcgtggtctcggattcgcgcggcatcagcagccggatcgtgcccgagcacgacgcctccggcgccgcgctcgatctgtcgttcgcgcacctgcgcagcatctcgccctgccatctcgaatttctgcggaacatgggcgtcagcgcctcgatgtcgctgtcgatcatcattgacggcacgctatggggattgatcatctgtcatcattacgagccgcgtgccgtgccgatggcgcagcgcgtcgcggccgaaatgttcgccgacttcttatcgctgcacttcaccgccgcccaccaccaacgcggatccggcgagggcagaggtagcctgctgacctgcggcgacgtggaggagaaccccggccccaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgaccgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaa

SEQ ID NO: 116 - protein, 1546 aa, synthetic construct
  REGION 1..1546: note = dEpiCas059m46-CBE
  SITE 1546: note = misc_feature - Xaa =
MKRTADGSEFESPKKKRKVSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGSETPGTSESATPESGSEETRTPEKIACQQDRSAFFKLGKLPQKTTSAIYHLMRCSKRYIDNVSETISNNIEKAAMTSDNHIFRSALKEEYKKIYREKFYPDVTGQPFPDKGPDNTHIVERTVENLCGQYISALKGVMKVRKPKWQDDIFEDESDKVIAKMLMASGNEADIHPKLEKKIASAKKKDKMDEWQKVLDRYYYIKSKTDIEWTYTTLLYDHIARECARSMFEDGNKPKFCYTNGNKFAKQVVDELWGSERVEYEYEKYLHCRTLEAYWNEENKYFFLENQKKSKKTYEARQIIDHFIENEDAVKAETEEYMAKVAKEFGENGGCKFRTENILYLVNSVSINKKQDEERCPIFKVTDIPQYAEAGIDRIGECKYVIKLKLFTVGERYGTTYMPEAFSGLYDRRTNQRYDLIDRACKGSYVYLPIYISDKDSRKWSDRLKNTFTMSAFSISERKTKKDGRVLCVNLPYKHIQHKPRNYFKVYPTYGVAINVKHSLFSTNATNLNGYIDVNGELIDFYNSIGRPDKLKNINLAVTLGDKCGTAGNHTNFLLVSDLGDWDDHGKKRNDRFVKREDGTYYSRPDLLADGLNFLKKKYSGDRKSYDYINTTIAFRNNQTRLRCIDKEWNDRKRAYDEEHGKPEKGERNGFYPTEDAERLTMERQALRREIAENTRQVIAYMLWLFQENGIKSIALAHLDCGSWKNFRDQLKPKWFCNNCKYWAEKYGIPYEATKYYRSNRKYFDIEDGELIPENVKLNALGEAENDKRYFQTSIAKAVGMSSIKQMFSIVSQNYDIQVNYTEQAYTSQTDSRNGTVDKDFRVSQECYIGSDGTKMNCAFNAAVNIADRISDDTLREEFNKELPMKEFVKYGATQIVSRNRRWNAENAERVTMAAFQKRKNADGGAHTAAVKPTSKTEESSLKLVAEPKKKRKVSGGSTNLSDIIEKETGKQLVIQESILMLPEEVEEVIGNKPESDILVHTAYDESTDENVMLLTSDAPEYKPWALVIQDSNGENKIKMLSGGSPKKKRKVGSKRTGSGATNFSLLKQAGDVEENPGPVNMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKX

SEQ ID NO: 117 - DNA, 8090 bp, synthetic construct
  misc_feature 1..8090: note = dEpiCas059m46-CBE plasmid DNA
  source 1..8090: mol_type = other DNA
tgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcataatacgactcactatagggcggccgccaaaaaacccctcaagacccgtttagaggccccaaggggttatgctagatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaggagagccaccatgaaacggactgctgacggcagcgaatttgagagccccaagaagaagaggaaagtcagctcagagactggcccagtggctgtggaccccacattgagacggcggatcgagccccatgagtttgaggtattcttcgatccgagagagctccgcaaggaaacctgcctgctttacgaaattaattgggggggccggcactccatttggcgacatacatcacagaacactaacaagcacgtcgaagtcaacttcatcgagaagttcacgacagaaagatatttctgtccgaacacaaggtgcagcattacctggtttctcagctggagcccatgcggcgaatgtagtagggccatcactgagttcctgtcaaggtatccccacgtcactctgtttatttacatcgcaaggctgtaccaccacgctgacccccgcaatcgacaaggcctgcgggatttgatctcttcaggtgtgactatccaaattatgactgagcaggagtcaggatactgctggagaaactttgtgaattatagcccgagtaatgaagcccactggcctaggtatccccatctgtgggtacgactgtacgttcttgaactgtactgcatcatactgggcctgcctccttgtctcaacattctgagaaggaagcagccacagctgacattctttaccatcgctcttcagtcttgtcattaccagcgactgcccccacacattctctgggccaccgggttgaaatccggcagcgagacgccaggcacctccgagagcgctacgcctgaatccggatccgaagagacacggacccctgagaagatcgcctgccagcaggatagaagcgccttcttcaagctgggcaagctgccccagaaaaccaccagcgccatctaccacctgatgcggtgtagcaagcggtacatcgacaacgtgtccgagacaatcagcaacaacatcgagaaggccgccatgaccagcgacaaccacatcttcagaagcgccctgaaagaagagtacaagaagatctaccgcgagaagttctaccccgacgtgaccggccagccttttccagataagggccccgacaacacccacatcgtggaacggaccgtggaaaacctgtgcggccagtacatctctgccctgaagggcgtgatgaaagtgcggaagcccaagtggcaggacgacatcttcgaggacgagagcgacaaagtgatcgccaagatgctgatggccagcggcaacgaggccgacattcaccccaagctggaaaagaagattgccagcgccaagaaaaaggacaagatggacgagtggcagaaggtgctggacagatactactacatcaagagcaagaccgacatcgagtggacctacaccacactgctgtacgaccacattgcccgcgagtgcgccagatccatgttcgaggatggcaacaagcctaagttctgctacaccaacggcaacaagttcgccaaacaggtggtggacgagctgtggggctctgagagagtggaatacgagtacgagaagtacctgcactgcagaaccctggaagcctactggaacgaggaaaacaagtacttcttcctggaaaatcaaaagaagtccaagaaaacctacgaggcccggcagatcatcgaccacttcatcgagaacgaggacgccgtgaaggccgaaaccgaagagtatatggccaaggtggccaaagagttcggcgagaacggcggctgcaagttcagaaccgagaacatcctgtacctggtcaacagcgtgtccatcaacaagaagcaggacgaggaacggtgccccatcttcaaagtgaccgacattccccagtatgccgaggccggcatcgatagaatcggcgagtgcaaatacgtgatcaagctgaagctgttcaccgtgggcgagagatacggcaccacctatatgcctgaggccttcagcggcctgtacgacagaaggaccaaccagagatacgacctgatcgacagagcctgcaagggcagctacgtgtacctgcctatctacatcagcgacaaggacagccggaagtggagcgacagactgaagaacaccttcaccatgtccgccttcagcatcagcgagcgcaagaccaagaaagacggcagagtgctgtgcgtgaacctgccttacaaacacatccagcacaagccccggaactacttcaaggtgtaccccacctacggcgtggctatcaacgtgaagcacagcctgttcagcaccaacgccaccaacctgaacggctacatcgacgtgaacggcgagctgatcgacttctacaacagcatcggcagacccgataagctcaagaacatcaacctggccgtgacactgggcgacaaatgcggcacagctggcaaccacaccaactttctgctggtgtccgacctcggcgactgggatgatcacggcaagaagcggaacgacagattcgtgaagagagaggacggcacctactacagcagacctgacctgctggccgacggcctgaacttcctgaagaagaagtatagcggcgaccggaagtcctacgattatatcaacaccacaatcgccttccggaacaaccagaccagactgagatgcatcgacaaagagtggaacgatcggaagcgggcctacgatgaggaacacggcaagcctgagaagggcgaaagaaacggcttctaccctaccgaggatgccgagcggctgacaatggaaagacaggccctgcggagagagattgccgagaacaccagacaagtgattgcctacatgctgtggctgttccaagagaacggcatcaagtctatcgccctggctcacctggattgcggcagctggaagaacttccgggaccagctgaagccaaagtggttctgcaacaactgcaagtactgggccgagaagtatggcatcccttacgaggccaccaagtactacagaagcaaccggaagtactttgacatcgaggacggggagctgattcccgagaatgtgaagctgaacgccctgggcgaagccgagaacgacaagagatacttccagacctctatcgccaaggccgtgggcatgagcagcatcaagcagatgttcagcatcgtgtcccagaactacgacatccaagtgaactacaccgagcaggcctacaccagccagaccgacagcagaaatggcaccgtggacaaggacttcagagtgtcccaagagtgctacatcggcagcgacggcaccaagatgaactgcgcttttaacgccgccgtgaatatcgccgaccggatctccgatgacaccctgcgggaagagttcaacaaagaactgcccatgaaggaatttgtgaagtacggcgccacacagatcgtgtctcggaacagaagatggaacgccgaaaacgccgagagggtcacaatggccgccttccagaagagaaagaacgctgacggcggagcccataccgccgctgtgaagcctacatccaagaccgaggaaagcagcctgaagctggtggccgaacccaagaagaaacggaaagtgtccggcggcagcaccaacctgtccgacatcatcgagaaggagacgggcaagcaactcgtgatccaggagagcatcctcatgctgccagaggaggtggaggaggtcatcggcaacaagccagagtccgacatcctggtgcacaccgcctacgacgagtccaccgacgagaacgtcatgctcctgaccagcgacgccccagagtacaagccctgggccctcgtcatccaggacagcaacggggagaacaagatcaagatgctgtctggtggttctcccaagaagaagaggaaagtcggatccaaaagaactggaagcggagctactaacttcagcctgctgaagcaggctggagacgtggaggagaaccctggacctgttaacatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaagctcgagcctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatg

SEQ ID NO: 118 - protein, 1863 aa, synthetic construct
  REGION 1..1863: note = dAsCas12a-CBE
  SITE 1863: note = misc_feature - Xaa =
MKRTADGSEFESPKKKRKVSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGSETPGTSESATPESGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALADLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVAFLISPVKNSDGIFYDSRNYEAQENAILPKNADANGAYNIARKVLWAIGQFKKAEDEKLDKVKIAISNKEWLEYAQTSVKHPKKKRKVSGGSTNLSDIIEKETGKQLVIQESILMLPEEVEEVIGNKPESDILVHTAYDESTDENVMLLTSDAPEYKPWALVIQDSNGENKIKMLSGGSPKKKRKVGSKRTGSGATNFSLLKQAGDVEENPGPVNMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKX

SEQ ID NO: 119 - DNA, 9041 bp, synthetic construct
  misc_feature 1..9041: note = dAsCas12a-CBE plasmid DNA
  source 1..9041: mol_type = other DNA
tgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcataatacgactcactatagggcggccgccaaaaaacccctcaagacccgtttagaggccccaaggggttatgctagatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaggagagccaccatgaaacggactgctgacggcagcgaatttgagagccccaagaagaagaggaaagtcagctcagagactggcccagtggctgtggaccccacattgagacggcggatcgagccccatgagtttgaggtattcttcgatccgagagagctccgcaaggaaacctgcctgctttacgaaattaattgggggggccggcactccatttggcgacatacatcacagaacactaacaagcacgtcgaagtcaacttcatcgagaagttcacgacagaaagatatttctgtccgaacacaaggtgcagcattacctggtttctcagctggagcccatgcggcgaatgtagtagggccatcactgagttcctgtcaaggtatccccacgtcactctgtttatttacatcgcaaggctgtaccaccacgctgacccccgcaatcgacaaggcctgcgggatttgatctcttcaggtgtgactatccaaattatgactgagcaggagtcaggatactgctggagaaactttgtgaattatagcccgagtaatgaagcccactggcctaggtatccccatctgtgggtacgactgtacgttcttgaactgtactgcatcatactgggcctgcctccttgtctcaacattctgagaaggaagcagccacagctgacattctttaccatcgctcttcagtcttgtcattaccagcgactgcccccacacattctctgggccaccgggttgaaatccggcagcgagacgccaggcacctccgagagcgctacgcctgaatccggatcctcaaagctggagaaattcaccaactgttatagcctgtctaagaccctgcgcttcaaggcaatcccagtgggcaagacacaagagaacattgacaacaaacggctcctggtggaggatgagaagagggctgaagattacaagggcgttaagaagctgctggataggtactatctgtcattcatcaacgatgtcctccacagtatcaagctgaagaatctgaacaattacatttctctgttccggaagaagacacggaccgagaaggagaacaaagagctggagaatctggagatcaacctgaggaaagaaatagctaaggctttcaaagggaacgagggttacaagtccctgttcaagaaagacattatcgagactattctgcctgagttcctggacgataaagatgagatcgccctcgtcaattccttcaatgggtttaccacagcctttaccggcttcttcgacaatagagagaatatgttctctgaagaggccaaatccactagcatcgcctttcgctgcataaacgagaacctgactaggtacatcagcaatatggacatctttgagaaagtcgatgccatattcgacaaacatgaggtgcaggagattaaggagaagatcctgaactcagattacgatgtcgaagatttcttcgagggagagttcttcaacttcgtgctcacacaagagggcattgatgtgtacaatgcaatcattggagggttcgtgacagagagtggcgagaagataaagggcctgaacgagtatatcaacctctacaaccagaaaaccaagcagaaactgcctaagttcaagccactgtacaaacaagtgctctcagatagggaaagcctgagcttctacggtgaagggtatacatcagatgaagaagtgctcgaagtgttccgcaacaccctcaataagaacagtgaaatcttctcttcaatcaagaagctggagaaactgttcaagaatttcgatgagtactcctctgccggaatctttgtgaagaatggccctgcaatatccactattagcaaagacatctttggcgagtggaacgttatcagggataagtggaatgccgagtacgatgatattcatctcaagaagaaagccgtggttacagagaaatacgaggatgatagacgcaagagctttaagaagattggtagcttctctctcgaacagctgcaggagtacgccgacgctgacctgtcagtcgtggagaaactcaaggagatcataatccagaaggtggatgaaatctacaaagtgtatggaagctctgagaaactcttcgatgcagactttgttctggagaagagtctgaagaagaacgacgcagtggttgctatcatgaaggacctgctggattctgttaagtctttcgagaattacattaaggcattctttggtgaagggaaggagacaaatagggacgagagcttctatggcgactttgttctggcctacgacatcctcctcaaggttgaccacatctatgacgctatacggaattacgttacccagaagccctatagcaaagacaagttcaagctgtatttccagaatccacagtttatgggtgggtgggataaagacaaagaaacagattacagggccactatcctgcggtacggcagcaaatactatctggctatcatggataagaagtacgccaaatgcctccagaagatcgacaaggacgacgtgaacggtaactacgagaagatcaattacaagctcctgccaggacctaacaagatgctgcccaaggtgttcttctccaagaaatggatggcctactataacccaagcgaggacattcagaagatatacaagaatgggacattcaagaagggcgatatgttcaacctcaacgactgccacaagctgattgatttcttcaaggatagcatttctcgctatcccaagtggtctaatgcatacgatttcaacttcagcgagactgagaagtacaaagacatcgctggcttctaccgggaggtggaagagcaaggctataaggtgtcattcgaatccgcttctaagaaggaagtggataagctcgtggaagagggtaagctgtacatgttccagatatacaacaaagacttcagcgataagagccacggcactccaaacctccatactatgtatttcaagctgctgtttgacgagaacaaccacggacagattaggctgtcaggaggcgcagaactcttcatgcgcagagcttcactgaagaaggaggaactcgttgtccacccagccaatagccctatagccaataagaatccagacaatcctaagaaaaccactactctgtcttacgatgtgtataaggataagagattctctgaagatcagtacgaactgcacatacccattgccattaacaagtgccctaagaacatcttcaagattaacacagaggttagagtgctcctgaaacacgacgataacccttatgttataggcattgctcgcggagagagaaacctgctgtacatcgtcgtggtggacggcaaaggcaacatcgtggaacagtacagtctcaatgaaatcattaacaatttcaacggaatccgcattaagaccgactaccattctctcctcgacaagaaggagaaagaaaggttcgaagcaagacagaattggacaagtatagagaatatcaaagaactgaaggctgggtacatctctcaggttgtgcacaagatatgtgagctggtggagaagtacgacgctgttatcgccctcgcggacctgaatagcggcttcaagaactccagggtgaaggtggagaagcaggtgtatcagaagttcgagaagatgctgatcgacaagctcaactatatggtggacaagaaatccaatccttgcgctactggtggagccctgaagggctatcaaatcaccaataagttcgaatctttcaagtctatgagcacccagaatggcttcatcttctacatacccgcatggctgacatccaagattgatccctctaccggatttgttaatctgctcaagactaagtacacctctattgctgactcaaagaagttcatatcatcatttgaccgcatcatgtacgtgccagaagaggacctgttcgagtttgccctggattacaagaatttctctcggactgacgccgactacatcaagaagtggaagctctactcttatggtaatcggattcgcatattccgcaatcccaagaagaataacgtgttcgattgggaggaagtttgcctcaccagcgcttacaaggagctgttcaataagtatgggattaactaccagcagggcgacataagagccctgctgtgcgaacaatctgataaggcattctattcctctttcatggcactgatgtcactgatgctgcaaatgcgcaattccatcaccggaagaacagacgtggcctttctgatctctcctgtcaagaactcagatggcatcttctacgattcccgcaactatgaagcacaggagaatgctatcctgcctaagaatgccgatgcaaatggagcctataacatcgccagaaaggtcctctgggccataggacaattcaagaaagctgaagatgagaagctggacaaggtgaagatcgccatttcaaacaaagagtggctcgaatatgctcagacctcagtgaagcatcccaagaagaaacggaaagtgtccggcggcagcaccaacctgtccgacatcatcgagaaggagacgggcaagcaactcgtgatccaggagagcatcctcatgctgccagaggaggtggaggaggtcatcggcaacaagccagagtccgacatcctggtgcacaccgcctacgacgagtccaccgacgagaacgtcatgctcctgaccagcgacgccccagagtacaagccctgggccctcgtcatccaggacagcaacggggagaacaagatcaagatgctgtctggtggttctcccaagaagaagaggaaagtcggatccaaaagaactggaagcggagctactaacttcagcctgctgaagcaggctggtgacgtggaggagaaccctggacctgttaacatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaagctcgagcctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatg

SEQ ID NO: 120 - DNA, 23 bp, synthetic construct
  misc_feature 1..23: note = Example7 CXCR4 Spacer-1
  source 1..23: mol_type = other DNA
gcaaggatggacgcgccacagag

SEQ ID NO: 121 - DNA, 23 bp, synthetic construct
  misc_feature 1..23: note = Example7 CXCR4 Spacer-2
  source 1..23: mol_type = other DNA
taaaagtccggccgcggccagaa

SEQ ID NO: 122 - DNA, 23 bp, synthetic construct
  misc_feature 1..23: note = Example7 CXCR4 Spacer-3
  source 1..23: mol_type = other DNA
agggaagcgggatgcgcctgaag

SEQ ID NO: 123 - DNA, 23 bp, synthetic construct
  misc_feature 1..23: note = Example7 CXCR4 Spacer-4
  source 1..23: mol_type = other DNA
cgggtggtcggtagtgagtccgg

SEQ ID NO: 124 - protein, 620 aa, synthetic construct
  REGION 1..620: note = Example7 scFV-P65-HSF1
  SITE 620: note = misc_feature - Xaa =
MGPDIVMTQSPSSLSASVGDRVTITCRSSTGAVTTSNYASWVQEKPGKLFKGLIGGTNNRAPGVPSRFSGSLIGDKATLTISSLQPEDFATYFCALWYSNHWVFGQGTKVELKRGGGGSGGGGSGGGGSSGGGSEVKLLESGGGLVQPGGSLKLSCAVSGFSLTDYGVNWVRQAPGRGLEWIGVIWGDGITDYNSALKDRFIISKDNGKNTVYLQMSKVRSDDTALYYCVTGLFDYWGQGTLVTVSSYPYDVPDYAGGGGGSGGGGSGGGGSGGGGSPKKKRKVAAAPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVSASEGRGSLLTCDYKDDDDKX

SEQ ID NO: 125 - protein, 1356 aa, synthetic construct
  REGION 1..1356: note = Example7 dEpiCas057-10xGCN4
  SITE 1356: note = misc_feature - Xaa =
MKRTADGSEFESPKKKRKVHRRRRVREGITMSDTTFRPYKSKLNPNERKMPMVVDTMNWCDNACSVFFDLFVGLYGGVKIGDVTNFLTEKQDTTTDADLLCAINWFRAMPVSNPDAVVGPEQLVVRFRDYSGHNPSPMASMYLSAPVNPRYTWVDCRFLYQQMCQTLGANLDVDISTLVQTGLLPIQKDTQLNAYSFCSNVFGEGVKEDPSAKVTSYTAAANALSGATNPIITYEDYRQILSSAITNRPSRKGAKDILAADLDSTKTGVLPGNYVSQRIAMYQASAASKARGVVLPNRLRVKAYLQNLVGLNYICTTWAAPMLRAVGDIKSKNSNNYRFVGERLERQQEIDNLLAVSNIRSAQAILQQFRANESNQFYIKKRHIGKGLPSLYDMWDLMDEKEGIDNYIQSLKGDKTERLPIRELLQIIYPHRKSVSAELFLSAAELTDVEDRQNRCMVHPTVRGSLVINFGKSSTIFGAITPPDTLIKFRGEMVPAGQTGMIWFTMHLVDGGKWVKHHIPCHSSRYFEEIYAWKKDLPTLPFPRRSLYGYAIGNKISDTRKIDLRRKKASKQYLRTLENMTHNVEFDPTTQFMVDQELNVTITARVKKIPPVRDVPVGSRIMGLNKNQTIRDCYSIWERVEANTSGSFQHPVVQDVWLRLVADGHVTSTVNGPNGQIDQMSYSGPDVANCGNWMQERSNFITSVDDADQSIMKHFLWNNRVKLYAWNCVYAGTLMKAISKQMTTANRNLFRNEILSFVGGRFGCRLGSLAQVSLDLLRRMKSVISCYFSRNNAFTIEERSAFDKELMELFESLDNKMNNKRKEKVNRTLSSIFQVAKQHNVVRLVVTKNLQTPDANVRSVMNQRSVDWCARKIEEKIVDLGNVIGIYVDLVEALDLSHLDPFIYIAKGDVGKECQFDEVESSSLLPIHTSMFRDWHGLVNKGRTTTPIYHSALRSFASHYGLRFDDLARIKAAELAKMLPDQKVFIPHRGGRFFMSTYPVTSTSRPVSYDGRQRFLNSGAMVAPVNTMLRGFRRARSHHHHHHPAAKRVKLDDYKDHDGDYKDHDIDYKDDDDKGSMEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGTAVNIGGGTGPMDLQRPQGGGGPKKKRKVGSGVRLQRRX

SEQ ID NO: 126 - DNA, 15951 bp, synthetic construct
  misc_feature 1..15951: note = Example7 dEpiCas057-10xGCN4/scFV-P65-HSF1 plasmid DNA
  source 1..15951: mol_type = other DNA
cttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgggatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgaattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggccctctagatgcatgctcgagcggccgccagtgtgatggatatctgcagaattcgcccttttggccggccttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttaggcgcgccggattcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagatccttaatatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagccctaagaaaaagaggaaggtggcggccgctccttcagggcagatcagcaaccaggccctggctctggcccctagctccgctccagtgctggcccagactatggtgccctctagtgctatggtgcctctggcccagccacctgctccagcccctgtgctgaccccaggaccaccccagtcactgagcgctccagtgcccaagtctacacaggccggcgaggggactctgagtgaagctctgctgcacctgcagttcgacgctgatgaggacctgggagctctgctggggaacagcaccgatcccggagtgttcacagatctggcctccgtggacaactctgagtttcagcagctgctgaatcagggcgtgtccatgtctcatagtacagccgaaccaatgctgatggagtaccccgaagccattacccggctggtgaccggcagccagcggccccccgaccccgctccaactcccctgggaaccagcggcctgcctaatgggctgtccggagatgaagacttctcaagcatcgctgatatggactttagtgccctgctgtcacagatttcctctagtgggcagggaggaggtggaagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctccgctagcgagggcagaggaagtctgctaacatgcgattacaaagacgatgacgataagtaaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgatcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggaattccgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaggagagccaccatgaaacggactgctgacggcagcgaatttgagagccccaagaagaagaggaaagtccaccggagacgacgcgttcgcgagggcatcaccatgagcgacaccaccttcagaccctacaagagcaagctgaaccccaacgagcggaagatgcctatggtggtggacaccatgaattggtgcgacaacgcctgcagcgtgttcttcgatctgttcgtgggcctgtacggcggcgtgaagatcggcgacgtgaccaatttcctgaccgagaagcaggacaccaccaccgatgccgatctgctgtgcgccatcaactggttcagagccatgcctgtgtctaaccccgatgccgttgtgggacctgagcagctggttgtgcggttcagagactacagcggccacaatccatctccaatggccagcatgtacctgagcgcccctgtgaaccctagatacacctgggtcgactgccggttcctgtaccagcagatgtgtcagaccctgggcgccaacctggatgtggacatctctaccctggtgcagaccggcctgctgcctatccagaaggatacacagctgaacgcctacagcttctgcagcaacgtgttcggcgagggcgtgaaagaagatcccagcgccaaagtgaccagctacacagccgctgccaatgctctgagcggcgccaccaatcctatcatcacctacgaggactaccggcagatcctgtccagcgccatcaccaacagacccagcagaaagggcgccaaggatattctggccgccgatctggatagcaccaagacaggtgttctgcccggcaactacgtgtcccagagaatcgccatgtaccaggcctctgccgcctctaaagctagaggcgtggtgctgcccaaccggctgagagtgaaagcctacctgcagaacctcgtgggactgaactacatctgcaccacctgggccgctcctatgctgagagccgtgggcgacatcaagagcaagaacagcaacaactaccgcttcgtgggcgagagactggaacggcagcaagagatcgataacctgctggccgtgtccaacatcagaagcgcccaggccatcctgcagcagttcagggccaatgagagcaaccagttctacatcaagaagcggcacatcggcaagggcctgcctagcctgtacgacatgtgggatctgatggacgagaaagagggcatcgacaactacatccagagcctgaagggcgacaagaccgagaggctgccaatcagagagctgctgcagatcatctaccctcacagaaagagcgtgtccgccgagctgtttctgtctgccgccgaactgaccgacgtggaagatagacagaaccggtgcatggtgcaccctacagtgcggggctctctggtcatcaacttcggcaagagcagcaccatcttcggcgctatcacccctcctgacacactgatcaagttccggggcgaaatggtgcctgctggacagacaggcatgatctggttcacaatgcacctggtggacggcggcaaatgggtcaagcaccacattccttgccacagcagccggtacttcgaggaaatctacgcctggaagaaggacctgcctacactgccctttccacggcggagcctgtatggctatgccatcggcaacaagatcagcgacacccggaagatcgacctgcggagaaagaaggccagcaagcagtacctgcggaccctggaaaacatgacccacaacgtggaatttgaccccaccacacagttcatggtggaccaagagctgaacgtgaccatcaccgccagagtgaagaaaatccctcctgtgcgggatgtgcccgtgggcagcagaattatgggcctgaacaagaaccagaccatccgggactgctacagcatctgggagagagtggaagccaacaccagcggcagctttcagcatcccgtggtccaggatgtgtggctgagactggtggccgatggccacgtgacctctacagtgaatggccccaacggccagatcgaccagatgagctactctggccccgatgtggccaactgcggcaactggatgcaagagcggagcaacttcatcaccagcgtggacgacgccgaccagtctatcatgaagcactttctgtggaacaaccgcgtgaagctgtatgcctggaactgcgtgtacgccggcacactgatgaaggccatctctaagcagatgaccaccgccaaccggaacctgttcagaaacgagatcctgagcttcgttggcggcagattcggctgcagactgggatctctggcccaggtttcactggacctgctgcggaggatgaagtccgtgatcagctgctacttcagccggaacaacgccttcaccatcgaggaaagaagcgccttcgacaaagaactgatggaactgttcgagagcctggacaacaagatgaacaacaagcggaaagagaaagtcaaccggacactcagcagcatcttccaggtggccaagcagcacaatgttgtgcggctggtggtcaccaagaacctgcagacccctgatgccaatgtgcggagcgtgatgaatcagagatccgtggattggtgcgctcggaagattgaagagaagatcgtggacctgggcaacgtgatcggaatctatgtggatctggtggaagccctggatctgagccatctggaccccttcatctatatcgccaagggcgacgtgggcaaagagtgccagtttgacgaggtggaaagcagctccctgctgccaattcacaccagcatgtttcgcgactggcacggcctggttaacaagggcagaaccaccacacctatctaccacagcgccctgaggagcttcgccagccactacggcctgagattcgatgacctggccagaatcaaggccgctgagctggccaaaatgctgccagaccagaaggtgttcatcccacacagaggcggacggttcttcatgagcacataccccgtgaccagcaccagcagacccgtgtcttatgatggccggcagcggttcctgaacagcggcgccatggtggccccagtgaacaccatgctgcgcggattcagaagggccagatctcatcatcatcaccatcaccctgctgctaagagagtgaaactggatgactacaaagaccatgacggtgattataaagatcatgatatcgattacaaggatgacgatgacaagggatccatggaagaacttttgagcaagaattatcatcttgagaacgaagtggctcgtcttaagaaaggttctggcagtggagaagaactgctttcaaagaattaccacctggaaaatgaggtagctagactgaaaaaggggagcggaagtggggaggagttgctgagcaaaaattatcatttggagaacgaagtagcacgactaaagaaagggtccggatcgggtgaggagttactctcgaaaaattatcatctcgaaaacgaagtggctcggctaaaaaagggcagtggttctggagaagagctattatctaaaaactaccacctcgaaaatgaggtggcacgcttaaaaaagggaagtggcagtggtgaagagctactatccaagaattatcatcttgagaacgaggtagcgcgtttgaagaagggttccggctcaggagaggaactgctctcgaagaactatcatcttgaaaatgaggtcgctcgattaaaaaagggatcgggcagtggtgaggaactactttcaaagaattaccacctcgaaaacgaagtagctcgattaaagaaaggttcagggtcgggtgaagaattactgagtaaaaattatcatctggaaaatgaggtagcgagactaaaaaaggggagtggttctggcgaggaattgctatcgaaaaattatcatcttgagaacgaagttgctaggctcaaaaagggctcaggctcaggcaccgcggtaaacataggtggtggaaccggtccgatggatctacagcggccgcaaggtggaggtggacccaagaagaagcgcaaggtgggaagcggagtaagattacaaagacgatgacgataagtaaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgatcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggaattccgaagttcctattctctagaaagtataggaacttcaggtctgaagaggagtttacgtccagcgcgatgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcctgttgacaattaatcatcggcatagtatatcggcatagtataatacgacaaggtgaggaactaaaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggaaacctccgcgccccgcaacctccccttctacgagcggctcggcttcaccgtcaccgccgacgtcgaggtgcccgaaggaccgcgcacctggtgcatgacccgcaagcccggtgcctgaaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcggcgcgccatcgatattaatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaaggtaccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgct

SEQ ID NO: 127 - protein, 1307 aa, synthetic construct
  REGION 1..1307: note = AsCas12a
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN